(19) United States
(12) Patent Application Publication
(10) Pub. No.: US 2026/0272932 A1
(43) Pub. Date: Sep. 17, 2026

(54) METHOD OF TREATING EARLY BREAST CANCER WITH RIBOCICLIB IN COMBINATION WITH AN AROMATASE INHIBITOR

(71) Applicant: Novartis AG, Basel (CH)

(72) Inventors: Arunava CHAKRAVARTTY, Whippany, NJ (US); Zheng LI, Livingston, NJ (US); Tetiana TARAN, Dornach (CH); Juan Pablo ZARATE, Westfield, NJ (US)

(21) Appl. No.: 19/167,114

(22) PCT Filed: Mar. 26, 2024
(86) PCT No.: PCT/IB2024/052897

§ 371 Date: Sep. 19, 2025

Related U.S. Application Data
(60) Provisional application No. 63/492,396, filed on Mar. 27, 2023.
(60) Provisional application No. 63/502,076, filed on May 12, 2023.
(60) Provisional application No. 63/505,313, filed on May 31, 2023.

(51) Int. Cl. A61K 31/519; A61K 31/4196; A61K 45/06; A61P 35/00

(52) U.S. Cl. CPC A61K 31/519; A61K 31/4196; A61K 45/06; A61P 35/00

(57) ABSTRACT

The present disclosure relates to the adjuvant treatment of HR+/HER2− stage II or III early breast cancer in adult patients. The method includes administering ribociclib (a CDK4/6 inhibitor) in combination with endocrine therapy.

### BACKGROUND OF THE INVENTION

Breast cancer (BC) is the most frequently diagnosed cancer worldwide. Approximately 1.7 million new cases of BC and 522,000 deaths attributed to this disease were estimated to occur in 2012 worldwide (CA Cancer J Clin 65:87-108, 2015). BC incidence varies between individuals of different ethnicities and in different geographic locations around the world, with rates ranging from 27 per 100,000 in Middle Africa and Eastern Asia to 92 in Northern America (GLOBOCAN: Estimated Cancer Incidence, Mortality and Prevalence Worldwide 2012 [Internet][cited 2018 Jun. 26], https://publications.iarc.fr/Databases/larc-Cancerbases/GLOBOCAN-2012-Estimated-Cancer-Incidence-Mortality-And-Prevalence-Worldwide-In-2012-V1.0-2012). In the United States, BC was projected to be the most common cancer diagnosed in 2018 with an estimated incidence of 268,670 new cases and 41,400 deaths (CA Cancer J Clin 68:7-30, 2018). Estimated incidence of BC in European countries in 2012 was 458,337 (Eur J Cancer Oxf Engl 1990 49:1374-1403, 2013). BC in men is not common, with a reported frequency of approximately 1% of all BC but its incidence is continuously rising (Breast Cancer Res Treat 137:465-470, 2013).

The vast majority of newly diagnosed BC cases are early breast cancers (EBC), localized to the breast tissue and regional lymphatics, which are potentially treatable with locoregional treatment modalities such as surgery and radiation therapy. Based on Surveillance, Epidemiology and End Results (SEER) Program data collected between the years 1975 and 2012, 93% of cases diagnosed were EBC, with 62% limited to the breast tissue and 31% localized within the breast tissue and regional lymph nodes (SEER Cancer Statistics Review, 1975-2015 [Internet]. Bethesda, MD, National Cancer Institute, 2018, https://seer.cancer.gov/csr/1975_2015/).

Besides primary surgery, management of EBC usually includes additional anti-neoplastic treatment modalities such as radiation therapy and adjuvant or neoadjuvant systemic therapy. Although many patients with EBC may be rendered disease-free with surgical resection and radiotherapy, distant recurrence due to micro-metastatic disease is common and is the primary cause of death in patients with EBC (BMC Med 13:195, 2015). According to the EBC Trialists' Collaborative Group (EBCTCG) meta-analysis of almost 150,000 women in 200 randomized clinical trials, approximately 36% and 20% of patients with EBC without any adjuvant systemic therapy will experience recurrence and death due to BC, respectively, during 5 years of follow-up (Lancet Lond Engl 365:1687-1717, 2005). Moreover, recurrences and BC-related deaths in patients with hormone receptor (HR)-positive EBC continue to occur after 5 years from surgery, with only 45% of patients reported to be recurrence-free at 15 years of follow-up.

Adjuvant systemic treatments that comprise cytotoxic, biological and endocrine therapies in patients with EBC decrease locoregional and distant recurrences, decrease BC-specific mortality and improve overall survival (OS) (Lancet Lond Engl 365:1687-1717, 2005). The need and selection of systemic adjuvant therapies is based on individual risk of recurrence and may be guided by several clinical, pathological and genomic predictive and prognostic factors of tumor and patient such as tumor stage, histopathological grade, tumor HR status, human epidermal growth factor receptor-2 (HER2) status, multi-gene testing recurrence scores, proliferation markers like Ki67, menopausal status, patient's comorbidities, age, and others.

Using these factors, EBC can be classified as having low, intermediate/moderate or high risk for recurrence after surgery (BMC Med 13:195, 2015). While there is no consensus on the definition of these risk groups, generally, patients with smaller tumors, no metastasis in regional lymph nodes, low tumor grade, HR-positive and HER2-negative status, and low recurrence genomic score have low risk of recurrence (i.e. 5-10% recurrences at 5-years). These patients are usually considered for adjuvant endocrine therapy (ET), without chemotherapy, due to a lower clinical benefit of latter versus the former. On the other hand, patients with metastases in multiple regional lymph nodes, high tumor grade, HER2-positive status or high recurrence genomic score have a higher risk of recurrence. These patients are usually considered for adjuvant chemotherapy (and HER2 targeting agents in patients with HER2-positive BC), and if the tumor expresses HR, adjuvant ET is considered too (normally delivered after the completion of chemotherapy).

It is estimated that 75% of BC express receptors for steroid hormones (estrogen receptor [ER] and/or progesterone receptor [PgR]), and therefore these patients may benefit from adjuvant ET with tamoxifen or aromatase inhibitors (Als) (letrozole, anastrozole, or exemestane) (J Clin Oncol Off J Am Soc Clin Oncol 28:2784-2795, 2010). ET, independent of chemotherapy, reduces the risk of recurrence and BC deaths in HR-positive EBC (Lancet Lond Engl 365:1687-1717, 2005).

Current clinical guidelines (see Ann Oncol Off J Eur Soc Med Oncol 26 Suppl 5:v8-30, 2015; and National Comprehensive Cancer Network. Breast Cancer (Version 1.2019) [Internet]. NCCN, 2019 [cited 2019 May 6], https://www.nccn.org/professionals/physician_gls/pdf/breast.pdf) for adjuvant ET in the HR-positive EBC recommend:
For premenopausal women:
   1.1. 5-10 years of tamoxifen with or without ovarian suppression, or
1.2. 5 years of AI with ovarian suppression (see N Engl J Med 379:122-137, 2018
For postmenopausal women:
   1.3. Initial AI for 5 years (or up to 10 years), based on results from the MA.17R trial (see N Engl J Med 375:209-219, 2016), or
   1.4. Initial tamoxifen for 2-3 years followed by AI either up to total 5 years, or up to 5 years of treatment with Al, or
   1.5. Tamoxifen for ~5 years followed by 5 years of Al, or
   1.6. Tamoxifen up to 10 years (Ann Oncol Off J Eur Soc Med Oncol 26 Suppl 5:v8-30, 2015; National Comprehensive Cancer Network. Breast Cancer (Version 1.2019) [Internet]. NCCN, 2019 [cited 2019 May 6], https://www.nccn.org/professionals/physician_gls/pdf/breast.pdf)
In men: Limited data suggest that tamoxifen or the combination of an AI with a gonadotropin-releasing hormone (GnRH) agonist should be the ET of choice for HR-positive, HER2-negative EBC (Breast Cancer Res Treat 151:141-147, 2015).

WO2015/022609 A1, incorporated herein by reference, discloses combination therapies for the treatment of cancer, including EBC. In some EBC patients, recurrences may still occur, especially in patients with adverse clinical, pathological, and genomic features. Approximately 25%-30% of patients with HR-positive, HER2-negative EBC with multiple (≥4) metastatic regional lymph nodes (largely corresponding to Anatomic Stage Group III in the AJCC 8^{th} edition Breast Cancer Staging) will recur within 5 years with ET that includes Als (Lancet Lond Engl 365:1687-1717, 2005). Only 48% of these patients will be distant recurrence-free within 20 years, with almost half of patients dying of BC within 20 years, despite ET for 5 years (N Engl J Med 377:1836-1846, 2017). While patients with 1-3 metastatic regional lymph nodes (corresponding generally to Anatomic Stage Group II in the AJCC 8^{th} edition Breast Cancer Staging) may have lower risk of recurrence than patients with Anatomic Stage Group III, still 31% of them will experience distant recurrence and 28% will die from BC within 20 years despite ET (N Engl J Med 377:1836-1846, 2017).

Adjuvant treatments for HR+ HER2− EBC remain limited. Although adjuvant treatments similar to those used in patients with HR+ HER2− advanced or metastatic breast cancer have been tried, results obtained from that approach have been variable. In one example, the cdk4/6 inhibitor abemaciclib was first approved for treatment of patients with HR+ HER2− advanced or metastatic breast cancer either alone or in combination with endocrine therapy, and was later shown in the monarchE clinical trial to be efficacious in treatment of patients with HR+ HER2− high risk early breast cancer (Johnston et al., 2023. Lancet 24(1), pp. 77-90). In contrast, the cdk4/6 inhibitor palbociclib combined with endocrine therapy demonstrated clinically relevant efficacy in patients with HR+ HER2− metastatic breast cancer (PALOMA3 trial, described in Turner et al., 2015. N Engl J Med 2015; 373:209-219), but the results did not bear out in trials in patients with HR+ HER2− early breast cancer (see the PENELOPE-B trial, where adding palbociclib to endocrine therapy in patients having HR+ HER2− early breast cancer and residual invasive disease after completing neoadjuvant chemotherapy did not improve invasive disease free survival (iFDS) as compared to placebo control when added to ET therapy (Loibl et al., 2021. Breast Cancer v39(14)). Similarly, results from the PALLAS clinical trial in patients with HR+ HER2− early-stage breast cancer indicated that combination of palbociclib with endocrine therapy did not improve DFS when compared with endocrine therapy alone (Mayer et al., 2021. Lancet, v22 (2), p212-222).

Therefore, new therapeutic strategies may improve clinical outcomes in patients with HR-positive, HER2-negative EBC.

### SUMMARY OF THE INVENTION

Accordingly, disclosed herein are methods of treating early breast cancer using the CDK4/6 inhibitor Kisqali® (ribociclib) plus endocrine therapy (ET). The present disclosure provides methods of treatment for patients with hormone receptor-positive/human epidermal growth factor receptor 2-negative (HR+/HER2−) early breast cancer (EBC). The disclosure relies, inter alia, on the results from the NATALEE trial, a positive Phase III study of a CDK4/6 inhibitor demonstrating consistent benefit in a broad population of patients with stage II and III HR+/HER2− early breast cancer (EBC) at risk of recurrence, including those with no nodal involvement.

### BRIEF DESCRIPTION OF THE DRAWINGS

FIG. 1 provides a schematic of the study design of the NATALEE clinical trial.

FIG. 2 shows simulated ANC Mean Profiles at Ribociclib 200 mg, 400 mg, and 600 mg QD, 3 weeks on/1 week off; mean ANC profiles are predicted by the ANC exposure-response model developed based on data from studies CLEE011X2101, CLEE011X1101, CLEE011X2107, CLEE011A2301, CLEE011E2301 and CLEE011F2301.

FIG. 3 provides a schematic of the inclusion according to anatomic stage group in the NATALEE clinical trial.

FIG. 4 provides a schematic of ECG and PK Relative to Dosing (C1D15).

FIG. 5 illustrates a questionnaire (i.e., EORTC QLQ-C30) for use, for example, of assessment of quality of life and healthcare resources utilization.

FIG. 6 illustrates a questionnaire (i.e., EORTC QLQ-BR23) for use, for example, of assessment of quality of life and healthcare resources utilization.

FIG. 7 illustrates a questionnaire (i.e., EQ-5D-5L) for use, for example, of assessment of quality of life and healthcare resources utilization.

FIG. 8 illustrates a questionnaire (i.e., Hospital Anxiety Depression Scale (HADS)) for use, for example, of assessment of quality of life and healthcare resources utilization.

FIG. 9 shows a Kaplan-Meier Plot (Full analysis set) for the Primary Invasive Disease-Free Survival Analysis.

FIG. 10 shows a (q) Forest Plot of iDFS by stratum (per eCRF) (Full analysis set).

FIG. 11A shows a Forest Plot of iDFS—subgroup analysis.

FIG. 11B shows a Forest Plot of iDFS—subgroup analysis.

FIG. 11C shows a Forest Plot of iDFS—subgroup analysis.

FIG. 11D shows a Forest Plot of iDFS—subgroup analysis.

FIG. 12 shows iDFS—Kaplan-Meier survival curves of iDFS by Anatomic Stage II (eCRF stratum) (Full analysis set).

FIG. 13 shows iDFS—Kaplan-Meier survival curves of iDFS by Anatomic Stage III (eCRF stratum) (Full analysis set).

FIG. 14 shows Kaplan-Meier curves for RFS (Full analysis set).

FIG. 15 shows Kaplan-Meier curves for DDFS (Full analysis set).

FIG. 16 shows Kaplan-Meier curves for OS (Full analysis set).

### DETAILED DESCRIPTION OF THE INVENTION

Herein after, the present disclosure is described in further detail and is exemplified.

The present disclosure relates to methods for treating patients suffering from breast cancer (BC). The methods are based, inter alia, on surprising results from the NATALEE clinical trial which indicate that a combination of a CDK inhibitor plus endocrine therapy can provide beneficial effects in patients with early breast cancer, for example, an early breast cancer that is hormone receptor-positive/human epidermal growth factor receptor 2-negative (HR+/HER2−).

### A. TREATMENT OF EARLY BREAST CANCER

One aspect of the present disclosure relates to a method of treatment for breast cancer in an adult patient in need thereof, comprising administering to the patient a treatment comprising a cyclin-dependent kinase (CDK) inhibitor in combination with endocrine therapy, wherein the breast cancer is early breast cancer (EBC).

EBCs may be characterized by localization of cancer cells (e.g., forming a tumor) in the breast tissue and regional lymphatics. In EBC, cancer cells are typically not detected beyond the breast or the axillary lymph nodes. Early breast cancer may be stage 0, stage I, stage II, or stage III cancer (e.g., stage 0, stage I, stage IIA, stage IIB, stage IIIIA, stage IIIB, or stage IIIC). Due to its localization, EBC may be differentiated from advanced breast cancer (also called metastatic cancer) where the cancer has spread to one or more parts of body, such as the bones, lungs, liver, etc.

In some embodiments, the EBC is a carcinoma of the breast, such as an adenocarcinoma. The EBC may be an invasive carcinoma. Invasive carcinomas, also known as infiltrating or invasive ductal carcinomas (IDC), are a type of breast cancer that starts in the milk ducts of the breast and moves into nearby tissue. In time, IDC may spread (metastasize) through the lymph nodes or bloodstream to other areas of the body. The EBC may be a noninvasive carcinoma, such as a ductal carcinoma in situ (DCIS) of the breast. The DCIS may have spread along the milk duct in the breast, but not outside of the duct system.

Signs and/or symptoms of early breast cancer may be one or more of a lump in the breast or armpit, thickening or swelling of part of the breast, irritation or dimpling of breast skin (e.g., dimpling that resembles an orange peel), redness or flaky skin in the nipple area of the breast, pulling in the nipple or pain in the nipple area, nipple discharge other than breast milk, including blood, any change in the size or shape of the breast, and/or pain in any area of the breast. Additional signs and/or symptoms of early breast cancer may comprise the presence of a breast cancer cell or cells, the presence of a breast cancer tumor, or the presence of biochemical or genetic markers in a patient's tissues or body fluids which indicate that the presence of breast cancer (e.g., breast cancer biomarkers in a tissue biopsy or a blood sample). The presence may be a physical presence, detected using tests such as ultrasound, mammogram, magnetic resonance imaging, analysis of tissue samples (e.g., a biopsy) and/or analysis of samples of body fluids (e.g., a blood sample). Any known test may be used for detecting signs and/or symptoms of cancer. The term “No evidence of disease” (NED or NEOD) may be used when there is no detectable sign and/or symptom of a cancer.

Breast cancers may be graded according to their histology. A histologic grade (or “grade”) may refer to the extent that a cancer cell differs from a normal cell, for example, by differing in the degree of cell differentiation. Grade 1 refers to breast cancer cells that resemble normal breast cells and are usually slow growing. Grade 2 refers to breast cancer cells that may not resemble normal breast cells and grow fast. Grade 3 refers to breast cancer cells that do not resemble normal breast cells and are usually fast growing. Grade 4 refers to breast cancer cells that least resemble normal breast cells and tend to grow and spread faster than lower grade tumors.

Breast cancers may be categorized according to a staging method. A staging method may be a pathologic staging method, e.g., based on a pathologist's study of the tumor tissue and any lymph nodes removed during surgery, or a clinical staging method, e.g., based on a clinician's physical exam, tests, and/or imaging.

In general, staging methods are used to categorize breast cancers based on the size of the cancer (e.g., a primary tumor) and how far the cancer has spread in the body.

One method used to categorize breast cancers or tumors based on their stage is the TNM classification or staging method. Using this method, a tumor may be classified as TO when there is no evidence of a tumor, while classifications of T1, T2, T3, or T4 may be used for to identify the size and extension of the tumor. Tx may indicate that the tumor cannot be assessed. N (node) describes the spread of cancer to nearby lymph nodes. A tumor may be classified as NO when there is no spread of the tumor to regional nodes, while classifications of N1-N3 may be used to indicate nodal spread.

M (metastasis) describes metastasis (e.g., spread of cancer to other parts of the body). A tumor may be classified as M0 when no distant metastasis is present, while a classification of M1 may be used when there is evidence of distant metastasis (Rosen and Sapra, TNM Classification, StatPearls Publishing; Treasure Island (FL), January 2023).

In breast cancer, T1 may be defined as a tumor 20 mm or less in diameter. T1a may be defined as a tumor more than 1 mm but not more than 5 mm in greatest dimension; T1b may be defined as a tumor more than 5 mm but not more than 10 mm in greatest dimension; T1c may be defined as a tumor more than 10 mm but not more than 20 mm in greatest dimension. T2 may be defined as a tumor more than 20 mm but not more than 50 mm in greatest dimension. T3 may be defined as a tumor more than 50 mm in greatest dimension. T4 may be defined as a tumor of any size with direct extension to the chest wall and/or the skin (e.g., ulceration or skin nodules). T4a may be extension to the chest wall. T4b may be defined as edema of the skin or ulceration of the skin of the breast, or satellite skin nodules confined to the same breast. T4c may be defined as both T4a and T4b. T4d may be defined as inflammatory cancer, e.g., cancer characterized by diffuse erythema and edema involving approximately a third or more of the skin of the breast.

Regional lymph nodes near the breast may comprise one or more of (1) axillary lymph nodes, the interpectoral (Rotter's nodes), and lymph nodes along the axillary vein and its tributaries. Axillary lymph nodes may be Level I (low-axilla), Level II (mid-axilla), or Level III (apical axilla); (2) internal mammary lymph nodes; (3) superclavical lymph nodes; and (4) intramammary lymph nodes.

For cancers that have or can spread to the lymph nodes, NO indicates that the cancer has not spread to nearby lymph nodes; N1 indicates that the cancer has spread to 1 to 3 axillary lymph nodes, and/or is found in internal mammary lymph nodes on sentinel lymph node biopsy; N2 indicates that the cancer has spread to 4 to 9 axillary lymph nodes, or has enlarged the internal mammary lymph nodes; and N3 indicates that the cancer has spread to 10 or more axillary lymph nodes, with at least one area of cancer spread greater than 2 mm, or has spread to infraclavicular nodes, with at least one area of cancer spread greater than 2 mm, or is found in at least one axillary lymph node (with at least one area of cancer spread greater than 2 mm) and has enlarged the internal mammary lymph nodes, or has spread to 4 or more axillary lymph nodes (with at least one area of cancer spread greater than 2 mm), and to the internal mammary lymph nodes on sentinel lymph node biopsy, or has spread to the supraclavicular nodes on the same side of the cancer with at least one area of cancer spread greater than 2 mm.

Node categorization may be a clinical categorization or may be a pathological categorization. Clinical categorization may comprise cN1 defined as metastasis in ipsilateral level I, II axillary lymph nodes; cN2a defined as metastasis in ipsilateral level I, II axillary lymph nodes fixed to one another (matted); cN2b defined as metastasis only in clinically detected ipsilateral internal mammary nodes and in the absence of clinically evident level I, II axillary lymph node metastasis; cN3a defined as metastasis in ipsilateral infraclavicular (level III axillary) lymph nodes with our without level I, II axillary lymph node involvement; cN3b defined as metastasis in clinically detected ipsilateral internal mammary lymph node(s) and clinically evident axillary lymph node(s); or cN3c defined as metastasis in ipsilateral superclavicular lymph node(s) with or without axillary or internal mammary lymph node involvement.

An add-on to the TMN classification system is the R classification system, or residual tumor classification system, which may be used to indicate the tumor status following treatment. The R classification may indicate the effects of treatment and may be used to predict prognosis. For example, a patient may be diagnosed with a tumor that is classified using the TMN classification system and then treated, after which any residual tumor may be classified according to the R classification system. Using this system, the extent of resection of a tumor can is classified as R0, R1, or R2. In R0, there is no residual tumor after surgery, e.g., the surgical margin is microscopically negative for residual tumor. In R1, there is no residual macroscopic tumor but microscopic margins still demonstrate the presence of tumor. In R2, the gross (macroscopically-visible) tumor remains post-surgery.

The TNM classification system predates many genetic tests and/or biochemical marker (or “biomarker”) tests that are now routinely used to provide additional predictive or prognostic information about cancer types, and may be used together with these and/or other tests for categorizing cancers. For example, the TNM classification system may be used in combination with measures such as tumor grade, estrogen receptor (ER) status, progesterone receptor (PR) status, and human epidermal growth factor receptor 2 (HER2) status. An example of such a combination is provided in Table 1 of Ann Surg Oncol. 2018 July; 25(7):1783-1785. doi: 10.1245/s10434-018-6486-6. Epub 2018 Apr. 18.

One example of a biomarker used in breast cancer is the nuclear antigen Ki-67. Ki-67 levels may be measured in breast cancer cells, for example, using immunohistochemical staining. The anti-human Ki-67 antibody MIB1 may be used for the immunohistochemistry. Ki-67 values are calculated as a percentage of positively marking malignant cells among the total number of malignant cells assessed (Breast Cancer Res Treat. 2013; 139(2): 539-552). In general, Ki-67 expression correlates with high levels of cell proliferation (J Clin Oncol. 2005 Oct. 1; 23(28):7212-20).

Genetic tests such as multigene or multiparameter expression assays may be used to evaluate characteristics of a breast cancer, risk of metastasis, and/or risk of recurrence. One exemplary test is the Oncotype DX® test, where a breast recurrence score of 26 (on a scale of 0-100) indicates a risk for recurrence. In another exemplary test, the Prosigna®/PAM50 (Prediction Analysis of Microarray 50) test, tumors are assigned a score of low risk, intermediate risk, or high risk for metastasis, depending on the expression levels of 50 genes in the tumor. Other exemplary tests include but are not limited to the MammaPrint®, EndoPredict®, and Breast Cancer Index® tests, which may be used to determine risk scores for cancer recurrence.

Another method for categorizing tumors is the “Stage Grouping” categorization system. This method, which may be used alone or together with the TNM classification system and other tests, groups breast cancers in Anatomic Stage Groups (or “stages”). Thus, breast cancer may be categorized according to a stage group (or “Anatomic Stage Group”) 0, I, II, III, or IV. In particular, the stage group may be stage 0, Ia, Ib, IIa, IIb, IIIIa, IIIb, IIIC, or IV.

Stage 0: Stage zero (0) may describe disease that is only in the ducts of the breast tissue and has not spread to the surrounding tissue of the breast. It is also called non-invasive or in situ cancer (Tis, N0, M0).

Stage IA may refer to a tumor that is small, invasive, and has not spread to the lymph nodes (T1, N0, M0).

Stage IB may refer to cancer that has spread to the lymph nodes and the cancer in the lymph node is larger than 0.2 mm but less than 2 mm in size. There may be either no evidence of a tumor in the breast or the tumor in the breast is 20 mm or smaller (TO or T1, N1mi (also called “N1 micrometastatic”), M0).

Stage IIA may refer to cancer meeting any one of these conditions:
a. There is no evidence of a tumor in the breast, but the cancer has spread to 1 to 3 axillary lymph nodes. It has not spread to distant parts of the body (T0, N1, M0).
b. The tumor is 20 mm or smaller and has spread to 1 to 3 axillary lymph nodes (T1, N1, M0).
c. The tumor is larger than 20 mm but not larger than 50 mm and has not spread to the axillary lymph nodes (T2, N0, M0).

Stage IIB may refer to either of these conditions:
a. The tumor is larger than 20 mm but not larger than 50 mm and has spread to 1 to 3 axillary lymph nodes (T2, N1, M0).
b. The tumor is larger than 50 mm but has not spread to the axillary lymph nodes (T3, N0, M0).

Stage IIIIA may refer to a tumor of any size that has spread to 4 to 9 axillary lymph nodes or to internal mammary lymph nodes. It has not spread to other parts of the body (T0, T1, T2, or T3; N2; M0). Stage IIIA may also be a tumor larger than 50 mm that has spread to 1 to 3 axillary lymph nodes (T3, N1, M0).

Stage IIIB may refer to a tumor that has spread to the chest wall or caused swelling or ulceration of the breast, or it is diagnosed as inflammatory breast cancer. It may or may not have spread to up to 9 axillary or internal mammary lymph nodes. It has not spread to other parts of the body (T4; N0, N1, or N2; M0).

Stage IIIC may refer to a tumor of any size that has spread to 10 or more axillary lymph nodes, the internal mammary lymph nodes, and/or the lymph nodes under the collarbone. It has not spread to other parts of the body (any T, N3, M0).

Stage IV (metastatic) may refer to a tumor that can be any size and has spread to other organs, such as the bones, lungs, brain, liver, distant lymph nodes, or chest wall (any T, any N, M1). Metastatic cancer found when the cancer is first diagnosed occurs about 6% of the time. This may be called de novo metastatic breast cancer. Most commonly, metastatic breast cancer is found after a previous diagnosis of early-stage breast cancer.

Recurrent cancer is cancer that has come back after a treatment and can be described as local, regional, and/or distant (Breast Cancer: Stages, from Cancer.Net).

Recurrence may be evaluated by medical imaging and confirmed histologically (or cytologically, when applicable). Recurrences may be classified into local, regional, or distant recurrence, or contralateral invasive breast cancer or second primary non-breast invasive cancer according to the following guidelines:
Local invasive breast cancer recurrence or ipsilateral invasive breast tumor recurrence: invasive breast cancer involving the same breast as the original primary tumor. Ductal and lobular carcinoma in situ and tumors in the contralateral breast and/or contralateral lymph nodes are not considered a local invasive recurrence. May be confirmed histologically.
Regional breast cancer recurrence: invasive breast cancer in the ipsilateral axilla, regional lymph nodes (all levels), chest wall, or skin of the ipsilateral breast. A tumor in the contralateral breast is not considered a regional recurrence. May be confirmed histologically (preferred) or cytologically.
Distant recurrence: distant metastasis of breast cancer (bones, distant lymph nodes, internal organs, CNS, bone marrow, etc.) or any areas of invasive breast cancer recurrence that are not local or regional. Distant recurrence may be confirmed histologically (preferred) or cytologically.
   If bone metastases were identified by a bone scan, it may be confirmed histologically (preferred) or radiographically (CT, MRI, or FDG-PET-CT) if biopsy confirmation is not possible.
   Metastases in the central nervous system may be confirmed histologically (preferred), cytologically, or radiographically (CT or MRI, both with IV contrast) if biopsy confirmation is not possible.
   All other sites of metastases may be confirmed histologically (preferred) or cytologically unless there is an unacceptable risk to the patient due to the procedure.
Contralateral invasive breast cancer: any invasive breast cancer in the contralateral breast with or without contralateral lymph nodes involvement. Contralateral invasive breast cancer has to be confirmed histologically. In situ/non-invasive contralateral breast cancers are not included in contralateral invasive breast cancers.
Second primary non-breast invasive cancer: any second primary non-breast invasive cancers. Second primary non-breast invasive cancer has to be confirmed histologically.

In situ/non-invasive cancers and basal or squamous cell carcinomas of the skin are not considered as second primary non-breast invasive cancers.

Table B1 summarizes Anatomic Stage Groups from the AJCC 8^{th} Edition

TABLE B1

| Anatomic Stage Group | T-category | N-category |
|---|---|---|
| 0 | Tis | N0 |
| IA | T1 | N0 |
| IB | T0-1 | N1mi |
| IIA | T0-1 | N1 |
| | T2 | N0 |
| IIB | T2 | N1 |
| | T3 | N0 |
| IIIA | T0-3 | N2 |
| | T3 | N1 |
| IIIB | T4 | N0-2 |
| IIIC | Any T | N3 |
| IV | T1-T3 | N1-N3 |

Note that T2, T3, and T4 tumors with nodal micrometastases (N1mi) may be staged using the N1 category.

Accordingly, in some embodiments, the adult patient treated with the methods herein has an EBC that is a ductal carcinoma in situ, a stage I breast cancer, a stage II breast cancer such as a IIA breast cancer or a stage IIB breast cancer, or a stage III breast cancer such as a stage IIIA, a stage IIIB, or a stage IIIC breast cancer. In some embodiments, the patient has a stage II or III early breast cancer. In some embodiments, the patient has a stage IIA cancer or a stage IIB cancer. In some embodiments, the patient has a stage IIIA cancer, a stage IIIB cancer, or a stage IIIC cancer. The patient may be node-positive (e.g., cancer has spread to their lymph nodes) or node-negative (e.g., cancer has not spread to their lymph nodes). In some embodiments, the treatment methods as described herein are performed irrespective of the nodal status of the patient's breast cancer.

In certain embodiments, the EBC is a hormone receptor-positive (HR+) breast cancer, e.g., a breast cancer comprising cells expressing one or more type of hormone receptor. Exemplary hormone receptors may be estrogen receptors (ERs), such as ERa or ER®3, and/or progesterone receptors (PRs) such as PRA and PRB. EBCs may comprise breast cancer cells expressing either estrogen receptors (ER+) or progesterone receptors (PR+), or a combination of both estrogen receptors and progesterone receptors (ER+/PR+). In some embodiments, the EBC is ER+/PR−, ER−/PR+, or ER+/PR+.

In certain embodiments, the EBC is positive for human epidermal growth factor receptor 2 (HER2).

Certain embodiments relate to a method of preventing or reducing signs or symptoms of early stage breast cancer, comprising administering to the patient a treatment comprising ribociclib, a freebase form thereof or a pharmaceutically acceptable salt thereof in combination with an aromatase inhibitor, preferably letrozole or anastrozole. In some embodiments, the ribociclib is a freebase form thereof or a pharmaceutically acceptable salt thereof, and is administered to the patient in a dose ranging from 150 mg/day to 450 mg/day on days 1-21 of a 28-day cycle. In some embodiments, the aromatase inhibitor is administered on every day of the 28-day cycle.

### B. CDK INHIBITORS AND ENDOCRINE THERAPY

One aspect of the present disclosure relates to a method of treatment for early breast cancer in an adult patient in need thereof, comprising administering to the patient a treatment comprising a cyclin-dependent kinase (CDK) inhibitor in combination with endocrine therapy (ET).

Cyclin-dependent kinases (CDKs) are serine/threonine protein kinases that bind to cyclins to form an active complex in cells. In healthy cells, various extra- and intra-cellular cues closely regulate the formation of the CDK/cyclin complex and control its phosphorylation of target proteins involved in the cell cycle, such as retinoblastoma (Rb) proteins, lamins, histone H 1, and components of the mitotic spindle. In cancer cells, however, CDKs can become hyperactivated, leading to uncontrolled tumor proliferation.

CDK4 and CDK6, which bind to D-cyclins, are exemplary CDKs that have been shown to promote growth of certain breast cancer tumors. In these tumors, D-cyclins are overexpressed, leading to activation of CDK4 and CDK6, subsequently, to phosphorylation of Rb, release of Rb's suppression of the E2F transcription factor, and rapid progression of tumor cells through the cell cycle (Shah et al., Oncology. 2018 May 15; 32(5): 216-222).

CDK inhibitors have been shown to block the unregulated cell growth and division resulting from hyperactivation of CDKs and/or overexpression of cyclin proteins. For example, inhibitors of CDK/cyclin complexes, and of CDK4/6 in particular, have been used for treating patients suffering from certain types of cancer. CDK4/6 inhibitors palbociclib, ribociclib, and abemaciclib have been approved by regulatory authorities for treatment of advanced or metastatic breast cancer that is hormone receptor positive (HR+ or HR-positive), and human epidermal growth factor receptor 2 negative (HER2−) (Fassi et al., Science. 2022 Jan. 14; 375(6577): eabc1495).

To date, ribociclib has been approved by a number of regulatory authorities including the United States Food and Drug Administration (FDA) and the European Commission. By the FDA it has been approved in combination with: (1) an AI for the treatment of pre/perimenopausal or postmenopausal women with HR-positive, HER2-negative advanced or metastatic breast cancer, as initial endocrine-based therapy; or (2) fulvestrant for the treatment of postmenopausal women with HR-positive, HER2-negative advanced or metastatic breast cancer, as initial endocrine based therapy or following disease progression on ET. In Europe, ribociclib is indicated for the treatment of women with HR-positive, HER2-negative locally advanced or metastatic breast cancer in combination with an AI or fulvestrant as initial endocrine-based therapy, or in women who have received prior ET. In pre- or perimenopausal women, the ET may be combined with a luteinizing hormone-releasing hormone agonist.

Accordingly, in some embodiments of the present disclosure, the CDK inhibitor used in the methods disclosed herein may be a CDK4/6 inhibitor, for example, an inhibitor of a CDK4/6/cyclin complex. The cyclin may be a D cyclin, such as cyclin-D1 in a CDK4/cyclin-D1 complex or cyclin-D3 in a CDK6/cyclin-D3 complex.

In some embodiments, the CDK4/6 inhibitor may be a compound described by Formula A1 below or a salt thereof.

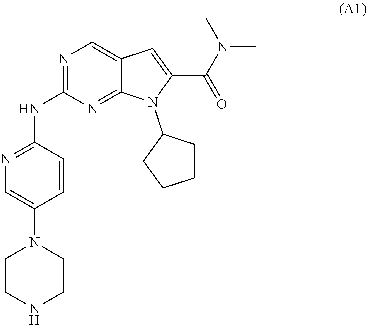 [SMILES: CN(C)C(=O)c1cc2cnc(Nc3ccc(N4CCNCC4)cn3)nc2n1C1CCCC1]

The compound of Formula A1 is known by the international non-proprietary name ribociclib. Thus, in some embodiments, the CDK4/6 inhibitor may be a compound having the international non-proprietary name ribociclib.

Ribociclib may be in the form of its free base or may be a pharmaceutically acceptable salt of ribociclib. Salts can be present alone or in mixture with free compound, e.g. ribociclib, and are preferably pharmaceutically acceptable salts. Such salts of ribociclib are formed, for example, as acid addition salts, preferably with organic or inorganic acids, from compounds of ribociclib with a basic nitrogen atom. Suitable inorganic acids are, for example, halogen acids, such as hydrochloric acid, sulfuric acid, or phosphoric acid. Suitable organic acids are, e.g., succinic acid, carboxylic acids, or sulfonic acids, such as fumaric acid or methansulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In some embodiments, ribociclib may be in the form of racemates, diastereoisomers, enantiomers, and tautomers, as well as the corresponding crystal modifications where present, e.g. solvates, hydrates, and polymorphs.

Ribociclib may be in the form of a pharmaceutically acceptable salt, for example, a succinic acid salt such as ribociclib succinate. The chemical name of ribociclib succinate is butanedioic acid-7-cyclopentyl-N,N-dimethyl-2-{[5-(piperazin1-yl)pyridin-2-yl]amino}-7H-pyrrolo[2,3-d]pyrimidine-6-carboxamide (1:1) corresponding to the molecular formula C₂₇H₃₆N₈O₅. It has a relative molecular mass of 552.6 g/mol (EMA Assessment report for Kisqali, Procedure No. EMEA/H/C/004213/0000, dated Jun. 22, 2017).

Oral tablet forms of ribociclib are known in the art (e.g. WO2016/166703) and have been approved as the KISQALI® product. Various ribociclib salts have been described (e.g. see claim 85 of WO2022/207788), and the approved KISQALI® product contains ribociclib succinate. Quantities of any salt will be adjusted to provide the desired quantity of ribociclib (e.g. 254.40 mg of ribociclib succinate corresponds to 200 mg of ribociclib). Various polymorphs of ribociclib succinate are known (e.g. see WO2019/040567, WO2019/082143, WO2019/150181, WO2019/166987, WO2020/225827, WO2021/038590, etc.). The methods described herein may be implemented using the approved KISQALI® product, e.g., along with approved endocrine therapies.

As described herein, the CDK inhibitor (e.g, a CDK4/6 inhibitor such as ribociclib or a pharmaceutically acceptable salt thereof, such as ribociclib succinate) may be administered with endocrine therapy. In some embodiments of the present disclosure, the endocrine therapy is an aromatase inhibitor.

Endocrine therapy (also called hormone therapy, hormonal therapy, or hormone treatment) has been used to slow or stop the growth of hormone-sensitive cancers (also called hormone-dependent cancers). Hormone-sensitive tumors express receptors for hormones such as estrogen and/or progesterone, and the hormones may promote growth of the cancer.

Accordingly, endocrine therapy refers to a therapy that either (1) interferes with the effects of the hormones on the cancer cells and/or (2) blocks the body's ability to produce hormones.

Exemplary compounds that interfere with the effects of hormones on breast cancer cells are selective estrogen receptor modulators (SERMs), which bind to estrogen receptors and prevent estrogen from binding. SERMs may mimic the effects of estrogen. SERMs approved by the FDA for treatment of breast cancer are tamoxifen (Nolvadex®) and toremifene (Fareston®). Other anti-estrogen drugs may bind to estrogen receptors without mimicking the effects of estrogen, and may instead target the estrogen receptor for destruction. An exemplary anti-estrogen compound is fulvestrant (Faslodex®).

The production of hormones may be blocked by ablating tissues or organs that produce the hormones. In premenopausal women, for example, where the ovaries are the main source of estrogen, ovarian ablation (e.g., by oophorectomy (also called ovariotomy or ovariectomy) or treatment with radiation) may be performed to remove or reduce estrogen levels.

Alternatively, the production of hormones may be blocked by administering compounds that suppress synthesis and/or release of the hormones. Gonadotropin-releasing hormone (GnRH) agonists administered over a prolonged period may cause desensitization and consequently suppression of gonadotropin secretion. GnRH antagonists, in contrast, inhibit GnRH signal transduction and gonadotropin secretion (Reprod Biomed Online. 2002; 5 Suppl 1:1-7. doi: 10.1016/s1472-6483(11)60210-1). Exemplary GnRH agonists are goserelin (Zoladex®) and leuprolide (Lupron®), which suppress release of estrogen from the ovaries in women and suppress the release of testosterone from the testicles in men. In some embodiments, goserelin is used in the methods disclosed herein.

A further class of compounds that block production of estrogen, in particular, are aromatase inhibitors. Aromatase inhibitors work by inhibiting the action of the enzyme aromatase, which converts androgens into estrogens in a process called aromatization. Notably, in premenopausal women, the ovaries produce levels of aromatase that are too high to be blocked by aromatase inhibitors alone. For premenopausal women, aromatase inhibitors may be combined with a compound that suppresses ovarian function (e.g., goserelin or leuprolide). In contrast, postmenopausal women produce estrogen primarily in peripheral tissues and not in the ovaries, so aromatase inhibitors may be sufficient to inhibit production of estrogen in these women.

There are two types of aromatase inhibitors: (1) steroidal inhibitors, such as exemestane (Aromasin®) which forms a permanent and deactivating bond with the aromatase enzyme; and (2) non-steroidal inhibitors (NSAls), such as anastrozole (Arimidex®) or letrozole (Femara®).

Letrozole is a nonsteroidal competitive inhibitor of the aromatase enzyme system. Letrozole acts by highly selective inhibition of conversion of androgens (mainly from adrenal glands, the primary source of estrogens in postmenopausal women) to estrogens. Letrozole induces a 75% to 95% decrease of estrogen levels after two weeks of treatment with daily doses of 0.1 to 5 mg, with no significant clinical and laboratory toxicities or changes in levels of other hormones of the endocrine system (Lancet Lond Engl 386:1341-1352, 2015; Cancer 75:2132-2138, 1995). Anastrozole, like letrozole, is a selective NSAI. It significantly lowers serum estradiol concentrations with no detectable effect on formation of adrenal corticosteroids or aldosterone.

In some embodiments, the endocrine therapy administered with the CDK inhibitor (e.g, a CDK4/6 inhibitor such as ribociclib or a pharmaceutically acceptable salt thereof such as ribociclib succinate) is an aromatase inhibitor, e.g., anastrozole or letrozole. In some embodiments, the therapy further comprises a gonadotropin-releasing hormone agonist such as goserelin.

Exemplary aromatase inhibitors are described in Breast Cancer Res Treat. 2007 October; 105(Suppl 1): 7-17.

The aromatase inhibitor may be a non-steroidal aromatase inhibitor described by one of the formulas below, or a pharmaceutically acceptable salt thereof.

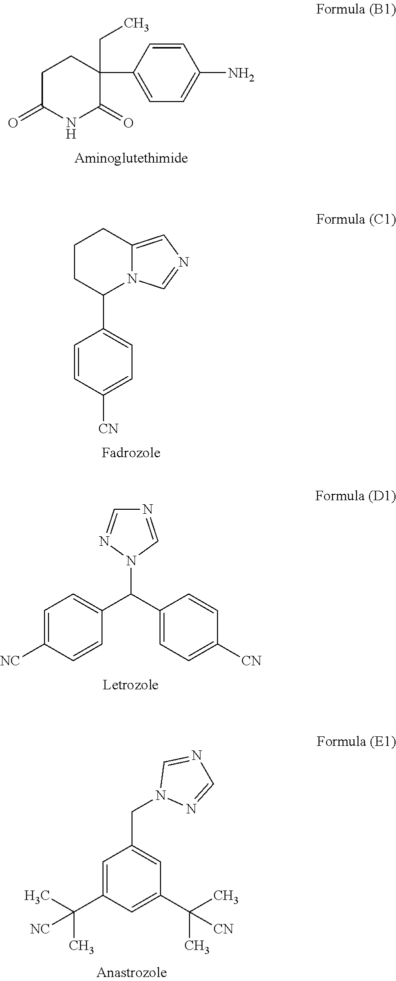 [SMILES: CCC1(c2ccc(N)cc2)CCC(=O)NC1=O ; N#Cc1ccc(C2CCCc3cncn32)cc1 ; N#Cc1ccc(C(c2ccc(C#N)cc2)n2cncn2)cc1 ; CC(C)(C#N)c1cc(Cn2cncn2)cc(C(C)(C)C#N)c1]

In some embodiments, the aromatase inhibitor may be letrozole. The aromatase inhibitor may be a pharmaceutically acceptable salt of letrozole. The chemical name of letrozole is 4,4′-(1H-1,2,4-Triazol-1-ylmethylene)dibenzonitrile. Letrozole may be freely soluble in dichloromethane, slightly soluble in ethanol, and practically insoluble in water. It has a molecular weight of 285.31 g/mol, empirical formula C₁₇H₁₁N₅, and a melting range of 184° C. to 185° C.

In some embodiments, letrozole may be in the form of racemates, diastereoisomers, enantiomers, or tautomers, as well as the corresponding crystal modifications where present, e.g. solvates, hydrates and polymorphs.

In some embodiments, the aromatase inhibitor may be anastrozole or a pharmaceutically acceptable salt thereof. The chemical name of anastrozole is α,α,α′,α′-tetramethyl-5-(1H-1,2,4-triazol-1-ylmethyl)-m-benzenediacetonitrile. Anastrozole is freely soluble in methanol, acetone, ethanol, and tetrahydrofuran, and very soluble in acetonitrile. It has a molecular weight of 293.374 g/mol, empirical formula C₁₇H₁₉N₅, and a melting range of 80° C. to 86° C. In some embodiments, anastrozole may be in the form of a solvate, hydrate or polymorph.

In some embodiments, endocrine therapy may comprise an aromatase inhibitor (such as letrozole or anastrozole) for postmenopausal women. For premenopausal women or men, the endocrine therapy may comprise an aromatase inhibitor (such as letrozole or anastrozole) and may further comprise a compound that suppresses gonadal function (e.g., a GnRH agonist such as goserelin).

The GnRH agonist may be a compound according to the formula below or a pharmaceutically acceptable salt thereof:

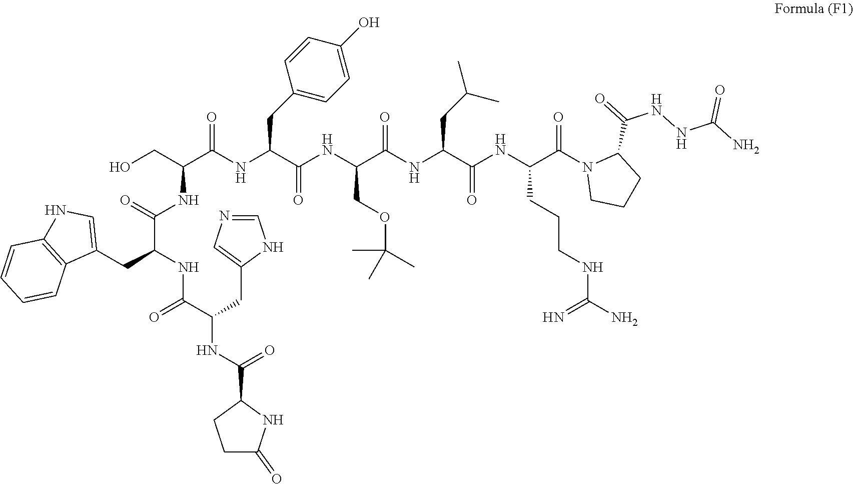 [SMILES: CC(C)C[C@H](NC(=O)[C@@H](COC(C)(C)C)NC(=O)[C@H](Cc1ccc(O)cc1)NC(=O)[C@H](CO)NC(=O)[C@H](Cc1c[nH]c2ccccc12)NC(=O)[C@H](Cc1cnc[nH]1)NC(=O)[C@@H]1CCC(=O)N1)C(=O)N[C@@H](CCCNC(=N)N)C(=O)N1CCC[C@H]1C(=O)NNC(N)=O]

In some embodiments, the aromatase inhibitor may be goserelin or a pharmaceutically acceptable salt thereof. Goserelin is sold under the tradename Zoladex®. It has a molecular weight of 1269.433 g/mol, and an empirical formula C₅₉H₈₄N₁₈O₁₄. In some embodiments, goserelin may be in the form of a solvate, hydrate or polymorph.

### C. ADMINISTRATION—DOSES AND TREATMENT SCHEDULE

A further aspect of the present disclosure relates to a method of treatment for early breast cancer in an adult patient in need thereof, comprising administering to the patient a treatment comprising a dose of a cyclin-dependent kinase (CDK) inhibitor, in combination with a dose of an endocrine therapy.

In some embodiments, the CDK inhibitor is a CDK4/6 inhibitor such as ribociclib or a pharmaceutically acceptable salt thereof (such as ribociclib succinate) and the endocrine therapy is an aromatase inhibitor such as letrozole. In some embodiments, the endocrine therapy is letrozole.

Ribociclib at a dose of 600 mg daily from days 1 to 21 of a 28-day cycle has been shown to be tolerable and efficacious when combined with ET in clinical trials in patients with advanced BC (N Engl J Med 375:1738-1748, 2016). However, due to the potential for later recurrence of HR-positive, HER2-negative EBC, where events may occur from a year after surgery and lasting for at least 15 years after diagnosis, a longer duration of therapy may also be beneficial for EBC. In some embodiments, ribociclib (in combination with ET) may be administered at a dose of less than 600 mg daily. For example, ribociclib (or a pharmaceutically acceptable salt thereof such as ribociclib succinate) may be administered at a dose of about 400 mg daily. This exemplary dose may be administered as 2×200 mg daily.

In some embodiments, the ribociclib (or a pharmaceutically acceptable salt thereof such as ribociclib succinate) may be administered at dose of about 400 mg/day. The dose of ribociclib or a pharmaceutically acceptable salt thereof (such as ribociclib succinate) may be administered once per day in a tablet form, for example, as 2×200 mg tablets (it being understood that these two tablets may be taken simultaneously or sequentially within the single daily dosing). The dose may be administered as an oral solution. The dose may be administered orally (by mouth). In some embodiments, the dose of the ribociclib or a pharmaceutically acceptable salt thereof (such as ribociclib succinate) is about 200 mg/day. This dose may be administered in a tablet form, for example, as 1×200 mg tablet. The dose may be administered orally (by mouth). The dose may be a flat fixed dose, e.g., not calculated by body weight or body surface area.

A dose adjustment may be made, for example, to administer a dose of ribociclib or a pharmaceutically acceptable salt thereof (such as ribociclib succinate) that is less than about 400 mg/day. For example, a dose of about 350 mg/day, about 300 mg/day, about 250 mg/day, about 200 mg/day, or about 150 mg/day of ribociclib or a pharmaceutically acceptable salt thereof (such as ribociclib succinate) may be administered. In some embodiments, a dose of about 200 mg/day is administered after an earlier dose of 400 mg/day has been administered. In some embodiments, the dose is administered in tablet form, such as 1×200 mg tablet. The dose of about 200 mg/day may be administered orally. Accordingly, a patient who had previously received a dose of about 400 mg dose of ribociclib or a pharmaceutically acceptable salt thereof (such as ribociclib succinate) (e.g., 2×200 mg tablets orally) may receive a dose of about 200 mg/day instead (e.g., 1×200 mg tablets orally). The dose may taken once daily (e.g., 1×200 mg tablets once daily, or 2×200 mg tablets once daily).

The dose adjustment may be made for patients who do not tolerate a dose of about 400 mg/day. For example, patients who receive a dose of about 400 mg/day of ribociclib (or a pharmaceutically acceptable salt thereof, such as ribociclib succinate) and experience one or more of thrombocytopenia, decreased absolute neutrophil count (ANC), febrile neutropenia, anemia, hepatoxicity (e.g., as indicated by bilirubin levels and/or levels of AST and ALT enzymes), drug-induced liver injury, heart abnormalities (e.g., as measured by QT interval), interstitial lung disease/pneumonitis, and/or other adverse events may receive an adjusted dose of about 200 mg/day dose of ribociclib (or a pharmaceutically acceptable salt thereof, such as ribociclib succinate).

Thus, the dose of ribociclib (or a pharmaceutically acceptable salt thereof, such as ribociclib succinate) may range from about 150 mg/day to about 450 mg/day. The dose may be about 150 mg/day, about 200 mg/day, about 250 mg/day, about 300 mg/day, about 350 mg/day, or about 450 mg/day. The dose may be taken once daily.

In some embodiments, the dose of ribociclib (or a pharmaceutically acceptable salt thereof, such as ribociclib succinate) is not about 600 mg/day.

In some embodiments, the aromatase inhibitor is letrozole. The dose of letrozole may be selected according to data (e.g., clinical data) indicating disease-free survival in patients, which may be combined with data about adverse effects such as renal impairment and/or hepatic impairment, to establish an effective dose. The dose may be selected by testing efficacy and tolerance in an individual patient.

The dose of letrozole may range from about 1 mg/day to about 4 mg/day. For example, the dose of letrozole may be about 1 mg/day, about 1.25 mg/day, about 1.5 mg/day, about 1.75 mg/day, about 2 mg/day, about 2.25 mg/day, about 2.5 mg/day, about 2.75 mg/day, about 3 mg/day, about 3.25 mg/day, about 3.5 mg/day, about 3.75 mg/day, or about 4 mg/day. In some embodiments, the dose of letrozole is about 2.5 mg/day. The dose may be a flat fixed dose, e.g., not calculated by body weight or body surface area.

The dose of letrozole may be administered orally (e.g., by mouth). Accordingly, any of the dosages listed above, such as a dose of letrozole that is about 2.5 mg/day, may be administered. The dose of letrozole may be administered orally. The dose may be administered in tablet form. The dose may be taken once daily.

In certain embodiments, the aromatase inhibitor is anastrozole. The dose of anastrozole may be selected according to data (e.g., clinical data) indicating disease-free survival in patients, which may be combined with data about adverse effects such as renal impairment and/or hepatic impairment, to establish an effective dose. The dose may be selected by testing efficacy and tolerance in an individual patient.

The dose of anastrozole may range from about 0.5 mg/day to about 1.5 mg/day. For example, the dose of anastrozole may be about 0.5 mg/day, about 0.75 mg/day, about 1 mg/day, about 1.25 mg/day, or about 1.50 mg/day. In some embodiments, the dose of anastrozole is about 1 mg/day.

The dose of anastrozole may be administered orally (e.g., by mouth). Accordingly, any of the dosages listed above, such as a dose of anastrozole that is about 1 mg/day may be administered. The dose of anastrozole may be administered orally. The dose may be administered in tablet form. The dose may be taken once daily.

In addition to the ribociclib (or a pharmaceutically acceptable salt thereof, such as ribociclib succinate) (e.g., about 400 mg/day, or about 200 mg/day) and letrozole (about 2.5 mg/day) or anastrozole (about 1 mg/day), patients may receive a gonadotropin-releasing hormone agonist such as goserelin. For example, patients who are premenopausal women and patients who are men may receive a dose of goserelin. The dose of goserelin may be selected according to data (e.g., clinical data) indicating efficacy in patients, or by testing efficacy and tolerance in an individual patient.

The dose of goserelin may range from about 2 mg to about 5 mg. The dose of goserelin may be about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, or about 5 mg. In some embodiments, the dose of goserelin is about 3.4 mg, 3.5 mg, 3.6 mg, or 3.7 mg. In some embodiments, the dose of goserelin is 3.6 mg. The dose may be a flat fixed dose, e.g., not calculated by body weight or body surface area.

The dose of goserelin may be administered subcutaneously. The dose may be administered subcutaneously at time intervals ranging from 2 weeks to 4 weeks.

In one embodiment, goserelin is administered subcutaneously at a dose of 3.6 mg.

The compounds may be administered separately, for example, when the compounds are administered by different routes of administration and/or administered according to different treatment schedules. If administered separately, the compounds may be administered simultaneously, or sequentially. In one exemplary example, two compounds are administered to the patient in two separate doses, but are administered on the same day as each other. In this example, the two compounds may be administered at the same time, or they may be administered at different times. The compounds may be administered separately in two or more separate unit doses (e.g., where a unit dose is the amount of the compound administered to the patient in a single dose) and/or unit dosage forms.

Compounds may be combined for administration together. Compounds may be administered together in a single pharmaceutical composition, for example, the compounds may be administered together in a single dose (e.g., a unit dose). The unit dosage form may also be a fixed combination.

In some embodiments, a dose of ribociclib (or a pharmaceutically acceptable salt thereof, such as ribociclib succinate) (about 400 mg/day, or about 200 mg/day) and a dose of endocrine therapy (e.g., letrozole (dose about 2.5 mg/day) or anastrozole (dose about 1 mg/day) are administered together. In general, the doses may be administered at the same time each day, such as in the morning. The doses may be administered in the afternoon or evening. In some embodiments, the doses are not administered in the evening.

A further aspect of the present disclosure relates to a method of treatment for early breast cancer in an adult patient in need thereof, comprising administering to the patient a treatment comprising a dose of a cyclin-dependent kinase (CDK) inhibitor, in combination with a dose of an endocrine therapy, according to a treatment schedule.

In some embodiments, the treatment schedule comprises a treatment cycle of about 28 days. In this cycle, the CDK inhibitor may be administered once daily on days 1 to 21 of the 28-day cycle, followed by 7 days off the CDK inhibitor, wherein during the off days the endocrine therapy is administered once daily continuously. Accordingly, the endocrine therapy (e.g., letrozole or anastrozole) may be administered on every day of the 28-day cycle.

Accordingly, in some embodiments the CDK inhibitor is ribociclib (or a pharmaceutically acceptable salt thereof, such as ribociclib succinate) administered at a dose of about 400 mg/day (or about 200 mg/day) and the endocrine therapy is letrozole administered at a dose of about 2.5 mg/day or anastrozole administered at a dose of about 1 mg/day, wherein the ribociclib (or a pharmaceutically acceptable salt thereof, such as ribociclib succinate) is administered once daily on days 1 to 21 of a 28-day cycle, followed by 7 days off ribociclib (or a pharmaceutically acceptable salt thereof, such as ribociclib succinate) (days 22-28 of the cycle), and the letrozole or anastrozole is administered once daily continuously during the 28-day period (e.g., on every day of the 28-day cycle). The ribociclib (or a pharmaceutically acceptable salt thereof, such as ribociclib succinate) may be administered orally, for example, in tablet form. The letrozole or the anastrozole may be administered orally, for example, in tablet form.

For patients whose treatment further comprises administration of goserelin (e.g., at a dose of about 3.6 mg), this may be administered once during the 28-day cycle. The goserelin may be administered every 2-4 weeks, for example, every 4 weeks. In some embodiments, the goserelin is administered on day 1 (±3 days) of each 28-day cycle. The goserelin may be administered on day −3, day −2, day −1, day 1, day 2, or day 3 of the 28-day cycle.

The treatment may be administered for at least about 12 months. The treatment may be administered for at least about 12 months, at least about 18 months, at least about 24 months, at least about 30 months, at least about 36 months, at least about 42 months, at least about 48 months, at least about 54 months, at least about 60 months, or longer. In some embodiments, the ribociclib and letrozole are administered for at least about 48 months (about 4 years), or at least about 60 months (about 5 years). In some embodiments, the treatment is administered for at least 36 months.

An exemplary dose and treatment schedule is shown in Table B1.1.

TABLE B1.1
Exemplary dose and treatment schedule

| | Pharmaceutical form and | | | |
|---|---|---|---|---|
| Trial Treatment | route of administration | Dose | | Frequency and/or Regimen |
| Ribociclib | Tablets for oral use | 400 | mg | Once daily on days 1 to 21 in each |
| (LEE011 200 mg) | | | | 28-day cycle |
| Letrozole 2.5 mg | Tablets for oral use | 2.5 | mg | Once daily given continuously |
| Anastrozole 1 mg | Tablets for oral use | 1 | mg | Once daily given continuously |
| Goserelin 3.6 mg | Implant, in pre-filled | 3.6 | mg | Day 1 ± 3 of each 28-day cycle |
| | syringe and Subcutaneous | | | |
| | use | | | |

In some embodiments, the patient has not received a loading dose prior to treatment. In some embodiments, the patient has received a loading dose prior to treatment. For example, the patient may have received a loading dose of ribociclib (e.g., or a ribociclib salt such as ribociclib succinate) and/or an endocrine therapy (e.g, letrozole or anastrozole).

A loading dose may be an initial dose of a drug that may be given at or prior to the beginning of a course of treatment before dropping down to a different, typically lower dose (e.g., a treatment or maintenance dose). A loading dose may be a single dose or short duration regimen of a compound which is administered to the subject to rapidly increase the blood concentration level of the drug. Suitably, a short duration regimen for use herein will be from: 1 to 14 days; e.g., from 1 to 7 days; e.g., from 1 to 3 days; e.g., for three days; e.g., for two days; e.g., for one day. In some embodiments, the “loading dose” can increase the blood concentration of the drug to a therapeutically effective level. In some embodiments, the “loading dose” can increase the blood concentration of the drug to a therapeutically effective level in conjunction with a treatment dose of the drug. The “loading dose” can be administered once per day, or more than once per day (e.g., up to 4 times per day). In some embodiments, a loading dosage may be used for drug molecules that have a long half-life or slow elimination in vivo.

In some embodiments, the treatment continues until the patient has no remaining signs and/or symptoms of breast cancer. In some embodiments, the treatment continues until the patient has no detectable cancer and/or no signs (e.g., indication) of cancer recurrence. In some embodiments, treatment is reinitiated if a patient exhibits one or more signs of cancer recurrence. In some embodiments, the treatment continues until the cancer recurs. Signs and/or symptoms of cancer and cancer recurrence may be determined by monitoring the patient over months and years following treatment, using tests such as physical examinations, scans and/or imaging of the site where the cancer was (e.g., x-rays, computed tomography, mammography), and blood tests. In an exemplary example, different modalities for follow up may be mammography, clinical examination, whole body or localized MRI and/or CT scans. In accordance with guidelines from the National Comprehensive Cancer Network, a breast cancer patient may have (1) medical history taken and physical exam performed 1-4 times per year as clinically appropriate for 5 years, (2) a mammography every 12 months, and (3) in the absence of clinical signs and symptoms suggestive of recurrent disease, there is no indication for laboratory or imaging studies for metastases screening. When no cancer is detected, patients may be assumed to be treated, but must be observed for recurrence of disease.

In some embodiments, the treatment as disclosed herein may be administered to patients who no longer have detectable signs and/or symptoms of cancer, but who may remain at risk for recurrence of cancer. The treatment may continue in these patients for a period of time, e.g., ranging from at least about 3 months to at least about 10 years, e.g., 3 months, 6 months, 1 year, 2 years, 3 years, 4 years, 5 years, 6 years, 7 years, 8 years, 9 years, or 10 years, or any time period in between. During this time, the patient may be monitored for recurrence of cancer, for example, according to clinical examination, mammography, scans, imaging, sample collection (e.g., biopsy of tissues), and/or blood tests. A patient who does not have detectable signs and/or symptoms of cancer in the period of time may discontinue the treatment described herein. In some embodiments, a patient may continue the treatment for an additional period of time. In some embodiments, a patient whose cancer recurs may discontinue the treatment, for example, to try another treatment option.

### D. PATIENTS

Another aspect of the present disclosure relates to a method of treatment for early breast cancer in an adult patient in need thereof, comprising administering to the patient a treatment comprising a cyclin-dependent kinase (CDK) inhibitor, in combination with endocrine therapy, wherein the adult patient is selected or characterized according to one or more criteria.

The criteria may relate to patient demographics, features of the patient's early breast cancer (e.g., type, stage, grade, hormone-receptor status, etc.), previous treatment, and/or other criteria as described herein.

### I. AGE

In some embodiments, the patient is an adult, e.g., 18 years of age or greater. In some embodiments, the patient is about 18 years to about 45 years of age. In some embodiments, the patient is about 45 years of age to about 54 years of age. In some embodiments, the patient is about 55 years of age to about 64 years of age. In some embodiments, the patient is about 64 years of age or older. The patient may belong to an age category that is less than the median age for a given population of EBC patients, or alternatively, the patient may belong to an aged category that is greater than or equal to the median age for a population of EBC patients.

### II. GENDER AND MENOPAUSAL STATUS

In some embodiments, the patient is a woman. In other embodiments, the patient is a man. For female patients, the patient's menopausal status may be premenopausal. Alternatively, the female patient's menopausal status may be postmenopausal.

A patient with postmenopausal status may be defined as (1) a patient who underwent bilateral oophorectomy; (2) a patient having an age more than or equal to 60 years; (3) a patient having an age of less than 60 years and amenorrhea for 12 or more months (in the absence of chemotherapy, tamoxifen, toremifene or ovarian suppression) and Follicle-stimulating hormone (FSH) and plasma estradiol in the postmenopausal ranges per local normal ranges; or (4) a patient having an age of less than 60 years old and who is taking tamoxifen or toremifene, and has FSH and plasma estradiol level in postmenopausal ranges.

Premenopausal status may be defined for patients who do not meet the criteria for postmenopausal status. For women with premenopausal status, amenorrhea may not be a reliable indicator of menopausal status as ovarian function may still be intact or resume despite anovulation/amenorrhea. For these women with therapy-induced amenorrhea, serial measurements of FSH and/or estradiol per local clinical guidelines may be used for a determination of postmenopausal status.

### III. CHILD BEARING POTENTIAL

In some embodiments, the patient is a woman of childbearing potential (CBP), defined as physiologically capable of becoming pregnant.

Women may be considered of CBP unless they have had 12 months or more than 12 months of natural (spontaneous) amenorrhea with an appropriate clinical profile (i.e. age appropriate, history of vasomotor symptoms) or have had surgical bilateral oophorectomy (with or without hysterectomy), total hysterectomy, or tubal ligation at least six weeks prior to randomization. In the case of oophorectomy alone, the woman may be considered not of CBP after confirmation by hormone level assessment.

In some embodiments, the patient is a woman of CBP who is not pregnant.

In some embodiments, the patient is not a pregnant or breast-feeding (lactating) woman or a woman who plans to become pregnant or breast-feed during the treatment as described herein.

### IV. DIAGNOSIS OF BREAST CANCER

In some embodiments, a patient in need of treatment for early breast cancer is a patient who has been diagnosed with early breast cancer. The patient may not have received a prior treatment for the early breast cancer. The patient may have received a prior treatment for the early breast cancer, such as surgery, chemotherapy, radiation therapy, and/or hormone therapy, but may still be in need of a treatment because the early breast cancer did not respond to the prior treatment. In some embodiments, the patient received a prior treatment for early breast cancer and has a recurrence of the early breast cancer. In some embodiments, the patient received a prior treatment for a cancer, but has been diagnosed with early breast cancer. In some embodiments, the patient is at risk for developing early breast cancer and/or at risk for a recurrence of early breast cancer. The patient at risk may not have detectable signs or symptoms or early breast cancer, but may be at risk due to a previous diagnosis of cancer.

In some embodiments, the patient received a prior treatment for early breast cancer, such as HR+ HER2− stage II or stage III early breast cancer, and has no detectable signs or symptoms of early breast cancer, but remains at risk for recurrence of early breast cancer. Risk factors may relate to the early breast cancer for which the patient received treatment, where a high tumor grade, large tumor size (e.g., greater than 2 mm), and axillary node involvement may correlate with a higher risk for recurrence.

In some embodiments, the patient has a breast cancer that is diagnosed as and/or confirmed (e.g., histologically confirmed) to be a carcinoma of the breast. The carcinoma may have been diagnosed within 18 months prior to starting treatment. The carcinoma may be a unilateral primary invasive adenocarcinoma. The carcinoma may be a multicentric and/or multifocal tumor.

### V. HORMONE RECEPTOR (HR) STATUS AND EXPRESSION OF HUMAN EPIDERMAL GROWTH FACTOR RECEPTOR 2 (HER2)

In some embodiments, the patient has breast cancer that is hormone receptor positive. The breast cancer may be positive for ER and/or PR. The breast cancer may be ER+/PR−, ER−/PR+, or ER+/PR+.

In some embodiments, the patient has a breast cancer that is a HER2− breast cancer. For example, the breast cancer may be defined as a cancer with a negative result from an in situ hybridization test and/or an immunohistochemistry (IHC) status of 0 or 1+. If the breast cancer has an IHC of 2+, a negative in situ hybridization (FISH, CISH, or SISH) may be used to confirm HER2− status.

### VI. TUMOR CHARACTERISTICS

In some embodiments, the patient has a breast cancer comprising a tumor that belongs to one of the following categories:
Anatomic Stage Group III, or
Anatomic Stage Group IIB, or
Anatomic Stage Group IIA that is either:
   N1, or
   N0, with:
      Grade 3, or
      Grade 2.

In some embodiments, patients having a tumor that Anatomic Stage Group IIA, N0, and Grade 2, may have additional measures to categorize the breast cancer. For example, the tumor may have a Ki67 percentage that is about 20% or greater than about 20%. The tumor may have scores in multiparameter tests that indicate a risk for recurrence and/or spread of cancer, such as a breast recurrence score of ≥26 (on a scale of 0-100) in the Oncotype DX® test, a high risk score in Prosigna®/PAM50 (Prediction Analysis of Microarray 50) test, a high risk score MammaPrint® test, or a high risk score EndoPredict® EPclin test.

In some embodiments, the patient has a stage IIA tumor. In some embodiments, the patient has a stage IIB tumor. In some embodiments, the patient has a stage IIIA tumor. In some embodiments, the patient has a stage IIIB tumor. In some embodiments, the patient has a stage IIIC tumor.

In some embodiments, the patient does not have a stage IV tumor, e.g., a breast cancer tumor with distant metastases beyond the lymph nodes.

In some embodiments, the patient has a tumor with a nodal status that is selected from NX, N0, N1, N2, or N3. For example, the patient may have a tumor with NO status. Alternatively, the patient may have a tumor with N1, N2, or N3 status, or a tumor with a status that is any one of N1, N2, or N3.

In some embodiments, the patient has a tumor of category TO, category Tis, T1, T2, T3, or a category T4 tumor. In some embodiments, the patient has a tumor of category T0. The patient may have a tumor of category T1, T2, or T3 status, or a tumor that is any one of T1, T2, or T3. In some embodiments, the patient has a tumor of category T4.

In some embodiments, the patient has a tumor with an M stage of M0.

In some embodiments, the patient has a tumor of histological grade GX, G1, G2, or G3 (e.g., Grade X, Grade 1, Grade 2, or Grade 3). The patient may have a tumor of histological grade of Grade 1. The patient may have a tumor of histological Grade 2. The patient may have a tumor of histological Grade 3.

In certain embodiments, the patient has a tumor with a Ki67 status of less than or equal to 20%. The Ki67 category may be less than or equal to 14%, or the Ki67 category may be greater than 14%. Alternatively, the patient may have a tumor with a Ki67 status of more than 20%. Ki67 may be measured in archival samples of resected tumors from the patient. Ki67 is a proliferation marker in normal and human cell populations, which may be used as a clinical marker for breast cancers and a predictive marker for success with endocrine therapy.

In some embodiments, the patient has a tumor that has been evaluated by a genomic test, such as one or more of an Endopredict® test, a Mammaprint® test, an Oncotype DX® test, a Prosigna/PAM50® test, and a Breast Cancer Index® test. The patient may have a risk score from one or more tests that indicates a high risk for cancer recurrence and/or metastasis.

In some embodiments, the tumor has an EndoPredict EPclin® risk score that indicates high risk. In some embodiments, the tumor has an Mammaprint® test result that indicates high risk. In some embodiments, the tumor has an Prosigna/PAM50® test result that indicates high risk. In some embodiments, the tumor has a Oncotype DX Breast Recurrence Score that is a greater than or equal to about 26.

Measures such as histopathological grade, T stage, N stage, M stage, and Ki67 may be determined at the time of initial diagnosis or on a surgical specimen.

In some embodiments, the patient has a tumor of a ductal subtype. The patient may have a tumor of a lobular subtype. Alternatively, the patient may have a tumor of a subtype that is neither ductal nor lobular.

In some embodiments, the predominant histology of the tumor is selected from invasive ductal carcinoma, no otherwise specified (NOS), invasive lobular, carcinoma medullary, mucinous, papillary, tubular, ductal carcinoma in situ, and lobular carcinoma in situ.

In some embodiments, the patient has a tumor comprising a HER2 ISH result comprising an IHC score of 0, 1+, 2+, or 3+. The HER2 ISH result may be obtained prior to surgery or from a surgical specimen.

In some embodiments, the patient has a positive hormone receptor status. For example, a positive ER status and/or a positive PR status. The positive status may be obtained prior to surgery or from a surgical specimen.

In certain embodiments, the patient has a tumor with no metastasis in sentinel lymph nodes (e.g., the patient is considered as pN0). The patient may have only micrometastasis in sentinel lymph nodes (e.g., the patient is considered as pN1mi). Patients may have tumors that are T1-2 and no clinically-evident nodes prior to surgery, no neoadjuvant chemotherapy, at least one macrometastasis in 1 or 2 sentinel lymph nodes, and/or no matted nodes or gross extranodal disease at the time of sentinel dissection (e.g., patient is considered as pN1).

In some embodiments, staging of breast cancer is performed by lymph node dissection, such as axillary lymph node dissection (ALND). In some embodiments, staging is performed by sentinel lymph node (SLN) dissection.

In some embodiments, the tumor is located in only the right breast and not the left breast.

Alternatively, the tumor may be located only in the left breast and not in the right breast. In some embodiments, the tumor is bilateral, e.g., comprising cancer cells located in both the right breast and the left breast.

### VII. BODY MASS INDEX

In some embodiments, the patient has a body mass index (BMI) that is more than 25 or equal to 25. In some embodiments, the patient has a BMI that is less than 25.

### VIII. PRIOR TREATMENT FOR CANCER

In some embodiments, the patient has received treatment (which may also be called therapy) for the breast cancer prior to undergoing the methods described herein.

The prior treatment may be a primary treatment (also called a primary therapy, main treatment or therapy, induction treatment or therapy, or first-line treatment or therapy) for breast cancer. In some embodiments, the patient has completed a primary treatment for breast cancer before the treatment as described herein. Accordingly, the treatment described herein may be an adjuvant treatment following a prior treatment.

In some embodiments, the prior treatment is not a primary treatment, but follows a primary treatment or other treatments. In these examples, the treatment described herein may be an adjuvant treatment to a prior treatment, wherein the prior treatment itself followed one or more additional prior treatments for breast cancer.

The prior treatment may itself be an adjuvant treatment for a primary treatment. The prior treatment may be a neoadjuvant treatment for a primary treatment. The prior treatment may be a neoadjuvant treatment for the treatment disclosed herein. In some embodiments, the patient completes the prior treatment (e.g., adjuvant and/or neoadjuvant treatment) before the treatment disclosed herein.

In a further exemplary example, the prior treatment is a neoadjvant treatment for a primary treatment. For example, the prior treatment may be administered to shrink a tumor before the primary treatment. In some embodiments, both the prior treatment and the primary treatment for which the prior treatment is a neoadjuvant treatment are completed before the treatment described herein.

In some embodiments, the prior treatment is neoadjuvant treatment for the treatment described herein.

A prior treatment may be radiotherapy (also “radiation therapy”), surgical treatment, or chemotherapy.

In some embodiments, the prior treatment is radiotherapy. Radiotherapy may be located at one or more of the following: breast, chest wall, axillary lymph node, supraclavicular lymph node, and internal mammary lymph node.

In some embodiments, the patient has undergone a surgical treatment. Accordingly, a prior treatment may be surgical resection of the cancer cells (e.g., tumor). For example, the patient may have had one or more of a mastectomy, a breast conserving surgery, an axillary lymph node dissection, or a sentinel lymph node biopsy. In some embodiments, the patient has had a mastectomy. In some embodiments, the patient has had a lumpectomy. In some embodiments, an entire organ or even the surrounding structures may be removed in order to achieve the necessary cancer (e.g., tumor) resection. A surrounding amount of normal, healthy tissue (called “surgical margin”) may be removed to increase the success of surgery. Accordingly, in some embodiments, the prior treatment comprises complete tumor resection. The cancer may be removed completely, with final surgical specimen microscope margins free from tumor.

In some embodiments, following surgical resection, the cancer may be R0 (for example, the surgical margin is microscopically negative for residual tumor).

In some embodiments, the patient is administered the treatment as described herein as an adjuvant treatment after a prior treatment of surgery. For example, the treatment described herein may start after surgical resection where the tumor was removed completely, with final surgical specimen microscopic margins free from tumor.

The prior treatment may be chemotherapy. In some embodiments, the patient has received a prior treatment that is a chemotherapy. The chemotherapy may be a neoadjuvant chemotherapy. For example, the neoadjuvant chemotherapy may have been administered before another treatment such as a surgical procedure, radiation therapy, or administration of another medication. The chemotherapy may be an adjuvant chemotherapy. For example, the prior treatment may be a chemotherapy that was administered after another treatment such as a surgical procedure, radiation therapy, or administration of another medication. The chemotherapy may be a neo-/adjuvant chemotherapy. In these exemplary examples, the prior treatment (e.g., neoadjuvant chemotherapy and/or adjuvant chemotherapy) occurs before the treatment according to the methods described herein.

In some embodiments, the patient has received a prior anti-neoplastic chemotherapy, for example, an anthracylcine or a taxane.

In some embodiments, the patient has received endocrine therapy (ET). The ET may be, for example, a neoadjuvant and/or adjuvant ET. The patient may have received ET within 12 months of beginning the present methods. In some embodiments, the patient has received tamoxifen or toremifene as an adjuvant ET, wherein the patient undergoes a washout period of 5 half-lives (e.g., 35 days) prior to beginning the present methods. The patient may receive aromatase inhibitors during the washout period.

In some embodiments, the patient has received an endocrine therapy selected from an aromatase inhibitor, an anti-estrogens, a gonadotropin-releasing hormone analogues, or and a biologic/targeted therapy.

In some embodiments, the patient has undergone previous treatment, but the breast cancer did not respond to treatment, for example, due to resistance of the breast cancer cells to a drug. In some embodiments, the patient has undergone previous treatment, but the breast cancer has recurred (or “relapsed”).

In some embodiments, the patient has received treatment (which may also be called therapy) for the breast cancer prior to undergoing the treatment described herein, and is in remission.

The patient may be in remission from early breast cancer, for example, HR+/HER2− early breast cancer. The HR+/HER2− early breast cancer may be stage II or stage III cancer.

A cancer may be in remission. A patient who had cancer may be in remission. In both cases, remission may refer to a decrease in or a disappearance of signs of cancer in a patient, following a treatment for cancer. Remission may be partial remission, in which a cancer (e.g., cancer cells and/or a tumor) has decreased in size or in the extent of spread in the body. For example, partial remission may reflect a 50% reduction in measurable parameters for cancer such as growth, size, or spread of cancer cells and/or a tumor. Remission may be complete remission, where signs of cancer (e.g., cancer cells and/or a tumor) are no longer detectable. In complete remission, there may be no evidence of disease.

In complete remission, there may be no detectable signs or symptoms of cancer.

Remission may be related to the period of time since the patient had detectable signs or symptoms of cancer. For example, after about 1 month without detectable signs or symptoms of cancer, the patient may be described as being in remission. In some embodiments, the period of time is about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, about 36 months, or longer. In some embodiments, the period of time is about 4 years, about 5 years, about 6 years, about 7 years, about 8 years, about 9 years, about 10 years, or longer.

Accordingly, the treatment as described herein may be administered to patients who are in remission. In some embodiments, the patients are in complete remission. In some embodiments, the patients are in partial remission.

In some embodiments, the patient is in full remission at the time that the treatment described herein begins. For example, the patient may not have detectable cancer cells and/or a tumor.

The patient may have been diagnosed with early breast cancer at a previous time and may have been treated with a prior treatment, with the result that cancer is no longer detectable.

Accordingly, a patient who does not have symptoms of cancer may be administered with the treatment described herein. In some embodiments, the patient may have been diagnosed with a HR+/HER2− early breast cancer and treated with surgery to remove the cancer, and HR+/HER2− early breast cancer may no longer be detectable. In some embodiments, the patient may be at risk for developing HR+/HER2− breast cancer based on the presence of a HR+/HER2− breast cancer diagnosed previously. For example, the patient may be at risk for recurrence of HR+/HER2− breast cancer based on a previous diagnosis of HR+/HER2− early breast cancer.

In some embodiments, the patient is in partial remission and shows detectable signs of cancer at the time that the treatment described herein begins. In some embodiments, the patient was diagnosed with a HR+/HER2− early breast cancer and treated with a prior treatment, but the cancer was not fully removed and/or the cancer recurred. In some embodiments, the patient in partial remission may be at risk for regrowth and/or spread of HR+/HER2− breast cancer based on a previous diagnosis of HR+/HER2− early breast cancer.

In some embodiments, the treatment described herein prevents growth of HR+/HER2− breast cancer cells in the patient, for example, a patient who is in remission (e.g., in full remission or in partial remission).

In some embodiments, the treatment described herein maintains the patient in remission (e.g., in full remission or in partial remission). The treatment may maintain the remission for at least about 3 months, at least about 6 months, at least about 12 months, at least about 18 months, at least about 24 months, at least about 30 months, at least about 36 months, at least about 42 months, at least about 48 months, at least about 54 months, at least about 60 months, at least about 66 months, at least about 72 months, at least about 78 months, at least about 84 months, at least about 90 months, at least about 96 months, at least about 102 months, at least about 108 months, at least about 114 months, at least about 120 months, or longer.

In some embodiments, the treatment reduces recurrence of breast cancer (e.g., an early breast cancer such as a HR+/HER2− early breast cancer). The treatment may reduce recurrence for at least about 3 months, at least about 6 months, at least about 12 months, at least about 18 months, at least about 24 months, at least about 30 months, at least about 36 months, at least about 42 months, at least about 48 months, at least about 54 months, at least about 60 months, at least about 66 months, at least about 72 months, at least about 78 months, at least about 84 months, at least about 90 months, at least about 96 months, at least about 102 months, at least about 108 months, at least about 114 months, at least about 120 months, or longer.

Recurrence of breast cancer may be an ipsilateral breast tumor recurrence (IBTR), defined herein as the reappearance of breast tumor in the same (i.e., ipsilateral) breast or chest wall.

Recurrence of breast cancer may be a contralateral breast cancer, defined herein as a primary breast cancer that occurs in the opposite (i.e., contralateral) breast at least three months after the first breast cancer.

Thus, in some embodiments, the method described herein comprises maintaining in remission an adult patient who had previously been diagnosed with HR+/HER2− stage II or stage III early breast cancer, comprising administering to the patient a treatment comprising a dose of ribociclib, a freebase form thereof or a pharmaceutically acceptable salt thereof, in combination with an aromatase inhibitor.

In some embodiments, the treatment described herein is an adjuvant therapy (also called an adjuvant treatment) to a prior treatment. The adjuvant therapy may follow any prior treatment, for example, the prior treatments described herein. The adjuvant therapy may follow more than one prior treatment. For example, one prior treatment may have been a neoadjuvant treatment preceding a primary treatment (e.g., first-line treatment) such as surgery with subsequent chemotherapy, radiation therapy and/or endocrine therapy, after which the treatment described herein is administered as an adjuvant therapy.

Accordingly, in some embodiments, the method described herein comprises preventing breast cancer recurrence in an adult patient, comprising providing adjuvant treatment to a patient who has received a prior treatment for HR+/HER2− stage II or III early breast cancer, wherein the adjuvant treatment comprises administering to the patient 400 mg ribociclib or a pharmaceutically acceptable salt thereof on days 1 to 21 of a 28-day cycle for at least 36 months, in combination with an aromatase inhibitor as defined herein on every day of the 28-day cycle.

Accordingly, in some embodiments, the method described herein comprises maintaining remission an adult patient who had previously been diagnosed with an HR+/HER2− stage II or stage III early breast cancer, comprising providing adjuvant treatment to a patient who has received a prior treatment for HR+/HER2− stage II or III early breast cancer, wherein the adjuvant treatment comprises administering to the patient 400 mg ribociclib or a pharmaceutically acceptable salt thereof on days 1 to 21 of a 28-day cycle for at least 36 months, in combination with an aromatase inhibitor as defined herein administered on every day of the 28-day cycle.

In some embodiments, the adjuvant treatment does not include administration of a 600 mg/day dose of ribocicilib.

A further aspect of the present disclosure relates to a method of treating an adult patient who has been diagnosed with HR+/HER2− stage II or stage III early breast cancer, comprising administering to the patient an adjuvant therapy comprising (i) a dose of ribociclib ranging from 150 mg/day to 450 mg/day administered on days 1-21 of a 28-day cycle, and (ii) an aromatase inhibitor administered on every day of the 28-day cycle, wherein the method results in an improvement in the patient in one or more of their overall survival (OS), distant disease-free survival (DDFS), or recurrence free survival (RFS).

In some embodiments, the early breast cancer may no longer be in stage II or stage III when the treatment described herein begins. For example, the patient may have received at least one prior treatment, for which the treatment described herein is an adjuvant treatment.

Another aspect of the present disclosure relates to a method for improving one or more of overall survival (OS), distant disease-free survival (DDFS), or recurrence free survival (RFS) in a patient who has been diagnosed with HR+/HER2− stage II or stage III early breast cancer, comprising administering to the patient an adjuvant treatment comprising (i) a dose of ribociclib ranging from 150 mg/day to 450 mg/day administered on days 1-21 of a 28-day cycle, (ii) an aromatase inhibitor administered on every day of the 28-day cycle.

A further aspect of the present disclosure relates to ribociclib for use in a method of improving one or more of overall survival (OS), distant disease-free survival (DDFS), or recurrence free survival (RFS) in an adult patient who has been diagnosed with HR+/HER2− stage II or stage III early breast cancer, wherein the method comprises administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.

The methods described herein may prevent local and/or regional invasive recurrence of early breast cancer.

Accordingly, an aspect of the present disclosure relates to a method for reducing the risk of local or regional invasive recurrence of early breast cancer in an adult patient who has been diagnosed with HR+/HER2− stage II or stage III early breast cancer, comprising administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.

An aspect of the present disclosure relates to ribociclib for use in a method of reducing the risk of local or regional invasive recurrence of early breast cancer in an adult patient who has been diagnosed with HR+/HER2− stage II or stage III early breast cancer, wherein the method comprises administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle

The method described herein may prevent recurrence of cancer in tissues outside of the breast, such as bone, liver, and lung or pleura.

Thus, a further aspect of the present disclosure relates to a method for reducing the risk of invasive recurrence of early breast cancer in one or more of the bone, liver, and lung or pleura of an adult patient, who has been diagnosed with HR+/HER2− stage II or stage III early breast cancer, comprising administering to the patient an adjuvant treatment comprising (i) a dose of ribociclib ranging from 150 mg/day to 450 mg/day on days 1-21 of a 28-day cycle, and (ii) an aromatase inhibitor administered on every day of the 28-day cycle.

A further aspect of the present disclosure relates to ribociclib for use in a method of reducing the risk of invasive recurrence of early breast cancer in one or more of the bone, liver, and lung or pleura of an adult patient who has been diagnosed with HR+/HER2− stage II or stage III early breast cancer, wherein the method comprises administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.

Treatment may be administered irrespective of the nodal status of the breast cancer.

Another aspect of the present disclosure relates to a method for reducing the risk of recurrence of early breast cancer in an adult patient who has been diagnosed with HR+/HER2− stage II or stage III early breast cancer, comprising administering an adjuvant treatment comprising (i) a dose of ribociclib ranging from 150 mg/day to 450 mg/day on days 1-21 of a 28-day cycle and (ii) an aromatase inhibitor administered on every day of the 28-day cycle, wherein the treatment is administered irrespective of the nodal status of the breast cancer.

An aspect of the present disclosure relates to ribociclib for use in a method of reducing the risk of recurrence of early breast cancer in an adult patient who has been diagnosed with HR+/HER2− stage II or stage III early breast cancer, comprising administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle, wherein the treatment is administered irrespective of the nodal status of the breast cancer.

For example, the breast cancer may have a nodal status of N0, N1, N2 or N3. The breast cancer may have a nodal status of NO.

In some embodiments, the early breast cancer is stage II, for example stage IIA. In some embodiments, the early breast cancer is stage III, for example stage IIIB or IIIC.

In certain embodiments, the breast cancer is of the ductal subtype.

In some embodiments, the patient is Asian.

An adjuvant treatment may be needed after a patient has received at least one prior treatment for breast cancer. In some embodiments, the method described herein is an adjuvant treatment because the patient has received at least one prior treatment for breast cancer selected from the group consisting of surgery, chemotherapy, and radiation therapy.

In some embodiments, the patient has not had a mastectomy.

In some embodiments, the dose of ribociclib is 400 mg/day. The ribociclib may be administered in the form of a salt, preferably as ribociclib succinate.

In some embodiments, the aromatase inhibitor is letrozole or anastrozole. For example, the aromatase inhibitor may be letrozole, preferably administered in a dose of 2.5 mg/day. The aromatase inhibitor may be anastrozole, preferably administered in a dose of 1 mg/day.

In some embodiments, the dose of ribociclib is 400 mg/day, the ribociclib is administered in the form of ribociclib succinate, and the aromatase inhibitor is letrozole or anastrozole, preferably 2.5 mg/day of letrozole or 1 mg/day anastrozole.

In some embodiments, the improvement in OS, DDFS and/or RFS, or the reduction in the risk of recurrence of the breast cancer, are achieved in the patient for at least 36 months.

In some embodiments, the patient has not previously received a CDK4/6 inhibitor.

In some embodiments, the patient has not received tamoxifen, raloxifene, or aromatase inhibitors for reduction in risk (“chemoprevention”) of breast cancer and/or treatment for osteoporosis within the last 2 years prior to undergoing the present methods.

In some embodiments, the patient has not received treatment with anthracyclines at cumulative doses of 450 mg/m² or more for doxorubicin, or 900 mg/m² or more for epirubicin prior to undergoing the present methods.

In some embodiments, the patient does not have a known hypersensitivity to any of the excipients of ribociclib and/or ET (e.g., the patient does not have rare hereditary problems of galactose intolerance, the Lapp lactase deficiency, glucose-galactose malabsorption, or a soy allergy).

In some embodiments, the patient does not receive other anti-neoplastic therapy, except for adjuvant ET as described herein.

In some embodiments, the patient has not undergone major surgery, chemotherapy, or radiotherapy within 14 days of undergoing the present methods.

In some embodiments, the patient has not recovered from clinical and laboratory acute toxicities related to prior anti-cancer therapies to a NCI CTCAE (National Cancer Institute Common Terminology Criteria for Adverse Events) version 4.03 Grade ≤1 at day of randomization.

Exceptions are patients with any grade of alopecia, amenorrhea, grade 2 neuropathy, or other toxicities not considered a safety risk for the patient.

In some embodiments, the patient does not have a concurrent invasive malignancy or a prior invasive malignancy whose treatment was completed within 2 years before randomization.

Patients may have been adequately treated for basal or squamous cell skin carcinoma or had a curatively resected cervical cancer in situ.

In some embodiments, the patient does not have a known history of human immunodeficiency virus (HIV) infection.

In some embodiments, the patient does not have a known active hepatitis B virus (HBV) or hepatitis C virus (HCV) infection (testing is not mandatory, unless required by local regulation).

In some embodiments, the patient does not have clinically significant, uncontrolled heart disease and/or cardiac repolarization abnormality, including any of the following:
History of documented myocardial infarction (MI), angina pectoris, symptomatic pericarditis, or coronary artery bypass graft within 6 months prior to undergoing the present methods.
Documented cardiomyopathy.
Left Ventricular Ejection Fraction (LVEF)<50% as determined by Multiple Gated acquisition (MUGA) scan or echocardiogram (ECHO).
Long QT syndrome or family history of idiopathic sudden death or congenital long QT syndrome, or any of the following:
   Risk factors for Torsades de Pointes (TdP) including uncorrected hypocalcemia, hypokalemia or hypomagnesemia, history of cardiac failure, or history of clinically significant/symptomatic bradycardia.
   Concomitant medication(s) with a known risk to prolong the QT interval and/or known to cause TdP that cannot be discontinued or replaced by safe alternative medication (e.g. within 5 half-lives or 7 days prior to undergoing the present methods).
   Inability to determine the QTcF interval.
Clinically significant cardiac arrhythmias (e.g. ventricular tachycardia), complete left bundle branch block, and/or high-grade Atrioventricular (AV) block (e.g. bifascicular block, Mobitz type II and third degree AV block).
Uncontrolled arterial hypertension with systolic blood pressure >160 mmHg.

In some embodiments, the patient has not received any of the following substances within 7 days before undergoing the present methods:
Concomitant medications, herbal supplements, and/or fruits (e.g. grapefruit, pummellos, starfruit, Seville oranges) and their juices that are known as strong inhibitors or inducers of CYP3A4/5.
Medications that have a narrow therapeutic window and are predominantly metabolized through CYP3A4/5.

In some embodiments, the patient does not to consume grapefruit (or grapefruit juice). In some embodiments, the patient does not consume pummellos, starfruit, and seville oranges (and their juices). In certain embodiments, the patient is not concomitantly using any of boceprevir, clarithromycin, conivaptan, indinavir, itraconazole, ketoconazole, lopinavir, ritonavir, nefazodone, nelfinavir, posaconazole, ritonavir, saquinavir, and voriconazole.

In some embodiments, the patient is not currently receiving or has received systemic corticosteroids ≤2 weeks prior to undergoing the present methods, or has not fully recovered from side effects of such treatment. In some embodiments, a patient may use corticosteroids for the following: a short duration (<5 days) of systemic corticosteroids; any duration of topical applications (e.g. for rash), inhaled sprays (e.g. for obstructive airways diseases), eye drops or local injections (e.g. intra-articular).

In some embodiments, the patient does not have an impairment of GI function or GI disease that may significantly alter the absorption of the oral trial treatments (e.g. uncontrolled ulcerative diseases, uncontrolled nausea, vomiting or diarrhea, malabsorption syndrome, or small bowel resection).

In some embodiments, the patient is not suffering from a concurrent severe and/or uncontrolled medical condition that may cause unacceptable safety risks, contraindicate patient treatment or compromise compliance with the methods (e.g. chronic pancreatitis, chronic active hepatitis, liver cirrhosis or any other significant liver disease, active untreated or uncontrolled fungal, bacterial or viral infections, active infection requiring systemic anti-bacterial therapy, etc.) or limit life expectancy to ≤5 years.

In some embodiments, the patient has not been involved in treatment involving investigational drug(s) within 30 days prior to undergoing the present methods or within 5 half-lives of the investigational drug(s) (whichever is longer).

### IX. ADDITIONAL CRITERIA

In some embodiments, the patient has an Eastern Cooperative Oncology Group (ECOG) performance status of 0 or 1.

In some embodiments, the patient has bone marrow and organ function as defined by the following values:
Absolute neutrophil count (ANC)≥1.5×10⁹/L.
Platelets ≥100×10⁹/L.
Hemoglobin ≥9.0 g/dL.
Estimated glomerular filtration rate (eGFR) ≥30 mL/min/1.73 m² according to the Modification of Diet in Renal Disease (MDRD) formula.

$$Alanine transaminase ( ALT ) < 2.5 \times Upper Limit Normal ( ULN ) .$$

$$Aspartate transaminase ( AST ) < 2.5 \times ULN .$$

Total serum bilirubin <ULN; or total bilirubin ≤3.0×ULN or direct bilirubin ≤1.5×ULN in patients with well documented Gilbert's Syndrome.
International normalized ratio (INR) ≤1.5 (unless the patient is receiving anticoagulants and the INR is within the therapeutic range of intended use for that anticoagulant within 7 days prior to randomization).
Laboratory values within normal limits or corrected to within normal limits with supplements (the local laboratory value should be documented within normal limits after the correction) for the following:
   Potassium
   Magnesium
   Total Calcium (corrected for serum albumin)

In some embodiments, the patient has standard 12-lead ECG values of QTcF interval (QT interval using Fridericia's correction) at screening <450 msec, and a resting heart rate 50-90 beats per minute (determined from the ECG).

### X. REGION AND RACE/ETHNICITY

In some embodiments, the patient is located in North America. The patient may be located in Europe, for example, Western Europe. The patient may be located in Oceania, for example, in Australia. In some embodiments, the patient is located in Asia. In some embodiments, the patient is located in Africa. In some embodiments, the patient is located in Latin America.

In some embodiments, the patient may be American Indian. In some embodiments, the patient may be Alaska Native. In some embodiments, the patient may be Asian. In some embodiments, the patient may be Black or African American. In some embodiments, the patient may be Native Hawaiian or Other Pacific Islander. In some embodiments, the patient may be White. In some embodiments, the patient may be a mixed race. In some embodiments, the patient may be non-Asian. In some embodiments, the patient may be Hispanic or Latino. In some embodiments, the patient is not Hispanic or Latino.

### E. EFFICACY READOUTS

An aspect of the present disclosure relates to a method of treatment for early breast cancer in an adult patient in need thereof, comprising administering to the patient a treatment comprising a cyclin-dependent kinase (CDK) inhibitor in combination with endocrine therapy (ET), wherein the treatment is characterized by an improvement in the patient's condition, as compared to a patient not receiving the treatment or as compared to the patient prior to treatment. In some embodiments, treatment regimens, e.g., the selection of a dose of ribociclib and/or aromatase inhibitor is chosen to achieve certain improvement in the patient's condition, as compared to a patient not receiving the treatment or as compared to the patient prior to treatment.

In some embodiments, a patient not receiving the treatment described herein may comprise a patient who does not receive ribociclib (or a pharmaceutically acceptable salt thereof, such as ribociclib succinate). In some embodiments, the patient not receiving the treatment described herein may comprise a patient who does not receive a combination of ribociclib (or a pharmaceutically acceptable salt thereof, such as ribociclib succinate) and endocrine therapy. For example, the patient may receive only endocrine therapy alone, without ribociclib (or a pharmaceutically acceptable salt thereof, such as ribociclib succinate). The patient may receive letrozole alone without ribociclib (or a pharmaceutically acceptable salt thereof, such as ribociclib succinate). The patient may receive anastrozole alone without ribociclib (or a pharmaceutically acceptable salt thereof, such as ribociclib succinate). In some embodiments, the patient not receiving the treatment described herein may receive another cancer treatment, wherein the other cancer treatment is not a combination of ribociclib (or a pharmaceutically acceptable salt thereof, such as ribociclib succinate) and endocrine therapy. In some embodiments, the patient not receiving the treatment described herein may be a patient who does not receive any cancer treatment.

The improvement in the patient's condition may relate to a reduction in signs and/or symptoms of breast cancer. For example, the improvement may comprise a reduction in breast cancer progression and/or breast cancer recurrence. The improvement may comprise prevention of breast cancer progression, early breast cancer recurrence, and/or death from the breast cancer. The improvement may comprise a reduction in the risk for cancer progression, cancer recurrence, and/or risk of (or avoidance of) death from the cancer.

In some embodiments, the improvement in the patient's condition is the maintenance of the existing signs and/or symptoms of breast cancer. For example, after treatment, the breast cancer may have about the same characteristics as before treatment, but no further progression of the breast cancer or worsening of the characteristics.

In some embodiments, the improvement in the patient's condition is an improvement as compared to a patient not receiving the treatment. In some embodiments, the patient not receiving the treatment receives another treatment for early breast cancer. In some embodiments, the patient not receiving the treatment receives only endocrine therapy alone.

In some embodiments, the improvement in the patient's condition may comprise one or more of a reduction in tumor size, prevention of spread of the tumor, prevention or reduction in recurrence of breast cancer, prevention of a second primary malignancy, and increased survival.

For the example, the improvement in the patient's condition may comprises an absence of (or a reduction in) locoregional relapse, distant relapse, ipsilateral and contralateral invasive breast cancers and second primary non-breast invasive cancer.

In some embodiments, the improvement in the patient's condition relates to measurements such as disease-free survival (DFS), invasive disease-free survival (iDFS), overall survival (OS), distant disease-free survival (DDFS), recurrence-free survival (RFS), and loco-regional recurrence-free survival (LLRFS).

Disease-free survival (DFS) may relate to the length of time that a patient survives without local recurrence, contralateral recurrence, distant disease, secondary cancers, or new primary cancers. DFS may be defined as the time from a first date (e.g., a date when the treatment for cancer begins or, alternatively, a date when the treatment for cancer ends) to a date that the patient shows recurrence of a cancer or death.

Invasive disease-free survival (iDFS) may be defined as DFS, but excluding ductal carcinoma in situ as a recurrence event.

Overall survival (OS) may relate to the length of time that a patient survives, which can include death from any cause, regardless of whether the breast cancer has recurred or spread. OS may be defined as the time from a first date (e.g., a date when the treatment for cancer begins or, alternatively, a date when the treatment for cancer ends) to a date of death due to any cause.

Distant disease-free survival (DDFS) may relate to the length of time during which a patient remains free of the cancer recurring at a distant site, e.g., not in the original breast. DDFS may be defined as the time from a first date (e.g., a date when the treatment for cancer begins or, alternatively, a date when the treatment for cancer ends) to a date of first event of distant recurrence, death (any cause), or second primary non-breast invasive cancer (excluding basal and squamous cell carcinomas of the skin).

Recurrence free survival (RFS) may relate to any recurrence (local, regional, or distant) of the original breast cancer, but does not include new cancer(s). RFS may be defined as the time from a first date (e.g., a date when the treatment for cancer begins or, alternatively, a date when the treatment for cancer ends) to a date of first event of local invasive breast recurrence, regional invasive recurrence, distant recurrence, or death (any cause).

Loco-regional recurrence-free survival (LRRFS) may be defined as the time from a first date (e.g., a date when the treatment for cancer begins or, alternatively, a date when the treatment for cancer ends) to a date of first event of local (ipsilateral) invasive breast recurrence, regional invasive recurrence, or death due to any cause.

Accordingly, in some embodiments, the present methods improve one or more of DFS, iDFS, OS, DDFS, RFS, and LLRFS in a patient who receives the treatment as compared to a patient who does not receive the treatment. For example, the improvement in the patient's condition may be a prolongation of the time period between a first date (e.g., a date when the treatment for cancer begins or, alternatively, a date when the treatment for cancer ends) and a date of occurrence of an event as defined in any of DFS, iDFS, OS, DDFS, RFS, and LLRFS.

In some embodiments, the improvement in the patient's condition is a decrease in the incidence (or decrease in the time until occurrence) of one or more of invasive ipsilateral breast tumor (IBTR) recurrence, local-regional invasive recurrence, distant recurrence (e.g., presence of metastasis), death from breast cancer, death from a non-breast cancer cause, death from an unknown cause, presence of invasive contralateral breast cancer, and presence of a second primary invasive cancer.

For example, the treatment may decrease the incidence (or the time until occurrence) of death from breast cancer, death from a non-breast cancer cause, and death from an unknown cause.

In some embodiments, the improvement in the patient's condition is a decrease in the incidence (or decrease in the time until occurrence) of distant recurrence (e.g., presence of metastasis), death from breast cancer, death from a non-breast cancer cause, death from an unknown cause, and presence of a second primary invasive cancer.

In some embodiments, the improvement in the patient's condition is a decrease in the incidence (or decrease in the time until occurrence) of invasive ipsilateral breast tumor (IBTR) recurrence, local-regional invasive recurrence, distant recurrence (e.g., presence of metastasis), death from breast cancer, death from a non-breast cancer cause, and death from an unknown cause.

In some embodiments, the improvement in the patient's condition is a decrease in the incidence (or decrease in the time until occurrence) of one or more of invasive ipsilateral breast tumor (IBTR) recurrence, local-regional invasive recurrence, death from breast cancer, death from a non-breast cancer cause, and death from an unknown cause.

Where the improvement in the patient's condition is a reduction in the risk of invasive disease or other events related to cancer, the risk may be determined using different measures. In some embodiments, the treatment reduces the risk of invasive disease corresponding to a hazard ratio of less than about 1 when the risk is calculated relative to patients not receiving the treatment. For example, the hazard ratio may be less than about 0.9, about 0.8, about 0.7, about 0.6, about 0.5, about 0.4, about 0.3, about 0.2, or about 0.1) For example, the hazard ratio for DFS may be calculated using model such as an unstratified and covariate unadjusted Cox model or a stratified and covariate adjusted Cox model, as described in Appendix A.

In some embodiments, the hazard ratio may be less than or equal to about 0.78. The hazard ratio may be less than or equal to about 0.72.

In some embodiments, the patient has HR+/HER2− stage III early breast cancer and the treatment reduces the risk of invasive disease corresponding to a hazard ratio of less than or equal to about 0.74 when the risk is calculated relative to patients not receiving the treatment.

The hazard ratio may be less than or equal to about 0.76. In some embodiments, the treatment yields at least a 25% reduction in the risk of invasive disease corresponding to a hazard ratio of 0.75 when the risk is calculated relative to patients not receiving the treatment.

In some embodiments, the treatment reduces the risk of invasive disease to a similar level for patient subgroups comprising patients with stage II early breast cancer, stage III early breast cancer, patients who are premenopausal women or men, and patients who are postmenopausal women.

In some embodiments, treatment as disclosed herein may achieve the following hazard ratios for particular patients with HR+/HER2− early breast cancer.

In some embodiments, the patient is a woman and the treatment may reduce the risk of invasive disease corresponding to a hazard ratio of less than about 0.76 when the risk is calculated relative to patients not receiving the treatment.

In some embodiments, the patient is a pre-menopausal woman or a man and the treatment may reduce the risk of invasive disease corresponding to a hazard ratio of less than about 0.72 when the risk is calculated relative to patients not receiving the treatment.

In some embodiments, the patient is a post-menopausal woman and the treatment may reduce the risk of invasive disease corresponding to a hazard ratio of less than about 0.78 when the risk is calculated relative to patients not receiving the treatment.

In some embodiments, the patient received a prior neo-/adjuvant chemotherapy and the treatment may reduce the risk of invasive disease corresponding to a hazard ratio of less than about 0.73 when the risk is calculated relative to patients not receiving the treatment.

In some embodiments, the patient is located in North America, Western Europe or Oceania, and the treatment may reduce the risk of invasive disease corresponding to a hazard ratio of less than about 0.76 when the risk is calculated relative to patients not receiving the treatment.

In some embodiments, the patient is not located in North America, Western Europe or Oceania, and the treatment may reduce the risk of invasive disease corresponding to a hazard ratio of less than about 0.76 when the risk is calculated relative to patients not receiving the treatment.

In some embodiments, the patient has stage II cancer and the treatment may reduce the risk of invasive disease corresponding to a hazard ratio of less than about 0.76 when the risk is calculated relative to patients not receiving the treatment.

In some embodiments, the patient has stage III cancer and the treatment may reduce the risk of invasive disease corresponding to a hazard ratio of less than about 0.74 when the risk is calculated relative to patients not receiving the treatment.

In some embodiments, the patient has stage IIA cancer and the treatment may reduce the risk of invasive disease corresponding to a hazard ratio of less than about 0.5 when the risk is calculated relative to patients not receiving the treatment.

In some embodiments, the patient has stage IIB cancer and the treatment may reduce the risk of invasive disease corresponding to a hazard ratio of less than about 0.93 when the risk is calculated relative to patients not receiving the treatment.

In some embodiments, the patient has stage IIIIA cancer and the treatment may reduce the risk of invasive disease corresponding to a hazard ratio of less than about 0.79 when the risk is calculated relative to patients not receiving the treatment.

In some embodiments, the patient has stage IIIB cancer and the treatment may reduce the risk of invasive disease corresponding to a hazard ratio of less than about 0.68 when the risk is calculated relative to patients not receiving the treatment.

In some embodiments, the patient has stage IIIC cancer and the treatment may reduce the risk of invasive disease corresponding to a hazard ratio of less than about 0.69 when the risk is calculated relative to patients not receiving the treatment.

In some embodiments, the patient received prior adjuvant chemotherapy and the treatment may reduce the risk of invasive disease corresponding to a hazard ratio of less than about 0.67 when the risk is calculated relative to patients not receiving the treatment.

In some embodiments, the patient did not receive prior adjuvant chemotherapy and the treatment may reduce the risk of invasive disease corresponding to a hazard ratio of less than about 0.81 when the risk is calculated relative to patients not receiving the treatment.

In some embodiments, the patient received prior neoadjuvant chemotherapy and the treatment may reduce the risk of invasive disease corresponding to a hazard ratio of less than about 0.79 when the risk is calculated relative to patients not receiving the treatment.

In some embodiments, the patient did not receive prior neoadjuvant chemotherapy and the treatment may reduce the risk of invasive disease corresponding to a hazard ratio of less than about 0.72 when the risk is calculated relative to patients not receiving the treatment.

In some embodiments, the patient received prior radiation therapy and the treatment may reduce the risk of invasive disease corresponding to a hazard ratio of less than about 0.74 when the risk is calculated relative to patients not receiving the treatment.

In some embodiments, the patient did not receive prior radiation therapy and the treatment may reduce the risk of invasive disease corresponding to a hazard ratio of less than about 0.98 when the risk is calculated relative to patients not receiving the treatment.

In some embodiments, the patient received prior endocrine therapy and the treatment may reduce the risk of invasive disease corresponding to a hazard ratio of less than about 0.76 when the risk is calculated relative to patients not receiving the treatment.

In some embodiments, the patient did not receive endocrine therapy and the treatment may reduce the risk of invasive disease corresponding to a hazard ratio of less than about 0.77 when the risk is calculated relative to patients not receiving the treatment.

In some embodiments, the patient had a prior mastectomy and the treatment may reduce the risk of invasive disease corresponding to a hazard ratio of less than about 0.85 when the risk is calculated relative to patients not receiving the treatment.

In some embodiments, the patient did not have a prior mastectomy and the treatment may reduce the risk of invasive disease corresponding to a hazard ratio of less than about 0.55 when the risk is calculated relative to patients not receiving the treatment.

In some embodiments, the patient is Asian and the treatment may reduce the risk of invasive disease corresponding to a hazard ratio of less than about 0.52 when the risk is calculated relative to patients not receiving the treatment.

In some embodiments, the patient is not Asian and the treatment may reduce the risk of invasive disease corresponding to a hazard ratio of less than about 0.81 when the risk is calculated relative to patients not receiving the treatment.

In some embodiments, the patient is located in Europe and the treatment may reduce the risk of invasive disease corresponding to a hazard ratio of less than about 0.88 when the risk is calculated relative to patients not receiving the treatment.

In some embodiments, the patient is located in North America or Australia and the treatment may reduce the risk of invasive disease corresponding to a hazard ratio of less than about 0.70 when the risk is calculated relative to patients not receiving the treatment.

In some embodiments, the patient is located in Asia and the treatment may reduce the risk of invasive disease corresponding to a hazard ratio of less than about 0.34 when the risk is calculated relative to patients not receiving the treatment.

In some embodiments, the patient is located in Latin America and the treatment may reduce the risk of invasive disease corresponding to a hazard ratio of less than about 0.69 when the risk is calculated relative to patients not receiving the treatment.

In some embodiments, the patient is less than 45 years old and the treatment may reduce the risk of invasive disease corresponding to a hazard ratio of less than about 0.68 when the risk is calculated relative to patients not receiving the treatment.

In some embodiments, the patient is 45 to 54 years old and the treatment may reduce the risk of invasive disease corresponding to a hazard ratio of less than about 0.75 when the risk is calculated relative to patients not receiving the treatment.

In some embodiments, the patient is 55 to 64 and the treatment may reduce the risk of invasive disease corresponding to a hazard ratio of less than about 0.84 when the risk is calculated relative to patients not receiving the treatment.

In some embodiments, the patient is 65 years old or older and the treatment may reduce the risk of invasive disease corresponding to a hazard ratio of less than about 0.72 when the risk is calculated relative to patients not receiving the treatment.

In some embodiments, the patient receives letrozole and the treatment may reduce the risk of invasive disease corresponding to a hazard ratio of less than about 0.79 when the risk is calculated relative to patients not receiving the treatment.

In some embodiments, the patient receives anastrozole and the treatment may reduce the risk of invasive disease corresponding to a hazard ratio of less than about 0.70 when the risk is calculated relative to patients not receiving the treatment.

In some embodiments, the patient has ER+/PR+ cancer and the treatment may reduce the risk of invasive disease corresponding to a hazard ratio of less than about 0.73 when the risk is calculated relative to patients not receiving the treatment.

In some embodiments, the patient has ER+/PR− cancer and the treatment may reduce the risk of invasive disease corresponding to a hazard ratio of less than about 0.91 when the risk is calculated relative to patients not receiving the treatment.

In some embodiments, the patient has a tumor with nodal status 0 and the treatment may reduce the risk of invasive disease corresponding to a hazard ratio of less than about 0.63 when the risk is calculated relative to patients not receiving the treatment.

In some embodiments, the patient has a tumor with nodal status N1-N3 and the treatment may reduce the risk of invasive disease corresponding to a hazard ratio of less than about 0.77 when the risk is calculated relative to patients not receiving the treatment.

In some embodiments, the patient has a tumor of category T1-T3 and the treatment may reduce the risk of invasive disease corresponding to a hazard ratio of less than about 0.74 when the risk is calculated relative to patients not receiving the treatment.

In some embodiments, the patient has a tumor of a category greater than T3 (e.g., T4) and the treatment may reduce the risk of invasive disease corresponding to a hazard ratio of less than about 0.83 when the risk is calculated relative to patients not receiving the treatment.

In some embodiments, the patient has a tumor with a histological grade 1 and the treatment may reduce the risk of invasive disease corresponding to a hazard ratio of less than about 0.78 when the risk is calculated relative to patients not receiving the treatment.

In some embodiments, the patient has a tumor with a histological grade 1 and the treatment may reduce the risk of invasive disease corresponding to a hazard ratio of less than about 0.78 when the risk is calculated relative to patients not receiving the treatment.

In some embodiments, the patient has a tumor with a histological grade 2 and the treatment may reduce the risk of invasive disease corresponding to a hazard ratio of less than about 0.75 when the risk is calculated relative to patients not receiving the treatment.

In some embodiments, the patient has a tumor with a histological grade 3 and the treatment may reduce the risk of invasive disease corresponding to a hazard ratio of less than about 0.78 when the risk is calculated relative to patients not receiving the treatment.

In some embodiments, the patient has a tumor with Ki67 status that is 20% or lower and the treatment may reduce the risk of invasive disease corresponding to a hazard ratio of less than about 0.80 when the risk is calculated relative to patients not receiving the treatment.

In some embodiments, the patient has a tumor with Ki67 status that is greater than 20% and the treatment may reduce the risk of invasive disease corresponding to a hazard ratio of less than about 0.75 when the risk is calculated relative to patients not receiving the treatment.

In some embodiments, the patient has a tumor of a ductal subtype and the treatment may reduce the risk of invasive disease corresponding to a hazard ratio of less than about 0.68 when the risk is calculated relative to patients not receiving the treatment.

In some embodiments, the patient has a tumor that is not a ductal subtype or a lobular subtype and the treatment may reduce the risk of invasive disease corresponding to a hazard ratio of less than about 0.89 when the risk is calculated relative to patients not receiving the treatment.

In some embodiments, the patient has a BMI of 25 or greater and the treatment may reduce the risk of invasive disease corresponding to a hazard ratio of less than about 0.85 when the risk is calculated relative to patients not receiving the treatment.

In some embodiments, the patient has a BMI of less than 25 and the treatment may reduce the risk of invasive disease corresponding to a hazard ratio of less than about 0.64 when the risk is calculated relative to patients not receiving the treatment.

For overall survival, the treatment may yield no deterioration in overall survival corresponding to a hazard ratio of 0.76 when the risk is calculated relative to patients not receiving the treatment.

In some embodiments, the treatment does not cause or worsen events such as pneumonia, pulmonary embolism, dyspnoea, increased alanine aminotransferase, increased aspartate aminotransferase, arthralgia, fatigue, neutropenia, decreased neutrophil count, and/or nausea.

In some embodiments, the treatment does not cause or worsen conditions related to hepatobiliary toxicity, for example, as measured by increases in alanine aminotransferase, aspartate aminotransferase, gamma-glutamyltransferase, blood alkaline phosphatase, and/or blood bilirubin.

In some embodiments, the treatment does not cause or worsen conditions related to myelosuppression (e.g., anemia and/or leukopenia). For example, the treatment does not cause of worsen leukopenia, or lead to decreased white blood cell count, lymphopenia, or decreased lymphocyte count.

In some embodiments, the treatment does not cause or worsen conditions related to myelosuppression (e.g., neutropenia, decreased neutrophil count, thrombocytopenia, decreased platelet count).

In some embodiments, the treatment does not prolong the QT interval in an electrocardiogram.

In some embodiments, the treatment does not cause or worsen conditions related to renal toxicity (e.g., increased blood creatinine, decreased glomerular filtration rate, increased blood urea).

In some embodiments, the treatment does not cause or worsen conditions related to reproductive toxicity, such as mastitis.

### F. A METHOD FOR TREATMENT OF HR+/HER2− EARLY BREAST CANCER

One aspect of the present disclosure relates to a method of treatment for HR+/HER2− stage II or III early breast cancer in an adult patient in need thereof, comprising administering to the patient a treatment comprising a dose of ribociclib (or a pharmaceutically acceptable salt thereof, such as ribociclib succinate) ranging from 150 mg/day to 450 mg/day on days 1-21 of a 28-day cycle in combination with an endocrine therapy (such as an aromatase inhibitor, preferably letrozole or anastrozole) administered on every day (e.g., days 1-28) of the 28-day cycle.

A further aspect of the present disclosure relates to a method of preventing or reducing signs or symptoms of early stage breast cancer, comprising administering to the patient a treatment comprising a dose of ribociclib, a freebase form thereof or a pharmaceutically acceptable salt thereof, ranging from 150 mg/day to 450 mg/day on days 1-21 of a 28-day cycle in combination with an aromatase inhibitor, preferably letrozole or anastrozole, administered on every day of the 28-day cycle.

Another aspect of the present disclosure relates to a method of maintaining in remission an adult patient who had previously been diagnosed with HR+/HER2− stage II or stage III early breast cancer, comprising administering to the patient a treatment comprising administering to the patient a treatment comprising a dose of ribociclib, a freebase form thereof or a pharmaceutically acceptable salt thereof, ranging from 150 mg/day to 450 mg/day on days 1-21 of a 28-day cycle in combination with an aromatase inhibitor, preferably letrozole or anastrozole, administered on every day of the 28-day cycle.

Still another aspect of the present disclosure relates to a method of preventing breast cancer recurrence in an adult patient, comprising providing adjuvant treatment to a patient who has received a prior treatment for HR+/HER2− stage II or III early breast cancer, wherein the adjuvant treatment comprises administering to the patient 400 mg ribociclib or a pharmaceutically acceptable salt thereof on days 1 to 21 of a 28-day cycle for at least 36 months, in combination with an aromatase inhibitor as defined in any one of embodiments 23 to 28 administered on every day of the 28-day cycle.

In some embodiments, the treatment comprises comprising administering to the patient a treatment comprising a dose of about 400 mg/day (or a dose of about 200 mg/day) ribociclib or a pharmaceutically acceptable salt thereof, such as ribociclib succinate, on days 1 to 21 of a 28-day cycle for 36 months, in combination with an endocrine therapy comprising a dose of letrozole ranging from about 1 mg/day to about 4 mg/day or a dose of anastrozole ranging from about 0.5 mg/day to about 1.5 mg/day administered on every day (e.g., days 1-28) of the 28-day cycle.

In some embodiments, the treatment comprises administering to the patient a treatment comprising a dose of about 400 mg/day (or a dose of about 200 mg/day) ribociclib or a pharmaceutically acceptable salt thereof, such as ribociclib succinate, on days 1 to 21 of a 28-day cycle for 36 months, in combination with an endocrine therapy, wherein the endocrine therapy comprises a dose of letrozole of about 2.5 mg/day or a dose of anastrozole of about 1 mg/day administered on days 1-28 of the 28-day cycle.

In some embodiments, the treatment reduces one or more of invasive ipsilateral breast tumor (IBTR) recurrence, local-regional invasive recurrence, distant recurrence (e.g., presence of metastasis), death from breast cancer, death from a non-breast cancer cause, death from an unknown cause, presence of invasive contralateral breast cancer, and presence of a second primary invasive cancer.

In some embodiments, the treatment reduces the risk of invasive disease corresponding to a hazard ratio of less than about 1 when the risk is calculated relative to patients not receiving the treatment. For example, the hazard ratio for iDFS may be calculated using model such as an unstratified and covariate unadjusted Cox model or a stratified and covariate adjusted Cox model, as described in Appendix A.

In some embodiments, the hazard ratio may be less than or equal to about 0.78. The hazard ratio may be less than or equal to about 0.72.

In some embodiments, the patient has HR+/HER2− stage III early breast cancer and the treatment reduces the risk of invasive disease corresponding to a hazard ratio of less than or equal to about 0.74 when the risk is calculated relative to patients not receiving the treatment. The hazard ratio may be less than or equal to about 0.76. In some embodiments, the treatment yields at least a 25% reduction in the risk of invasive disease corresponding to a hazard ratio of 0.75 when the risk is calculated relative to patients not receiving the treatment.

In some embodiments, the treatment reduces the risk of invasive disease to a similar level for patient subgroups comprising patients with stage II early breast cancer, stage 111 early breast cancer, patients who are premenopausal women or men, and patients who are postmenopausal women.

For overall survival, the treatment may yield no deterioration in overall survival corresponding to a hazard ratio of 0.76 when the risk is calculated relative to patients not receiving the treatment.

In some embodiments, the patient is a postmenopausal woman. In some embodiments, the patient is a premenopausal woman or a man.

In some embodiments, the early breast cancer comprises a tumor of stage II. In some embodiments, the tumor may have an anatomic stage IIA. In some embodiments, the tumor may have an anatomic stage IIB. In some embodiments, the early breast cancer comprises a tumor of stage III. In some embodiments, the tumor may have an anatomic stage IIIA. In some embodiments, the tumor may have an anatomic stage IIIB. In some embodiments, the tumor may have an anatomic stage IIIC.

In some embodiments, the patient has received prior neo-adjuvant chemotherapy. In some embodiments, the patient has received prior adjuvant chemotherapy.

In some embodiments, the patient has received prior radiation therapy.

In some embodiments, the patient has undergone a mastectomy. In some embodiments, the patient has not undergone a mastectomy.

In some embodiments, the patient is located in North America. In some embodiments, the patient is located in Europe, for example, in Western Europe. In some embodiments, the patient is located in Oceania, for example, in Australia. In some embodiments, the patient is located in Latin America. In some embodiments, the patient is located in Asia.

In some embodiments, the patient is Asian. In some embodiments, the patient is non-Asian.

In some embodiments, the patient may be American Indian. In some embodiments, the patient may be Alaska Native. In some embodiments, the patient may be Black or African American. In some embodiments, the patient may be Native Hawaiian or Other Pacific Islander. In some embodiments, the patient may be White. In some embodiments, the patient may be a mixed race. In some embodiments, the patient may be non-Asian. In some embodiments, the patient may be Hispanic or Latino. In some embodiments, the patient is not Hispanic or Latino.

In some embodiments, the patient is 18 years or older. The patient may be 18-44 years old. The patient may be 45-54 years old. The patient may be 55 to 64 years old. The patient may be 65 years or older.

In some embodiments, the endocrine therapy is a non-steroidal aromatase inhibitor (NSAI). In some embodiments, the NSAI may be letrozole. In some embodiments, the NSAI may be anastrozole.

In some embodiments, the early breast cancer is selected from a ER+/PR+, a ER−/PR+, and a ER+/PR− breast cancer.

In some embodiments, the treatment is administered irrespective of the nodal status of the breast cancer.

In some embodiments, the early breast cancer has a nodal status of N0, N1, N2, or N3.

In some embodiments, the early breast cancer comprises a tumor of category T0, category T1, T2, T3, or T3.

In some embodiments, the early breast cancer has a histological grade at time of surgery of G1, G2, or G3.

In some embodiments, the early breast cancer has a Ki67 status (e.g., from an archival tumor) of 20 or less than 20. Alternatively, the early breast cancer may have a Ki67 status of greater than 20.

In some embodiments, the early breast cancer has a ductal histological subtype or a lobular histological subtype.

An aspect of the present disclosure relates to a method of preventing recurrence of breast cancer in an adult patient who has received a prior treatment for HR+/HER2− stage II or stage III early breast cancer, comprising administering to the patient (i) a dose of ribociclib, a freebase form thereof, or a pharmaceutically acceptable salt thereof, and (ii) a dose of an aromatase inhibitor, preferably letrozole or anastrozole.

A further aspect of the present disclosure relates to a method of treating an adult patient in remission from HR+/HER2− stage II or stage III early breast cancer, comprising administering to the patient (i) a dose of ribociclib, a freebase form thereof, or a pharmaceutically acceptable salt thereof, and (ii) a dose of an aromatase inhibitor, preferably letrozole or anastrozole.

Another aspect of the present disclosure relates to a method of treating HR+/HER2− stage II or stage III early breast cancer in an adult patient in need thereof, comprising administering to the patient (i) a dose of ribociclib, a freebase form thereof, or a pharmaceutically acceptable salt thereof, ranging from 150 mg/day to 450 mg/day administered on days 1-21 of a 28-day cycle and (ii) an aromatase inhibitor, preferably letrozole or anastrozole, administered on every day of the 28-day cycle.

One aspect of the present disclosure relates to a method of treating breast cancer recurrence in an adult patient who has received at least one prior treatment for HR+/HER2− stage II or III early breast cancer and who has no detectable signs or symptoms of breast cancer, comprising administering to the patient an adjuvant treatment comprising (i) a dose of ribociclib succinate at a total dose of ribociclib of 400 mg/day administered on days 1 to 21 of a 28-day cycle, and (ii) a dose of either 2.5 mg/day letrozole or 1 mg/day anastrozole administered on every day of the 28-day cycle.

An additional aspect of the present disclosure relates to a method of treating an adult patient who is in remission from HR+/HER2− stage II or stage III early breast cancer and in need of adjuvant treatment, comprising administering to the patient an adjuvant treatment comprising (i) a dose of ribociclib succinate at a total dose of ribociclib of 400 mg/day administered on days 1-21 of a 28-day cycle and (ii) a dose of either 2.5 mg/day letrozole or 1 mg/day anastrozole administered on every day of the 28-day cycle.

In some embodiments, the patient has a BMI of 25 or greater. In some embodiments, the patient has a BMI of less than 25.

A further aspect of the present disclosure relates to a method of preventing or reducing signs or symptoms of early stage breast cancer, comprising administering to the patient a treatment comprising ribociclib, a freebase form thereof or a pharmaceutically acceptable salt thereof in combination with an aromatase inhibitor, preferably letrozole or anastrozole. In some embodiments, the ribociclib is a freebase form thereof or a pharmaceutically acceptable salt thereof, and is administered to the patient in a dose ranging from 150 mg/day to 450 mg/day on days 1-21 of a 28-day cycle. In some embodiments, the aromatase inhibitor is administered on every day of the 28-day cycle.

### G. A PATIENT IN REMISSION

One aspect of the present disclosure relates a method of treating HR+/HER2− stage II or stage III early breast cancer in an adult patient in remission, comprising administering to the adult patient ribociclib in combination with an aromatase inhibitor whereby the administration maintains the adult patient in remission for at least about 3 months.

A further aspect relates to a method of treating HR+/HER2− stage II or stage III early breast cancer in an adult patient, comprising providing adjuvant treatment by administering ribociclib in combination with aromatase inhibitor, wherein the patient at start of the adjuvant treatment is in complete remission, and the administration of ribociclib in combination with an aromatase inhibitor maintains the adult patient in complete remission for at least about 3 months.

The administration of ribociclib in combination with an aromatase inhibitor may maintain the adult patient in remission (e.g., complete remission) for a period of time that is at least about 3 months. In some embodiments, the administration maintains the adult patient in remission for at least about 6 months. In some embodiments, the administration maintains the adult patient in remission for at least about 9 months. In some embodiments, the administration maintains the adult patient in remission for at least about 12 months. In some embodiments, the administration maintains the adult patient in remission for at least about 15 months. In some embodiments, the administration maintains the adult patient in remission for at least about 18 months. In some embodiments, the administration maintains the adult patient in remission for at least about 21 months. In some embodiments, the administration maintains the adult patient in remission for at least about 24 months. In some embodiments, the administration maintains the adult patient in remission for at least about 27 months. In some embodiments, the administration maintains the adult patient in remission for at least about 30 months. In some embodiments, the administration maintains the adult patient in remission for at least about 33 months. In some embodiments, the administration maintains the adult patient in remission for at least about 36 months.

In certain embodiments, ribociclib and an aromatase inhibitor are administered for a treatment duration, and the administration maintains the adult patient in remission for at least the treatment duration.

For the methods described herein, the dose and treatment schedule for the combination of ribociclib and an aromatase inhibitor may follow the exemplary embodiments as follows. In some embodiments, a dose of 400 mg ribociclib is orally administered once daily on days 1-21 of a 28-day cycle and aromatase inhibitor is administered once daily on each day of the 28-day cycle.

A dose of 400 mg ribociclib is orally administered once daily on days 1-21 of a 28-day cycle for a treatment duration of up to three years and aromatase inhibitor is administered once daily on each day of the 28-day cycle for the up to three years. For example, a dose of 400 mg ribociclib may be orally administered once daily on days 1-21 of a 28-day cycle for a treatment duration of up to five years and aromatase inhibitor is administered once daily on each day of the 28-day cycle for the up to five years.

In some embodiments, a pharmaceutically acceptable salt of ribociclib is orally administered in an amount corresponding to 400 mg ribociclib. The pharmaceutically acceptable salt may be ribociclib succinate.

In some embodiments, the adult patient was never treated with a 600 mg dose of ribociclib.

In some embodiments, the adult patient has never been diagnosed with HR+/HER2− advanced or metastatic breast cancer.

Ribociclib and aromatase inhibitor may be administered as adjuvant therapy. In some embodiments, the ribociclib is not administered at a dose of 600 mg/day.

The dose of ribociclib may be administered in tablet form. For example, two tablets may be orally administered to the adult patient once daily on days 1-21 of a 28-day cycle repeating for a treatment duration, and each tablet contains an amount of a pharmaceutically acceptable ribociclib salt corresponding to 200 mg ribociclib. In some embodiments, the ribociclib salt is ribociclib succinate. In some embodiments, the aromatase inhibitor is letrozole. In some embodiments, the aromatase inhibitor is anastrazole.

In some embodiments, the aromatase inhibitor is letrozole, and the letrozole is administered in a dose ranging from 1 mg to 4 mg once daily. For example, the aromatase inhibitor may be letrozole, and the letrozole may be administered in a dose of 2.5 mg once daily.

In some embodiments, the aromatase inhibitor is anastrozole, and the anastrozole is administered in a dose ranging from 0.5 mg once daily to 1.5 mg once daily. For example, anastrozole may be administered in a dose of 1 mg once daily. The method of any one of embodiments 117 to 148, wherein the patient is a postmenopausal woman.

In some embodiments, the patient is a premenopausal woman or a man.

The patient may be a premenopausal woman or a man. Some embodiments, for example, where the patient is a premenopausal woman or man, further comprise administering to the adult patient a gonadotropin-releasing hormone agonist. In some embodiments, the gonadotropin-releasing hormone agonist is goserelin. In some embodiments, the goserelin is administered in a dose ranging from 2 mg to 5 mg. For example, the dose of goserelin may be 3.6 mg. In some embodiments, the goserelin is administered subcutaneously. In some embodiments, the goserelin is administered once every 4 weeks.

In some embodiments, the breast cancer has a histological subtype that is ductal or a histological subtype that is lobular.

In some embodiments, the breast cancer is a stage IIA cancer or a stage IIB cancer. For example, the breast cancer may be a stage IIA cancer. The breast cancer may be a stage IIB cancer.

In some embodiments, the breast cancer is a stage IIIIA cancer, a stage IIIB cancer, or a stage IIIC cancer. For example, the breast cancer may be a stage IIIIA cancer. The breast cancer may be a stage IIIB cancer. The breast cancer may be a stage IIIC cancer.

In some embodiments, the treatment is administered irrespective of the nodal status of the breast cancer. In some embodiments, treatment with ribociclib and aromatase inhibitor is not adjusted on the basis of the nodal status of the early breast cancer.

In some embodiments, the breast cancer has a nodal status selected from N0, N1, N2, and N3.

For example, the breast cancer may have a nodal status of NO. The breast cancer may have a nodal status of N1-N3. The breast cancer may have a nodal status of N1. The breast cancer may have a nodal status of N2. The breast cancer may have a nodal status of N3.

In some embodiments, the breast cancer comprises one or more cells having a histological grade selected from G1, G2, or G3. For example, the breast cancer may comprise one or more cells having the histological grade G1. The breast cancer may comprise one or more cells having the histological grade G2. The breast cancer may comprise one or more cells having the histological grade G3.

In some embodiments, the breast cancer comprises a tumor of category T0, T1, T2, T3, or T4. For example, the breast cancer may comprise a tumor of category T1, T2, or T3. The breast cancer may comprise a tumor of category T0. The breast cancer may comprise a tumor of category T1. The breast cancer may comprise a tumor of category T2. The breast cancer may comprise a tumor of category T3. The breast cancer may comprise a tumor of category T4.

In some embodiments, the breast cancer has a Ki67 status of 20 or lower. In some embodiments, the breast cancer has a Ki67 status of greater than 20.

In some embodiments, prior to administering ribociclib and aromatase inhibitor to the adult patient, the patient had surgery for the early breast cancer.

In some embodiments, prior to administering ribociclib and aromatase inhibitor to the adult patient, the patient had (1) surgery for the early breast cancer, followed by (2) chemotherapy.

In some embodiments, prior to administering ribociclib and aromatase inhibitor to the adult patient, the patient had (1) surgery for the early breast cancer, followed by (2) chemotherapy and/or endocrine therapy.

In some embodiments, the endocrine therapy was therapy with a prior aromatase inhibitor. The prior aromatase inhibitor may have been letrozole or anastrozole.

In some embodiments, immediately prior to administering ribociclib and aromatase inhibitor to the adult patient, the patient had surgery for the early breast cancer. In some embodiments, wherein the surgery comprised a complete surgical resection of the cancer. In some embodiments, the surgery was a mastectomy.

In some embodiments, the patient received neoadjuvant therapy. For example, the neoadjuvant therapy may have been chemotherapy.

In some embodiments, the adult patient is located in a geographic region selected from North America, Western Europe, or Oceania.

In some embodiments, the patient is Asian.

In some embodiments, the patient is 18 to 45 years of age. In some embodiments, the patient is 45 to 54 years of age. In some embodiments, the patient is 54 to 64 years of age. In some embodiments, the patient is greater than 64 years of age.

In some embodiments, wherein the patient has a BMI of 25 or higher. In some embodiments, the patient has a BMI lower than 25.

In some embodiments, the treatment improves a condition of the patient relative to a patient not receiving the treatment and/or relative to the condition in the patient prior to treatment.

In some embodiments, the treatment reduces the risk of invasive disease. For example, The treatment may reduces the risk of invasive disease in the adult patient corresponding to a hazard ratio of less than 1 when the risk is calculated relative to other patients having received the same treatment as the adult patient except that the other patients did not receive the ribociclib.

In some embodiments, the treatment reduces the risk of invasive disease in the adult patient corresponding to a hazard ratio of less than or equal to 0.78 when the risk is calculated relative to other patients having received the same treatment as the adult patient except that the other patients did not receive the ribociclib.

In some embodiments, the adult patient is a premenopausal woman or a man and the treatment reduces the risk of invasive disease corresponding to a hazard ratio of less than or equal to 0.72 when the risk is calculated relative to other premenopausal women or men, respectively, having received the same treatment as the premenopausal woman or man, respectively, except that the other premenopausal women or men did not receive the ribociclib.

In some embodiments, the adult patient was diagnosed with HR+/HER2− stage III early breast cancer and the treatment reduces the risk of invasive disease in the adult patient corresponding to a hazard ratio of less than or equal to 0.74 when the risk is calculated relative to other patients having received the same treatment as the adult patient except that the other patients did not receive the ribociclib.

In some embodiments, the adult patient was diagnosed with HR+/HER2− stage II early breast cancer and the treatment reduces the risk of invasive disease corresponding to a hazard ratio of less than or equal to 0.76 when the risk is calculated relative to other patients having received the same treatment as the adult patient except that the other patients did not receive the ribociclib.

In some embodiments, the adult patient was diagnosed with HR+/HER2− stage III early breast cancer, and the treatment yields at least a 25% reduction in the risk of invasive disease corresponding to a hazard ratio of 0.75 when the risk is calculated relative to other patients having received the same treatment as the adult patient except that the other patients did not receive the ribociclib.

In some embodiments, the treatment reduces the risk of invasive disease to a similar level for patient subgroups comprising patients with stage II early breast cancer, stage III early breast cancer, patients who are premenopausal women or men, and patients who are postmenopausal women.

In some embodiments, the treatment yields no deterioration in overall survival corresponding to a hazard ratio of 0.76 when the risk is calculated relative to other patients having received the same treatment as the adult patient except that the other patients did not receive the ribociclib.

In some embodiments, the treatment reduces and/or prevents one or more of recurrence of the cancer, spread of the cancer, development and/or growth of an additional cancer, and risk of death from the cancer.

In some embodiments, the treatment reduces the recurrence of cancer, wherein the recurrence is one or more of invasive ipsilateral breast tumor (IBTR) recurrence, local-regional invasive recurrence, and distant recurrence.

In some embodiments, the treatment reduces the spread of cancer, wherein the spread of cancer is an invasive contralateral breast cancer or an additional primary invasive cancer.

A further aspect of the present disclosure relates to ribociclib or a pharmaceutically acceptable salt thereof and an aromatase inhibitor for use in a method of treatment for HR+/HER2− stage II or III early breast cancer in an adult patient, wherein the method is any one of the methods described herein.

Another aspect of the present disclosure relates to the use of ribociclib or a pharmaceutically acceptable salt thereof and an aromatase inhibitor in the manufacture of a medicament for treating HR+/HER2− stage II or III early breast cancer in an adult patient, wherein the medicament is administered by any one of the methods described herein.

Another aspect of the present disclosure relates to a kit for performing a method of treating HR+/HER2− stage II or stage III early breast cancer in an adult patient according to any one of the methods described herein.

### H. A KIT FOR USES AND METHODS

One aspect of the present disclosure provides a kit for performing the methods disclosed herein, for example, a kit for performing a method of treating HR+/HER2− stage II or stage III early breast cancer in an adult patient. The kit may be promoted, distributed, or sold as a unit for performing the methods of the present invention.

In some embodiments, the kit comprises a dose of ribiciclib, a freebase form thereof or a pharmaceutically acceptable salt thereof, and a dose of an aromatase inhibitor such as letrozole or anastrozole. The kit may comprise guidance and/or instructions, e.g., a schedule for administration of the ribociclib and/or the aromatase inhibitor.

In some embodiments, the kit comprises a dose of ribociclib ranging from about 150 mg/day to 450, and guidance and/or instructions to administer the ribociclib on days 1-21 of a 28-day cycle. The kit may further comprise a dose of an aromatase inhibitor such as letrozole or anastrozole, for example, a dose of about 2.5 mg/day letrozole or about 1 mg/day anastrozole, and may further comprise guidance and/or instructions to administer the aromatase inhibitor on every day of the 28-day cycle.

In some embodiments, the kit comprises a dose of ribociclib succinate at a total dose of ribociclib of 400 mg/day and a dose of either 2.5 mg/day letrozole or 1 mg/day anastrozole. The kit may further comprise guidance and/or instructions to administer the dose of ribociclib succinate on days 1 to 21 of a 28-day cycle, and to administer the dose of letrozole or anastrozole administered on every day of the 28-day cycle.

Exemplary kits may comprise more than 1 daily dose, for example, a kit may comprise 21 doses of the ribociclib, or freebase form thereof or a pharmaceutically acceptable salt thereof, and 28 doses of the aromatase inhibitor (e.g., letrozole or anastrozole), for administration over the whole 28-day cycle. Other exemplary kits may comprise doses of ribociclib, or freebase form thereof or a pharmaceutically acceptable salt thereof, and the aromatase inhibitor (letrozole or anastrozole) for multiple 28-day cycles.

### I. EXEMPLARY EMBODIMENTS

The methods of treatment and medical uses of the present disclosure are provided in the following embodiments.
1. A method of treating HR+/HER2− stage II or stage III early breast cancer in an adult patient in need thereof, comprising administering to the patient a treatment comprising a dose of ribociclib, a freebase form thereof or a pharmaceutically acceptable salt thereof, ranging from 150 mg/day to 450 mg/day on days 1-21 of a 28-day cycle in combination with an aromatase inhibitor, preferably letrozole or anastrozole, administered on every day of the 28-day cycle.
2. A method of preventing or reducing signs or symptoms of early stage breast cancer, comprising administering to the patient a treatment comprising ribociclib, a freebase form thereof or a pharmaceutically acceptable salt thereof in combination with an aromatase inhibitor, preferably letrozole or anastrozole.
3. The method of embodiment 2, wherein the the ribociclib, a freebase form thereof or a pharmaceutically acceptable salt thereof, is administered to the patient in a dose ranging from 150 mg/day to 450 mg/day on days 1-21 of a 28-day cycle.
4. The method of embodiment 2 or 3, wherein the aromatase inhibitor is administered on every day of the 28-day cycle.
5. A method of preventing or reducing signs or symptoms of early breast cancer, comprising administering to the patient a treatment comprising a dose of ribociclib, a freebase form thereof or a pharmaceutically acceptable salt thereof, ranging from 150 mg/day to 450 mg/day on days 1-21 of a 28-day cycle in combination with an aromatase inhibitor, preferably letrozole or anastrozole, administered on every day of the 28-day cycle.
6. The method of any one of embodiments 1 to 5, wherein the patient is in remission from HR+/HER2− stage II or stage III early breast cancer.
7. A method of maintaining remission in an adult patient who had previously been diagnosed with HR+/HER2− stage II or stage III early breast cancer, comprising administering to the patient a treatment comprising a dose of ribociclib, a freebase form thereof or a pharmaceutically acceptable salt thereof, in combination with an aromatase inhibitor, preferably letrozole or anastrozole.
8. The method of embodiment 6 or embodiment 7, wherein the remission is complete remission.
9. The method of embodiment 6 or embodiment 7, wherein the remission is partial remission.
10. The method of any one of embodiments 1 to 9, wherein the treatment reduces recurrence of HR+/HER2− stage II or stage III early breast cancer.
11. The method of embodiment 10, wherein the treatment prevents recurrence for at least 3 months.
12. The method of embodiment 11, wherein the treatment prevents recurrence for at least 6 months.
13. The method of embodiment 12, wherein the treatment prevents recurrence for at least 1 year.
14. The method of any one of embodiments 1 to 13, wherein the patient has no signs or symptoms of cancer prior to receiving the treatment.
15. The method of any one of embodiments 1 to 14, wherein the treatment prevents growth of HR+/HER2− breast cancer cells.
16. The method of any one of embodiments 1 to 15, wherein the treatment is an adjuvant therapy.
17. The method of any one of embodiments 1 to 16, wherein the ribociclib is a pharmaceutically acceptable ribociclib salt.
18. The method of embodiment 17, wherein the ribociclib salt is ribociclib succinate.
19. The method of any one of embodiments 1 to 18, wherein the dose of ribociclib is 200 mg/day or 400 mg/day.
20. The method of any one of embodiments 1 to 18, wherein the ribociclib is not administered at a dose of 600 mg/day.
21. The method of any one of embodiments 1 to 19, wherein ribociclib is administered as ribociclib succinate, and the total dose of ribociclib is 200 mg/day.
22. The method of any one of embodiments 1 to 19, wherein ribociclib is administered as ribociclib succinate, and the total dose of ribociclib is 400 mg/day.
23. The method of any one of embodiments 1 to 19, wherein the ribociclib is administered at a dose of 400 mg/day for a period of time, after which a dose of 200 mg/day ribociclib is administered.
24. The method of any one of embodiments 1 to 23, wherein the dose of ribociclib is administered orally.
25. The method of any one of embodiments 1 to 24, wherein the dose of ribociclib is administered in tablet form.
26. The method of any one of embodiments 1 to 24, wherein the aromatase inhibitor is letrozole or anastrozole.
27. The method of any one of embodiments 1 to 26, wherein the aromatase inhibitor is administered orally.
28. The method of embodiment 26 or 27, wherein the letrozole is administered in a dose ranging from 1 mg/day to 4 mg/day.
29. The method of embodiment 28, wherein the letrozole is administered in a dose of 2.5 mg/day.
30. The method of embodiment 26 or 27, wherein the anastrozole is administered in a dose ranging from 0.5 mg/day to 1.5 mg/day.
31. The method of embodiment 30, wherein the anastrozole is administered in a dose of 1 mg/day.
32. The method of any one of embodiments 1 to 31, wherein the treatment comprises a gonadotropin-releasing hormone agonist.
33. The method of embodiment 32, wherein the gonadotropin-releasing hormone agonist is goserelin.
34. The method of embodiment 33, wherein the goserelin is administered in a dose ranging from 2 mg to 5 mg.
35. The method of embodiment 34, wherein the dose of goserelin is 3.6 mg.
36. The method of any one of embodiments 33 to 35, wherein the goserelin is administered subcutaneously.
37. The method of any one of embodiments 33 to 36, wherein the goserelin is administered once every 4 weeks.
38. The method of any one of embodiments 1 to 31, wherein the patient is a postmenopausal woman.
39. The method of any one of embodiments 1 to 37, wherein the patient is a premenopausal woman or a man.
40. The method of any one of embodiments 1 to 39, wherein the treatment is administered to the patient for at least 12 months.
41. The method of embodiment 40, wherein the treatment is administered to the patient for at least 24 months.
42. The method of embodiment 40 or embodiment 41, wherein the treatment is administered to the patient for at least 36 months.
43. The method of any one of embodiments 40 to 41, wherein the treatment is administered to the patient for at least 48 months.
44. The method of any one of embodiments 40 to 41, wherein the treatment is administered to the patient for at least 60 months.
45. The method of any one of embodiments 1 to 44, wherein the treatment continues until the patient has no detectable cancer.
46. The method of any one of embodiments 1 to 45, wherein the breast cancer is ER+ and PR+.
47. The method of any one of embodiments 1 to 45, wherein the breast cancer is ER- and PR+.
48. The method of any one of embodiments 1 to 45, wherein the breast cancer is ER+ and PR−.
49. The method of any one of embodiments 1 to 48, wherein the breast cancer has a histological subtype that is ductal or a histological subtype that is lobular.
50. The method of any one of embodiments 1 to 49, wherein the breast cancer is a stage IIA cancer or a stage IIB cancer.
51. The method of embodiment 50, wherein the breast cancer is a stage IIA cancer.
52. The method of embodiment 50, wherein the breast cancer is a stage IIB cancer.
53. The method of any one of embodiments 1 to 49, wherein the breast cancer is a stage IIIA cancer, a stage IIIB cancer, or a stage IIIC cancer.
54. The method of embodiment 53, wherein the breast cancer is a stage IIIIA cancer.
55. The method of embodiment 53, wherein the breast cancer is a stage IIIB cancer.
56. The method of embodiment 53, wherein the breast cancer is a stage IIIC cancer.
57. The method of any one of embodiments 1 to 56, wherein the treatment is administered irrespective of the nodal status of the breast cancer.
58. The method of any one of embodiments 1 to 57, wherein the breast cancer has a nodal status selected from N0, N1, N2, and N3.
59. The method of embodiment 57 or 58, wherein the breast cancer has a nodal status of NO.
60. The method of embodiment 57 or 58, wherein the breast cancer has a nodal status of N1 to N3.
61. The method of embodiment 57 or 58, wherein the breast cancer has a nodal status of N1.
62. The method of embodiment 57 or 58, wherein the breast cancer has a nodal status of N2.
63. The method of embodiment 57 or 58, wherein the breast cancer has a nodal status of N3.
64. The method of any one of embodiments 1 to 63, wherein the breast cancer comprises one or more cells having a histological grade selected from G1, G2, or G3.
65. The method of embodiment 64, wherein the breast cancer comprises one or more cells having the histological grade G1.
66. The method of embodiment 64, wherein the breast cancer comprises one or more cells having the histological grade G2.
67. The method of embodiment 64, wherein the breast cancer comprises one or more cells having the histological grade G3.
68. The method of any one of embodiments 1 to 67, wherein the breast cancer comprises a tumor of category T0, T1, T2, T3, or T4.
69. The method of embodiment 68, wherein the breast cancer comprises a tumor of category T1, T2, or T3.
70. The method of embodiment 68, wherein the breast cancer comprises a tumor of category T0.
71. The method of embodiment 68 or 69, wherein the breast cancer comprises a tumor of category T1.
72. The method of embodiment 68 or 69, wherein the breast cancer comprises a tumor of category T2.
73. The method of embodiment 68 or 69, wherein the breast cancer comprises a tumor of category T3.
74. The method of embodiment 68, wherein the breast cancer comprises a tumor of category T4.
75. The method of any one of embodiments 1 to 74, wherein the breast cancer has a Ki67 status of 20 or lower.
76. The method of any one of embodiments 1 to 74, wherein the breast cancer has a Ki67 status of greater than 20.
77. The method of any one of embodiments 1 to 76, wherein prior to the administration, the patient has received a loading dose of (i) ribociclib and/or (ii) an endocrine therapy.
78. The method of any one of embodiments 1 to 77, wherein the patient has received at least one prior treatment for cancer.
79. The method of embodiment 78, wherein the prior treatment is an adjuvant treatment after another prior treatment.
80. The method of embodiment 78 or 79, wherein the prior treatment is surgery.
81. The method of embodiment 80, wherein the surgery comprises a complete surgical resection of the cancer.
82. The method of embodiment 80 or 81, wherein the surgery is a mastectomy.
83. The method of embodiment 80 or 81, wherein the prior treatment is a chemotherapy.
84. The method of embodiment 83, wherein the prior treatment is an adjuvant chemotherapy.
85. The method of embodiment 83, wherein the prior treatment is a neoadjuvant chemotherapy.
86. The method of embodiment 78 or 79, wherein the prior treatment is an endocrine therapy.
87. The method of embodiment 78 or 79, wherein the prior treatment is radiation therapy.
88. The method of any one of embodiments 78 to 87, wherein the patient did not respond to the prior treatment.
89. The method of any one of embodiments 1 to 88, wherein the patient is located in a geographic region selected from North America, Western Europe, or Oceania.
90. The method of any one of embodiments 1 to 89, wherein the patient is Asian.
91. The method of any one of embodiments 1 to 90, wherein the patient is 18 to 45 years of age.
92. The method of any one of embodiments 1 to 90, wherein the patient is 45 to 54 years of age.
93. The method of any one of embodiments 1 to 90, wherein the patient is 54 to 64 years of age.
94. The method of any one of embodiments 1 to 90, wherein the patient is greater than 64 years of age.
95. The method of any one of embodiments 1 to 94, wherein the patient has a BMI of 25 or higher.
96. The method of any one of embodiments 1 to 94, wherein the patient has a BMI lower than 25.
97. The method of any one of embodiments 1 to 96, wherein the treatment improves a condition of the patient relative to a patient not receiving the treatment and/or relative to the condition in the patient prior to treatment.
98. The method of any one of embodiments 1 to 97, wherein the treatment reduces the risk of invasive disease.
99. The method of embodiment 98, wherein the treatment reduces the risk of invasive disease corresponding to a hazard ratio of less than 1 when the risk is calculated relative to patients not receiving the treatment.
100. The method of embodiment 99, wherein the treatment reduces the risk of invasive disease corresponding to a hazard ratio of less than or equal to 0.78 when the risk is calculated relative to patients not receiving the treatment.
101. The method of any one of embodiments 1 to 37 and 39 to 100, wherein the patient is a premenopausal woman or a man and the treatment reduces the risk of invasive disease corresponding to a hazard ratio of less than or equal to 0.72 when the risk is calculated relative to patients receiving the treatment.
102. The method of any one of embodiments 98 to 100, wherein the patient has HR+/HER2− stage III early breast cancer and the treatment reduces the risk of invasive disease corresponding to a hazard ratio of less than or equal to 0.74 when the risk is calculated relative to patients not receiving the treatment.
103. The method of any one of embodiments 98 to 100, wherein the patient has HR+/HER2− stage II early breast cancer and the treatment reduces the risk of invasive disease corresponding to a hazard ratio of less than or equal to 0.76 when the risk is calculated relative to patients not receiving the treatment.
104. The method of any one of embodiments 98 to 100, wherein the patient has HR+/HER2− stage III early breast cancer, and the treatment yields at least a 25% reduction in the risk of invasive disease corresponding to a hazard ratio of 0.75 when the risk is calculated relative to patients not receiving the treatment.
105. The method of any one of embodiments 1 to 104, wherein the treatment reduces the risk of invasive disease to a similar level for patient subgroups comprising patients with stage II early breast cancer, stage III early breast cancer, patients who are premenopausal women or men, and patients who are postmenopausal women.
106. The method of any one of embodiments 1 to 105, wherein the treatment yields no deterioration in overall survival corresponding to a hazard ratio of 0.76 when the risk is calculated relative to patients not receiving the treatment.
107. The method of one of embodiments 1 to 106, wherein the treatment reduces and/or prevents one or more of recurrence of the cancer, spread of the cancer, development and/or growth of an additional cancer, and risk of death from the cancer.
108. The method of embodiment 107, wherein the treatment reduces the recurrence of cancer, wherein the recurrence is one or more of invasive ipsilateral breast tumor (IBTR) recurrence, local-regional invasive recurrence, and distant recurrence.
109. The method of embodiment 107 or embodiment 108, wherein the treatment reduces the spread of cancer, wherein the spread of cancer is an invasive contralateral breast cancer or an additional primary invasive cancer.
110. The method of any one of embodiments 1 to 109, wherein the treatment prevents death from cancer.
111. A method of maintaining in remission an adult patient who had previously been diagnosed with HR+/HER2− stage II or stage III early breast cancer, comprising administering to the patient a treatment comprising administering to the patient a treatment comprising a dose of ribociclib, a freebase form thereof or a pharmaceutically acceptable salt thereof, ranging from 150 mg/day to 450 mg/day on days 1-21 of a 28-day cycle in combination with an aromatase inhibitor, preferably letrozole or anastrozole, administered on every day of the 28-day cycle.
112. A method of preventing breast cancer recurrence in an adult patient, comprising providing adjuvant treatment to a patient who has received a prior treatment for HR+/HER2− stage II or III early breast cancer, wherein the adjuvant treatment comprises administering to the patient 400 mg ribociclib or a pharmaceutically acceptable salt thereof on days 1 to 21 of a 28-day cycle for at least 36 months, in combination with an aromatase inhibitor as defined in any one of embodiments 26 to 31 administered on every day of the 28-day cycle.
113. The method of embodiment 112, wherein the prior treatment is surgical resection of the cancer.
114. Ribociclib or a pharmaceutically acceptable salt thereof and an aromatase inhibitor for use in a method of treatment for HR+/HER2− stage II or III early breast cancer in an adult patient, wherein the method is the method of any one of embodiments 1 to 112.
115. The use of ribociclib or a pharmaceutically acceptable salt thereof and an aromatase inhibitor in the manufacture of a medicament for treating HR+/HER2− stage II or III early breast cancer in an adult patient, wherein the medicament is administered by the method of any one of embodiments 1 to 112.
116. A kit for performing a method of treating HR+/HER2− stage II or stage III early breast cancer in an adult patient according to any one of embodiments 1 to 112.
117. A method of treating HR+/HER2− stage II or stage III early breast cancer in an adult patient in remission, comprising administering to the adult patient ribociclib in combination with an aromatase inhibitor whereby the administration maintains the adult patient in remission for at least 3 months.
118. A method of treating HR+/HER2− stage II or stage III early breast cancer in an adult patient, comprising providing adjuvant treatment by administering ribociclib in combination with aromatase inhibitor, wherein the patient at start of the adjuvant treatment is in complete remission, and the administration of ribociclib in combination with aromatase inhibitor maintains the adult patient in complete remission for at least 3 months.
119. The method of embodiment 117 or 118, wherein the administration maintains the adult patient in remission (e.g., complete remission) for at least 6 months.
120. The method of embodiment 117 or 118, wherein the administration maintains the adult patient in remission (e.g., complete remission) for at least 9 months.
121. The method of embodiment 117 or 118, wherein the administration maintains the adult patient in remission (e.g., complete remission) for at least 12 months.
122. The method of embodiment 117 or 118, wherein the administration maintains the adult patient in remission (e.g., complete remission) for at least 15 months.
123. The method of embodiment 117 or 118, wherein the administration maintains the adult patient in remission (e.g., complete remission) for at least 18 months.
124. The method of embodiment 117 or 118, wherein the administration maintains the adult patient in remission (e.g., complete remission) for at least 21 months.
125. The method of embodiment 117 or 118, wherein the administration maintains the adult patient in remission (e.g., complete remission) for at least 24 months.
126. The method of embodiment 117 or 118, wherein the administration maintains the adult patient in remission (e.g., complete remission) for at least 27 months.
127. The method of embodiment 117 or 118, wherein the administration maintains the adult patient in remission (e.g., complete remission) for at least 30 months.
128. The method of embodiment 117 or 118, wherein the administration maintains the adult patient in remission (e.g., complete remission) for at least 33 months.
129. The method of embodiment 117 or 118, wherein the administration maintains the adult patient in remission (e.g., complete remission) for at least 36 months.
130. The method of any one of embodiments 117 to 129, wherein ribociclib and aromatase inhibitor are administered for a treatment duration, and the administration maintains the adult patient in remission (e.g., complete remission) for at least the treatment duration.
131. The method of any one of embodiments 117 to 129, wherein a dose of 400 mg ribociclib is orally administered once daily on days 1-21 of a 28-day cycle and aromatase inhibitor is administered once daily on each day of the 28-day cycle.
132. The method of any one of embodiments 117 to 129, wherein a dose of 400 mg ribociclib is orally administered once daily on days 1-21 of a 28-day cycle for a treatment duration of up to three years and aromatase inhibitor is administered once daily on each day of the 28-day cycle for the up to three years.
133. The method of any one of embodiments 117 to 129, wherein a dose of 400 mg ribociclib is orally administered once daily on days 1-21 of a 28-day cycle for a treatment duration of up to five years and aromatase inhibitor is administered once daily on each day of the 28-day cycle for the up to five years.
134. The method of any one of embodiments 117 to 133, wherein a pharmaceutically acceptable salt of ribociclib is orally administered in an amount corresponding to 400 mg ribociclib.
135. The method of embodiment 134, wherein the pharmaceutically acceptable salt is ribociclib succinate.
136. The method of any one of embodiments 117 to 135, wherein the adult patient was never treated with a 600 mg dose of ribociclib.
137. The method of any one of embodiments 117 to 136, wherein the adult patient has never been diagnosed with HR+/HER2− advanced or metastatic breast cancer.
138. The method of any one of embodiments 117 to 137, wherein ribociclib and aromatase inhibitor are administered as adjuvant therapy.
139. The method of embodiment any one of embodiments 117 to 138, wherein the ribociclib is not administered at a dose of 600 mg/day.
140. The method of any one of embodiments 117 to 139, wherein the dose of ribociclib is administered in tablet form.
141. The method of any one of embodiments 117 to 139, wherein two tablets are orally administered to the adult patient once daily on days 1-21 of a 28-day cycle repeating for a treatment duration, and each tablet contains an amount of a pharmaceutically acceptable ribociclib salt corresponding to 200 mg ribociclib.
142. The method of embodiment 141, wherein the ribociclib salt is ribociclib succinate.
143. The method of any one of embodiments 117 to 142, wherein the aromatase inhibitor is letrozole.
144. The method of any one of embodiments 117 to 142, wherein the aromatase inhibitor is anastrazole.
145. The method of any one of embodiments 117 to 144, wherein the aromatase inhibitor is letrozole, and the letrozole is administered in a dose ranging from 1 mg to 4 mg once daily.
146. The method of any one of embodiments 117 to 144, the aromatase inhibitor is letrozole, and the letrozole is administered in a dose of 2.5 mg once daily.
147. The method of any one of embodiments 117 to 144, wherein the aromatase inhibitor is anastrozole, and the anastrozole is administered in a dose ranging from 0.5 mg once daily to 1.5 mg once daily.
148. The method of any one of embodiments 117 to 144, wherein the aromatase inhibitor is anastrozole, and the anastrozole is administered in a dose of 1 mg once daily.
149. The method of any one of embodiments 114 to 148, further comprising administering to the adult patient a gonadotropin-releasing hormone agonist.
150. The method of embodiment 149, wherein the gonadotropin-releasing hormone agonist is goserelin.
151. The method of embodiment 150, wherein the goserelin is administered in a dose ranging from 2 mg to 5 mg.
152. The method of embodiment 151, wherein the dose of goserelin is 3.6 mg.
153. The method of any one of embodiments 150 to 152, wherein the goserelin is administered subcutaneously.
154. The method of any one of embodiments 150 to 153, wherein the goserelin is administered once every 4 weeks.
155. The method of any one of embodiments 117 to 148, wherein the patient is a postmenopausal woman.
156. The method of any one of embodiments 117 to 154, wherein the patient is a premenopausal woman or a man.
157. The method of any one of embodiments 117 to 156, wherein the breast cancer has a histological subtype that is ductal or a histological subtype that is lobular.
158. The method of any one of embodiments 117 to 156, wherein the breast cancer is a stage IIA cancer or a stage IIB cancer.
159. The method of any one of embodiments 117 to 156, wherein the breast cancer is a stage IIA cancer.
160. The method of any one of embodiments 117 to 156, wherein the breast cancer is a stage IIB cancer.
161. The method of any one of embodiments 117 to 156, wherein the breast cancer is a stage IIIA cancer, a stage IIIB cancer, or a stage IIIC cancer.
162. The method of any one of embodiments 117 to 156, wherein the breast cancer is a stage IIIA cancer.
163. The method of any one of embodiments 117 to 156, wherein the breast cancer is a stage IIIB cancer.
164. The method of any one of embodiments 117 to 156, wherein the breast cancer is a stage IIIC cancer.
165. The method of any one of embodiments 117 to 156, wherein the treatment is administered irrespective of the nodal status of the breast cancer.
166. The method of any one of embodiments 117 to 156, wherein treatment with ribociclib and aromatase inhibitor is not adjusted on the basis of the nodal status of the early breast cancer.
167. The method of any one of embodiments 117 to 156, wherein the breast cancer has a nodal status selected from N0, N1, N2, and N3.
168. The method of any one of embodiments 117 to 156, wherein the breast cancer has a nodal status of NO.
169. The method of any one of embodiments 117 to 156, wherein the breast cancer has a nodal status of N1-N3.
170. The method of any one of embodiments 117 to 156, wherein the breast cancer has a nodal status of N1.
171. The method of any one of embodiments 117 to 156, wherein the breast cancer has a nodal status of N2.
172. The method of any one of embodiments 117 to 156, wherein the breast cancer has a nodal status of N3.
173. The method of any one of embodiments 117 to 156, wherein the breast cancer comprises one or more cells having a histological grade selected from G1, G2, or G3.
174. The method of any one of embodiments 117 to 156, wherein the breast cancer comprises one or more cells having the histological grade G1.
175. The method of any one of embodiments 117 to 156, wherein the breast cancer comprises one or more cells having the histological grade G2.
176. The method of any one of embodiments 117 to 156, wherein the breast cancer comprises one or more cells having the histological grade G3.
177. The method of any one of embodiments 117 to 156, wherein the breast cancer comprises a tumor of category T0, T1, T2, T3, or T4.
178. The method of any one of embodiments 117 to 156, wherein the breast cancer comprises a tumor of category T1, T2, or T3.
179. The method of any one of embodiments 117 to 156, wherein the breast cancer comprises a tumor of category T0.
180. The method of any one of embodiments 117 to 156, wherein the breast cancer comprises a tumor of category T1.
181. The method of any one of embodiments 117 to 156, wherein the breast cancer comprises a tumor of category T2.
182. The method of any one of embodiments 117 to 156, wherein the breast cancer comprises a tumor of category T3.
183. The method of any one of embodiments 117 to 156, wherein the breast cancer comprises a tumor of category T4.
184. The method of any one of embodiments 117 to 156, wherein the breast cancer has a Ki67 status of 20 or lower.
185. The method of any one of embodiments 117 to 156, wherein the breast cancer has a Ki67 status of greater than 20.
186. The method of any one of embodiments 117 to 185, wherein prior to administering ribociclib and aromatase inhibitor to the adult patient, the patient had surgery for the early breast cancer.
187. The method of any one of embodiments 117 to 185, wherein prior to administering ribociclib and aromatase inhibitor to the adult patient, the patient had (1) surgery for the early breast cancer, followed by (2) chemotherapy.
188. The method of any one of embodiments 117 to 185, wherein prior to administering ribociclib and aromatase inhibitor to the adult patient, the patient had (1) surgery for the early breast cancer, followed by (2) chemotherapy and/or endocrine therapy.
189. The method of embodiment 188, wherein the endocrine therapy was therapy with a prior aromatase inhibitor.
190. The method of embodiment 188, wherein the prior aromatase inhibitor was letrozole or anastrozole.
191. The method of any one of embodiments 117 to 185, wherein immediately prior to administering ribociclib and aromatase inhibitor to the adult patient, the patient had surgery for the early breast cancer.
192. The method of any one of embodiments 186 to 191, wherein the surgery comprised a complete surgical resection of the cancer.
193. The method of any one of embodiments 186 to 191, wherein the surgery was a mastectomy.
194. The method of any one of embodiments 186 to 191, wherein the patient received neoadjuvant therapy.
195. The method of embodiment 194, wherein the neoadjuvant therapy was chemotherapy.
196. The method of any one of embodiments 117 to 195, wherein the adult patient is located in a geographic region selected from North America, Western Europe, or Oceania.
197. The method of any one of embodiments 117 to 195, wherein the patient is Asian.
198. The method of any one of embodiments 117 to 195, wherein the patient is 18 to 45 years of age.
199. The method of any one of embodiments 117 to 195, wherein the patient is 45 to 54 years of age.
200. The method of any one of embodiments 117 to 195, wherein the patient is 54 to 64 years of age.
201. The method of any one of embodiments 117 to 195, wherein the patient is greater than
64 years of age.
202. The method of any one of embodiments 117 to 195, wherein the patient has a BMI of 25 or higher.
203. The method of any one of embodiments 117 to 195, wherein the patient has a BMI lower than 25.
204. The method of any one of embodiments 117 to 203, wherein the treatment improves a condition of the patient relative to a patient not receiving the treatment and/or relative to the condition in the patient prior to treatment.
205. The method of any one of embodiments 117 to 203, wherein the treatment reduces the risk of invasive disease.
206. The method of any one of embodiments 117 to 203, wherein the treatment reduces the risk of invasive disease in the adult patient corresponding to a hazard ratio of less than 1 when the risk is calculated relative to other patients having received the same treatment as the adult patient except that the other patients did not receive the ribociclib.
207. The method any one of embodiments 117 to 203, wherein the treatment reduces the risk of invasive disease in the adult patient corresponding to a hazard ratio of less than or equal to 0.78 when the risk is calculated relative to other patients having received the same treatment as the adult patient except that the other patients did not receive the ribociclib.
208. The method of any one of embodiments 117 to 203, wherein the adult patient is a premenopausal woman or a man and the treatment reduces the risk of invasive disease corresponding to a hazard ratio of less than or equal to 0.72 when the risk is calculated relative to other premenopausal women or men, respectively, having received the same treatment as the premenopausal woman or man, respectively, except that the other premenopausal women or men did not receive the ribociclib.
209. The method of any one of embodiments 117 to 203, wherein the adult patient was diagnosed with HR+/HER2− stage III early breast cancer and the treatment reduces the risk of invasive disease in the adult patient corresponding to a hazard ratio of less than or equal to 0.74 when the risk is calculated relative to other patients having received the same treatment as the adult patient except that the other patients did not receive the ribociclib.
210. The method of any one of embodiments 117 to 203, wherein the adult patient was diagnosed with HR+/HER2− stage II early breast cancer and the treatment reduces the risk of invasive disease corresponding to a hazard ratio of less than or equal to 0.76 when the risk is calculated relative to other patients having received the same treatment as the adult patient except that the other patients did not receive the ribociclib.
211. The method of any one of embodiments 117 to 203, wherein the adult patient was diagnosed with HR+/HER2− stage III early breast cancer, and the treatment yields at least a 25% reduction in the risk of invasive disease corresponding to a hazard ratio of 0.75 when the risk is calculated relative to other patients having received the same treatment as the adult patient except that the other patients did not receive the ribociclib.
212. The method of any one of embodiments 117 to 203, wherein the treatment reduces the risk of invasive disease to a similar level for patient subgroups comprising patients with stage II early breast cancer, stage III early breast cancer, patients who are premenopausal women or men, and patients who are postmenopausal women.
213. The method of any one of embodiments 117 to 203, wherein the treatment yields no deterioration in overall survival corresponding to a hazard ratio of 0.76 when the risk is calculated relative to other patients having received the same treatment as the adult patient except that the other patients did not receive the ribociclib.
214. The method of one of embodiments 117 to 203, wherein the treatment reduces and/or prevents one or more of recurrence of the cancer, spread of the cancer, development and/or growth of an additional cancer, and risk of death from the cancer.
215. The method of one of embodiments 117 to 203, wherein the treatment reduces the recurrence of cancer, wherein the recurrence is one or more of invasive ipsilateral breast tumor (IBTR) recurrence, local-regional invasive recurrence, and distant recurrence.
216. The method of one of embodiments 117 to 203, wherein the treatment reduces the spread of cancer, wherein the spread of cancer is an invasive contralateral breast cancer or an additional primary invasive cancer.
217. Ribociclib or a pharmaceutically acceptable salt thereof and an aromatase inhibitor for use in a method of treatment for HR+/HER2− stage II or III early breast cancer in an adult patient, wherein the method is the method of any one of embodiments 117 to 216.
218. The use of ribociclib or a pharmaceutically acceptable salt thereof and an aromatase inhibitor in the manufacture of a medicament for treating HR+/HER2− stage II or III early breast cancer in an adult patient, wherein the medicament is administered by the method of any one of embodiments 117 to 216.
219. A kit for performing a method of treating HR+/HER2− stage II or stage III early breast cancer in an adult patient according to any one of embodiments 117 to 216.
220. A method of treating an adult patient who has been diagnosed with HER+/HER2− stage II or stage III early breast cancer, comprising administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle, wherein the method results in an improvement in the patient in one or more of their overall survival (OS), distant disease-free survival (DDFS), or recurrence free survival (RFS).
221. A method for improving one or more of overall survival (OS), distant disease-free survival (DDFS), or recurrence free survival (RFS) in a patient who has been diagnosed with HR+/HER2− stage II or stage III early breast cancer, comprising administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.
222. A method for reducing the risk of local or regional invasive recurrence of early breast cancer in an adult patient who has been diagnosed with HR+/HER2− stage II or stage III early breast cancer, comprising administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.
223. A method for reducing the risk of invasive recurrence of early breast cancer in one or more of the bone, liver, and lung or pleura of an adult patient, who has been diagnosed with HR+/HER2− stage II or stage III early breast cancer, comprising administering to the patient an adjuvant treatment comprising (i) a dose of ribociclib ranging from 150 mg/day to 450 mg/day on days 1-21 of a 28-day cycle, and (ii) an aromatase inhibitor administered on every day of the 28-day cycle.
224. The method of any of embodiments 220 to 223, wherein the treatment is administered irrespective of the nodal status of the breast cancer.
225. A method for reducing the risk of recurrence of early breast cancer in an adult patient who has been diagnosed with HR+/HER2− stage II or stage III early breast cancer, comprising administering to the patient an adjuvant treatment comprising (i) a dose of ribociclib ranging from 150 mg/day to 450 mg/day on days 1-21 of a 28-day cycle and (ii) an aromatase inhibitor administered on every day of the 28-day cycle, wherein the treatment is administered irrespective of the nodal status of the breast cancer.
226. The method of any of embodiments 220 to 225, wherein the breast cancer has a nodal status of N0, N1, N2 or N3. 227. The method of embodiment 226, wherein the breast cancer has a nodal status of NO.
228. The method of any of embodiments 220 to 227, wherein the early breast cancer is stage II, for example stage IIA.
229. The method of any of embodiments 220 to 227, wherein the early breast cancer is stage III, for example stage IIIB or IIIC.
230. The method of any of embodiments 220 to 229, wherein the breast cancer is of the ductal subtype.
231. The method of any of embodiments 220 to 230, wherein the patient is Asian.
232. The method of any of embodiments 220 to 231, wherein the method is an adjuvant treatment because the patient has received at least one prior treatment for breast cancer selected from the group consisting of surgery, chemotherapy, and radiation therapy.
233. The method of embodiment 232, wherein the patient has not had a mastectomy.
234. The method of any of embodiments 220 to 233, wherein the dose of ribociclib is 400 mg/day.
235. The method of any of embodiments 220 to 234, wherein the ribociclib is administered in the form of a salt, preferably as ribociclib succinate.
236. The method of any of embodiments 220 to 235, wherein the aromatase inhibitor is letrozole or anastrozole.
237. The method of embodiment 236, wherein the aromatase inhibitor is letrozole, preferably administered in a dose of 2.5 mg/day.
238. The method of embodiment 236, wherein the aromatase inhibitor is anastrozole, preferably administered in a dose of 1 mg/day.
239. The method of any of embodiments 220 to 238, wherein the dose of ribociclib is 400 mg/day, the ribociclib is administered in the form of ribociclib succinate, and the aromatase inhibitor is letrozole or anastrozole, preferably 2.5 mg/day of letrozole or 1 mg/day anastrozole.
240. The method of any of embodiments 220 to 239, wherein the improvement in OS, DDFS and/or RFS, or the reduction in the risk of recurrence of the breast cancer, are achieved in the patient for at least 36 months.
241. Ribociclib for use in a method of improving one or more of overall survival (OS), distant disease-free survival (DDFS), or recurrence free survival (RFS) in an adult patient who has been diagnosed with HR+/HER2− stage II or stage III early breast cancer, wherein the method comprises administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.
242. Ribociclib for use in a method of reducing the risk of local or regional invasive recurrence of early breast cancer in an adult patient who has been diagnosed with HR+/HER2− stage II or stage III early breast cancer, wherein the method comprises administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.
243. Ribociclib for use in a method of reducing the risk of invasive recurrence of early breast cancer in one or more of the bone, liver, and lung or pleura of an adult patient who has been diagnosed with HR+/HER2− stage II or stage III early breast cancer, wherein the method comprises administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.
244. Ribociclib for use in accordance with any of embodiments 241 to 243, wherein the treatment is administered irrespective of the nodal status of the breast cancer.
245. Ribociclib for use in a method of reducing the risk of recurrence of early breast cancer in an adult patient who has been diagnosed with HR+/HER2− stage II or stage III early breast cancer, comprising administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle, wherein the treatment is administered irrespective of the nodal status of the breast cancer.
246. Ribociclib for use according to any of embodiments 241 to 245, wherein the breast cancer has a nodal status of N0, N1, N2 or N3.
247. Ribociclib for use according to embodiment 246, wherein the breast cancer has a nodal status of NO.
248. Ribociclib for use according to any of embodiments 241 to 247, wherein the early breast cancer is stage II, such as stage IIA.
249. Ribociclib for use according to any of embodiments 241 to 247, wherein the early breast cancer is stage III, such as stage IIIB or IIIC.
250. Ribociclib for use according to any of embodiments 241 to 249, wherein the breast cancer is of the ductal subtype.
251. Ribociclib for use according to any of embodiments 241 to 250, wherein the patient is Asian.
252. Ribociclib for use according to any of embodiments 241 to 251, wherein the method is an adjuvant treatment because the patient has received at least one prior treatment for breast cancer selected from the group consisting of surgery, chemotherapy, and radiation therapy.
253. Ribociclib for use according to embodiment 252, wherein the patient has not had a mastectomy.
254. Ribociclib for use according to any of embodiments 241 to 253, wherein the dose of ribociclib is 400 mg/day.
255. Ribociclib for use according to any of embodiments 241 to 254, wherein the ribociclib is administered in the form of a salt, preferably as ribociclib succinate.
256. Ribociclib for use according to any of embodiments 241 to 255, wherein the aromatase inhibitor is letrozole or anastrozole.
257. Ribociclib for use according to embodiment 256, wherein the aromatase inhibitor is letrozole, preferably administered in a dose of 2.5 mg/day.
258. Ribociclib for use according to embodiment 256, wherein the aromatase inhibitor is anastrozole, preferably administered in a dose of 1 mg/day.
259. Ribociclib for use according to any of embodiments 241 to 258, wherein the dose of ribociclib is 400 mg/day, the ribociclib is administered in the form of ribociclib succinate, and the aromatase inhibitor is letrozole or anastrozole, preferably 2.5 mg/day of letrozole or 1 mg/day anastrozole.
260. Ribociclib for use according to any of embodiments 241 to 259, wherein the improvement in OS, DDFS and/or RFS, or the reduction in the risk of recurrence of the breast cancer, are achieved in the patient for at least 36 months.
261. Ribociclib succinate for use in a method of treatment of HR+/HER2− stage II or stage III early breast cancer in an adult patient who has received at least one prior treatment for early breast cancer and has no signs or symptoms of cancer, wherein the method comprises administering to the patient an adjuvant treatment comprising (i) a dose of ribociclib succinate at a total dose of ribociclib of 400 mg/day administered on days 1 to
21 of a 28-day cycle, and (ii) a dose of either 2.5 mg/day letrozole or 1 mg/day anastrozole administered on every day of the 28-day cycle.
262. Ribociclib succinate for use according to embodiment 261, wherein the adjuvant treatment comprises a dose of 2.5 mg/day letrozole.
263. Ribociclib succinate for use according to embodiment 261, wherein the adjuvant treatment comprises a dose of 1 mg/day anastrozole.
264. Ribociclib succinate for use according to any one of embodiments 261 to 263, wherein the breast cancer has a nodal status of N0, N1, N2 or N3.
265. Ribociclib succinate for use according to embodiment 264, wherein the breast cancer has a nodal status of NO.
266. Ribociclib succinate for use according to any one of embodiments 261 to 265, wherein the early breast cancer is stage II, such as stage IIA.
267. Ribociclib succinate for use according to any one of embodiments 261 to 265, wherein the early breast cancer is stage III, such as stage IIIB.
268. Ribociclib succinate for use according to any one of embodiments 261 to 265, wherein the early breast cancer is stage III, such as IIIC.
269. Ribociclib succinate for use according to any one of embodiments 261 to 268, wherein the breast cancer is of the ductal subtype.
270. Ribociclib succinate for use according to any one of embodiments 261 to 269, wherein the patient is Asian.
271. Ribociclib succinate for use according to any one of embodiments 261 to 270, wherein the method is an adjuvant treatment because the patient has received at least one prior treatment for breast cancer selected from the group consisting of surgery, chemotherapy, endocrine therapy, and radiation therapy.
272. Ribociclib succinate for use according to embodiment 271, wherein the patient has not had a mastectomy.
273. Ribociclib succinate for use according to any one of embodiments 261 to 272, wherein the method improves overall survival (OS), distant disease-free survival (DDFS), and/or recurrence free survival (RFS) of the breast cancer in the patient for at least 36 months; or the method reduces the risk of recurrence of the breast cancer in the patient for at least 36 months.
274. A method of preventing recurrence of breast cancer in an adult patient who has received a prior treatment for HR+/HER2− stage II or stage III early breast cancer, comprising administering to the patient (i) a dose of ribociclib, a freebase form thereof, or a pharmaceutically acceptable salt thereof, and (ii) a dose of an aromatase inhibitor, preferably letrozole or anastrozole.
275. A method of treating an adult patient in remission from HR+/HER2− stage II or stage III early breast cancer, comprising administering to the patient (i) a dose of ribociclib, a freebase form thereof, or a pharmaceutically acceptable salt thereof, and (ii) a dose of an aromatase inhibitor, preferably letrozole or anastrozole.
276. A method of treating HR+/HER2− stage II or stage III early breast cancer in an adult patient in need thereof, comprising administering to the patient (i) a dose of ribociclib, a freebase form thereof, or a pharmaceutically acceptable salt thereof, ranging from 150 mg/day to 450 mg/day administered on days 1-21 of a 28-day cycle and (ii) an aromatase inhibitor, preferably letrozole or anastrozole, administered on every day of the 28-day cycle.
277. The method of any one of embodiments 274 to 276, wherein the ribociclib is a pharmaceutically acceptable ribociclib salt.
278. The method of embodiment 277, wherein the ribociclib salt is ribociclib succinate.
279. The method of any one of embodiments 274, 275, 277, and 278, wherein the dose of ribociclib is not 600 mg/day.
280. The method of any one of embodiments 274 to 278, wherein the dose of ribociclib is 200 mg/day or 400 mg/day.
281. The method of embodiment 280, wherein ribociclib is administered as ribociclib succinate, and the total dose of ribociclib is 200 mg/day.
282. The method of embodiment 280, wherein ribociclib is administered as ribociclib succinate, and the total dose of ribociclib is 400 mg/day.
283. The method of embodiment 280, wherein the ribociclib is administered at a dose of 400 mg/day for a period of time, after which a dose of 200 mg/day ribociclib is administered.
284. The method of any one of embodiments 274 to 283, wherein the dose of ribociclib is administered orally.
285. The method of any one of embodiments 274 to 284, wherein the dose of ribociclib is administered in tablet form.
286. The method of any one of embodiments 274 to 285, wherein the aromatase inhibitor is letrozole or anastrozole.
287. The method of any one of embodiments 274 to 286, wherein the aromatase inhibitor is administered orally.
288. The method of embodiment 286 or 287, wherein the letrozole is administered in a dose ranging from 1 mg/day to 4 mg/day.
289. The method of embodiment 288, wherein the letrozole is administered in a dose of 2.5 mg/day.
290. The method of embodiment 286 or 287, wherein the anastrozole is administered in a dose ranging from 0.5 mg/day to 1.5 mg/day.
291. The method of embodiment 290, wherein the anastrozole is administered in a dose of 1 mg/day.
292. A method of treating breast cancer recurrence in an adult patient who has received at least one prior treatment for HR+/HER2− stage II or III early breast cancer and who has no detectable signs or symptoms of breast cancer, comprising administering to the patient an adjuvant treatment comprising (i) a dose of ribociclib succinate at a total dose of ribociclib of 400 mg/day administered on days 1 to 21 of a 28-day cycle, and (ii) a dose of either 2.5 mg/day letrozole or 1 mg/day anastrozole administered on every day of the 28-day cycle.
293. A method of treating an adult patient who is in remission from HR+/HER2− stage II or stage III early breast cancer and in need of adjuvant treatment, comprising administering to the patient an adjuvant treatment comprising (i) a dose of ribociclib succinate at a total dose of ribociclib of 400 mg/day administered on days 1-21 of a 28-day cycle and (ii) a dose of either 2.5 mg/day letrozole or 1 mg/day anastrozole administered on every day of the 28-day cycle.
294. The method of any one of embodiments 274 to 293, wherein the treatment further comprises administering a gonadotropin-releasing hormone agonist.
295. The method of embodiment 294, wherein the gonadotropin-releasing hormone agonist is goserelin.
296. The method of embodiment 295, wherein the goserelin is administered in a dose ranging from 2 mg to 5 mg.
297. The method of embodiment 296, wherein the dose of goserelin is 3.6 mg.
298. The method of any one of embodiments 294 to 297, wherein the goserelin is administered subcutaneously.
299. The method of any one of embodiments 294 to 298, wherein the goserelin is administered once every 4 weeks.
300. The method of any one of embodiments 274 to 299, wherein the patient is a postmenopausal woman.
301. The method of any one of embodiments 274 to 293, wherein the patient is a premenopausal woman or a man.
302. The method of any one of embodiments 274 to 301, wherein the treatment is administered to the patient for at least 12 months.
303. The method of embodiment 302, wherein the treatment is administered to the patient for at least 24 months.
304. The method of embodiment 302 or 303, wherein the treatment is administered to the patient for at least 36 months.
305. The method of any one of embodiments 302 to 304, wherein the treatment is administered to the patient for at least 48 months.
306. The method of any one of embodiments 302 to 305, wherein the treatment is administered to the patient for at least 60 months.
307. The method of any one of embodiments 274 to 306, wherein the breast cancer is ER+ and PR+.
308. The method of any one of embodiments 274 to 306, wherein the breast cancer is ER− and PR+.
309. The method of any one of embodiments 274 to 306, wherein the breast cancer is ER+ and PR−.
310. The method of any one of embodiments 274 to 309, wherein the breast cancer has a histological subtype that is ductal.
311. The method of any one of embodiments 274 to 310, wherein the breast cancer has a histological subtype that is lobular.
312. The method of any one of embodiments 274 to 311, wherein the breast cancer is a stage IIA cancer or a stage IIB cancer.
313. The method of embodiment 312, wherein the breast cancer is a stage IIA cancer.
314. The method of embodiment 312, wherein the breast cancer is a stage IB cancer.
315. The method of any one of embodiments 274 to 311, wherein the breast cancer is a stage IIIA cancer, a stage IIIB cancer, or a stage IIIC cancer.
316. The method of embodiment 315, wherein the breast cancer is a stage IIIA cancer.
317. The method of embodiment 315, wherein the breast cancer is a stage IIIB cancer.
318. The method of embodiment 315, wherein the breast cancer is a stage IIIC cancer.
319. The method of any one of embodiments 274 to 318, wherein the treatment is administered irrespective of the nodal status of the breast cancer.
320. The method of any one of embodiments 274 to 319, wherein the breast cancer has a nodal status selected from N0, N1, N2, and N3.
321. The method of embodiment 319 or 320, wherein the breast cancer has a nodal status of NO.
322. The method of embodiment 319 or 320, wherein the breast cancer has a nodal status of N1 to N3.
323. The method of embodiment 319 or 320, wherein the breast cancer has a nodal status of N1.
324. The method of embodiment 319 or 320, wherein the breast cancer has a nodal status of N2.
325. The method of embodiment 319 or 320, wherein the breast cancer has a nodal status of N3.
326. The method of any one of embodiments 274 to 325, wherein the breast cancer comprises one or more cells having a histological grade selected from G1, G2, or G3.
327. The method of embodiment 326, wherein the breast cancer comprises one or more cells having the histological grade G1.
328. The method of embodiment 326, wherein the breast cancer comprises one or more cells having the histological grade G2.
329. The method of embodiment 326, wherein the breast cancer comprises one or more cells having the histological grade G3.
330. The method of any one of embodiments 274 to 329, wherein the breast cancer comprises a tumor of category T0, T1, T2, T3, or T4.
331. The method of embodiment 330, wherein the breast cancer comprises a tumor of category T1, T2, or T3.
332. The method of embodiment 330, wherein the breast cancer comprises a tumor of category T0.
333. The method of embodiment 330 or embodiment 331, wherein the breast cancer comprises a tumor of category T1.
334. The method of embodiment 330 or embodiment 331, wherein the breast cancer comprises a tumor of category T2.
335. The method of embodiment 330 or embodiment 331, wherein the breast cancer comprises a tumor of category T3.
336. The method of embodiment 330, wherein the breast cancer comprises a tumor of category T4.
337. The method of any one of embodiments 274 to 336, wherein the breast cancer has a Ki67 status of 20 or lower.
338. The method of any one of embodiments 274 to 336, wherein the breast cancer has a Ki67 status of greater than 20.
339. The method of any one of embodiments 274 to 338, wherein prior to the administration, the patient has received a loading dose of (i) ribociclib and/or (ii) an endocrine therapy.
340. The method of any one of embodiments 274 to 291 and 293 to 339, wherein the patient has received at least one prior treatment for cancer.
341. The method of embodiment 292 or embodiment 340, wherein the prior treatment is an adjuvant treatment after another prior treatment.
342. The method of embodiment 340 or 341, wherein the prior treatment is surgery.
343. The method of embodiment 342, wherein the surgery comprises a complete surgical resection of the cancer.
344. The method of embodiment 342 or embodiment 343, wherein the surgery is a mastectomy.
345. The method of embodiment 340 or embodiment 341, wherein the prior treatment is a chemotherapy.
346. The method of embodiment 345, wherein the prior treatment is an adjuvant chemotherapy.
347. The method of embodiment 345, wherein the prior treatment is a neoadjuvant chemotherapy.
348. The method of embodiment 340 or embodiment 341, wherein the prior treatment is an endocrine therapy.
349. The method of embodiment 340 or embodiment 341, wherein the prior treatment is radiation therapy.
350. The method of any one of embodiments 292 and 340 to 349, wherein the patient did not respond to the prior treatment.
351. The method of any one of embodiments 274 to 350, wherein the patient is located in a geographic region selected from North America, Western Europe, or Oceania.
352. The method of any one of embodiments 274 to 351, wherein the patient is Asian.
353. The method of any one of embodiments 274 to 352, wherein the patient is 18 to 45 years of age.
354. The method of any one of embodiments 274 to 352, wherein the patient is 45 to 54 years of age.
355. The method of any one of embodiments 274 to 352, wherein the patient is 54 to 64 years of age.
356. The method of any one of embodiments 274 to 352, wherein the patient is greater than 64 years of age.
357. The method of any one of embodiments 274 to 357, wherein the patient has a BMI of 25 or higher.
358. The method of any one of embodiments 274 to 357, wherein the patient has a BMI lower than 25.
359. The method of any one of embodiments 274 to 358, wherein the treatment improves a condition of the patient relative to a patient not receiving the treatment and/or relative to the condition in the patient prior to treatment.
360. The method of any one of embodiments 274 to 359, wherein the treatment reduces the risk of invasive disease.
361. The method of embodiment 360, wherein the treatment reduces the risk of invasive disease corresponding to a hazard ratio of less than 1 when the risk is calculated relative to patients not receiving the treatment.
362. The method of embodiment 361, wherein the treatment reduces the risk of invasive disease corresponding to a hazard ratio of less than or equal to 0.78 when the risk is calculated relative to patients not receiving the treatment.
363. The method of any one of embodiments 274 to 299 and 301 to 362, wherein the patient is a premenopausal woman or a man and the treatment reduces the risk of invasive disease corresponding to a hazard ratio of less than or equal to 0.72 when the risk is calculated relative to patients receiving the treatment.
364. The method of any one of embodiments 360 to 362, wherein the cancer is HR+/HER2− stage III early breast cancer and the treatment reduces the risk of invasive disease corresponding to a hazard ratio of less than or equal to 0.74 when the risk is calculated relative to patients not receiving the treatment.
365. The method of any one of embodiments 360 to 362, wherein the cancer is HR+/HER2− stage II early breast cancer and the treatment reduces the risk of invasive disease corresponding to a hazard ratio of less than or equal to 0.76 when the risk is calculated relative to patients not receiving the treatment.
366. The method of any one of embodiments 360 to 362, wherein the cancer is HR+/HER2− stage III early breast cancer, and the treatment yields at least a 25% reduction in the risk of invasive disease corresponding to a hazard ratio of 0.75 when the risk is calculated relative to patients not receiving the treatment.
367. The method of any one of embodiments 360 to 366, wherein the treatment reduces the risk of invasive disease to a similar level for patient subgroups comprising patients with stage II early breast cancer, stage III early breast cancer, patients who are premenopausal women or men, and patients who are postmenopausal women.
368. The method of any one of embodiments 274 to 367, wherein the treatment yields no deterioration in overall survival corresponding to a hazard ratio of 0.76 when the risk is calculated relative to patients not receiving the treatment.
369. The method of any one of embodiments 274 to 368, wherein the treatment reduces and/or prevents one or more of recurrence of the cancer, spread of the cancer, development and/or growth of an additional cancer, and risk of death from the cancer.
370. The method of embodiment 369, wherein the treatment reduces the recurrence of cancer, wherein the recurrence is one or more of invasive ipsilateral breast tumor (IBTR) recurrence, local-regional invasive recurrence, and distant recurrence.
371. The method of embodiment 369, wherein the treatment reduces the spread of cancer, wherein the spread of cancer is an invasive contralateral breast cancer or an additional primary invasive cancer.
372. The method of any one of embodiments 274 to 371, wherein the treatment prevents death from cancer.

### J. FURTHER EXEMPLARY EMBODIMENTS

Further methods of treatment and uses of ribociclib are provided in the following embodiments.
1. A method of treating an adult patient who has been diagnosed with HER+/HER2− stage II or stage III early breast cancer, comprising administering to the patient an adjuvant therapy comprising (i) a dose of ribociclib ranging from 150 mg/day to 450 mg/day administered on days 1-21 of a 28-day cycle, and (ii) an aromatase inhibitor administered on every day of the 28-day cycle, wherein the method results in an improvement in the patient in one or more of their overall survival (OS), distant disease-free survival (DDFS), or recurrence free survival (RFS).
2. A method for improving one or more of overall survival (OS), distant disease-free survival (DDFS), or recurrence free survival (RFS) in a patient who has been diagnosed with HR+/HER2− stage II or stage III early breast cancer, comprising administering to the patient an adjuvant treatment comprising (i) a dose of ribociclib ranging from 150 mg/day to 450 mg/day administered on days 1-21 of a 28-day cycle, (ii) an aromatase inhibitor administered on every day of the 28-day cycle.
3. A method for reducing the risk of local or regional invasive recurrence of early breast cancer in an adult patient who has been diagnosed with HR+/HER2− stage II or stage III early breast cancer, comprising administering to the patient an adjuvant treatment comprising (i) a dose of ribociclib ranging from 150 mg/day to 450 mg/day administered on days 1-21 of a 28-day cycle and (ii) an aromatase inhibitor administered on every day of the 28-day cycle.
4. A method for reducing the risk of invasive recurrence of early breast cancer in one or more of the bone, liver, and lung or pleura of an adult patient, who has been diagnosed with HR+/HER2− stage II or stage III early breast cancer, comprising administering to the patient an adjuvant treatment comprising (i) a dose of ribociclib ranging from 150 mg/day to 450 mg/day on days 1-21 of a 28-day cycle, and (ii) an aromatase inhibitor administered on every day of the 28-day cycle.
5. The method of any of claims 1 to 4, wherein the treatment is administered irrespective of the nodal status of the breast cancer.
6. A method for reducing the risk of recurrence of early breast cancer in an adult patient who has been diagnosed with HR+/HER2− stage II or stage III early breast cancer, comprising administering an adjuvant treatment comprising (i) a dose of ribociclib ranging from 150 mg/day to 450 mg/day on days 1-21 of a 28-day cycle and (ii) an aromatase inhibitor administered on every day of the 28-day cycle, wherein the treatment is administered irrespective of the nodal status of the breast cancer.
7. The method of any of claims 1 to 6, wherein the breast cancer has a nodal status of N0, N1, N2 or N3.
8. The method of claim 7, wherein the breast cancer has a nodal status of NO.
9. The method of any of claims 1 to 8, wherein the early breast cancer is stage II, for example stage IIA.
10. The method of any of claims 1 to 8, wherein the early breast cancer is stage III, for example stage IIB or IIIC.
11. The method of any of claims 1 to 10, wherein the breast cancer is of the ductal subtype.
12. The method of any of claims 1 to 11, wherein the patient is Asian.
13. The method of any of claims 1 to 12, wherein the method is an adjuvant treatment because the patient has received at least one prior treatment for breast cancer selected from the group consisting of surgery, chemotherapy, and radiation therapy.
14. The method of claim 13, wherein the patient has not had a mastectomy.
15. The method of any of claims 1 to 14, wherein the dose of ribociclib is 400 mg/day.
16. The method of any of claims 1 to 15, wherein the ribociclib is administered in the form of a salt, preferably as ribociclib succinate.
17. The method of any of claims 1 to 16, wherein the aromatase inhibitor is letrozole or anastrozole.
18. The method of claim 17, wherein the aromatase inhibitor is letrozole, preferably administered in a dose of 2.5 mg/day.
19. The method of claim 17, wherein the aromatase inhibitor is anastrozole, preferably administered in a dose of 1 mg/day.
20. The method of any of claims 1 to 19, wherein the dose of ribociclib is 400 mg/day, the ribociclib is administered in the form of ribociclib succinate, and the aromatase inhibitor is letrozole or anastrozole, preferably 2.5 mg/day of letrozole or 1 mg/day anastrozole.
21. The method of any of claims 1 to 20, wherein the improvement in OS, DDFS and/or RFS, or the reduction in the risk of recurrence of the breast cancer, are achieved in the patient for at least 36 months.
22. Ribociclib for use in a method of improving one or more of overall survival (OS), distant disease-free survival (DDFS), or recurrence free survival (RFS) in an adult patient who has been diagnosed with HR+/HER2− stage II or stage III early breast cancer, comprising administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.
23. Ribociclib for use in a method of reducing the risk of local or regional invasive recurrence of early breast cancer in an adult patient who has been diagnosed with HR+/HER2− stage II or stage III early breast cancer, comprising g administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.
24. Ribociclib for use in a method of reducing the risk of invasive recurrence of early breast cancer in one or more of the bone, liver, and lung or pleura of an adult patient who has been diagnosed with HR+/HER2− stage II or stage III early breast cancer and the method is an adjuvant treatment comprising administering to the patient an adjuvant treatment comprising (i) a dose of ribociclib ranging from 150 mg/day to 450 mg/day administered on days 1-21 of a 28-day cycle, and (ii) an aromatase inhibitor administered on every day of the 28-day cycle.
25. Ribociclib for use in accordance with any of claims 22 to 24, wherein the treatment is administered irrespective of the nodal status of the breast cancer.
26. Ribociclib for use in a method of reducing the risk of recurrence of early breast cancer in an adult patientwho has been diagnosed with HR+/HER2− stage II or stage III early breast cancer, comprising administering to the patient an adjuvant treatment comprising (i) a dose of ribociclib ranging from 150 mg/day to 450 mg/day administered on days 1-21 of a
28-day cycle, and (ii) an aromatase inhibitor administered on every day of the 28-day cycle, wherein the treatment is administered irrespective of the nodal status of the breast cancer.
27. Ribociclib for use according to any of claims 22 to 26, wherein the breast cancer has a nodal status of N0, N1, N2 or N3. 28. Ribociclib for use according to claim 27, wherein the breast cancer has a nodal status of NO.
29. Ribociclib for use according to any of claims 22 to 28, wherein the early breast cancer is stage II, such as stage IIA.
30. Ribociclib for use according to any of claims 22 to 28, wherein the early breast cancer is stage III, such as stage IIIB or IIIC.
31. Ribociclib for use according to any of claims 22 to 30, wherein the breast cancer is of the ductal subtype.
32. Ribociclib for use according to any of claims 22 to 31, wherein the patient is Asian.
33. Ribociclib for use according to any of claims 22 to 32, wherein the method is an adjuvant treatment because the patient has received at least one prior treatment for breast cancer selected from the group consisting of surgery, chemotherapy, and radiation therapy.
34. Ribociclib for use according to claim 33, wherein the patient has not had a mastectomy.
35. Ribociclib for use according to any of claims 22 to 34, wherein the dose of ribociclib is 400 mg/day.
36. Ribociclib for use according to any of claims 22 to 35, wherein the ribociclib is administered in the form of a salt, preferably as ribociclib succinate.
37. Ribociclib for use according to any of claims 22 to 36, wherein the aromatase inhibitor is letrozole or anastrozole.
38. Ribociclib for use according to claim 37, wherein the aromatase inhibitor is letrozole, preferably administered in a dose of 2.5 mg/day.
39. Ribociclib for use according to claim 37, wherein the aromatase inhibitor is anastrozole, preferably administered in a dose of 1 mg/day.
40. Ribociclib for use according to any of claims 22 to 39, wherein the dose of ribociclib is 400 mg/day, the ribociclib is administered in the form of ribociclib succinate, and the aromatase inhibitor is letrozole or anastrozole, preferably 2.5 mg/day of letrozole or 1 mg/day anastrozole.
41. Ribociclib for use according to any of claims 22 to 40, wherein the improvement in OS, DDFS and/or RFS, or the reduction in the risk of recurrence of the breast cancer, are achieved in the patient for at least 36 months.

### K. MORE EXEMPLARY EMBODIMENTS

More methods of treatment and uses of ribociclib are provided in the following embodiments.
1. Ribociclib succinate for use in a method of treatment of HR+/HER2− stage II or stage III early breast cancer in an adult patient who has received at least one prior treatment for early breast cancer and has no signs or symptoms of cancer, wherein the method comprises administering to the patient an adjuvant treatment comprising (i) a dose of ribociclib succinate at a total dose of ribociclib of 400 mg/day administered on days 1 to 21 of a 28-day cycle, and (ii) a dose of either 2.5 mg/day letrozole or 1 mg/day anastrozole administered on every day of the 28-day cycle.
2. Ribociclib succinate for use according to embodiment 1, wherein the adjuvant treatment comprises a dose of 2.5 mg/day letrozole.
3. Ribociclib succinate for use according to embodiment 1, wherein the adjuvant treatment comprises a dose of 1 mg/day anastrozole.
4. Ribociclib succinate for use according to any one of embodiments 1 to 3, wherein the breast cancer has a nodal status of N0, N1, N2 or N3.
5. Ribociclib succinate for use according to embodiment 4, wherein the breast cancer has a nodal status of NO.
6. Ribociclib succinate for use according to any one of embodiments 1 to 5, wherein the early breast cancer is stage II, such as stage IIA.
7. Ribociclib succinate for use according to any one of embodiments 1 to 5, wherein the early breast cancer is stage III, such as stage IIIB.
8. Ribociclib succinate for use according to any one of embodiments 1 to 5, wherein the early breast cancer is stage III, such as IIIC.
9. Ribociclib succinate for use according to any one of embodiments 1 to 8, wherein the breast cancer is of the ductal subtype.
10. Ribociclib succinate for use according to any one of embodiments 1 to 9, wherein the patient is Asian.
11. Ribociclib succinate for use according to any one of embodiments 1 to 10, wherein the method is an adjuvant treatment because the patient has received at least one prior treatment for breast cancer selected from the group consisting of surgery, chemotherapy, endocrine therapy, and radiation therapy.
12. Ribociclib succinate for use according to embodiment 11, wherein the patient has not had a mastectomy.
13. Ribociclib succinate for use according to any one of embodiments 1 to 12, wherein the method improves overall survival (OS), distant disease-free survival (DDFS), and/or recurrence free survival (RFS) of the breast cancer in the patient for at least 36 months; or the method reduces the risk of recurrence of the breast cancer in the patient for at least 36 months.

### L. ADDITIONAL EXEMPLARY EMBODIMENTS

Additional methods of treatment and uses of ribociclib are provided in the following embodiments.
1. A method of preventing recurrence of breast cancer in an adult patient who has received a prior treatment for HR+/HER2− stage II or stage III early breast cancer, comprising administering to the patient (i) a dose of ribociclib, a freebase form thereof, or a pharmaceutically acceptable salt thereof, and (ii) a dose of an aromatase inhibitor, preferably letrozole or anastrozole.
2. A method of treating an adult patient in remission from HR+/HER2− stage II or stage III early breast cancer, comprising administering to the patient (i) a dose of ribociclib, a freebase form thereof, or a pharmaceutically acceptable salt thereof, and (ii) a dose of an aromatase inhibitor, preferably letrozole or anastrozole.
3. A method of treating HR+/HER2− stage II or stage III early breast cancer in an adult patient in need thereof, comprising administering to the patient (i) a dose of ribociclib, a freebase form thereof, or a pharmaceutically acceptable salt thereof, ranging from 150 mg/day to 450 mg/day administered on days 1-21 of a 28-day cycle and (ii) an aromatase inhibitor, preferably letrozole or anastrozole, administered on every day of the 28-day cycle.
4. The method of any one of embodiments 1 to 3, wherein the ribociclib is a pharmaceutically acceptable ribociclib salt.
5. The method of embodiment 4, wherein the ribociclib salt is ribociclib succinate.
6. The method of any one of embodiments 1, 2, 4, and 5, wherein dose of ribociclib is not
600 mg/day.
7. The method of any one of embodiments 1 to 5, wherein the dose of ribociclib is 200 mg/day or 400 mg/day.
8. The method of embodiment 7, wherein ribociclib is administered as ribociclib succinate, and the total dose of ribociclib is 200 mg/day.
9. The method of embodiment 7, wherein ribociclib is administered as ribociclib succinate, and the total dose of ribociclib is 400 mg/day.
10. The method of embodiment 7, wherein the ribociclib is administered at a dose of 400 mg/day for a period of time, after which a dose of 200 mg/day ribociclib is administered.
11. The method of any one of embodiments 1 to 10, wherein the dose of ribociclib is administered orally.
12. The method of any one of embodiments 1 to 11, wherein the dose of ribociclib is administered in tablet form.
13. The method of any one of embodiments 1 to 12, wherein the aromatase inhibitor is letrozole or anastrozole.
14. The method of any one of embodiments 1 to 13, wherein the aromatase inhibitor is administered orally.
15. The method of embodiment 13 or 14, wherein the letrozole is administered in a dose ranging from 1 mg/day to 4 mg/day.
16. The method of embodiment 15, wherein the letrozole is administered in a dose of 2.5 mg/day.
17. The method of embodiment 13 or 14, wherein the anastrozole is administered in a dose ranging from 0.5 mg/day to 1.5 mg/day.
18. The method of embodiment 17, wherein the anastrozole is administered in a dose of 1 mg/day.
19. A method of treating breast cancer recurrence in an adult patient who has received at least one prior treatment for HR+/HER2− stage II or III early breast cancer and who has no detectable signs or symptoms of breast cancer, comprising administering to the patient an adjuvant treatment comprising (i) a dose of ribociclib succinate at a total dose of ribociclib of 400 mg/day administered on days 1 to 21 of a 28-day cycle, and (ii) a dose of either 2.5 mg/day letrozole or 1 mg/day anastrozole administered on every day of the 28-day cycle.
20. A method of treating an adult patient who is in remission from HR+/HER2− stage II or stage III early breast cancer and in need of adjuvant treatment, comprising administering to the patient an adjuvant treatment comprising (i) a dose of ribociclib succinate at a total dose of ribociclib of 400 mg/day administered on days 1-21 of a 28-day cycle and (ii) a dose of either 2.5 mg/day letrozole or 1 mg/day anastrozole administered on every day of the 28-day cycle.
21. The method of any one of embodiments 1 to 20, wherein the treatment comprises administering a gonadotropin-releasing hormone agonist.
22. The method of embodiment 21, wherein the gonadotropin-releasing hormone agonist is goserelin.
23. The method of embodiment 22, wherein the goserelin is administered in a dose ranging from 2 mg to 5 mg.
24. The method of embodiment 23, wherein the dose of goserelin is 3.6 mg.
25. The method of any one of embodiments 22 to 24, wherein the goserelin is administered subcutaneously.
26. The method of any one of embodiments 22 to 25, wherein the goserelin is administered once every 4 weeks.
27. The method of any one of embodiments 1 to 20, wherein the patient is a postmenopausal woman.
28. The method of any one of embodiments 1 to 26, wherein the patient is a premenopausal woman or a man.
29. The method of any one of embodiments 1 to 28, wherein the treatment is administered to the patient for at least 12 months.
30. The method of embodiment 29, wherein the treatment is administered to the patient for at least 24 months.
31. The method of embodiment 29 or embodiment 30, wherein the treatment is administered to the patient for at least 36 months.
32. The method of any one of embodiments 29 to 31, wherein the treatment is administered to the patient for at least 48 months.
33. The method of any one of embodiments 29 to 32, wherein the treatment is administered to the patient for at least 60 months.
34. The method of any one of embodiments 1 to 33, wherein the breast cancer is ER+ and PR+.
35. The method of any one of embodiments 1 to 33, wherein the breast cancer is ER- and PR+.
36. The method of any one of embodiments 1 to 33, wherein the breast cancer is ER+ and PR−.
37. The method of any one of embodiments 1 to 36, wherein the breast cancer has a histological subtype that is ductal.
38. The method of any one of embodiments 1 to 37, wherein the breast cancer has a histological subtype that is lobular.
39. The method of any one of embodiments 1 to 38, wherein the breast cancer is a stage IIA cancer or a stage IIB cancer.
40. The method of embodiment 39, wherein the breast cancer is a stage IIA cancer.
41. The method of embodiment 39, wherein the breast cancer is a stage IIB cancer.
42. The method of any one of embodiments 1 to 38, wherein the breast cancer is a stage IIA cancer, a stage IIIB cancer, or a stage IIIC cancer.
43. The method of embodiment 42, wherein the breast cancer is a stage IIIA cancer.
44. The method of embodiment 42, wherein the breast cancer is a stage IIIB cancer.
45. The method of embodiment 42, wherein the breast cancer is a stage IIIC cancer.
46. The method of any one of embodiments 1 to 45, wherein the treatment is administered irrespective of the nodal status of the breast cancer.
47. The method of any one of embodiments 1 to 46, wherein the breast cancer has a nodal status selected from N0, N1, N2, and N3.
48. The method of embodiment 46 or 47, wherein the breast cancer has a nodal status of NO.
49. The method of embodiment 46 or 47, wherein the breast cancer has a nodal status of N1 to N3.
50. The method of embodiment 46 or 47, wherein the breast cancer has a nodal status of N1.
51. The method of embodiment 46 or 47, wherein the breast cancer has a nodal status of N2.
52. The method of embodiment 46 or 47, wherein the breast cancer has a nodal status of N3.
53. The method of any one of embodiments 1 to 52, wherein the breast cancer comprises one or more cells having a histological grade selected from G1, G2, or G3.
54. The method of embodiment 53, wherein the breast cancer comprises one or more cells having the histological grade G1.
55. The method of embodiment 53, wherein the breast cancer comprises one or more cells having the histological grade G2.
56. The method of embodiment 53, wherein the breast cancer comprises one or more cells having the histological grade G3.
57. The method of any one of embodiments 1 to 56, wherein the breast cancer comprises a tumor of category T0, T1, T2, T3, or T4.
58. The method of embodiment 57, wherein the breast cancer comprises a tumor of category T1, T2, or T3.
59. The method of embodiment 57, wherein the breast cancer comprises a tumor of category T0.
60. The method of embodiment 57 or 58, wherein the breast cancer comprises a tumor of category T1.
61. The method of embodiment 57 or 58, wherein the breast cancer comprises a tumor of category T2.
62. The method of embodiment 57 or 58, wherein the breast cancer comprises a tumor of category T3.
63. The method of embodiment 57, wherein the breast cancer comprises a tumor of category T4.
64. The method of any one of embodiments 1 to 63, wherein the breast cancer has a Ki67 status of 20 or lower.
65. The method of any one of embodiments 1 to 63, wherein the breast cancer has a Ki67 status of greater than 20.
66. The method of any one of embodiments 1 to 65, wherein prior to the administration, the patient has received a loading dose of (i) ribociclib and/or (ii) an endocrine therapy.
67. The method of any one of embodiments 1 to 18 and 20 to 66, wherein the patient has received at least one prior treatment for cancer.
68. The method of embodiment 19 or 67, wherein the prior treatment is an adjuvant treatment after another prior treatment.
69. The method of embodiment 67 or 68, wherein the prior treatment is surgery.
70. The method of embodiment 69, wherein the surgery comprises a complete surgical resection of the cancer.
71. The method of embodiment 69 or 70, wherein the surgery is a mastectomy.
72. The method of embodiment 67 or 68, wherein the prior treatment is a chemotherapy.
73. The method of embodiment 72, wherein the prior treatment is an adjuvant chemotherapy.
74. The method of embodiment 72, wherein the prior treatment is a neoadjuvant chemotherapy.
75. The method of embodiment 67 or 68, wherein the prior treatment is an endocrine therapy.
76. The method of embodiment 67 or 68, wherein the prior treatment is radiation therapy.
77. The method of any one of embodiments 19 and 67 to 76, wherein the patient did not respond to the prior treatment.
78. The method of any one of embodiments 1 to 77, wherein the patient is located in a geographic region selected from North America, Western Europe, or Oceania.
79. The method of any one of embodiments 1 to 78, wherein the patient is Asian.
80. The method of any one of embodiments 1 to 79, wherein the patient is 18 to 45 years of age.
81. The method of any one of embodiments 1 to 79, wherein the patient is 45 to 54 years of age.
82. The method of any one of embodiments 1 to 79, wherein the patient is 54 to 64 years of age.
83. The method of any one of embodiments 1 to 79, wherein the patient is greater than 64 years of age.
84. The method of any one of embodiments 1 to 83, wherein the patient has a BMI of 25 or higher.
85. The method of any one of embodiments 1 to 83, wherein the patient has a BMI lower than 25.
86. The method of any one of embodiments 1 to 85, wherein the treatment improves a condition of the patient relative to a patient not receiving the treatment and/or relative to the condition in the patient prior to treatment.
87. The method of any one of embodiments 1 to 86, wherein the treatment reduces the risk of invasive disease.
88. The method of embodiment 87, wherein the treatment reduces the risk of invasive disease corresponding to a hazard ratio of less than 1 when the risk is calculated relative to patients not receiving the treatment.
89. The method of embodiment 88, wherein the treatment reduces the risk of invasive disease corresponding to a hazard ratio of less than or equal to 0.78 when the risk is calculated relative to patients not receiving the treatment.
90. The method of any one of embodiments 1 to 26 and 28 to 89, wherein the patient is a premenopausal woman or a man and the treatment reduces the risk of invasive disease corresponding to a hazard ratio of less than or equal to 0.72 when the risk is calculated relative to patients receiving the treatment.
91. The method of any one of embodiments 87 to 89, wherein the cancer is HR+/HER2− stage III early breast cancer and the treatment reduces the risk of invasive disease corresponding to a hazard ratio of less than or equal to 0.74 when the risk is calculated relative to patients not receiving the treatment.
92. The method of any one of embodiments 87 to 89, wherein the cancer is HR+/HER2− stage II early breast cancer and the treatment reduces the risk of invasive disease corresponding to a hazard ratio of less than or equal to 0.76 when the risk is calculated relative to patients not receiving the treatment.
93. The method of any one of embodiments 87 to 89, wherein the cancer is HR+/HER2− stage III early breast cancer, and the treatment yields at least a 25% reduction in the risk of invasive disease corresponding to a hazard ratio of 0.75 when the risk is calculated relative to patients not receiving the treatment.
94. The method of any one of embodiments 1 to 93, wherein the treatment reduces the risk of invasive disease to a similar level for patient subgroups comprising patients with stage II early breast cancer, stage III early breast cancer, patients who are premenopausal women or men, and patients who are postmenopausal women.
95. The method of any one of embodiments 1 to 94, wherein the treatment yields no deterioration in overall survival corresponding to a hazard ratio of 0.76 when the risk is calculated relative to patients not receiving the treatment.
96. The method of any one of embodiments 1 to 95, wherein the treatment reduces and/or prevents one or more of recurrence of the cancer, spread of the cancer, development and/or growth of an additional cancer, and risk of death from the cancer.
97. The method of embodiment 96, wherein the treatment reduces the recurrence of cancer, wherein the recurrence is one or more of invasive ipsilateral breast tumor (IBTR) recurrence, local-regional invasive recurrence, and distant recurrence.
98. The method of embodiment 96, wherein the treatment reduces the spread of cancer, wherein the spread of cancer is an invasive contralateral breast cancer or an additional primary invasive cancer.
99. The method of any one of embodiments 1 to 98, wherein the treatment prevents death from cancer.

### XI. DEFINITIONS

Other than in the examples, or where otherwise indicated, all numbers expressing quantities of ingredients, dosages or reaction conditions used herein should be understood as modified in all instances by the term “about” as that term would be interpreted by the person skilled in the relevant art. The term “about” as used herein is equivalent to ±10% of a given numerical value, unless otherwise stated.

“Adjuvant therapy” (or adjuvant treatment) refers to a treatment given after a primary treatment, e.g., in order to lower the risk that the disease will recur. In cancer, an adjuvant therapy may be administered, inter alia, to lower the risk of cancer recurrence, for example, by destroying cancer cells that remain after a primary treatment. For example, a treatment comprising a dose of ribociclib in combination with an aromatase inhibitor may be administered as an adjuvant after a primary treatment, e.g., comprising surgery, radiation therapy, and/or chemotherapy.

“Neoadjuvant therapy” (or neoadjuvant treatment) refers to a treatment delivered before a primary treatment. In cancer, a neoadjuvant therapy may, inter alia, reduce the size of a tumor or kill cancer cells that have spread.

“Co-administer,” “co-administration,” or “combined administration” or the like are meant to encompass administration of the selected therapeutic agents to a single patient, and encompass treatment regimens in which the agents are not necessarily administered by the same route of administration or at the same time.

“Combination” refers to either a fixed combination of two or more agents (also referred to as co-agents or combination partners), e.g., in one dosage unit form, or a nonfixed combination (or kit of parts) for the combined administration where a first agent (also referred to as a first therapeutic agent) and a second agent (also referred to as a second therapeutic agent) may be administered independently at the same time or separately within time intervals, e.g., where these time intervals allow the combination partners to provide a cooperative, e.g. synergistic effect. The term “combined administration” or the like as utilized herein is meant to encompass administration of the selected combination partners to a single subject in need thereof (e.g. a patient), and is intended to include treatment regimens in which the agents are not necessarily administered by the same route of administration or at the same time. The term “fixed combination” means that the active ingredients, e.g. combination partners, are both administered to a patient simultaneously in the form of a single dosage. The terms “non-fixed combination” or “kit of parts” mean that the active ingredients, e.g. combination partners, are provided separately, e.g., within a kit, and/or are both administered to a patient as separate dosage forms either concurrently or sequentially with no specific time limits, wherein such administration provides therapeutically effective levels of the two compounds in the body of the patient.

“Dose range” refers to an upper and a lower limit of an acceptable variation of the amount of therapeutic agent specified. Typically, a dose of the agent in any amount within the specified range can be administered to patients undergoing treatment.

A “loading dose” may be an initial dose of a drug that may be given at or prior to the beginning of a course of treatment before dropping down to a different, typically lower dose (e.g., a treatment or maintenance dose). A loading dose may be a single dose or short duration regimen of a compound which is administered to the subject to rapidly increase the blood concentration level of the drug. Suitably, a short duration regimen for use herein will be from: 1 to 14 days; e.g., from 1 to 7 days; e.g., from 1 to 3 days; e.g., for three days; e.g., for two days; e.g., for one day. In some embodiments, the “loading dose” can increase the blood concentration of the drug to a therapeutically effective level. In some embodiments, the “loading dose” can increase the blood concentration of the drug to a therapeutically effective level in conjunction with a treatment dose of the drug. The “loading dose” can be administered once per day, or more than once per day (e.g., up to 4 times per day). In some embodiments, a loading dosage may be used for drug molecules that have a long half-life or slow elimination in vivo.

“Pharmaceutical preparation” or “pharmaceutical composition” refers to a mixture or solution containing at least one therapeutic agent suitable to be administered to a warm-blooded animal, e.g., a human.

“Pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

“Subject,” “patient,” or “warm-blooded animal” is intended to include animals. Examples of subjects include mammals, e.g., humans, dogs, cows, horses, pigs, sheep, goats, cats, mice, rabbits, rats, and transgenic non-human animals. In certain embodiments, the subject is a human.

“Therapeutically effective” preferably relates to an amount of a therapeutic agent that is capable of providing a therapeutic response in a subject or prophylactically effective against the progression of a cancer. A therapeutically effective treatment or amount of treatment need not completely treat a subject, but may partially or completely delay, ameliorate, reverse, or reduce at least one or more signs, symptoms, or manifestations of a disease.

“Treatment” or “treating” can be prophylactic and/or therapeutic (including but not limited to palliative, symptom-alleviating, symptom-reducing) as well as the delay of progression of a disease or disorder such as cancer. The term “prophylactic” means the prevention or delay of the onset or recurrence of a disease such as cancer. The term “delay of progression” as used herein means administration of the combination to patients being in a pre-stage or in an early phase of the cancer to be treated, a pre-form of the corresponding cancer is diagnosed and/or in a patient diagnosed with a condition under which it is likely that a corresponding cancer will develop.

“Tumor” refers to an abnormal mass of tissue. Tumors may comprise cancer cells. Tumors may be described as benign if they do not spread into or invade nearby tissues or other parts of the body. Tumors may be described as malignant if they spread into other tissues or parts of the body.

XII. ABBREVIATIONS

| | |
|---|---|
| AE | Adverse Event |
| AI | Aromatase Inhibitor |
| AJCC | American Joint Committee on Cancer |
| ALND | Axillary Lymph Node Dissection |
| ALP | Alkaline Phosphatases |
| ALT | Alanine Transaminase |
| ANC | Absolute Neutrophil Count |
| aPTT | Activated Partial Thromboplastin Time |
| AST | Aspartate Transaminase |
| ATC | Anatomical Therapeutic Chemical |
| AUC | Area Under the Curve |
| AV | Atrioventricular |
| BC | Breast Cancer |
| BCRP | Breast Cancer Resistance Protein |
| BSEP | Bile Salt Export Pump |
| CA | Competent Authority |
| CBP | Childbearing Potential |
| CCND1 | Cyclin D1 |
| CDK | Cyclin-Dependent Serine-Threonine Protein Kinases |
| CI | Confidence Interval |
| CISH | Chromogenic In Situ Hybridization |
| Ctrough | Trough concentration |
| Cmax | Maximum Serum Concentration |
| CMO&PS | Chief Medical Office and Patient Safety |
| COSMIC | Catalogue of Somatic Mutations in Cancer |
| CRF | Case Report Form; the term CRF can be applied to either |
| | EDC or Paper |
| CSR | Clinical Study Report |
| CT | Computed Tomography |
| CTCAE | Common Terminology Criteria for Adverse Events |
| ctDNA | Circulating Tumor DNA |
| ctRNA | Circulating Tumor RNA |
| CV | Coefficient of Variation |
| CYP | Cytochrome P450 |
| DCIS | Ductal Carcinoma in situ |
| DDFS | Distant Disease Free Survival |
| DDI | Drug-Drug Interaction |
| DHEA | Dehydroepiandrosterone |
| DILI | Drug-Induced Liver Injury |
| DNA | Deoxyribonucleic Acid |
| DX | Diagnosis |
| EBC | Early Breast Cancer |
| EBCTCG | EBC Trialists' Collaborative Group |
| ECG | Electrocardiogram |
| ECHO | Echocardiogram |
| ECOG | Eastern Cooperative Oncology Group |
| EDC | Electronic Data Capture |
| eGFR | Estimated Glomerular Filtration Rate |
| EORTC-QLQ | European Organization for Research and Treatment of |
| | Cancer's Core |
| | Quality of Life Questionnaire |
| EOT | End of Treatment |
| EQ-SD-SL | EuroQoL 5-Level Instrument |
| ER | Estrogen Receptor |
| ET | Endocrine Therapy |
| FAS | Full Analysis Set |
| FDA | Food and Drug Administration |
| FDG-PET | Fludeoxyglucose Positron Emission Tomography |
| FISH | Fluorescence In Situ Hybridization |
| FSH | Follicle-stimulating hormone |
| G2-3 | Histologic Grade 2-3 |
| GCP | Good Clinical Practice |
| GDPR | General Data Protection Regulation (EU) 2016/679 |
| GGT | Gamma-glutamyl Transferase |
| GI | Gastrointestinal |
| GnRH | Gonadotropin-releasing Hormone |
| GWAS | Genome-Wide Association Studies |
| HADS | Hospital Anxiety and Depression Scale |
| HBV | Hepatitis B virus |
| β-hCG | Beta Human Chorionic Gonadotropin |
| HCV | Hepatitis C Virus |
| HER2 | Human Epidermal Growth Factor Receptor 2 |
| HIPAA | Health Insurance Portability and Accountability Act (US) |
| HIV | Human Immunodeficiency Virus |
| HLA | Human Leukocyte Antigen |
| HR | Hormone Receptor |
| IB | Investigator's Brochure |
| IBTR | Invasive ipsilateral breast tumor |
| ICH | International Council for Harmonization |
| iDFS | Invasive Disease-Free Survival |
| IDMC | Independent Data Monitoring Committee |
| IEC | Independent Ethics Committee |
| IHC | Immunohistochemistry |
| ILD | Interstitial Lung Disease |
| IMP | Investigational Medicinal Product |
| IN | Investigator Notification |
| INR | International Normalized Ratio |
| IRB | Institutional Review Board |
| IRT | Interactive Response Technology |
| IUD | Intrauterine Device |
| i.v. | Intravenous(ly) |
| LC-MS/MS | Liquid Chromatography-tandem Mass Spectrometry |
| LDH | Lactate Dehydrogenase |
| LFT | Liver Function Test |
| LLOQ | Lower Limit of Quantification |
| LPLV | Last Patient Last Visit |
| LRRFS | Loco-regional Recurrence-Free Survival |
| LVEF | Left Ventricular Ejection Fraction |
| MATE1 | Multidrug and Toxin Extrusion Protein-1 |
| MDRD | Modification of Diet in Renal Disease |
| MI | Myocardial Infarction |
| MRI | Magnetic Resonance Imaging |
| msec | Milliseconds |
| MUGA | Multiple Gated Acquisition |
| NaF-PET | Sodium Fluoride Positron Emission Tomography |
| NCI | National Cancer Institute |
| NSAI | Non-Steroidal Aromatase Inhibitor |
| OCT2 | Organic Cation Transporter 2 |
| ORR | Overall Response Rate |
| OS | Overall survival |
| PAS | Pharmacokinetics Analysis Set |
| PFS | Progression-Free Survival |
| P-gp | P-glycoprotein |
| PgR or PR | Progesterone Receptor |
| PICF | Patient Informed Consent Form |
| PK | Pharmacokinetics |
| PK-ANC | PK-Absolute Neutrophil Count |
| PK-QTcF | PK-QT Interval in the ECG corrected according to the |
| | formula of Fridericia |
| PPS | Per-Protocol Set |
| PRO | Patient Reported Outcomes |
| PT | Prothrombin Time |
| QD | Once a day |
| QoL | Quality of Life |
| QT | QT Interval in the ECG |
| QTc | QT Interval in the ECG (corrected) |
| QTcF | QT Interval in the ECG (corrected according to the |
| | formula of Fridericia) |
| Rb | Retinoblastoma |
| RFS | Recurrence-Free Survival |
| RNA | Ribonucleic Acid |
| RoW | Rest of the World |
| SAE | Serious Adverse Event |
| SAP | Statistical Analysis Plan |
| SC | Steering Committee |
| SEER | Surveillance, Epidemiology, and End Results |
| SERM | Selective estrogen receptor modulators |
| SGOT | Serum Glutamic Oxaloacetic Transaminase |
| SGPT | Serum Glutamic Pyruvic Transaminase |
| SISH | Silver in Situ Hybridization |
| SLN | Sentinel Lymph Node |
| STEEP | Standardized Definitions for Efficacy End Points (in |
| | Adjuvant Breast Cancer Trials) |
| SUSAR | Suspected Unexpected Serious Adverse Reaction |
| T_{1/2} | Half-life |
| TBIL | Total Bilirubin |
| TdP | Torsades de Pointes |
| TEAE | Treatment-emergent adverse effects |
| TEN | Toxic Epidermal Necrolysis |
| Tₘₐₓ | The time at which the maximum observed concentration |
| | (Cₘₐₓ) occurs |
| TNM | Tumor, Node and Metastasis |
| ULN | Upper Limit of Normal |
| US | Ultrasound |
| TRIO | Translational Research in Oncology |
| VAS | Visual Analogue Scale |
| WBC | White Blood Cell |
| WHO | World Health Organization |

### XIII. REFERENCES

All publications, patents and patent applications referred to herein are incorporated by reference in their entirety to the same extent as if each individual publication, patent, or patent application was specifically and individually indicated to be incorporated by reference in its entirety.

### XIV. EXAMPLES

Hereinafter, the present invention is described in more detail and specifically with reference to the examples, which however are not intended to limit the present invention.

### a. EXAMPLE 1. CLINICAL TRIAL OVERVIEW

The clinical phase 3 trial with the protocol title “A phase III, multicenter, randomized, open-label trial to evaluate efficacy and safety of ribociclib with endocrine therapy as an adjuvant treatment in patients with hormone receptor-positive, HER2-negative, early breast cancer” is described at ClinicalTrials.gov with the Identifier: NCT03701334 (the entire disclosure of that webpage is incorporated herein by reference). The trial has been and is being performed according to the protocol described below.

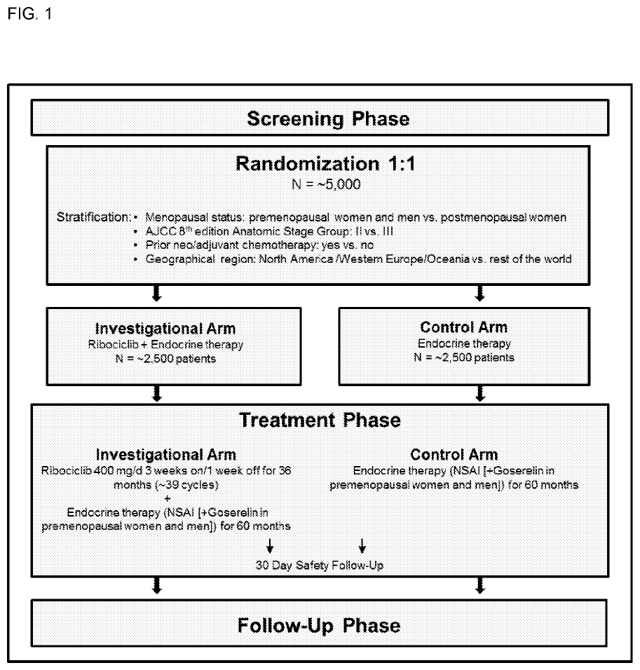

Study design: See FIG. 1.

Synopsis of the clinical protocol for the NATALEE trial (New Adjuvant TriAl with Ribociclib [LEE011])

| Protocol Title | A phase III, multicenter, randomized, open-label trial to evaluate efficacy | |
|---|---|---|
| | and safety of ribociclib with endocrine therapy as an adjuvant treatment | |
| | in patients with hormone receptor-positive, HER2-negative, early breast | |
| | cancer (New Adjuvant TriAl with Ribociclib [LEE011]: NATALEE). | |
| Trial Number | CLEE011O12301C (TRIO033) | |
| Trial Sponsor | Novartis | |
| Participating | Approximately 425 sites worldwide will participate in the trial. | |
| Sites | | |
| Investigational | Ribociclib (LEE011) | |
| Drug | | |
| Indication | Adjuvant treatment of hormone receptor (HR)-positive, HER2-negative, | |
| | early breast cancer (EBC). | |
| Population | Pre and postmenopausal women and men with HR-positive, HER2- | |
| | negative EBC, after adequate surgical resection, who are eligible for | |
| | adjuvant non-steroidal aromatase inhibitor (NSAI) for at least 5 years. | |
| | Objective | Endpoint |
| Objectives/ | Primary | |
| Endpoints | To compare iDFS for ribociclib + ET | iDFS using STEEP criteria |
| | versus ET in patients with HR- | (Standardized Definitions for |
| | positive, HER2-negative, EBC | Efficacy End Points in Adjuvant |
| | | Breast Cancer Trials), as assessed |
| | | by Investigator |
| | Secondary | |
| | 1. To evaluate the two treatment | RFS using STEEP criteria |
| | arms with respect to recurrence-free | |
| | survival (RFS) | |
| | 2. To evaluate the two treatment | DDFS using STEEP criteria |
| | arms with respect to distant disease- | |
| | free survival (DDFS) | |
| | 3. To evaluate the two treatment | OS |
| | arms with respect to overall survival | |
| | (OS) | |
| | 4. To evaluate patient reported | Change from baseline in the |
| | outcomes (PRO) for health-related | physical functioning sub-scale |
| | quality of life (QoL) in the two | score and global health status/QoL |
| | treatment arms | scale score as assessed by |
| | | EORTC QLQ-C30 |
| | 5. To evaluate safety and tolerability | Frequency and severity of adverse |
| | of the treatment regimen | events (AEs), laboratory and |
| | | electrocardiogram (ECG) |
| | | abnormalities |
| | 6. To characterize the | PK parameters such as C_{trough} and |
| | pharmacokinetics (PK) of ribociclib | other applicable parameters for |
| | when given in combination with NSAI | ribociclib |
| | (and goserelin if applicable) | |

| | |
|---|---|
| Trial Design | This is a phase III, multicenter, randomized, open-label trial to evaluate |
| and Treatment | the efficacy and safety of ribociclib with ET as an adjuvant treatment in |
| | women and men with HR-positive, HER2-negative EBC. iDFS is the |
| | primary endpoint of the trial, as defined per the STEEP System. |
| | The trial will include pre and postmenopausal women and men with HR- |
| | positive, HER2-negative EBC, with an Anatomic Stage Group III, IIB or a |
| | subset of Stage IIA cases (as defined in Inclusion Criterion #8, see |
| | below), after adequate surgical resection, radiotherapy (if indicated), |
| | adjuvant or neoadjuvant chemotherapy (if indicated), and who are |
| | deemed to be eligible for adjuvant ET for at least 60 months of duration. |
| | See FIG. 1. |
| | Approximately 5,000 patients will be randomized (using an Interactive |
| | Response Technology system [IRT]) into two treatment arms in a 1:1 |
| | ratio to: |
| | Investigational arm: |
| | Ribociclib 400 mg by mouth once daily on days 1 to 21 of a 28-day |
| | cycle, for 36 months since randomization (approximately 39 cycles). |
| | And |
| | ET consisting of: |
| | For postmenopausal women: letrozole 2.5 mg by mouth once |
| | daily continuously or anastrozole 1 mg by mouth once daily |
| | continuously. |
| | For premenopausal women and men: letrozole 2.5 mg by mouth |
| | once daily continuously or anastrozole 1 mg by mouth once daily |
| | continuously, combined with goserelin 3.6 mg subcutaneously |
| | once every 4 weeks. |
| | Duration of ET in the trial will be 60 months from the |
| | randomization date. |
| | Control arm: |
| | ET: Same as in the Investigational arm. |
| | In both arms, ET will be administered according to the local clinical |
| | guidelines and current local prescribing information. Subsequent ET (or |
| | any other anti-cancer treatment) given after the protocol-required 60 |
| | months of ET (or after premature discontinuation of ET in the trial) will be |
| | administered according to the Investigator's clinical judgment and is not |
| | considered a trial treatment. |
| | Randomization will be stratified by the following factors: |
| | Menopausal status: premenopausal women and men vs. |
| | postmenopausal women |
| | AJCC 8^{th} edition Anatomic Stage Group: Anatomic Stage Group II vs. |
| | Anatomic Stage Group III |
| | Prior neoadjuvant/adjuvant chemotherapy: yes vs. no |
| | Geographical region: North America/Western Europe/Oceania vs. rest |
| | of the world |
| | Enrollment of patients with Anatomic Stage Group II is capped at |
| | approximately 2,000 patients. |
| | The trial will include screening, treatment, and follow up phases. The trial |
| | includes an exploratory component that requires collection of tumor and |
| | blood samples (except for patients enrolled in China). |
| Eligibility | Inclusion Criteria |
| Criteria | Patients eligible for inclusion in this trial must meet all of the following |
| | criteria: |
| | 1. Signed and dated Patient Informed Consent Form (PICF) obtained |
| | prior to any trial-specific screening procedure. |
| | 2. Patient is ≥18 years-old at the time of PICF signature. |
| | 3. Patient is female with known menopausal status at the time of |
| | randomization or initiation of adjuvant ET (whichever occurs earlier), |
| | or male. |
| | Postmenopausal status is defined as: |
| | Patient underwent bilateral oophorectomy, or |
| | Age ≥60 years, or |
| | Age <60 years and amenorrhea for 12 or more months (in the |
| | absence of chemotherapy, tamoxifen, toremifene or ovarian |
| | suppression) and Follicle-stimulating hormone (FSH) and plasma |
| | estradiol are in the postmenopausal ranges per local normal |
| | ranges. |
| | If taking tamoxifen or toremifene and age <60 years, then FSH |
| | and plasma estradiol level in postmenopausal ranges. |
| | Notes |
| | In women who are premenopausal at the beginning of |
| | adjuvant chemotherapy, amenorrhea is not a reliable |
| | indicator of menopausal status as ovarian function may still |
| | be intact or resume despite anovulation/amenorrhea. For |
| | these women with therapy-induced amenorrhea, serial |
| | measurements of FSH and/or estradiol per local clinical |
| | guidelines are required for determination of postmenopausal |
| | status. |
| | All women who do not meet the criteria for postmenopausal |
| | status are considered premenopausal for the purpose of this |
| | trial. |
| | 4. Patient with histologically confirmed unilateral primary invasive |
| | adenocarcinoma of the breast with a date of initial cytologic or |
| | histologic diagnosis (i.e. date of the pathology report that confirmed |
| | the BC diagnosis) within 18 months prior to randomization. Patient |
| | with a multicentric and/or multifocal tumor is eligible if all |
| | histopathologically examined lesions meet the pathologic criteria in |
| | inclusion criteria 5 and 6. |
| | 5. Patient has breast cancer that is positive for ER and/or PgR according |
| | to the local laboratory as determined on the most recently analyzed |
| | tissue sample. |
| | 6. Patient has HER2-negative breast cancer defined as a negative in situ |
| | hybridization test or an immunohistochemistry (IHC) status of 0 or 1+. |
| | If IHC is 2+, a negative in situ hybridization (FISH, CISH, or SISH) test |
| | is required to confirm the HER2-negative status (based on the most |
| | recently analyzed tissue sample tested by a local laboratory). |
| | 7. Patient (except those enrolled in China) has available archival tumor |
| | tissue from the surgical specimen, for submission to a central |
| | laboratory (Note: in patients that underwent neoadjuvant systemic |
| | therapy and had a pathologic complete response, archival tumor |
| | tissue at the time of the initial diagnosis or before the administration |
| | of neoadjuvant therapy is mandatory). |
| | 8. Patient, after surgical resection where tumor was removed |
| | completely, with the final surgical specimen microscopic margins free |
| | from tumor, and belongs to one of the following categories: |
| | Anatomic Stage Group III, or |
| | Anatomic Stage Group IIB, or |
| | Anatomic Stage Group IIA that is either: |
| | N1, or |
| | N0, with: |
| | Grade 3, or |
| | Grade 2, with any of the following criteria: |
| | Ki67 ≥20%, or |
| | Oncotype DX Breast Recurrence Score ≥26, or |
| | Prosigna/PAM50 categorized as high risk, or |
| | MammaPrint categorized as high risk, or |
| | EndoPredict EPclin Risk Score categorized as high |
| | risk |
| | Notes: |
| | For patients whose tumors are Anatomic Stage IIA, N0: |
| | If Grade is 1 or unknown (Gx), patient is not eligible. |
| | If Grade 2, the gene expression test results (by Oncotype |
| | DX, Prosigna/PAM50, MammaPrint or EndoPredict |
| | EPclin) or Ki67 levels should be used if obtained as per |
| | local practice (i.e. are not mandatory for the purpose of |
| | the trial). Results must be available at screening. |
| | Patients that received neoadjuvant treatment must meet the |
| | above criteria (for stage, and if Stage IIA, N0, also for grade |
| | and Ki67 or gene expression test) in any presurgical |
| | staging/sample and/or in the surgical specimen. |
| | Categorization into the AJCC 8^{th} edition Anatomic Stage |
| | Groups requires determination of the T, N and M categories. |
| | ALND is the preferred method for axillary lymph node staging, |
| | however SLN dissection can be used to determine the N |
| | category in the following cases: |
| | No metastasis in SLN (patient is considered as pN0). |
| | Only micrometastasis in SLN (patient is considered as |
| | pN1mi). |
| | Patients with T1-2 and no clinically-evident nodes prior to |
| | surgery, no neoadjuvant chemotherapy, at least one |
| | macrometastasis in 1 or 2 SLNs, no matted nodes or |
| | gross extranodal disease at the time of SLN dissection |
| | (patient is considered as pN1). |
| | In all other cases, ALND is required to determine the N |
| | category. |
| | 9. If indicated, patient has completed adjuvant and/or neoadjuvant |
| | chemotherapy according to the institutional guidelines, prior to |
| | screening. |
| | 10. If indicated, patient has completed adjuvant radiotherapy according to |
| | the institutional guidelines, prior to screening. |
| | 11. Patient has no contraindication for the adjuvant ET in the trial and is |
| | planned to be treated with ET for 5 years (since randomization date) |
| | or more. |
| | 12. Patient may have already received any standard neoadjuvant and/or |
| | adjuvant ET at the time of PICF signature, but randomization should |
| | occur within 12 months of the initial start date of ET. Ovarian |
| | suppression or short term ET for fertility preservation is not considered |
| | neoadjuvant/adjuvant ET. If patient was receiving tamoxifen or |
| | toremifene as adjuvant ET, a washout period of 5 half-lives (i.e. 35 |
| | days) prior to randomization is required (during that period patient can |
| | take AI). |
| | 13. Patient has an Eastern Cooperative Oncology Group (ECOG) |
| | Performance Status of 0 or 1. |
| | 14. Patient has adequate bone marrow and organ function as defined by |
| | the following local laboratory values: |
| | Absolute neutrophil count (ANC) ≥ 1.5 × 10⁹/L |
| | Platelets ≥ 100 × 10⁹/L |
| | Hemoglobin ≥9.0 g/dL |
| | Estimated glomerular filtration rate (eGFR) ≥30 mL/min/1.73 m² |
| | according to the Modification of Diet in Renal Disease (MDRD) |
| | formula |
| | Alanine transaminase (ALT) < 2.5 × Upper Limit Normal (ULN) |
| | Aspartate transaminase (AST) < 2.5 × ULN |
| | Total serum bilirubin <ULN; or total bilirubin ≤ 3.0 × ULN or direct |
| | bilirubin ≤ 1.5 × ULN in patients with well documented Gilbert's |
| | Syndrome |
| | International normalized ratio (INR) ≤1.5 (unless the patient is |
| | receiving anticoagulants and the INR is within the therapeutic |
| | range of intended use for that anticoagulant within 7 days prior to |
| | randomization) |
| | Patient must have the following laboratory values within normal |
| | limits or corrected to within normal limits with supplements (the |
| | local laboratory value should be documented within normal limits |
| | after the correction) before randomization: |
| | Potassium |
| | Magnesium |
| | Total Calcium (corrected for serum albumin) |
| | 15. Standard 12-lead ECG values assessed by a central laboratory, as: |
| | QTcF interval (QT interval using Fridericia's correction) at |
| | screening <450 milliseconds (msec) |
| | Resting heart rate 50-90 beats per minute (determined from the |
| | ECG) |
| | 16. Patient must be willing and able to comply with scheduled visits, |
| | treatment plans, laboratory tests, and other trial procedures. |
| | 17. Women of childbearing potential (CBP), defined as all women |
| | physiologically capable of becoming pregnant (see Inclusion Criterion |
| | #18 for additional information), must have confirmed negative serum |
| | pregnancy test (for β-hCG) within 14 days prior to randomization. |
| | 18. Women of CBP must be willing to use highly effective methods of |
| | contraception. Contraception must continue during the trial treatment |
| | and for 21 days after stopping the treatment. Highly effective |
| | contraception methods include: |
| | Total abstinence (when this is in line with the preferred and usual |
| | lifestyle of the patient). Periodic abstinence (e.g. calendar, |
| | ovulation, symptothermal, post-ovulation methods) and |
| | withdrawal are not acceptable methods of contraception. |
| | Female sterilization (have had surgical bilateral oophorectomy |
| | with or without hysterectomy), total hysterectomy or tubal ligation |
| | at least 6 weeks before taking trial treatment. In case of |
| | oophorectomy alone, only when the reproductive status of the |
| | woman has been confirmed by follow up hormone level |
| | assessment. |
| | Male partner sterilization (at least 6 months prior to |
| | randomization). For female patients on the trial the vasectomized |
| | male partner should be the sole partner for that patient. If |
| | vasectomy of the male partner is the highly effective method of |
| | contraception chosen, the success of the vasectomy should be |
| | medically confirmed according to local practice. |
| | Placement of an intrauterine device (IUD). |
| | Notes: |
| | Use of oral (estrogen and progesterone), transdermal, |
| | injected, implanted, hormone containing intrauterine system |
| | or any other hormonal method of contraception is not allowed |
| | in this trial. |
| | Women are considered of CBP unless: they have had ≥12 |
| | months of natural (spontaneous) amenorrhea with an |
| | appropriate clinical profile (i.e. age appropriate, history of |
| | vasomotor symptoms) or have had surgical bilateral |
| | oophorectomy (with or without hysterectomy), total |
| | hysterectomy, or tubal ligation at least six weeks prior to |
| | randomization. In the case of oophorectomy alone, only when |
| | the reproductive status of the woman has been confirmed by |
| | follow-up hormone level assessment she is considered not of |
| | CBP. |
| | After the end of trial treatment, patients should use effective |
| | contraception according to local guidelines. |
| | Exclusion Criteria |
| | Patients eligible for this trial must not meet any of the following criteria: |
| | 2. Patient has received any CDK4/6 inhibitor. |
| | 3. Patient has received prior treatment with tamoxifen, raloxifene or AIs |
| | for reduction in risk (“chemoprevention”) of breast cancer and/or |
| | treatment for osteoporosis within the last 2 years prior to |
| | randomization. Patient is concurrently using hormone replacement |
| | therapy. |
| | 4. Patient has received prior treatment with anthracyclines at cumulative |
| | doses of 450 mg/m² or more for doxorubicin, or 900 mg/m² or more |
| | for epirubicin. |
| | 5. Patient with a known hypersensitivity to any of the excipients of |
| | ribociclib and/or ET (e.g. rare hereditary problems of galactose |
| | intolerance, the Lapp lactase deficiency, glucose-galactose |
| | malabsorption, and soy allergy). |
| | 6. Patient with distant metastases of breast cancer beyond regional |
| | lymph nodes (stage IV according to AJCC 8^{th} edition) and/or evidence |
| | of recurrence after curative surgery. |
| | 7. Patient is concurrently using other anti-neoplastic therapy with the |
| | exception of adjuvant ET (see Inclusion Criterion #12). |
| | 8. Patient has had major surgery, chemotherapy or radiotherapy within |
| | 14 days prior to randomization. |
| | 9. Patient has not recovered from clinical and laboratory acute toxicit |
| | related to prior anti-cancer therapies to a NCI CTCAE (National |
| | Cancer Institute Common Terminology Criteria for Adverse Events) |
| | version 4.03 Grade ≤1 at day of randomization. Exceptions to this |
| | criterion: patients with any grade of alopecia, amenorrhea, grade 2 |
| | neuropathy are allowed to enter the trial or other toxicities not |
| | considered a safety risk for the patient as per Investigator's discretion, |
| | are allowed to enter the trial. |
| | 10. Patient has a concurrent invasive malignancy or a prior invasive |
| | malignancy whose treatment was completed within 2 years before |
| | randomization. Note: Patients with adequately treated, basal or |
| | squamous cell skin carcinoma or curatively resected cervical cancer |
| | in situ are eligible. |
| | 11. Patient has known history of human immunodeficiency virus (HIV) |
| | infection (testing is not mandatory, unless required by local |
| | regulation). |
| | 12. Patient has known active hepatitis B virus (HBV) or hepatitis C virus |
| | (HCV) infection (testing is not mandatory, unless required by local |
| | regulation). |
| | 13. Clinically significant, uncontrolled heart disease and/or cardiac |
| | repolarization abnormality, including any of the following: |
| | History of documented myocardial infarction (MI), angina pectoris, |
| | symptomatic pericarditis, or coronary artery bypass graft within 6 |
| | months prior to trial entry. |
| | Documented cardiomyopathy. |
| | Left Ventricular Ejection Fraction (LVEF) <50% as determined by |
| | Multiple Gated acquisition (MUGA) scan or echocardiogram |
| | (ECHO) (testing not mandatory) |
| | Long QT syndrome or family history of idiopathic sudden death or |
| | congenital long QT syndrome, or any of the following: |
| | Risk factors for Torsades de Pointes (TdP) including |
| | uncorrected hypocalcemia, hypokalemia or |
| | hypomagnesemia, history of cardiac failure, or history of |
| | clinically significant/symptomatic bradycardia. |
| | Concomitant medication(s) with a known risk to prolong the |
| | QT interval and/or known to cause TdP that cannot be |
| | discontinued or replaced by safe alternative medication (e.g. |
| | within 5 half-lives or 7 days prior to starting trial treatment). |
| | Inability to determine the QTcF interval. |
| | Clinically significant cardiac arrhythmias (e.g. ventricular |
| | tachycardia), complete left bundle branch block, high-grade |
| | Atrioventricular (AV) block (e.g. bifascicular block, Mobitz type II |
| | and third degree AV block). |
| | Uncontrolled arterial hypertension with systolic blood pressure >160 |
| | mmHg. |
| | 14. Patient is currently receiving any of the following substances within 7 |
| | days before randomization: |
| | Concomitant medications, herbal supplements, and/or fruits (e.g. |
| | grapefruit, pummellos, starfruit, Seville oranges) and their juices |
| | that are known as strong inhibitors or inducers of CYP3A4/5 |
| | Medications that have a narrow therapeutic window and are |
| | predominantly metabolized through CYP3A4/5 |
| | 15. Patient is currently receiving or has received systemic corticosteroids ≤2 |
| | weeks prior to starting trial treatment, or has not fully recovered |
| | from side effects of such treatment. |
| | Note: The following uses of corticosteroids are permitted: a short |
| | duration (<5 days) of systemic corticosteroids; any duration of topical |
| | applications (e.g. for rash), inhaled sprays (e.g. for obstructive |
| | airways diseases), eye drops or local injections (e.g. intra-articular). |
| | 16. Patient has impairment of gastrointestinal (GI) function or GI disease |
| | that may significantly alter the absorption of the oral trial treatments |
| | (e.g. uncontrolled ulcerative diseases, uncontrolled nausea, vomiting |
| | or diarrhea, malabsorption syndrome, or small bowel resection). |
| | 17. Patient has any other concurrent severe and/or uncontrolled medical |
| | condition that would, in the Investigator's judgment, cause |
| | unacceptable safety risks, contraindicate patient participation in the |
| | clinical trial or compromise compliance with the protocol (e.g. chronic |
| | pancreatitis, chronic active hepatitis, liver cirrhosis or any other |
| | significant liver disease, active untreated or uncontrolled fungal, |
| | bacterial or viral infections, active infection requiring systemic anti- |
| | bacterial therapy, etc.) or limit life expectancy to ≤5 years. |
| | 18. Participation in other studies involving investigational drug(s) within 30 |
| | days prior to randomization or within 5 half-lives of the investigational |
| | drug(s) (whichever is longer), or participation in any other type of |
| | medical research judged not to be scientifically or medically |
| | compatible with this trial. If the patient is enrolled or planned to be |
| | enrolled in another study that does not involve an investigational drug, |
| | the agreement of the Medical Monitor is required to establish eligibility. |
| | 19. Pregnant or breast-feeding (lactating) women or women who plan to |
| | become pregnant or breast-feed during the trial. |
| Assessments | Trial visits will occur at screening, randomization, during treatment, |
| | approximately 30 days after discontinuation of ribociclib (Investigational |
| | arm only), End of Treatment (EOT), approximately 30 days after |
| | discontinuation of all trial treatments and during Follow-up. |
| | Efficacy assessments |
| | Efficacy assessment for the primary endpoint of iDFS will include |
| | detection of locoregional relapse, distant relapse, ipsilateral and |
| | contralateral invasive BC, second primary invasive non-BCs and deaths. |
| | Assessments for events of recurrence will be done clinically every 12 |
| | weeks (±2 weeks) for the first 24 months from randomization and every |
| | 24 weeks (±3 weeks) thereafter. Mammography will be done annually. |
| | Recurrences suspected clinically will be confirmed by additional imaging |
| | and every effort should be done to also confirm them |
| | histologically/cytologically (unless unsafe per Investigator's discretion). |
| | Survival status will be assessed up to 60 months after the last patient |
| | has been randomized, until death, withdrawal of consent, loss to follow- |
| | up or end of trial, whichever is earliest. Survival information can be |
| | obtained by clinical visits, telephone calls, or other means. |
| | Safety assessments |
| | Safety will be assessed for each patient and will include routine safety |
| | monitoring (AEs collection, laboratory testing of hematology, serum |
| | chemistry, measurement of vital signs, performance status, physical |
| | examination, and ECG). |
| | Assessments of QoL and healthcare resources utilization |
| | QoL will be evaluated by periodic questionnaires (i.e. EORTC QLQ-C30, |
| | EORTC QLQ-BR23, EQ-5D-5L and HADS), at screening and during the |
| | Treatment and Follow up Phases. |
| | Use of subsequent anti-cancer therapy, time to first subsequent anti- |
| | cancer therapy and healthcare resources utilization will be evaluated for |
| | each arm. |
| | PK assessments |
| | Approximately 130 patients from the Investigational arm will be part of |
| | the PK subset. Plasma samples for ribociclib determination will be |
| | obtained pre- and post-dose on C1D15. |
| Statistical | The primary efficacy analysis will be the comparison of the distribution of |
| Methods | iDFS between the two treatment arms using a stratified log-rank test at |
| | one-sided 2.5% level of significance, using strata information at the time |
| | of randomization. Distribution of iDFS will be estimated using the Kaplan- |
| | Meier method. The iDFS at the end of each year along with 95% |
| | confidence intervals will be presented for each of the two treatment arms. |
| | The stratified Cox regression will be used to estimate the hazard ratio of |
| | iDFS, along with 95% confidence interval, using strata information as per |
| | IRT at the time of randomization. |

### Results

### Overview of Results

Results from an interim analysis of the NATALEE trial:

Kisqali plus endocrine therapy (ET) significantly reduced the risk of disease recurrence compared to standard ET alone in the adjuvant setting.

NATALEE is the first and only positive Phase III study of a CDK4/6 inhibitor demonstrating consistent benefit in a broad population of patients with stage II and III HR+/HER2− early breast cancer (EBC) at risk of recurrence, including those with no nodal involvement.

The primary endpoint of invasive disease-free survival (iDFS) has been met. Kisqali plus ET significantly reduced the risk of disease recurrence, compared to standard adjuvant ET alone, with consistent benefit in patients with stage II and stage III EBC regardless of nodal involvement.
Study met its Primary Endpoint of iDFS at Interim Analysis 3 (426/500 events)
   1-sided p-value of 0.0014 met the early efficacy stopping boundary (p<0.0128)
   Approx 25% reduction in the risk of iDFS (HR: 0.748, 95% C01(0.619-0.906))
   3y iDFS rate:RIB+ET:90.4% vs ET alone:87.1%; Δ:3.3%
Overall Consistent iDFS effect in key subgroups
   Stage III—HR: 0.74 (0.59-0.92); Stage II—HR: 0.76 (0.52-1.1)
   Premenopausal & Men—HR 0.72 (0.53-0.98); Postmenopausal—HR: 0.78 (0.613-0.997)
Positive trend seen for all 2ry efficacy endpoints: RFS, DDFS & OS
   Any deterioration in overall survival (OS) can be ruled out: HR 0.76 (0.54-1.07), p-value: 0.0559

In terms of safety, the trial demonstrated a well-tolerated safety profile. Furthermore, the 400 mg ribociclib dose demonstrated an improved profile with less overall toxicity, particularly dose-dependent adverse events (cardiac QT interval & neutropenia). There were no new safety findings compared to the known and established safety profile of ribociclib therapy. Discontinuation rates due to adverse events over a 3 year period were similar in early breast cancer patients as in metastatic breast cancer. Compared to the earlier ‘monarch-E’ trial, diarrhea was not a frequent (≥5%) grade 3 or 4 adverse reaction, and no patients discontinued or reduced their ribociclib dose for this reason.

In summary, patients in the RIB+ET arm demonstrated a significantly longer iDFS than ET alone (HR 0.748, p=0.0014), with 3 year iDFS rates increasing to 90.4% (vs. 87.1%). The iDFS benefit was consistent across stratification factors and other subgroups. Secondary endpoints of overall survival, recurrence-free survival, and distant disease-free survival were consistently in favor of the RIB+ET arm. Moreover, addition of RIB had a favorable safety profile with no new signals. Overall, addition of RIB to the standard-of-care ET demonstrated a statistically significant and clinically meaningful improvement in iDFS, with a well-tolerated safety profile. The combined therapy can therefore be seen as suitable for use in a broad population of patients who have stage II or stage III HR+/HRE2− early breast cancer, including NO patients whose cancer has not spread to nearby lymph nodes. This is unexpected based on the results of other parallel studies.

### Assessment of Health-Related Quality of Life (HRQOL) in NATALEE

QoL was analyzed after a median follow-up of 34 months. About 20% of patients had completed 3 years of ribociclib plus endocrine therapy.

The prespecified analysis of HRQOL was based on patient-reported outcomes based on a number of survey instruments: the EORTC QLQ-C30 (European Organisation for Research and

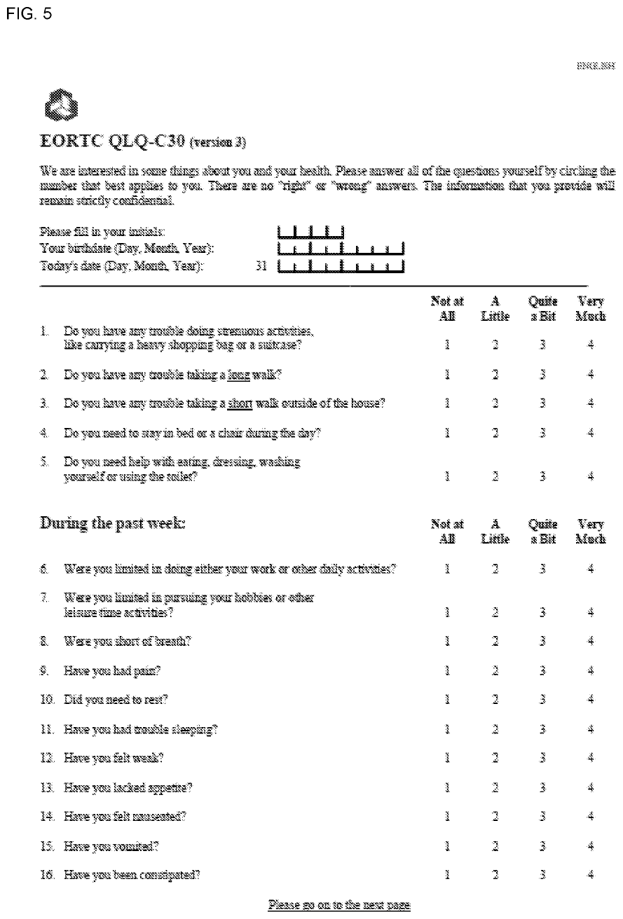

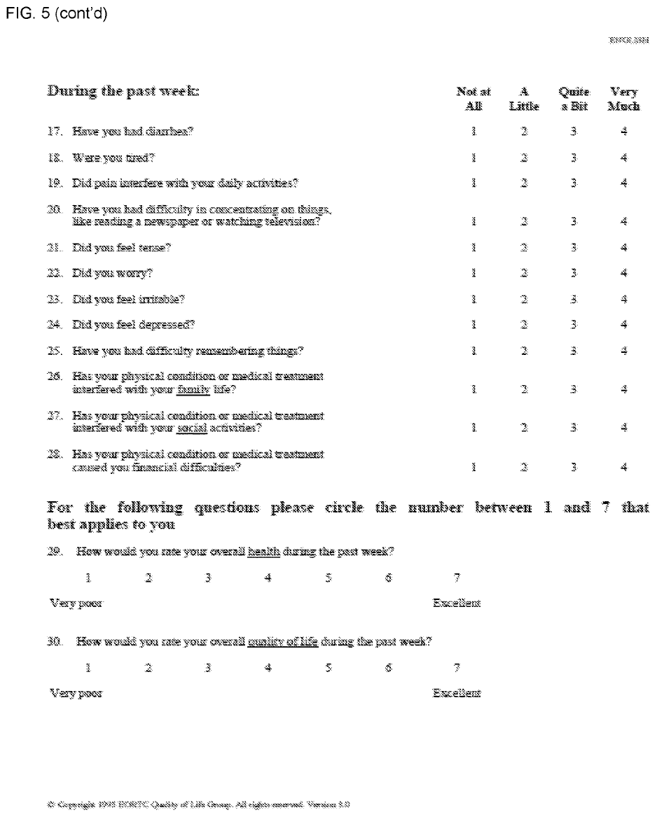

Treatment of Cancer Quality-of-Life questionnaire) for function (physical, social, and emotional) and global health status; EORTC QLQ-BR23 for breast cancer symptoms; the EQ-VAS (Euro-QoL visual analog scale) of the EQ-5D-5L; and the Hamilton Anxiety and Depression Scale (See also FIGS. 5-8 for exemplary questionaires). The HRQOL of patients with HR+/HER2− EBC was maintained with the addition of ribociclib to standard-of-care adjuvant NSAI vs NSAI alone. Physical functioning and Global Health scores were maintained over time in both the ribociclib+NSAI and NSAI alone arms.

### XV. EXAMPLE 2. TRIAL PROTOCOL

The NATALEE trial has been and is being performed according to the protocol described in Appendix A.

### XVI. EXAMPLE 3. FIRST INTERPRETABLE RESULTS AND UPDATED ANALYSIS

First interpretable results are described in Appendix B.

With 5.6 months of additional follow-up (median follow-up of 33.3 months) and 78.3% of patients having completed Kisqali® (ribociclib) investigational treatment, the updated analysis shows sustained iOFS benefit and stability in secondary endpoints including overall survival (OS).

iDFS benefit remains consistent across key patient subgroups; among patients with stage II and stage III tumors, Kisqali lowered risk by 30% and 24.5%, respectively.

Results reinforce the benefit seen at the earlier interim analysis, with a 25.1% (HR=0.749; 95% Cl: 0.628, 0.892; p=0.0006) reduction in risk of disease recurrence in patients with stage II and III hormone receptor-positive/human epidermal growth factor receptor 2-negative (HR+/HER2−) early breast cancer (EBC) treated with adjuvant Kisqali plus a non-steroidal aromatase inhibitor as standard endocrine therapy (ET) compared to ET alone.

Kisqali iDFS benefit across pre-specified subgroups:

| Subgroup | 3-year iDFS rate, % | HR (95% CI) |
|---|---|---|
| ITT population | Kisqali + ET: 90.7 | 0.749 |
| | ET alone: 87.6 | (0.628, 0.892) |
| AJCC Stage II | Kisqali + ET: 94.2 | 0.700 |
| | ET alone: 92.6 | (0.496, 0.986) |
| AJCC Stage III | Kisqali + ET: 88.1 | 0.755 |
| | ET alone: 83.8 | (0.616, 0.926) |
| Node-negative (NO) | Kisqali + AI: 93.2 | 0.723 |
| | ET alone: 90.6 | (0.412, 1.266) |

Kisqali data across all secondary efficacy endpoints was also consistent, including distant disease-free survival (DDFS) (25.1% risk reduction) and recurrence-free survival (RFS) (27.3% risk reduction). With fewer than 4% of events in both treatment arms (3.3% in the Kisqali-ET arm and 3.4% in the ET only arm), overall survival (OS) results will continue to evolve in the longer term.

The safety profile of Kisqali at the 400 mg dose remained consistent with previously reported results, with generally low-grade adverse events (AEs), other than laboratory abnormalities. AEs of special interest (grade 3 or higher) were neutropenia (44.3%), liver-related AEs (e.g., elevated transaminases) (8.6%), and QT interval prolongation (1.0%)1,2. No new safety signals were identified.

### XVII. APPENDIX A—CLINICAL TRIAL PROTOCOL

Approximately 425 sites will participate in the trial globally. The list of Investigators participating in the trial will be maintained by Translational Research in Oncology (TRIO).

List of Abbreviations

| | |
|---|---|
| AE | Adverse Event |
| AI | Aromatase Inhibitor |
| AJCC | American Joint Committee on Cancer |
| ALND | Axillary Lymph Node Dissection |
| ALP | Alkaline Phosphatases |
| ALT | Alanine Transaminase |
| ANC | Absolute Neutrophil Count |
| aPTT | Activated Partial Thromboplastin Time |
| AST | Aspartate Transaminase |
| ATC | Anatomical Therapeutic Chemical |
| AUC | Area Under the Curve |
| AV | Atrioventricular |
| BC | Breast Cancer |
| BCRP | Breast Cancer Resistance Protein |
| BSEP | Bile Salt Export Pump |
| CA | Competent Authority |
| CBP | Childbearing Potential |
| CCND1 | Cyclin D1 |
| CDK | Cyclin-Dependent Serine-Threonine Protein |
| | Kinases |
| CI | Confidence Interval |
| CISH | Chromogenic In Situ Hybridization |
| Ctrough | Trough concentration |
| Cmax | Maximum Serum Concentration |
| CMO&PS | Chief Medical Office and Patient Safety |
| COSMIC | Catalogue of Somatic Mutations in Cancer |
| CRF | Case Report Form; the term CRF can be applied |
| | to either EDC or Paper |
| CSR | Clinical Study Report |
| CT | Computed Tomography |
| CTCAE | Common Terminology Criteria for Adverse Events |
| ctDNA | Circulating Tumor DNA |
| ctRNA | Circulating Tumor RNA |
| CV | Coefficient of Variation |
| CYP | Cytochrome P450 |
| DDFS | Distant Disease Free Survival |
| DDI | Drug-Drug Interaction |
| DHEA | Dehydroepiandrosterone |
| DILI | Drug-Induced Liver Injury |
| DNA | Deoxyribonucleic Acid |
| EBC | Early Breast Cancer |
| EBCTCG | EBC Trialists' Collaborative Group |
| ECG | Electrocardiogram |
| ECHO | Echocardiogram |
| ECOG | Eastern Cooperative Oncology Group |
| EDC | Electronic Data Capture |
| eGFR | Estimated Glomerular Filtration Rate |
| EORTC- | European Organization for Research and Treatment |
| QLQ | of Cancer's Core Quality of Life Questionnaire |
| EOT | End of Treatment |
| EQ-SD-SL | EuroQoL 5-Level Instrument |
| ER | Estrogen Receptor |
| ET | Endocrine Therapy |
| FAS | Full Analysis Set |
| FDA | Food and Drug Administration |
| FDG-PET | Fludeoxyglucose Positron Emission Tomography |
| FISH | Fluorescence In Situ Hybridization |
| FSH | Follicle-stimulating hormone |
| G2-3 | Histologic Grade 2-3 |
| GCP | Good Clinical Practice |
| GDPR | General Data Protection Regulation (EU) 2016/679 |
| GGT | Gamma-glutamyl Transferase |
| GI | Gastrointestinal |
| GnRH | Gonadotropin-releasing Hormone |
| GWAS | Genome-Wide Association Studies |
| HADS | Hospital Anxiety and Depression Scale |
| HBV | Hepatitis B virus |
| β-hCG | Beta Human Chorionic Gonadotropin |
| HCV | Hepatitis C Virus |
| HER2 | Human Epidermal Growth Factor Receptor 2 |
| HIPAA | Health Insurance Portability and Accountability |
| | Act (US) |
| HIV | Human Immunodeficiency Virus |
| HLA | Human Leukocyte Antigen |
| HR | Hormone Receptor |
| IB | Investigator's Brochure |
| ICH | International Council for Harmonization |
| iDFS | Invasive Disease-Free Survival |
| IDMC | Independent Data Monitoring Committee |
| IEC | Independent Ethics Committee |
| IHC | Immunohistochemistry |
| ILD | Interstitial Lung Disease |
| IMP | Investigational Medicinal Product |
| IN | Investigator Notification |
| INR | International Normalized Ratio |
| IRB | Institutional Review Board |
| IRT | Interactive Response Technology |
| IUD | Intrauterine Device |
| i.v. | Intravenous(ly) |
| LC-MS/MS | Liquid Chromatography-tandem Mass Spectrometry |
| LDH | Lactate Dehydrogenase |
| LFT | Liver Function Test |
| LLOQ | Lower Limit of Quantification |
| LPLV | Last Patient Last Visit |
| LRRFS | Loco-regional Recurrence-Free Survival |
| LVEF | Left Ventricular Ejection Fraction |
| MATE1 | Multidrug and Toxin Extrusion Protein-1 |
| MDRD | Modification of Diet in Renal Disease |
| MI | Myocardial Infarction |
| MRI | Magnetic Resonance Imaging |
| msec | Milliseconds |
| MUGA | Multiple Gated Acquisition |
| NaF-PET | Sodium Fluoride Positron Emission Tomography |
| NCI | National Cancer Institute |
| NSAI | Non-Steroidal Aromatase Inhibitor |
| OCT2 | Organic Cation Transporter 2 |
| ORR | Overall Response Rate |
| OS | Overall survival |
| PAS | Pharmacokinetics Analysis Set |
| PFS | Progression-Free Survival |
| P-gp | P-glycoprotein |
| PgR | Progesterone Receptor |
| PICF | Patient Informed Consent Form |
| PK | Pharmacokinetics |
| PK-ANC | PK-Absolute Neutrophil Count |
| PK-QTcF | PK-QT Interval in the ECG corrected according |
| | to the formula of Fridericia |
| PPS | Per-Protocol Set |
| PRO | Patient Reported Outcomes |
| PT | Prothrombin Time |
| QD | Once a day |
| QoL | Quality of Life |
| QT | QT Interval in the ECG |
| QTc | QT Interval in the ECG (corrected) |
| QTcF | QT Interval in the ECG (corrected according |
| | to the formula of Fridericia) |
| RFS | Recurrence-Free Survival |
| RNA | Ribonucleic Acid |
| RoW | Rest of the World |
| SAE | Serious Adverse Event |
| SAP | Statistical Analysis Plan |
| SC | Steering Committee |
| SEER | Surveillance, Epidemiology, and End Results |
| SGOT | Serum Glutamic Oxaloacetic Transaminase |
| SGPT | Serum Glutamic Pyruvic Transaminase |
| SISH | Silver in Situ Hybridization |
| SLN | Sentinel Lymph Node |
| STEEP | Standardized Definitions for Efficacy End Points |
| | (in Adjuvant Breast Cancer Trials) |
| SUSAR | Suspected Unexpected Serious Adverse Reaction |
| T_{1/2} | Half-life |
| TBIL | Total Bilirubin |
| TdP | Torsades de Pointes |
| TEN | Toxic Epidermal Necrolysis |
| Tₘₐₓ | The time at which the maximum observed |
| | concentration (Cₘₐₓ) occurs |
| TNM | Tumor, Node and Metastasis |
| ULN | Upper Limit of Normal |
| US | Ultrasound |
| TRIO | Translational Research in Oncology |
| VAS | Visual Analogue Scale |
| WBC | White Blood Cell |
| WHO | World Health Organization |

### Protocol Synopsis

| Protocol Title | A phase III, multicenter, randomized, open-label trial to evaluate efficacy | |
|---|---|---|
| | and safety of ribociclib with endocrine therapy as an adjuvant treatment in | |
| | patients with hormone receptor-positive, HER2-negative, early breast | |
| | cancer (New Adjuvant TriAI with Ribociclib [LEE011]: NATALEE). | |
| Trial Number | CLEE011012301C (TRIO033) | |
| Trial Sponsor | Novartis | |
| Participating | Approximately 425 sites worldwide will participate in the trial. | |
| Sites | | |
| Investigational | Ribociclib (LEE011) | |
| Drug | | |
| Indication | Adjuvant treatment of hormone receptor (HR)-positive, HER2-negative, | |
| | early breast cancer (EBC). | |
| Population | Pre and postmenopausal women and men with HR-positive, HER2- | |
| | negative EBC, after adequate surgical resection, who are eligible for | |
| | adjuvant non-steroidal aromatase inhibitor (NSAI) for at least 5 years. | |
| Background | EBC and adjuvant treatment of HR-positive, HER2-negative EBC | |
| and Rationale | Breast cancer (BC) is the most frequently diagnosed cancer worldwide. | |
| | Approximately 1.7 million new cases of BC and 522,000 deaths attributed | |
| | to BC were estimated to occur in 2012 worldwide. In the United States, | |
| | BC was projected to be the most common cancer diagnosed in 2018 with | |
| | an estimated incidence of 268,670 new cases and 41,400 deaths. | |
| | Based on Surveillance, Epidemiology, and End Results Program (SEER) | |
| | data collected between years 1975 and 2012, 93% of cases of BC | |
| | diagnosed were EBC, with 62% limited to the breast tissue and 31% | |
| | localized within the breast tissue and regional lymph nodes. Although | |
| | many patients with EBC may be rendered disease-free with local | |
| | treatments, distant recurrence due to micro-metastatic disease is | |
| | common and is the primary cause of death in patients with EBC. | |
| | It is estimated that 75% of BC express receptors for steroid hormones | |
| | (estrogen receptor [ER] and/or progesterone receptor [PgR]), and | |
| | therefore may benefit from adjuvant endocrine therapy (ET) with | |
| | tamoxifen or aromatase inhibitors (AI) (letrozole, anastrozole or | |
| | exemestane). ET, independent of chemotherapy, reduces the risk of | |
| | recurrence and BC deaths in HR-positive EBC. Current clinical guidelines | |
| | for adjuvant ET in the HR-positive EBC recommend, for premenopausal | |
| | women, the use of either (a) 5-10 years of tamoxifen with or without | |
| | ovarian suppression (ovarian suppression is recommended for women | |
| | with high risk for recurrence after adjuvant chemotherapy), or (b) 5 years | |
| | of AI with ovarian suppression. Clinical guidelines and expert opinions | |
| | recommend for postmenopausal women, either (a) initial AI for 5 years | |
| | (or up to 10 years), or (b) initial tamoxifen for 2-3 years followed by AI | |
| | either up to total 5 years, or up to 5 years of treatment with AI, or (c) | |
| | tamoxifen for ~5 years followed by 5 years of AI, or (d) tamoxifen up to | |
| | 10 years. Limited data on EBC in men suggest that tamoxifen or the | |
| | combination of an AI with gonadotropin-releasing hormone (GnRH) | |
| | agonist should be the adjuvant ET of choice. | |
| | In spite of extensive research and recent advances in a multimodality | |
| | management of EBC, recurrences are still common, especially in patients | |
| | with adverse clinical, pathological and genomic features. Approximately | |
| | 25%-30% of patients with HR-positive, HER2-negative EBC with multiple | |
| | (≥4) metastatic regional lymph nodes (largely corresponding to Anatomic | |
| | Stage Group III in the AJCC 8^{th} edition Breast Cancer Staging) will recur | |
| | within 5 years regardless of treatment; only 48% of these patients will be | |
| | distant recurrence-free within 20 years and almost half of the patients will | |
| | die of BC within 20 years despite ET for 5 years. While patients with 1-3 | |
| | metastatic regional lymph nodes (corresponding generally to Anatomic | |
| | Stage Group II in the AJCC 8^{th} edition Breast Cancer Staging) may have | |
| | lower risk of recurrence than patients with Anatomic Stage Group III, still | |
| | 31% of them will experience distant recurrence and 28% will die from BC | |
| | within 20 years despite ET. Therefore, new therapeutic strategies are | |
| | required to improve clinical outcomes in patients with HR-positive, HER2- | |
| | negative EBC. | |
| | Clinical experience with ribociclib in advanced BC | |
| | Ribociclib is an orally bioavailable and highly selective small molecule | |
| | inhibitor with highly specific nanomolar inhibitory activity against | |
| | CDK4/cyclin-D1 and CDK6/cyclin-D3 enzyme complexes. Ribociclib acts | |
| | directly on the cyclin D-CDK4/6-p16-Rb pathway to block cell-cycle | |
| | progression, leading to cell cycle arrest. | |
| | Ribociclib has been approved by a number of regulatory authorities | |
| | including the United States Food and Drug Administration (FDA) and the | |
| | European Commission. By the FDA it has been approved in combination | |
| | with: (1) an AI for the treatment of pre/perimenopausal or | |
| | postmenopausal women with HR-positive, HER2-negative advanced or | |
| | metastatic BC, as initial endocrine-based therapy; or (2) fulvestrant for | |
| | the treatment of postmenopausal women with HR-positive, HER2- | |
| | negative advanced or metastatic BC, as initial endocrine based therapy | |
| | or following disease progression on ET. In Europe, ribociclib is indicated | |
| | for the treatment of women with HR-positive, HER2-negative locally | |
| | advanced or metastatic BC in combination with an AI or fulvestrant as | |
| | initial endocrine-based therapy, or in women who have received prior ET. | |
| | In pre- or perimenopausal women, the ET should be combined with a | |
| | luteinizing hormone-releasing hormone agonist. | |
| | Rationale for conducting the trial and for trial design | |
| | While adjuvant ET for HR-positive EBC is effective in reducing risk of | |
| | recurrence and improving survival, recurrences are still common, | |
| | especially in patients with features indicative of an intermediate or high | |
| | risk of recurrence, like those with Anatomic Stage Groups II and III, | |
| | among other features. | |
| | The addition of the CDK4/6 inhibitor ribociclib to ET, has proven clinical | |
| | efficacy with tolerable toxicity profile in HR-positive, HER2-negative | |
| | advanced BC; therefore, the addition of ribociclib in the adjuvant setting | |
| | may prolong invasive disease-free survival (iDFS) in patients with HR- | |
| | positive, HER2-negative EBC with intermediate and high risk for | |
| | recurrence, by enhancing primary endocrine responsiveness and | |
| | preventing or delaying the development of acquired resistance for ET. | |
| | Therefore, the purpose of this randomized, open-label trial is to evaluate | |
| | the effect of the addition of ribociclib to standard adjuvant ET on iDFS, in | |
| | patients with HR-positive, HER2-negative, Anatomic Stage Group III, IIB | |
| | or a subset of Stage IIA (as defined in Inclusion Criterion #8) EBC. | |
| | In this trial, ribociclib will be administered at a dose of 400 mg once daily | |
| | for 3 weeks on/1 week off. The 400 mg ribociclib dose has consistent | |
| | efficacy (based on post hoc exploratory analyses) and a potentially | |
| | improved safety profile in terms of QTc prolongation and hematologic | |
| | toxicity, as compared with the standard 600 mg dose, so it is expected to | |
| | be well tolerated for the proposed 3 year treatment duration. | |
| | Objective | Endpoint |
| | Primary | |
| Objectives/ | To compare iDFS for ribociclib + ET | iDFS using STEEP criteria |
| Endpoints | versus ET in patients with HR- | (Standardized Definitions for |
| | positive, HER2-negative, EBC | Efficacy End Points in Adjuvant |
| | | Breast Cancer Trials), as assessed |
| | | by Investigator |
| | Secondary | |
| | 1. To evaluate the two treatment | RFS using STEEP criteria |
| | arms with respect to recurrence-free | |
| | survival (RFS) | |
| | 2. To evaluate the two treatment | DDFS using STEEP criteria |
| | arms with respect to distant disease- | |
| | free survival (DDFS) | |
| | 3. To evaluate the two treatment | OS |
| | arms with respect to overall survival | |
| | (OS) | |
| | 4. To evaluate patient reported | Change from baseline in the |
| | outcomes (PRO) for health-related | physical functioning sub-scale score |
| | quality of life (QoL) in the two | and global health status/QoL scale |
| | treatment arms | score as assessed by EORTC |
| | | QLQ-C30 |
| | 5. To evaluate safety and tolerability | Frequency and severity of adverse |
| | of the treatment regimen | events (AEs), laboratory and |
| | | electrocardiogram (ECG) |
| | | abnormalities |
| | 6. To characterize the | PK parameters such as C_{trough} and |
| | pharmacokinetics (PK) of ribociclib | other applicable parameters for |
| | when given in combination with | ribociclib |
| | NSAI (and goserelin if applicable) | |
| | Exploratory | |
| | 1. To explore the two treatment arms | LRRFS |
| | with respect to loco-regional | |
| | recurrence-free survival (LRRFS) | |
| | 2. To explore use of subsequent anti- | Incidence of subsequent anti- |
| | neoplastic therapy | neoplastic therapy and time to first |
| | | subsequent anti-neoplastic therapy |
| | 3. To explore healthcare resource | Number of patients hospitalized, |
| | utilization | total number of hospitalizations, and |
| | | length of stay in hospitals, number |
| | | of patients with Emergency Room |
| | | and additional visits |
| | 4. To explore prognostic and | Assessment of expression and |
| | predictive biomarkers of treatment | alterations of genes related but not |
| | with ribociclib and ET | limited to CDK and ER pathways in |
| | | baseline tumor and circulating |
| | | tumor (ct) DNA and ctRNA |
| | | samples, and their correlation with |
| | | efficacy endpoints |
| | | Expression of markers such as Ki67 |
| | | by immunochemistry may be |
| | | evaluated |
| | 5. To explore potential molecular | Assessment of gene expression |
| | mechanisms of resistance to | and alterations in tumor biopsy and |
| | treatment with ribociclib and ET | ctDNA/ctRNA collected at baseline |
| | | and at time of recurrence |
| | 6. To explore the role of | Assessment of gene expression |
| | ctDNA/ctRNA for their suitability to | and alterations in ctDNA/ctRNA |
| | monitor and predict disease | collected serially at baseline, on |
| | recurrence | treatment, post treatment and at |
| | | time of recurrence |

| | |
|---|---|
| Trial Design | This is a phase III, multicenter, randomized, open-label trial to evaluate |
| and Treatment | the efficacy and safety of ribociclib with ET as an adjuvant treatment in |
| | women and men with HR-positive, HER2-negative EBC. iDFS is the |
| | primary endpoint of the trial, as defined per the STEEP System. |
| | The trial will include pre and postmenopausal women and men with HR- |
| | positive, HER2-negative EBC, with an Anatomic Stage Group III, IIB or a |
| | subset of Stage IIA cases (as defined in Inclusion Criterion #8), after |
| | adequate surgical resection, radiotherapy (if indicated), adjuvant or |
| | neoadjuvant chemotherapy (if indicated), and who are deemed to be |
| | eligible for adjuvant ET for at least 60 months of duration. |
| | See FIG. 1. |
| | Approximately 5,000 patients will be randomized (using an Interactive |
| | Response Technology system [IRT]) into two treatment arms in a 1:1 |
| | ratio to: |
| | Investigational arm: |
| | Ribociclib 400 mg by mouth once daily on days 1 to 21 of a 28-day |
| | cycle, for 36 months since randomization (approximately 39 cycles). |
| | And |
| | ET consisting of: |
| | For postmenopausal women: letrozole 2.5 mg by mouth once |
| | daily continuously or anastrozole 1 mg by mouth once daily |
| | continuously. |
| | For premenopausal women and men: letrozole 2.5 mg by mouth |
| | once daily continuously or anastrozole 1 mg by mouth once daily |
| | continuously, combined with goserelin 3.6 mg subcutaneously |
| | once every 4 weeks. |
| | Duration of ET in the trial will be 60 months from the |
| | randomization date. |
| | Control arm: |
| | ET: Same as in the Investigational arm. |
| | In both arms, ET will be administered according to the local clinical |
| | guidelines and current local prescribing information. Subsequent ET (or |
| | any other anti-cancer treatment) given after the protocol-required 60 |
| | months of ET (or after premature discontinuation of ET in the trial) will |
| | be administered according to the Investigator's clinical judgment and is |
| | not considered a trial treatment. |
| | Randomization will be stratified by the following factors: |
| | Menopausal status: premenopausal women and men vs. |
| | postmenopausal women |
| | AJCC 8^{th} edition Anatomic Stage Group: Anatomic Stage Group II vs. |
| | Anatomic Stage Group III |
| | Prior neoadjuvant/adjuvant chemotherapy: yes vs. no |
| | Geographical region: North America/Western Europe/Oceania vs. rest |
| | of the world |
| | Enrollment of patients with Anatomic Stage Group Il is capped at |
| | approximately 2,000 patients. |
| | The trial will include screening, treatment, and follow up phases. The trial |
| | includes an exploratory component that requires collection of tumor and |
| | blood samples (except for patients enrolled in China). |
| Eligibility | Inclusion Criteria |
| Criteria | Patients eligible for inclusion in this trial must meet all of the following |
| | criteria: |
| | 1. Signed and dated Patient Informed Consent Form (PICF) obtained |
| | prior to any trial-specific screening procedure. |
| | 2. Patient is ≥18 years-old at the time of PICF signature. |
| | 3. Patient is female with known menopausal status at the time of |
| | randomization or initiation of adjuvant ET (whichever occurs earlier), |
| | or male. |
| | Postmenopausal status is defined as: |
| | Patient underwent bilateral oophorectomy, or |
| | Age ≥ 60 years, or |
| | Age < 60 years and amenorrhea for 12 or more months (in the |
| | absence of chemotherapy, tamoxifen, toremifene or ovarian |
| | suppression) and Follicle-stimulating hormone (FSH) and plasma |
| | estradiol are in the postmenopausal ranges per local normal |
| | ranges. |
| | If taking tamoxifen or toremifene and age < 60 years, then FSH |
| | and plasma estradiol level in postmenopausal ranges. |
| | Notes |
| | In women who are premenopausal at the beginning of |
| | adjuvant chemotherapy, amenorrhea is not a reliable indicator |
| | of menopausal status as ovarian function may still be intact or |
| | resume despite anovulation/amenorrhea. For these women |
| | with therapy-induced amenorrhea, serial measurements of |
| | FSH and/or estradiol per local clinical guidelines are required |
| | for determination of postmenopausal status. |
| | All women who do not meet the criteria for postmenopausal |
| | status are considered premenopausal for the purpose of this |
| | trial. |
| | 4. Patient with histologically confirmed unilateral primary invasive |
| | adenocarcinoma of the breast with a date of initial cytologic or |
| | histologic diagnosis (i.e. date of the pathology report that confirmed |
| | the BC diagnosis) within 18 months prior to randomization. Patient |
| | with a multicentric and/or multifocal tumor is eligible if all |
| | histopathologically examined lesions meet the pathologic criteria in |
| | inclusion criteria 5 and 6. |
| | 5. Patient has breast cancer that is positive for ER and/or PgR |
| | according to the local laboratory as determined on the most recently |
| | analyzed tissue sample. |
| | 6. Patient has HER2-negative breast cancer defined as a negative in |
| | situ hybridization test or an immunohistochemistry (IHC) status of 0 |
| | or 1+. If IHC is 2+, a negative in situ hybridization (FISH, CISH, or |
| | SISH) test is required to confirm the HER2-negative status (based on |
| | the most recently analyzed tissue sample tested by a local |
| | laboratory). |
| | 7. Patient (except those enrolled in China) has available archival tumor |
| | tissue from the surgical specimen, for submission to a central |
| | laboratory (Note: in patients that underwent neoadjuvant systemic |
| | therapy and had a pathologic complete response, archival tumor |
| | tissue at the time of the initial diagnosis or before the administration |
| | of neoadjuvant therapy is mandatory). |
| | 8. Patient, after surgical resection where tumor was removed |
| | completely, with the final surgical specimen microscopic margins free |
| | from tumor, and belongs to one of the following categories: |
| | Anatomic Stage Group III, or |
| | Anatomic Stage Group IIB, or |
| | Anatomic Stage Group IIA that is either: |
| | N1, or |
| | N0, with: |
| | Grade 3, or |
| | Grade 2, with any of the following criteria: |
| | Ki67 ≥ 20%, or |
| | Oncotype DX Breast Recurrence Score ≥ 26, or |
| | Prosigna/PAM50 categorized as high risk, or |
| | MammaPrint categorized as high risk, or |
| | EndoPredict EPclin Risk Score categorized as high |
| | risk |
| | Notes: |
| | For patients whose tumors are Anatomic Stage IIA, NO: |
| | If Grade is 1 or unknown (Gx), patient is not eligible. |
| | If Grade 2, the gene expression test results (by Oncotype |
| | DX, Prosigna/PAM50, MammaPrint or EndoPredict |
| | EPclin) or Ki67 levels should be used if obtained as per |
| | local practice (i.e. are not mandatory for the purpose of the |
| | trial). Results must be available at screening. |
| | Patients that received neoadjuvant treatment must meet the |
| | above criteria (for stage, and if Stage IIA, N0, also for grade |
| | and Ki67 or gene expression test) in any presurgical |
| | staging/sample and/or in the surgical specimen. |
| | Categorization into the AJCC 8^{th} edition Anatomic Stage |
| | Groups requires determination of the T, N and M categories. |
| | ALND is the preferred method for axillary lymph node staging, |
| | however SLN dissection can be used to determine the N |
| | category in the following cases: |
| | No metastasis in SLN (patient is considered as pN0). |
| | Only micrometastasis in SLN (patient is considered as |
| | pN1mi). |
| | Patients with T1-2 and no clinically-evident nodes prior to |
| | surgery, no neoadjuvant chemotherapy, at least one |
| | macrometastasis in 1 or 2 SLNs, no matted nodes or gross |
| | extranodal disease at the time of SLN dissection (patient |
| | is considered as pN1). |
| | In all other cases, ALND is required to determine the N |
| | category. |
| | 9. If indicated, patient has completed adjuvant and/or neoadjuvant |
| | chemotherapy according to the institutional guidelines, prior to |
| | screening. |
| | 10. If indicated, patient has completed adjuvant radiotherapy according |
| | to the institutional guidelines, prior to screening. |
| | 11. Patient has no contraindication for the adjuvant ET in the trial and is |
| | planned to be treated with ET for 5 years (since randomization date) |
| | or more. |
| | 12. Patient may have already received any standard neoadjuvant and/or |
| | adjuvant ET at the time of PICF signature, but randomization should |
| | occur within 12 months of the initial start date of ET. Ovarian |
| | suppression or short term ET for fertility preservation is not |
| | considered neoadjuvant/adjuvant ET. If patient was receiving |
| | tamoxifen or toremifene as adjuvant ET, a washout period of 5 half- |
| | lives (i.e. 35 days) prior to randomization is required (during that |
| | period patient can take AI). |
| | 13. Patient has an Eastern Cooperative Oncology Group (ECOG) |
| | Performance Status of 0 or 1. |
| | 14. Patient has adequate bone marrow and organ function as defined by |
| | the following local laboratory values: |
| | Absolute neutrophil count (ANC) ≥ 1.5 × 10⁹/L |
| | Platelets ≥ 100 × 10⁹/L |
| | Hemoglobin ≥ 9.0 g/dL |
| | Estimated glomerular filtration rate (eGFR) ≥ 30 mL/min/1.73 m² |
| | according to the Modification of Diet in Renal Disease (MDRD) |
| | formula |
| | Alanine transaminase (ALT) < 2.5 × Upper Limit Normal (ULN) |
| | Aspartate transaminase (AST) < 2.5 × ULN |
| | Total serum bilirubin < ULN; or total bilirubin ≤ 3.0 × ULN or direct |
| | bilirubin ≤ 1.5 × ULN in patients with well documented Gilbert's |
| | Syndrome |
| | International normalized ratio (INR) ≤ 1.5 (unless the patient is |
| | receiving anticoagulants and the INR is within the therapeutic |
| | range of intended use for that anticoagulant within 7 days prior to |
| | randomization) |
| | Patient must have the following laboratory values within normal |
| | limits or corrected to within normal limits with supplements (the |
| | local laboratory value should be documented within normal limits |
| | after the correction) before randomization: |
| | Potassium |
| | Magnesium |
| | Total Calcium (corrected for serum albumin) |
| | 15. Standard 12-lead ECG values assessed by a central laboratory, as: |
| | QTcF interval (QT interval using Fridericia's correction) at |
| | screening < 450 milliseconds (msec) |
| | Resting heart rate 50-90 beats per minute (determined from the |
| | ECG) |
| | 16. Patient must be willing and able to comply with scheduled visits, |
| | treatment plans, laboratory tests, and other trial procedures. |
| | 17. Women of childbearing potential (CBP), defined as all women |
| | physiologically capable of becoming pregnant (see Inclusion Criterion |
| | #18 for additional information), must have confirmed negative serum |
| | pregnancy test (for β-hCG) within 14 days prior to randomization. |
| | 18. Women of CBP must be willing to use highly effective methods of |
| | contraception. Contraception must continue during the trial treatment |
| | and for 21 days after stopping the treatment. Highly effective |
| | contraception methods include: |
| | Total abstinence (when this is in line with the preferred and usual |
| | lifestyle of the patient). Periodic abstinence (e.g. calendar, |
| | ovulation, symptothermal, post-ovulation methods) and withdrawal |
| | are not acceptable methods of contraception. |
| | Female sterilization (have had surgical bilateral oophorectomy with |
| | or without hysterectomy), total hysterectomy or tubal ligation at |
| | least 6 weeks before taking trial treatment. In case of |
| | oophorectomy alone, only when the reproductive status of the |
| | woman has been confirmed by follow up hormone level |
| | assessment. |
| | Male partner sterilization (at least 6 months prior to randomization). |
| | For female patients on the trial the vasectomized male partner |
| | should be the sole partner for that patient. If vasectomy of the male |
| | partner is the highly effective method of contraception chosen, the |
| | success of the vasectomy should be medically confirmed |
| | according to local practice. |
| | Placement of an intrauterine device (IUD). |
| | Notes: |
| | Use of oral (estrogen and progesterone), transdermal, |
| | injected, implanted, hormone containing intrauterine system |
| | or any other hormonal method of contraception is not allowed |
| | in this trial. |
| | Women are considered of CBP unless: they have had ≥12 |
| | months of natural (spontaneous) amenorrhea with an |
| | appropriate clinical profile (i.e. age appropriate, history of |
| | vasomotor symptoms) or have had surgical bilateral |
| | oophorectomy (with or without it hysterectomy), total |
| | hysterectomy, or tubal ligation at least six weeks prior to |
| | randomization. In the case of oophorectomy alone, only when |
| | the reproductive status of the woman has been confirmed by |
| | follow-up hormone level assessment she is considered not of |
| | CBP. |
| | After the end of trial treatment, patients should use effective |
| | contraception according to local guidelines. |
| | Exclusion Criteria |
| | Patients eligible for this trial must not meet any of the following criteria: |
| | 1. Patient has received any CDK4/6 inhibitor. |
| | 2. Patient has received prior treatment with tamoxifen, raloxifene or AIs |
| | for reduction in risk (“chemoprevention”) of breast cancer and/or |
| | treatment for osteoporosis within the last 2 years prior to |
| | randomization. Patient is concurrently using hormone replacement |
| | therapy. |
| | 3. Patient has received prior treatment with anthracyclines at |
| | cumulative doses of 450 mg/m² or more for doxorubicin, or 900 |
| | mg/m² or more for epirubicin. |
| | 4. Patient with a known hypersensitivity to any of the excipients of |
| | ribociclib and/or ET (e.g. rare hereditary problems of galactose |
| | intolerance, the Lapp lactase deficiency, glucose-galactose |
| | malabsorption, and soy allergy). |
| | 5. Patient with distant metastases of breast cancer beyond regional |
| | lymph nodes (stage IV according to AJCC 8^{th} edition) and/or |
| | evidence of recurrence after curative surgery. |
| | 6. Patient is concurrently using other anti-neoplastic therapy with the |
| | exception of adjuvant ET (see Inclusion Criterion #12). |
| | 7. Patient has had major surgery, chemotherapy or radiotherapy within |
| | 14 days prior to randomization. |
| | 8. Patient has not recovered from clinical and laboratory acute toxicitys |
| | related to prior anti-cancer therapies to a NCI CTCAE (National |
| | Cancer Institute Common Terminology Criteria for Adverse Events) |
| | version 4.03 Grade ≤1 at day of randomization. Exceptions to this |
| | criterion: patients with any grade of alopecia, amenorrhea, grade 2 |
| | neuropathy are allowed to enter the trial or other toxicities not |
| | considered a safety risk for the patient as per Investigator's |
| | discretion, are allowed to enter the trial. |
| | 9. Patient has a concurrent invasive malignancy or a prior invasive |
| | malignancy whose treatment was completed within 2 years before |
| | randomization. Note: Patients with adequately treated, basal or |
| | squamous cell skin carcinoma or curatively resected cervical cancer |
| | in situ are eligible. |
| | 10. Patient has known history of human immunodeficiency virus (HIV) |
| | infection (testing is not mandatory, unless required by local |
| | regulation). |
| | 11. Patient has known active hepatitis B virus (HBV) or hepatitis C virus |
| | (HCV) infection (testing is not mandatory, unless required by local |
| | regulation). |
| | 12. Clinically significant, uncontrolled heart disease and/or cardiac |
| | repolarization abnormality, including any of the following: |
| | History of documented myocardial infarction (MI), angina pectoris, |
| | symptomatic pericarditis, or coronary artery bypass graft within 6 |
| | months prior to trial entry. |
| | Documented cardiomyopathy. |
| | Left Ventricular Ejection Fraction (LVEF) < 50% as determined by |
| | Multiple Gated acquisition (MUGA) scan or echocardiogram |
| | (ECHO) (testing not mandatory) |
| | Long QT syndrome or family history of idiopathic sudden death or |
| | congenital long QT syndrome, or any of the following: |
| | Risk factors for Torsades de Pointes (TdP) including |
| | uncorrected hypocalcemia, hypokalemia or |
| | hypomagnesemia, history of cardiac failure, or history of |
| | clinically significant/symptomatic bradycardia. |
| | Concomitant medication(s) with a known risk to prolong the |
| | QT interval and/or known to cause TdP that cannot be |
| | discontinued or replaced by safe alternative medication (e.g. |
| | within 5 half-lives or 7 days prior to starting trial treatment). |
| | Inability to determine the QTcF interval. |
| | Clinically significant cardiac arrhythmias (e.g. ventricular |
| | tachycardia), complete left bundle branch block, high-grade |
| | Atrioventricular (AV) block (e.g. bifascicular block, Mobitz type II |
| | and third degree AV block). |
| | Uncontrolled arterial hypertension with systolic blood pressure |
| | >160 mmHg. |
| | 13. Patient is currently receiving any of the following substances within 7 |
| | days before randomization: |
| | Concomitant medications, herbal supplements, and/or fruits (e.g. |
| | grapefruit, pummellos, starfruit, Seville oranges) and their juices |
| | that are known as strong inhibitors or inducers of CYP3A4/5 |
| | Medications that have a narrow therapeutic window and are |
| | predominantly metabolized through CYP3A4/5 |
| | 14. Patient is currently receiving or has received systemic |
| | corticosteroids ≤2 weeks prior to starting trial treatment, or has not fully |
| | recovered from side effects of such treatment. |
| | Note: The following uses of corticosteroids are permitted: a short |
| | duration (<5 days) of systemic corticosteroids; any duration of topical |
| | applications (e.g. for rash), inhaled sprays (e.g. for obstructive |
| | airways diseases), eye drops or local injections (e.g. intra-articular). |
| | 15. Patient has impairment of gastrointestinal (GI) function or GI disease |
| | that may significantly alter the absorption of the oral trial treatments |
| | (e.g. uncontrolled ulcerative diseases, uncontrolled nausea, vomiting |
| | or diarrhea, malabsorption syndrome, or small bowel resection). |
| | 16. Patient has any other concurrent severe and/or uncontrolled medical |
| | condition that would, in the Investigator's judgment, cause |
| | unacceptable safety risks, contraindicate patient participation in the |
| | clinical trial or compromise compliance with the protocol (e.g. chronic |
| | pancreatitis, chronic active hepatitis, liver cirrhosis or any other |
| | significant liver disease, active untreated or uncontrolled fungal, |
| | bacterial or viral infections, active infection requiring systemic anti- |
| | bacterial therapy, etc.) or limit life expectancy to ≤5 years. |
| | 17. Participation in other studies involving investigational drug(s) within |
| | 30 days prior to randomization or within 5 half-lives of the |
| | investigational drug(s) (whichever is longer), or participation in any |
| | other type of medical research judged not to be scientifically or |
| | medically compatible with this trial. If the patient is enrolled or |
| | planned to be enrolled in another study that does not involve an |
| | investigational drug, the agreement of the Medical Monitor is required |
| | to establish eligibility. |
| | 18. Pregnant or breast-feeding (lactating) women or women who plan to |
| | become pregnant or breast-feed during the trial. |
| Assessments | Trial visits will occur at screening, randomization, during treatment, |
| | approximately 30 days after discontinuation of ribociclib (Investigational |
| | arm only), End of Treatment (EOT), approximately 30 days after |
| | discontinuation of all trial treatments and during Follow-up. |
| | Efficacy assessments |
| | Efficacy assessment for the primary endpoint of iDFS will include |
| | detection of locoregional relapse, distant relapse, ipsilateral and |
| | contralateral invasive BC, second primary invasive non-BCs and deaths. |
| | Assessments for events of recurrence will be done clinically every 12 |
| | weeks (±2 weeks) for the first 24 months from randomization and every |
| | 24 weeks (±3 weeks) thereafter. Mammography will be done annually. |
| | Recurrences suspected clinically will be confirmed by additional imaging |
| | and every effort should be done to also confirm them |
| | histologically/cytologically (unless unsafe per Investigator's discretion). |
| | Survival status will be assessed up to 60 months after the last patient has |
| | been randomized, until death, withdrawal of consent, loss to follow-up or |
| | end of trial, whichever is earliest. Survival information can be obtained by |
| | clinical visits, telephone calls, or other means. |
| | Safety assessments |
| | Safety will be assessed for each patient and will include routine safety |
| | monitoring (AEs collection, laboratory testing of hematology, serum |
| | chemistry, measurement of vital signs, performance status, physical |
| | examination, and ECG). |
| | Assessments of QoL and healthcare resources utilization |
| | QoL will be evaluated by periodic questionnaires (i.e. EORTC QLQ-C30, |
| | EORTC QLQ-BR23, EQ-5D-5L and HADS), at screening and during the |
| | Treatment and Follow up Phases. |
| | Use of subsequent anti-cancer therapy, time to first subsequent anti- |
| | cancer therapy and healthcare resources utilization will be evaluated for |
| | each arm. |
| | PK assessments |
| | Approximately 130 patients from the Investigational arm will be part of |
| | the PK subset. Plasma samples for ribociclib determination will be |
| | obtained pre- and post-dose on C1D15. |
| | Exploratory biomarkers assessment |
| | Tumor tissue samples will be collected (except for patients enrolled in |
| | China) to identify biomarkers that may be predictive of benefit from |
| | ribociclib and to assess molecular alterations of genes shown to be |
| | associated with HR-positive BC and the cell cycle. |
| | A mandatory archival paraffin tumor tissue from the curative surgery |
| | specimen is required from all patients (except for patients enrolled in |
| | China). If available, an additional tumor tissue sample collection is |
| | requested from the biopsy specimen of the tumor at the time of the initial |
| | diagnosis or before the administration of neoadjuvant chemotherapy (if |
| | administered). |
| | An additional tumor biopsy collection is mandatory at the time of |
| | recurrence (unless unsafe for the patient per Investigator's discretion), for |
| | identifying potential biomarkers and exploring mechanisms underlying |
| | disease recurrence (except for patients enrolled in China). |
| | Mandatory blood samples for ctDNA/ctRNA will be collected at baseline, |
| | at regular intervals until distant recurrence and at time of recurrence |
| | (except for patients enrolled in China). |
| | Optional blood samples for pharmacogenetic analysis will be collected |
| | in patients that consent to this (except for patients enrolled in China). In |
| | addition, for consenting patients and if allowed per local regulation, |
| | optional additional biomarker analyses are planned. |
| Statistical | The primary efficacy analysis will be the comparison of the distribution of |
| Methods | iDFS between the two treatment arms using a stratified log-rank test at |
| | one-sided 2.5% level of significance, using strata information at the time |
| | of randomization. Distribution of iDFS will be estimated using the Kaplan- |
| | Meier method. The iDFS at the end of each year along with 95% |
| | confidence intervals will be presented for each of the two treatment arms. |
| | The stratified Cox regression will be used to estimate the hazard ratio of |
| | iDFS, along with 95% confidence interval, using strata information as per |
| | IRT at the time of randomization. |
| | Three interim efficacy analyses are planned. The first interim analysis, |
| | that allows the trial to stop for futility (non-binding), is planned after |
| | approximately 40% iDFS events (approximately 200 events) are |
| | documented. No efficacy will be declared in the first interim analysis. The |
| | second and third interim analyses, that allow the trial to stop for superior |
| | efficacy, are planned after all patients have been randomized and |
| | approximately 70% and 85% iDFS events (approximately 350 and 425 |
| | events) are documented, respectively. |
| | The Final Analysis for iDFS will be performed after approximately 500 |
| | iDFS events have been documented, if the trial fails to stop at the interim |
| | analyses. |
| | The sample size calculation is based on the primary variable, iDFS. The |
| | 5-year iDFS rate for the patients with Anatomic Stage II (excluding low |
| | risk patients) is assumed to be approximately 79% (based on data from a |
| | retrospective study assessing the prognostic effect of Ki67 and other |
| | disease characteristics in HR-positive, HER2-negative EBC) and 72% for |
| | patients with Anatomic Stage III (based on data for patients with ≥4 |
| | lymph nodes treated with AIs in the EBC Trialists' Collaborative Group |
| | meta-analysis). Based on these assumptions and the expected |
| | distribution of patients across the anatomical stages, the overall 5-year |
| | iDFS of the control arm is expected to be approximately 74.8%. It is |
| | expected that treatment with ribociclib in addition to standard ET will |
| | result in a 27% reduction in the hazard rate for iDFS, i.e. an expected |
| | hazard ratio of 0.73. 500 iDFS events will provide a power of |
| | approximately 93% and 85% when the overall hazard ratio is 0.73 and |
| | 0.76, respectively. Assuming a 15% dropout rate by the time of the final |
| | iDFS analysis (i.e. dropout hazard rate of 0.00629 per month), a total of |
| | 5,000 patients will need to be randomized including approximately 3,000 |
| | Anatomic Stage III patients. |

### 1. BACKGROUND AND RATIONALE

### 1.1. Disease Overview

### 1.1.1. Epidemiology

Breast cancer (BC) is the most frequently diagnosed cancer worldwide. Approximately 1.7 million new cases of BC and 522,000 deaths attributed to this disease were estimated to occur in 2012 worldwide.¹ BC incidence varies between individuals of different ethnicities and in different geographic locations around the world, with rates ranging from 27 per 100,000 in Middle Africa and Eastern Asia to 92 in Northern America.² In the United States, BC was projected to be the most common cancer diagnosed in 2018 with an estimated incidence of 268,670 new cases and 41,400 deaths.³ Estimated incidence of BC in European countries in 2012 was 458,337.⁴ BC in men is not common, with a reported frequency of approximately 1% of all BC but its incidence is continuously rising.⁵

The vast majority of newly diagnosed BC cases are early breast cancers (EBC), localized to the breast tissue and regional lymphatics, which are potentially curable with locoregional treatment modalities such as surgery and radiation therapy. Based on Surveillance, Epidemiology and End Results (SEER) Program data collected between the years 1975 and 2012, 93% cases diagnosed were EBC, with 62% limited to the breast tissue and 31% localized within the breast tissue and regional lymph nodes.⁶

### 1.1.2. Treatment of EBC

Besides primary surgery, management of EBC usually includes additional anti-neoplastic treatment modalities such as radiation therapy and adjuvant or neoadjuvant systemic therapy. Although many patients with EBC may be rendered disease-free with surgical resection and radiotherapy, distant recurrence due to micro-metastatic disease is common and is the primary cause of death in patients with EBC.⁷ According to the EBC Trialists' Collaborative Group (EBCTCG) meta-analysis of almost 150,000 women in 200 randomized clinical trials, approximately 36% and 20% of patients with EBC without any adjuvant systemic therapy will experience recurrence and death due to BC, respectively, during 5 years of follow-up.⁸ Moreover, recurrences and BC-related deaths in patients with hormone receptor (HR)-positive EBC continue to occur after 5 years from surgery, with only 45% of patients reported to be recurrence-free at 15 years of follow-up.

Adjuvant systemic treatments that comprise cytotoxic, biological and endocrine therapies in patients with EBC decrease locoregional and distant recurrences, decrease BC-specific mortality and improve overall survival (OS).⁸ The need and selection of systemic adjuvant therapies is based on individual risk of recurrence and is guided by several clinical, pathological and genomic predictive and prognostic factors of tumor and patient such as tumor stage, histopathological grade, tumor HR status, human epidermal growth factor receptor-2 (HER2) status, multi-gene testing recurrence scores, proliferation markers like Ki67, menopausal status, patient's comorbidities, age, and others. Using these factors, EBC can be classified as having low, intermediate/moderate or high risk for recurrence after surgery.⁷ While there is no consensus on the definition of these risk groups, generally, patients with smaller tumors, no metastasis in regional lymph nodes, low tumor grade, HR-positive and HER2-negative status, and low recurrence genomic score have low risk of recurrence (i.e. 5-10% recurrences at 5-years). These patients are usually considered for adjuvant endocrine therapy (ET), without chemotherapy, due to a lower clinical benefit of latter versus the former. On the other hand, patients with metastases in multiple regional lymph nodes, high tumor grade, HER2-positive status or high recurrence genomic score have a higher risk of recurrence. These patients are usually considered for adjuvant chemotherapy (and HER2 targeting agents in patients with HER2-positive BC), and if the tumor expresses HR, adjuvant ET is considered too (normally delivered after the completion of chemotherapy).

It is estimated that 75% of BC express receptors for steroid hormones (estrogen receptor [ER] and/or progesterone receptor [PgR]), and therefore these patients may benefit from adjuvant ET with tamoxifen or aromatase inhibitors (Als) (letrozole, anastrozole or exemestane).⁹ ET, independent of chemotherapy, reduces the risk of recurrence and BC deaths in HR-positive EBC.⁸

Current clinical guidelines for adjuvant ET in the HR-positive EBC recommend:^{10,11}
For premenopausal women:
   5-10 years of tamoxifen with or without ovarian suppression, or
   5 years of A1 with ovarian suppression.¹²
For postmenopausal women:
   Initial A1 for 5 years (or up to 10 years, based on results from the MA.17R trial¹³), or
   Initial tamoxifen for 2-3 years followed by AI either up to total 5 years, or up to 5 years of treatment with A1, or
   Tamoxifen for ~5 years followed by 5 years of Al, or
   Tamoxifen up to 10 years.^{10,11}
In men: Limited data suggest that tamoxifen or the combination of an AI with a gonadotropin-releasing hormone (GnRH) agonist should be the ET of choice for HR-positive, HER2-negative EBC.¹⁴

In spite of extensive research and recent advances in the multimodality management of EBC, recurrences are still common, especially in patients with adverse clinical, pathological and genomic features. Approximately 25%-30% of patients with HR-positive, HER2-negative EBC with multiple (≥4) metastatic regional lymph nodes (largely corresponding to Anatomic Stage Group III in the AJCC 8^{th} edition Breast Cancer Staging) will recur within 5 years with ET that includes Als.⁸ Only 48% of these patients will be distant recurrence-free within 20 years, with almost half of patients dying of BC within 20 years, despite ET for 5 years.¹⁵ While patients with 1-3 metastatic regional lymph nodes (corresponding generally to Anatomic Stage Group II in the AJCC 8^{th} edition Breast Cancer Staging) may have lower risk of recurrence than patients with Anatomic Stage Group III, still 31% of them will experience distant recurrence and 28% will die from BC within 20 years despite ET.¹⁵

Therefore, new therapeutic strategies are required to improve clinical outcomes in patients with HR-positive, HER2-negative EBC.

### 1.1.3. Role of the CDK4/6 Pathway in BC

Dysregulation of the CDK4/6-Rb-E2F pathway is an important contributor to ET resistance in BC. The luminal A and B subtypes of BC (85% of which are ER-positive and HER2-negative) have high rates of cyclin D/CDK activation; in the luminal A and B subtypes, cyclin D1 (CCND1) amplifications were observed in 29% and 58%, and CDK4 amplifications were observed in 14% and 25%, respectively.^{16,17} Luminal A subtype tumors also have loss of CDKN2A, which encodes p16^{INK4A}, a CDK inhibitor.¹⁸ The luminal subtypes also maintain expression of Rb, which is essential for benefit from treatment with a CDK4/6 inhibitor.¹⁹

Dysregulation of cell cycle checkpoints may have clinical and therapeutic significance. For example, patients with HR-positive BC exhibiting a gene expression signature of Rb loss had a shorter recurrence-free survival (RFS) following adjuvant tamoxifen.²⁰ A tumor gene expression signature of E2F activation is also associated with higher residual tumor cell proliferation following neoadjuvant A1 therapy. Therefore, activation of the CDK4/6-Rb-E2F pathway promotes endocrine resistance, and treatment with a CDK4/6 inhibitor or knockdown of CDK4 expression leads to reactivation of Rb, binding back of E2F and subsequent cell cycle arrest, thus abrogating endocrine-resistant cell proliferation.

Selective inhibitors of CDK4/6, such as palbociclib, abemaciclib and ribociclib, demonstrated synergy with ET in preclinical studies and efficacy in clinical studies and have been approved as initial therapy (in combination with an A1) or after disease progression following prior ET (in combination with fulvestrant) in patients with HR-positive, HER2-negative advanced BC²¹⁻²⁶ (refer to the most recent ribociclib Investigator's Brochure (IB) for more information about the efficacy of ribociclib).

Considering the demonstrated efficacy of CDK4/6 inhibitors in the HR-positive, HER2-negative advanced BC, co-targeting the CDK4/6-Rb-E2F pathway with CDK4/6 inhibitors may be a viable strategy to enhance endocrine responsiveness and prevent or delay the development of acquired resistance, and should be explored in the adjuvant setting.

Emerging data from ongoing clinical trials (PALLAS and monarchE) that investigate other CDK4/6 inhibitors in HR-positive, HER2-negative EBC, suggest that there may be an increased treatment benefit in the adjuvant setting for Anatomic Stage Group III, when compared to Anatomic Stage Group II patients.^{27,28} PALLAS trial, which investigates palbociclib in Stage II (including a proportion of patients with lower risk stage IIA) and Stage III EBC, recently reported a negative outcome while monarchE, which investigates abemaciclib and appears to include Stage III patients, reported a positive outcome. Though there are differences between the two study designs and the biochemical properties between the two CDK4/6 inhibitors investigated, early signs of greater treatment effect favouring the high risk stage III EBC from CDK4/6 inhibition cannot be discounted.

### 1.2. Overview of Investigational Treatment and Other Trial Treatments

This trial includes treatment with ribociclib (LEE011) and ET using non-steroidal aromatase inhibitor (NSAI; anastrozole or letrozole)+/−goserelin.

### 1.2.1. Overview of Ribociclib

Ribociclib is an orally bioavailable and highly selective small molecule inhibitor with highly specific nanomolar inhibitory activity against CDK4/cyclin-D1 and CDK6/cyclin-D3 enzyme complexes. As of the date of this document, ribociclib has been approved by a number of regulatory authorities including the United States Food and Drug Administration (FDA) and the European Commission. By the FDA it has been approved in combination with: (1) an A1 for the treatment of pre/perimenopausal or postmenopausal women with HR-positive, HER2-negative advanced or metastatic BC, as initial endocrine-based therapy; or (2) fulvestrant for the treatment of postmenopausal women with HR-positive, HER2-negative advanced or metastatic BC, as initial endocrine based therapy or following disease progression on ET. In Europe, ribociclib is indicated for the treatment of women with HR-positive, HER2-negative locally advanced or metastatic BC in combination with an A1 or fulvestrant as initial endocrine-based therapy, or in women who have received prior ET. In pre- or perimenopausal women, the ET should be combined with a luteinizing hormone-releasing hormone agonist.

### 1.2.1.1. Non-Clinical Data

Refer to the most recent ribociclib IB for details.

### 1.2.1.2. Clinical Experience

Ribociclib is being investigated in patients with BC and other solid tumors in multiple clinical trials at different phases of development. Refer to the most recent ribociclib IB for details on clinical trials.

### 1.2.1.2.1. Clinical Safety of Ribociclib

Clinical safety of ribociclib with endocrine agents such as letrozole, tamoxifen, exemestane, fulvestrant and goserelin has been evaluated in several combination trials. The safety profile of ribociclib in combination with NSAI (+/−goserelin) was investigated in two phase III trials in advanced BC (MONALEESA-2 and MONALEESA-7).^{24,29}

Ribociclib is not recommended for use in combination with tamoxifen, due to the increased risk of QT prolongation, based on the results from MONALEESA-7 trial.²⁹

For a comprehensive review of the safety profile of ribociclib in combination with endocrine agents, refer to the most recent ribociclib IB.

### 1.2.1.2.2. Clinical Efficacy of Ribociclib

Efficacy of ribociclib with endocrine agents has been evaluated in three published phase III combination trials in patients with advanced BC, two of them evaluating the combination of ribociclib with NSAI (+/−goserelin).^{24,29}

Refer to the most recent ribociclib IB for additional details on the efficacy profile of ribociclib.

### 1.2.1.2.3. Clinical Pharmacokinetics of Ribociclib

The clinical pharmacokinetics (PK) of ribociclib has been evaluated in a phase I study in patients with advanced solid tumors or lymphomas (study CLEE011X2101). Following a single oral dose of the capsule formulation at 400 mg, ribociclib is absorbed with median Tₘₐₓ of 4.00 h (range: 0.58 to 4.20 h). Following repeated daily oral administration, steady-state of ribociclib was achieved by approximately Day 8. At the steady state, geometric mean of ribociclib plasma Cₘₐₓ was 1040 ng/mL (geometric coefficient of variation [CV]49.3%) and AUC₀₋₂₄ₕ was 11400 ng*h/mL (geometric CV 57.8%). The geometric mean of effective T_{1/2} of ribociclib was 31.6 h (geometric CV 33.2%) and accumulation ratio was 2.46 (geometric CV 24.6%). LEQ803, an active metabolite of ribociclib, has similar PK characteristics as parent drug. Neither ribociclib nor LEQ803 accumulate substantially following repeated daily administration.

Ribociclib undergoes extensive hepatic metabolism via CYP3A in humans based on in vitro and in vivo studies. Ribociclib is mainly eliminated via hepatic clearance, with renal clearance playing a lesser role in humans. The majority of the administered dose was excreted in feces (69.1%), with a minor amount excreted in urine (22.6%). Ribociclib accounted for approximately 23% of the total radioactivity in plasma (CLEE011A2102). The most prominent metabolites in plasma are CC1284 (N-hydroxylation), LEQ803 (N-demethylation), and M1 (secondary glucuronide), each representing <10% of total radioactivity. The clinical activity (pharmacological and safety) following ribociclib treatment is primarily due to parent drug, with a negligible contribution from circulating metabolites.

Concomitant use of ribociclib with strong CYP3A4 inhibitors or strong CYP3A4 inducers should be avoided as ribociclib exposure may be markedly affected. Co-administration of a strong CYP3A4 inhibitor (ritonavir) increased ribociclib AUC_{inf} by 3.2-fold following a single oral dose of 400 mg ribociclib (CLEE011A2101). Co-administration of a strong CYP3A4 inducer (rifampicin) decreased ribociclib AUC_{inf} by 89% following a single oral dose of 600 mg ribociclib (CLEE011A2101).

Ribociclib is a moderate to strong inhibitor of CYP3A4, but did not have a substantial effect on CYP1A2 substrates in humans (CLEE011A2106). Co-administration of midazolam (CYP3A4 substrate) with multiple doses of ribociclib (400 mg) increased midazolam exposure by 3.8-fold. Co-administration of caffeine (CYP1A2 substrate) with multiple doses of ribociclib (400 mg) increased caffeine exposure by 20% (1.2-fold). Concurrent use of sensitive CYP3A4 substrates with a narrow therapeutic index should be avoided. Concurrent use of CYP1A2 substrates is not expected to lead to clinically important Drug-Drug Interaction (DDI).

Food does not affect the PK of ribociclib administered as a capsule or tablet formulation, therefore, ribociclib capsules or tablets can be taken without regard to meals (CLEE011A2111, CLEE011A2103).

No apparent DDI was observed between ribociclib and the combination partner letrozole or anastrozole based on PK data in the MONALEESA-2 and -7 trials. Based on the population PK analysis, concomitant use of letrozole or anastrozole had no impact on ribociclib exposure.

Refer to the most recent ribociclib IB for additional details.

### 1.2.2. Overview of Adjuvant Endocrine Therapy

Tamoxifen, NSAIs (i.e. letrozole, anastrozole) and steroidal A1 (i.e. exemestane) are used for adjuvant ET in women with HR-positive EBC. Als can be used either as an upfront therapy or after 2-3 or 5 years of prior tamoxifen (see Section 1.1.2). The long-term efficacy of both approaches of Als administration is similar.³⁰ Clinical treatment guidelines suggest that there is no compelling evidence showing meaningful clinical efficacy or toxicity differences between letrozole, anastrozole and exemestane, therefore similar precautions and monitoring activities should apply irrespective of type of AI administered.¹¹ GnRH agonists are used to achieve gonadal suppression in premenopausal women or men.

The following sections provide general information on NSAI and goserelin. Refer to the current local prescribing information and local clinical guidelines for comprehensive safety and efficacy information and guidance for each medication.

### 1.2.2.1. Overview of Letrozole

Letrozole is a nonsteroidal competitive inhibitor of the aromatase enzyme system. Letrozole acts by highly selective inhibition of conversion of androgens (mainly from adrenal glands, the primary source of estrogens in postmenopausal women) to estrogens. Letrozole induces a 75% to 95% decrease of estrogen levels after two weeks of treatment with daily doses of 0.1 to 5 mg, with no significant clinical and laboratory toxicities or changes in levels of other hormones of the endocrine system.^{31,32}

Letrozole is administered orally once daily at a dose of 2.5 mg. It is rapidly and completely absorbed from the gastrointestinal (GI) tract. Concomitant intake of food has no effect on the extent of letrozole absorption. Letrozole is metabolized via CYP3A4 to a pharmacologically-inactive metabolite and renal excretion of the glucuronide conjugate of this metabolite is the major pathway of letrozole clearance.

Letrozole (2.5 mg daily) and ribociclib (600 mg daily, 3 weeks on/1 week off) did not affect metabolism of each other in a phase Ib/II dose escalation/expansion study (CLEE011X2107). The most frequently reported adverse events (AE) for letrozole in the adjuvant and extended adjuvant clinical trials were hot flushes, arthralgia/arthritis and myalgia. In general, the observed adverse reactions were mild to moderate in intensity. Adjuvant use of letrozole (or anastrozole) is associated with a decrease in bone mass density, which may lead to osteoporosis and associated bone fractures. Consideration should be given to monitoring bone mass density. Due to its mechanisms of action, letrozole, as well as other Als, should not be used for above-mentioned indications in women with an intact ovarian function or in non-castrated men.

Refer to the current local prescribing information for more information on letrozole.

### 1.2.2.2. Overview of Anastrozole

Anastrozole, like letrozole, is a selective NSAI. It significantly lowers serum estradiol concentrations with no detectable effect on formation of adrenal corticosteroids or aldosterone. Anastrozole is administered orally once daily at a dose of 1 mg, taken with or without food. Anastrozole is metabolized by N-dealkylation, hydroxylation and glucuronidation. Hepatic metabolism accounts for approximately 85% of anastrozole elimination. Renal elimination accounts for approximately 10% of total clearance. The major circulating metabolite of anastrozole lacks pharmacologic activity. Anastrozole metabolism occurs mainly via CYP3A4 and UGT1A4 based on in vitro data.³³ Therefore, anastrozole metabolism may potentially be affected by co-administration with ribociclib. However, anastrozole has been studied up to doses of 10 mg/day (10-times daily dose) and all doses evaluated were well tolerated with no serious acute toxicities attributed to anastrozole.³⁴

Refer to the current local prescribing information for more information on anastrozole.

### 1.2.2.3. Overview of GnRH Agonists

GnRH agonists are synthetic analogues of gonadotropin-releasing hormone that by continuous stimulation of the GnRH receptor achieve desensitization of the pituitary gland to GnRH. GnRH agonists differ from the naturally-occurring GnRH by modifications in the decapeptide structure (usually by amino acid substitution in position 6, but also in positions 9 and 10) to decrease degradation of the molecule.

Goserelin is the GnRH agonist to be used in this trial. The most common AEs occurring in women treated with goserelin includes hot flushes, headache, sweating, acne, emotional liability, depression, decreased libido, vaginitis, breast atrophy, seborrhea and peripheral edema. In men, goserelin can be associated with hot flushes, sexual dysfunction, decreased erections and lower urinary tract symptoms.

Refer to the current local prescribing information and/or clinical guidelines for more information on goserelin.

### 1.3. Rationale

### 1.3.1. Rationale for Conducting the Trial

While adjuvant ET for HR-positive EBC is effective in reducing risk of recurrence and improving survival, recurrences are still common, especially in patients with features indicative of an intermediate or high risk of recurrence, like those with Anatomic Stage Groups II and III, among other features. These recurrences, mostly in the form of distant metastases, are usually incurable and will eventually translate into BC deaths.¹⁵

The addition of ribociclib to ET has proven clinical efficacy with tolerable toxicity profile in HR-positive, HER2-negative advanced BC; therefore, the addition of ribociclib in the adjuvant setting may prolong invasive disease-free survival (iDFS) in patients with HR-positive, HER2-negative EBC with intermediate and high risk for recurrence, by enhancing primary endocrine responsiveness and preventing or delaying the development of acquired resistance to ET.

The purpose of this randomized, open-label trial is to evaluate the effect of the addition of ribociclib to standard adjuvant ET on iDFS in patients with HR-positive, HER2-negative Anatomic Stage Group III, IIB or a subset of Stage IIA cases (as defined in Inclusion Criterion #8) EBC.

### 1.3.2. Rationale for Trial Design

This is a phase III, multicenter, randomized, open-label trial to evaluate the addition of ribociclib to standard adjuvant ET in women and men with HR-positive, HER2-negative EBC. Adjuvant ET in the trial will be NSAI (letrozole, anastrozole) for postmenopausal women and NSAI combined with goserelin in premenopausal women and men.

The randomized, stratified, multicenter design of this trial minimizes allocation bias, balancing both known and unknown prognostic factors in the assignment of treatments.

Appropriate adjudication of outcomes, especially the primary endpoint, is of critical importance to the validity of trial results in this open-label trial. To reduce bias, the primary endpoint chosen is objective (iDFS), and a standardized definition will be used (per STEEP System for Standardized Definitions for Efficacy End Points in Adjuvant Breast Cancer Trials).³⁵ Also, iDFS events will not only be based on clinical or radiological assessments but also will be confirmed histologically or cytologically (unless there is an unacceptable risk to the patient due to the procedure), therefore requiring an objective confirmation to consider a recurrence as an iDFS event. Besides, if a patient discontinues trial treatment for reasons other than distant recurrence, assessment for recurrence should continue until distant relapse per STEEP criteria.

All these elements support the validity and objectiveness of the underlying recurrence assessment for the iDFS endpoint and ensure that the results of the trial would not be affected by the open-label design. Furthermore, since ribociclib is associated with a rate of neutropenia of approximately 75%,³⁶ concealment of treatment allocation is not likely to be effective.

The trial will include pre and postmenopausal women and men with HR-positive, HER2-negative EBC, with an Anatomical Stage Group (according to AJCC 8^{th} edition) III, IIB or a subset of Stage IIA cases (as defined in Inclusion Criterion #8). These Anatomic Stage Groups include (mostly) tumors with metastatic regional lymph nodes and/or large primary lesions, which have increased risk of recurrence despite ET in which the addition of ribociclib could be of higher benefit.

Ki67 or gene expression tests will be used (if these are available) to identify patients with node-negative Anatomic Stage IIA and histological grade 2, that are considered of higher risk.

Although Ki67 index suffers from poor reproducibility, it is commonly used globally for the estimation of prognosis and guiding the decision on adjuvant treatment choice in ER-positive, HER2-negative EBC. Despite the lack of standardization and the variability in the cut-offs used to define a high Ki67 index, its prognostic or predictive value has been demonstrated in a number of studies.³⁷ The 20% cut-off has been identified as an appropriated to stratify high-risk patients in ER-positive, HER2-negative EBC.^{38,39} Gene expression tests evaluating the BC genomic risk category provide clinicians with prognostic information useful to individualize treatment and have been widely adopted, especially to identify those patients with good prognosis that would not require adjuvant chemotherapy.^{10,11,40,41} Given the good prognosis in some patients with Anatomic Stage IIA compared to those with IIB or III, patients with node-negative stage IIA tumors will only be included if they are grade 3, or grade 2 with a high Ki67 index (≥20%) or considered high risk by a validated gene expression test (as defined in Inclusion Criterion #8).

Based on emerging data from other CDK4/6 inhibitor EBC trials that suggest an increased treatment benefit in Anatomic Stage Group III patients, the number of patients with Stage II disease is capped at approximately 2,000, out of a total study population of approximately 5,000 patients.

Both pre and postmenopausal women will be included since differences in efficacy and safety of ribociclib are not expected in these populations. To mitigate potential differences in clinical outcomes between pre and postmenopausal women, stratification by menopausal status will be employed.

Categorization into the AJCC 8^{th} edition Anatomic Stage Groups requires determination of the T, N and M categories. Axillary lymph node dissection (ALND) is the preferred method for axillary lymph node staging; however sentinel lymph node (SLN) dissection can be used to determine the N-category in certain patients (see Inclusion Criterion #8).

SLN dissection without subsequent ALND in case of positive SLN became acceptable practice in selected patients following the results of the ACOSOG Z0011 clinical trial where patients with metastatic SLN were randomized to either ALND or no additional surgery. The trial demonstrated comparable 5-year disease-free survival (83.9% vs. 82.2% in SLN dissection only and SLN dissection followed by ALND, respectively) and OS (92.5% vs. 91.8%, respectively) between the two arms and less surgical co-morbidity with SLN dissection only.⁴²

While this approach has been recommended by several clinical guidelines and consensuses and included as an option for the surgical management of the axilla in eligible patients,^{10, 11, 43, 44} omitting ALND makes precise staging of BC more challenging. Since in the ALND arm of Z0011 ~85% of patients had N1 disease, patients who conform with the trial eligibility criteria (T1-T2, <3 metastatic SLNs, no clinically-evident nodal disease before the surgery, no gross extranodal tumor extension, no neoadjuvant systemic therapy, and were treated with lumpectomy with adjuvant radiotherapy) can be considered as having N-category N1.

Randomization will be stratified by menopausal status, Anatomical Stage Group, use of prior neoadjuvant/adjuvant chemotherapy, and geographical region (see Section 3.1). Menopausal status was selected as a stratification factor since it may reflect different treatment choices and prognosis. Premenopausal women will be combined with men in the same stratum since both men and premenopausal women may have higher recurrence rate compared to postmenopausal women and the number of men expected is too small to make it as a separate stratum. Lastly, since neoadjuvant or adjuvant chemotherapy decreases recurrences in HR-positive EBC (with no significant difference between adjuvant and neoadjuvant chemotherapy regarding recurrence-free survival), randomization will also be stratified according to whether or not the patient has received any prior neoadjuvant/adjuvant chemotherapy.

The primary efficacy of the investigational intervention will be evaluated by its effect on the iDFS as it is defined in the STEEP (Standardized Definitions for Efficacy End Points) System.³⁵ The definition of DFS in the STEEP System is broad, clinically-relevant and includes the most commonly accepted DFS events used in published trials for EBC.

### 1.3.3. Rationale for Regimen and Dose Selection

Ribociclib at a dose of 600 mg daily from Days 1 to 21 of a 28-day cycle has been the regimen shown to be tolerable and efficacious when combined with ET in clinical trials in patients with advanced BC (see Section 1.2.1.2).

Due to the later occurrence of recurrences of HR-positive, HER2-negative EBC, with the majority of events observed starting from a year after surgery and lasting for at least 15 years after diagnosis, a short duration of adjuvant ribociclib treatment might not be sufficient to detect a meaningful impact on the recurrence rate.⁴⁵ Therefore, in order to provide sufficient ribociclib exposure in this trial, while balancing potential toxicity of a prolonged treatment, ribociclib (in combination with ET) will be administered for 36 months at a dose of 400 mg daily on Days 1-21 of a 28-day cycle. This schedule and duration of ribociclib therapy should be sufficient to detect a meaningful impact of the recurrence rate.

PK-QTcF (PK-QT Interval in the ECG corrected according to the formula of Fridericia) and PK-ANC (PK-Absolute Neutrophil Count) modeling has demonstrated that QTcF interval prolongation and neutropenia are ribociclib concentration-dependent. Post hoc exploratory analyses suggest that patients who started ribociclib at 600 mg, that subsequently dose reduced to 400 mg and 200 mg continued to benefit in terms of efficacy (Progression-Free Survival [PFS] and Overall Response Rate [ORR]). In an attempt to decrease the incidence of QTc prolongation, neutropenia and other AEs, a lower ribociclib dose of 400 mg once daily for 3 weeks on/1 week off in a 28-day cycle will be explored in this trial in patients with HR-positive, HER2 negative EBC.

With regard to the risk of QTcF prolongation, the mean ΔQTcF values in MONALEESA-2, -3 and -7 are consistent across these trials. The ΔQTcF prolongation from baseline at Cycle 1 Day 15 (C1 D15) at 2 hours post-dose were 19.6 milliseconds (msec), 20.6 msec, and 18.6 msec for MONALEESA-2, -3 and -7 (NSAI subgroup), respectively. Based on the highest ECG value at any assessment, a newly occurring post baseline QTcF of >480 msec was observed among 23 (6.9%) patients, 27 (5.6%) patients and 13 (5.3%) patients in ribociclib arms in MONALEESA 2,-3 and -7 (NSAI subgroup), respectively. While clinical sequelae were not common, since the clinical trials show that ribociclib-induced QTcF interval prolongation is concentration-dependent (ribociclib IB 2017) there is an opportunity to lower the risk for this event. Based on PK-QTcF modeling, lower daily doses of ribociclib are estimated to result in a lower mean ΔQTcF (Table A1).

TABLE A1
Estimated Cₘₐₓ and Mean QTcF Change From Baseline
at Different Dose Regimens of Ribociclib.

| | Estimated | Estimated |
|---|---|---|
| | geometric | mean ΔQTcF |
| | mean Cₘₐₓ | (msec) (90% CI) of |
| Ribociclib dosing regimen | (ng/mL) | ribociclib + NSAI^{a} |
| 600 mg QD 3 weeks on/1 week | 1870 | 22.6 (21.06, 24.06) |
| off (approved dosing regimen) | | |
| 400 mg QD 3 weeks on/1 week | 1080 | 18.9 (17.65, 20.11) |
| off | | |

^{a}Estimated by PK-QT model based on the pop-PK model predicted C_{max, ss}. Both models were developed based on data from studies CLEE011X2101, CLEE011X1101, CLEE011X2107, CLEE011A2301, CLEE011E2301 and CLEE011F2301.
Abbreviations:
CI—confidence interval;
Cₘₐₓ—maximum serum concentration of ribociclib;
NSAI—non-steroidal aromatase inhibitors;
QD—once a day;
ΔQTcF—change from baseline in QT interval in the ECG (corrected according to the formula of Fridericia)

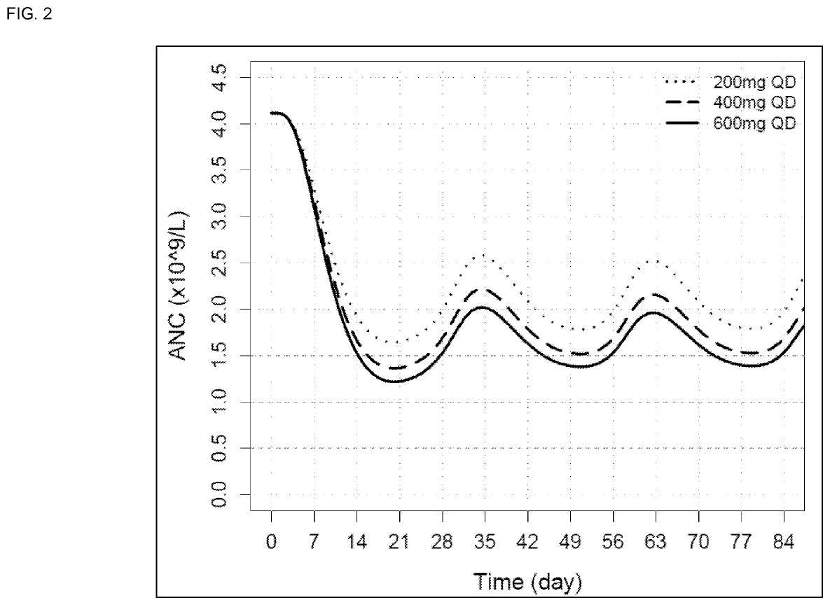

Another identified risk for ribociclib, which is a CDK4/6 inhibitor class effect, is neutropenia. Simulation of ANC profiles shows that 400 mg once daily ribociclib dose leads to a smaller ANC decrease than 600 mg once daily dose with ANC nadir close or higher than clinically significant ANC of 1.5×10⁹/L (see FIG. 2).

Therefore, a ribociclib dose of 400 mg would be expected to have less severe neutropenia associated with its use.

Post hoc exploratory analyses of MONALEESA-2, -3 and -7 also suggest that patients who started ribociclib at 600 mg but had their dose reduced (e.g. due to AE or any other reason) to 400 mg and 200 mg continued to experience treatment benefit in terms of the PFS (Table A2) and ORR (Table A3).

TABLE A2
Median PFS by Ribociclib Dose Reduction and Placebo

| | Ribociclib | Ribociclib 600 | Ribociclib 600 mg to | |
|---|---|---|---|---|
| Trial | 600 mg | mg to 400 mg | 400 mg to 200 mg | Placebo |
| MONALEESA-2 | 27.7 months | 24.9 months | 27.6 months | 16.0 months |
| MONALEESA-3 | 18.8 months | 22.1 months | NE* | 12.8 months |
| MONALEESA-7 | 22.1 months | 24.7 months | 27.5 months | 13.0 months |

*NE: Not Estimable

TABLE A3
ORR by Ribociclib Dose Reduction and Placebo

| | Ribociclib | Ribociclib 600 | Ribociclib 600 mg to | | |
|---|---|---|---|---|---|
| Trial | 600 mg | mg to 400 mg | 400 mg to 200 mg | | Placebo |
| MONALEESA-2 | 35.1 (27.9, 42.3) | 44.0 (34.3, 53.7) | 60.3 | (48.2, 72.4) | 28.7 (23.9, 33.6) |
| MONALEESA-3 | 29.8 (24.7, 35.0) | 33.6 (25.7, 41.5) | 41.9 | (24.6, 59.3) | 21.5 (16.3, 26.7) |
| MONALEESA-7 | 40.6 (34.0, 47.1) | 40.9 (30.6, 51.2) | 52 | (32.4, 71.6) | 29.7 (24.8, 34.6) |

¹ORR (95% CI) as per local Investigator's assessment in MONALEESA-2 (CLEE011A2301), MONALEESA-3 (CLEE011F2301) and MONALEESA-7 (CLEE011E2301).

While these PFS and ORR results should be interpreted with caution due to the potential confounding factor of the 600 mg daily starting dose on these efficacy outcomes, the results do provide insights regarding the potential effectiveness of lower ribociclib doses regimens. Of note, over 20% of ribociclib dose reduction occurred within the first 3 months of treatment.

In conclusion, based on the PK-QTcF modeling, post hoc exploratory PFS analyses by dose reduction and the PK-ANC modeling, a ribociclib 400 mg dose may be associated with less toxicity (e.g. lower ΔQTcF, lower incidence of neutropenia and possibly other adverse events) than the 600 mg dose, while having the potential to provide efficacy (in terms of PFS and ORR). Therefore, the ribociclib 400 mg daily dose (400 mg once-daily 3 weeks on/1 week off), with dose reductions to 200 mg if needed based on severity of toxicity, will be investigated as a starting dose in this trial.

### 1.3.4. Rationale for Choice of Combination Drugs

Ribociclib will be combined with standard ET approved for adjuvant treatment of EBC and will include NSAI, either letrozole or anastrozole. In premenopausal women and men, ET will include gonadal suppression by goserelin that will be administered every 4 weeks subcutaneously.

Preclinical and clinical data of co-administration of ET with ribociclib showed that combinations with ET are efficacious in HR-positive, HER2-negative advanced BC, and have reversible and manageable toxicities (see Section 1.2).

### 1.3.5. Rationale for Choice of Comparator Drugs

ET to be used in this trial is standard ET in patients with ER-positive EBC. Patients in the control arm will be treated with standard NSAI, either letrozole or anastrozole, administered for 60 months from the randomization date, according to the local clinical guidelines and current prescribing information. In premenopausal women and men, ET will include gonadal suppression by GnRH agonist (goserelin) as recommended in patients with intact ovarian or testicular function. Goserelin will be administered every 4 weeks subcutaneously. One-month depot formulation of goserelin must be used to suppress gonadal function in this trial (both for women and men) as the 3-month depot formulations do not reliably suppress estrogen levels in all female patients.¹¹

### 1.3.6. Risks and Benefits

Based on preclinical and clinical data, treatment of ribociclib in combination with ET is expected to be tolerable and toxicities of the treatment are expected to be manageable and reversible with treatment interruption, ribociclib dose reduction, or discontinuation.

Patients in this trial will be carefully monitored for key toxicities that have been observed with ribociclib (refer to the latest ribociclib IB) or ETs (see Section 1.2.2). Risks will be further minimized by adherence to inclusion/exclusion selection criteria (see Section 4), avoidance of prohibited medications (see Section 5.4), close safety monitoring (see Section 7) and dose adjustment guidelines (see Section 5.2.1.2).

An Independent Data Monitoring Committee (IDMC) (see Section 7.6) will be constituted and will monitor safety and efficacy. A Steering Committee (SC) (see Section 9.5) will be established comprising of Investigators, Novartis and Translational Research in Oncology (TRIO) personnel participating in the trial, to ensure transparent management of the trial according to the protocol.

The TRIO and Novartis teams will evaluate data from the trial with safety signal detection purposes on an ongoing basis. Additionally, Novartis Safety Management Team will review and evaluate all emerging data across the ribociclib program for potential safety signal assessment in a timely manner.

### 2. OBJECTIVES AND ENDPOINTS

Objectives and related endpoints are described in Table A4 below.

TABLE A4
Objectives and Related Endpoints

| Objective | Endpoint | Analysis |
|---|---|---|
| Primary | | Refer to Section 8.4 |
| To compare iDFS for ribociclib + ET | iDFS using STEEP criteria, as assessed | |
| versus ET in patients with HR- | by Investigator³⁵ | |
| positive, HER2-negative, EBC | | |
| Secondary | | Refer to Section 8.5 |
| 1. To evaluate the two treatment | RFS using STEEP criteria³⁵ | |
| arms with respect to recurrence-free | | |
| survival (RFS) | | |
| 2. To evaluate the two treatment | DDFS using STEEP criteria³⁵ | |
| arms with respect to distant | | |
| disease-free survival (DDFS) | | |
| 3. To evaluate the two treatment | OS defined as time from date of | |
| arms with respect to OS | randomization to date of death due to | |
| | any cause | |
| 4. To evaluate patient reported | Change from baseline in the physical | |
| outcomes (PRO) for health-related | functioning sub-scale score and global | |
| quality of life (QoL) in the two | health status/QoL scale score as | |
| treatment arms | assessed by EORTC QLQ-C30 | |
| 5. To evaluate safety and | Frequency and severity of AEs, | |
| tolerability of the treatment regimen | laboratory and Electrocardiogram (ECG) | |
| | abnormalities | |
| 6. To characterize the PK of | PK parameters such as C_{trough} and other | |
| ribociclib when given in combination | applicable parameters for ribociclib | |
| with NSAI (and goserelin if | | |
| applicable) | | |
| Exploratory | | Refer to Section 8.6 |
| 1. To explore the two treatment | LRRFS defined as time from date of | |
| arms with respect to loco-regional | randomization to date of first event of | |
| recurrence-free survival (LRRFS) | local invasive breast recurrence, regional | |
| | invasive recurrence or death due to any | |
| | cause | |
| 2. To explore use of subsequent | Incidence of subsequent anti-neoplastic | |
| anti-neoplastic therapy | therapy and time to first subsequent anti- | |
| | neoplastic therapy | |
| 3. To explore healthcare resource | Number of patients hospitalized, total | |
| utilization | number of hospitalizations, and length of | |
| | stay in hospitals, number of patients with | |
| | Emergency Room and additional visits | |
| 4. To explore prognostic and | Assessment of expression and | |
| predictive biomarkers of treatment | alterations of genes related but not | |
| with ribociclib and ET | limited to CDK and ER pathways in | |
| | baseline tumor and circulating tumor | |
| | (ct)DNA and ctRNA samples, and their | |
| | correlation with efficacy endpoints. | |
| | Expression of markers such as Ki67 by | |
| | immunochemistry may be evaluated | |
| 5. To explore potential molecular | Assessment of gene expression and | |
| mechanisms of resistance to | alterations in tumor biopsy and | |
| treatment with ribociclib and ET | ctDNA/ctRNA collected at baseline and | |
| | at time of recurrence | |
| 6. To explore the role of | Assessment of gene expression and | |
| ctDNA/ctRNA for their suitability to | alterations in ctDNA/ctRNA collected | |
| monitor and predict disease | serially at baseline, on treatment, post | |
| recurrence | treatment and at time of recurrence | |

### 3. TRIAL DESIGN

### 3.1. Overall Trial Design

This is a phase III, multicenter, randomized, open-label trial to evaluate efficacy and safety of ribociclib with ET as an adjuvant treatment in women and men with HR-positive, HER2-negative EBC. iDFS is the primary endpoint of the trial, as defined per the STEEP System.³⁵ The trial will include pre and postmenopausal women and men with HR-positive, HER2-negative EBC, with an AJCC 8^{th} edition Anatomic Stage Group III, IIB or a subset of Stage IIA cases (as defined in Inclusion Criterion #8), after adequate surgical resection, radiotherapy (if indicated), adjuvant or neoadjuvant chemotherapy (if indicated), and who are deemed to be eligible for adjuvant ET for at least 60 months from the date of randomization. Patients who previously started any standard neoadjuvant/adjuvant ET are included in the trial provided that the ET was started within 12 months prior to randomization.

The trial will include the following phases:
Screening Phase
Treatment Phase
Follow-up Phase

Refer to 1 for the trial design schema and to Section 6.3 for a detailed description of the trial phases.

Approximately 5,000 patients will be randomized at a 1:1 ratio (through an Interactive Response

Technology system [IRT]) using the following stratification factors:
Menopausal status: premenopausal women and men vs. postmenopausal women
AJCC 8^{th} edition Anatomic Stage Group: Anatomic Stage Group II vs. Anatomic Stage Group III
Prior neoadjuvant/adjuvant chemotherapy: yes vs. no
Geographical region: North America/Western Europe/Oceania vs. rest of the world (RoW) Enrollment of patients with Anatomic Stage Group II is capped at approximately 2,000 patients.

Randomized patients will receive, starting from Cycle 1 Day 1 (C1D1) either:
Investigational arm:
   Ribociclib 400 mg by mouth once daily on days 1 to 21 of a 28-day cycle, for 36 months since the randomization date (approximately 39 cycles).
   and
   ET consisting of:
      For postmenopausal women: letrozole 2.5 mg by mouth once daily continuously or anastrozole 1 mg by mouth once daily continuously.
      For premenopausal women and men: letrozole 2.5 mg by mouth once daily continuously or anastrozole 1 mg by mouth once daily continuously, combined with goserelin 3.6 mg subcutaneously once every 4 weeks.
   Duration of ET in the trial will be 60 months from the randomization date.
Control arm:
   ET: Same as in the Investigational arm.

In both arms, ET will be administered according to the local clinical guidelines and current local prescribing information. Subsequent ET (or any other anti-cancer treatment) given after the protocol-required 60 months of ET (or after premature discontinuation of ET in the trial) will be administered according to the Investigator's clinical judgment and is not considered a trial treatment.

Safety will be assessed for each patient (see Sections 6.4.2 and 7.1) and will include routine safety monitoring. Assessments for events of recurrence (see Appendix 3: Recurrence Detection Guidelines) will be done every 12 weeks for the first 24 months from randomization and every 24 weeks thereafter until confirmed distant recurrence, death, withdrawal of consent, lost to follow-up, or end of trial, whichever is earliest (see Table A12).

Patients who discontinue all trial treatment will enter a Follow-up Phase in which recurrence and survival will be recorded. Survival status will be assessed up to 60 months after the last patient has been randomized, until death, withdrawal of consent, loss to follow-up or end of trial, whichever is earliest.

Three interim iDFS analyses and a final analysis are planned (see Section 8). See FIG. 1 for the trial design.

### 3.2. Number of Patients and Investigational Sites

Approximately 5,000 patients will be randomized in the trial, from approximately 425 sites globally.

### 3.3. End of Trial

If the primary endpoint, iDFS, is statistically significant at the second, third interim or at final analysis, data collection will continue and end of trial will be declared when 60 months+30 days have elapsed from the date the last patient has been randomized.

If the primary endpoint, iDFS, is futile at the first interim analysis, or iDFS is not statistically significant at the final iDFS analysis, end of trial will be declared after all patients have discontinued trial treatment and completed the 30-Day Safety Follow-up visit. Refer to Section 8.7 for details about the interim and final iDFS analyses.

The end of trial participation for a given patient is reached at time of (whichever occurs first):
End of trial
Withdrawal of consent by the patient
Patient is lost to follow-up
Death
Early trial termination at a country or site level (see Section 3.3.1)
Early trial termination by Novartis (see Section 3.3.1).

### 3.3.1. Early Trial Termination

The trial can be terminated at any time for any reason by Novartis. Should this be decided, the Investigator will be informed of the procedures to be followed. Institutional Review Board/Independent Ethics Committee (IRB/IEC) and Competent Authority (CA) will be informed of the early termination of the trial according to applicable regulations.

In addition, CA decision may prematurely terminate the trial within a country, or a change in opinion of the IRB/IEC may lead to its termination at the local level.

### 4. PATIENT SELECTION

This trial can fulfill its objectives only if appropriate patients are enrolled. Patients who fail to meet all inclusion criteria or fulfill at least one exclusion criterion must not be randomized in the trial. Novartis or TRIO will not grant any eligibility waivers.

See Section 6.1 for additional information on patient screening and randomization.

### 4.1. Inclusion Criteria

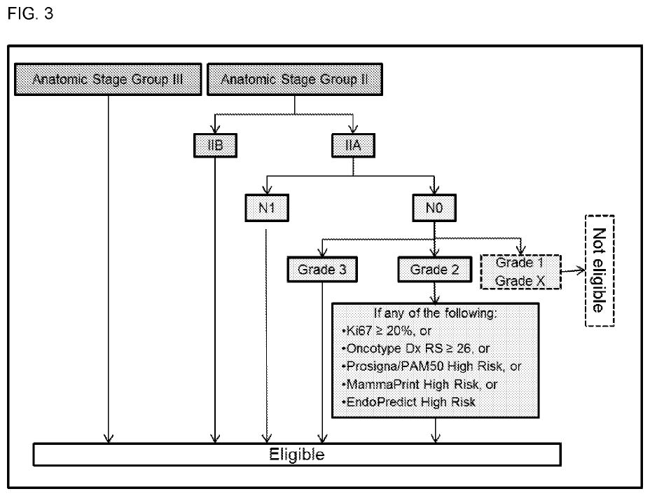

Patients eligible for inclusion in this trial must meet all of the following criteria:
1. Signed and dated Patient Informed Consent Form (PICF) obtained prior to any trial-specific screening procedure.
2. Patient is ≥18 years-old at the time of PICF signature.
3. Patient is female with known menopausal status at the time of randomization or initiation of adjuvant ET (whichever occurs earlier), or male.
   Postmenopausal status is defined as:
      Patient underwent bilateral oophorectomy, or
      Age ≥60 years, or
      Age <60 years and amenorrhea for 12 or more months (in the absence of chemotherapy, tamoxifen, toremifene or ovarian suppression) and Follicle-stimulating hormone (FSH) and plasma estradiol are in the postmenopausal ranges per local normal ranges.
      If taking tamoxifen or toremifene and age <60 years, then FSH and plasma estradiol level in postmenopausal ranges.
   Notes:
      In women who are premenopausal at the beginning of adjuvant chemotherapy, amenorrhea is not a reliable indicator of menopausal status as ovarian function may still be intact or resume despite anovulation/amenorrhea. For these women with therapy-induced amenorrhea, serial measurements of FSH and/or estradiol per local clinical guidelines are required for determination of postmenopausal status.
      All women who do not meet the criteria for postmenopausal status are considered premenopausal for the purpose of this trial.
4. Patient with histologically confirmed unilateral primary invasive adenocarcinoma of the breast with a date of initial cytologic or histologic diagnosis (i.e. date of the pathology report that confirmed the BC diagnosis) within 18 months prior to randomization. Patient with a multicentric and/or multifocal tumor is eligible if all the histopathologically examined lesions meet the pathologic criteria in inclusion criteria 5 and 6.
5. Patient has breast cancer that is positive for ER and/or PgR according to the local laboratory as determined on the most recently analyzed tissue sample.
6. Patient has HER2-negative breast cancer defined as a negative in situ hybridization test or an immunohistochemistry (IHC) status of 0 or 1+. If IHC is 2+, a negative in situ hybridization (FISH, CISH, or SISH) test is required to confirm the HER2-negative status (based on the most recently analyzed tissue sample tested by a local laboratory).
7. Patient (except those enrolled in China) has available archival tumor tissue from the surgical specimen, for submission to a central laboratory (Note: in patients that underwent neoadjuvant systemic therapy and had a pathologic complete response, archival tumor tissue at the time of the initial diagnosis or before the administration of neoadjuvant therapy is mandatory).
8. Patient after surgical resection where tumor was removed completely, with the final surgical specimen microscopic margins free from tumor, and belongs to one of the following categories (see FIG. 3):
   Anatomic Stage Group III, or
   Anatomic Stage Group IIB, or
   Anatomic Stage Group IIA that is either:
      N1, or
      N0. with:
         Grade 3, or
         Grade 2, with any of the following criteria:
         Ki67 ≥20%, or
         Oncotype DX Breast Recurrence Score 26, or
         Prosigna/PAM50 categorized as high risk, or
         MammaPrint categorized as high risk, or
         EndoPredict EPclin Risk Score categorized as high risk.
Notes:
   For patients whose tumors are Anatomic Stage IIA, N0:
      If Grade is 1 or unknown (Gx), patient is not eligible.
      If Grade 2, the gene expression test results (Oncotype DX, Prosigna/PAM50, MammaPrint or EndoPredict EPclin) or Ki67 levels should be used if obtained as per local practice (i.e. are not mandatory for the purpose of the trial). Results must be available at screening.
   Patients that received neoadjuvant treatment must meet the above criteria (for stage, and if Stage IIA, N0, also for grade and Ki67 or gene expression test), in any presurgical staging/sample and/or in the surgical specimen.
   Categorization into the AJCC 8^{th} edition Anatomic Stage Groups (see Appendix 1: Guidelines for Anatomic Stage Groups for Breast Cancer Staging) requires determination of the T, N and M categories. ALND is the preferred method for axillary lymph node staging, however SLN dissection can be used to determine the N category in the following cases:
      No metastasis in SLN (patient is considered as pN0).
      Only micrometastasis in SLN (patient is considered as pN1mi).
      Patients with T1-2 and no clinically-evident nodes prior to surgery, no neoadjuvant chemotherapy, at least one macrometastasis in 1 or 2 SLNs, no matted nodes or gross extranodal disease at the time of SLN dissection (patient is considered as pN1).

In all other cases, ALND is required to determine the N category.
9. If indicated, patient has completed adjuvant and/or neoadjuvant chemotherapy according to the institutional guidelines, prior to screening.
10. If indicated, patient has completed adjuvant radiotherapy according to the institutional guidelines, prior to screening.
11. Patient has no contraindication for the adjuvant ET in the trial and is planned to be treated with ET for 5 years (since randomization date) or more.
12. Patient may have already received any standard neoadjuvant and/or adjuvant ET at the time of PICF signature, but randomization should occur within 12 months of the initial start date of ET. Ovarian suppression or short term ET for fertility preservation is not considered neoadjuvant/adjuvant ET. If patient was receiving tamoxifen or toremifene as adjuvant ET, a washout period of 5 half-lives (i.e. 35 days) prior to randomization is required (during that period patient can take AI).
13. Patient has an Eastern Cooperative Oncology Group (ECOG) Performance Status of 0 or 1.
14. Patient has adequate bone marrow and organ function as defined by the following local laboratory values:
   Absolute neutrophil count (ANC) ≥1.5×10⁹/L.
   Platelets ≥100×10⁹/L.
   Hemoglobin ≥9.0 g/dL.
   Estimated glomerular filtration rate (eGFR) ≥30 mL/min/1.73 m² according to the Modification of Diet in Renal Disease (MDRD) formula.
   Alanine transaminase (ALT)<2.5× Upper Limit Normal (ULN).
   Aspartate transaminase (AST)<2.5×ULN.
   Total serum bilirubin <ULN; or total bilirubin ≤3.0×ULN or direct bilirubin ≤1.5×ULN in patients with well documented Gilbert's Syndrome.
   International normalized ratio (INR) ≤1.5 (unless the patient is receiving anticoagulants and the INR is within the therapeutic range of intended use for that anticoagulant within 7 days prior to randomization).
   Patient must have the following laboratory values within normal limits or corrected to within normal limits with supplements (the local laboratory value should be documented within normal limits after the correction) before randomization:
      Potassium
      Magnesium
      Total Calcium (corrected for serum albumin)
15. Standard 12-lead ECG values assessed by a central laboratory, as:
   QTcF interval (QT interval using Fridericia's correction) at screening <450 msec.
   Resting heart rate 50-90 beats per minute (determined from the ECG).
16. Patient must be willing and able to comply with scheduled visits, treatment plans, laboratory tests, and other trial procedures.
17. Women of childbearing potential (CBP), defined as all women physiologically capable of becoming pregnant (see Inclusion Criterion #18 for additional information), must have confirmed negative serum pregnancy test (for β-hCG) within 14 days prior to randomization.
18. Women of CBP must be willing to use highly effective methods of contraception.
   Contraception must continue during the trial treatment and for 21 days after stopping the treatment. Highly effective contraception methods include:
      Total abstinence (when this is in line with the preferred and usual lifestyle of the patient). Periodic abstinence (e.g. calendar, ovulation, symptothermal, post-ovulation methods) and withdrawal are not acceptable methods of contraception.
      Female sterilization (have had surgical bilateral oophorectomy with or without hysterectomy), total hysterectomy or tubal ligation at least 6 weeks before taking trial treatment. In case of oophorectomy alone, only when the reproductive status of the woman has been confirmed by follow up hormone level assessment.
      Male partner sterilization (at least 6 months prior to randomization). For female patients on the trial the vasectomized male partner should be the sole partner for that patient. If vasectomy of the male partner is the highly effective method of contraception chosen, the success of the vasectomy should be medically confirmed according to local practice.
      Placement of an intrauterine device (IUD).
   Notes:
      Use of oral (estrogen and progesterone), transdermal, injected, implanted, hormone containing intrauterine system, or any other hormonal methods of contraception is not allowed in this trial.
      Women are considered of CBP unless: they have had ≥12 months of natural (spontaneous) amenorrhea with an appropriate clinical profile (i.e. age appropriate, history of vasomotor symptoms) or have had surgical bilateral oophorectomy (with or without hysterectomy), total hysterectomy, or tubal ligation at least six weeks prior to randomization. In the case of oophorectomy alone, only when the reproductive status of the woman has been confirmed by follow-up hormone level assessment she is considered not of CBP.
      After the end of trial treatment, patients should use effective contraception according to local guidelines.

See FIG. 3 which provides a schematic view of the inclusion according to anatomic stage group.

### 4.2. Exclusion Criteria

Patients eligible for this trial must not meet any of the following criteria:
1. Patient has received any CDK4/6 inhibitor.
2. Patient has received prior treatment with tamoxifen, raloxifene or Als for reduction in risk (“chemoprevention”) of breast cancer and/or treatment for osteoporosis within the last 2 years prior to randomization. Patient is concurrently using hormone replacement therapy.
3. Patient has received prior treatment with anthracyclines at cumulative doses of 450 mg/m2 or more for doxorubicin, or 900 mg/m² or more for epirubicin.
4. Patient with a known hypersensitivity to any of the excipients of ribociclib and/or ET (e.g. rare hereditary problems of galactose intolerance, the Lapp lactase deficiency, glucose-galactose malabsorption, and soy allergy).
5. Patient with distant metastases of breast cancer beyond regional lymph nodes (stage IV according to AJCC 8^{th} edition) and/or evidence of recurrence after curative surgery.
6. Patient is concurrently using other anti-neoplastic therapy with the exception of adjuvant ET (see Inclusion Criterion #12).
7. Patient has had major surgery, chemotherapy or radiotherapy within 14 days prior to randomization.
8. Patient has not recovered from clinical and laboratory acute toxicities related to prior anti-cancer therapies to a NCI CTCAE (National Cancer Institute Common Terminology Criteria for Adverse Events) version 4.03 Grade ≤1 at day of randomization. Exceptions to this criterion: patients with any grade of alopecia, amenorrhea, grade 2 neuropathy, or other toxicities not considered a safety risk for the patient as per Investigator's discretion, are allowed to enter the trial.
9. Patient has a concurrent invasive malignancy or a prior invasive malignancy whose treatment was completed within 2 years before randomization. Note: patients with adequately treated, basal or squamous cell skin carcinoma or curatively resected cervical cancer in situ are eligible.
10. Patient has known history of human immunodeficiency virus (HIV) infection (testing is not mandatory, unless required by local regulation).
11. Patient has known active hepatitis B virus (HBV) or hepatitis C virus (HCV) infection (testing is not mandatory, unless required by local regulation).
12. Clinically significant, uncontrolled heart disease and/or cardiac repolarization abnormality, including any of the following:
   History of documented myocardial infarction (MI), angina pectoris, symptomatic pericarditis, or coronary artery bypass graft within 6 months prior to trial entry.
   Documented cardiomyopathy.
   Left Ventricular Ejection Fraction (LVEF)<50% as determined by Multiple Gated acquisition (MUGA) scan or echocardiogram (ECHO) (testing not mandatory).
   Long QT syndrome or family history of idiopathic sudden death or congenital long QT syndrome, or any of the following:
      Risk factors for Torsades de Pointes (TdP) including uncorrected hypocalcemia, hypokalemia or hypomagnesemia, history of cardiac failure, or history of clinically significant/symptomatic bradycardia.
      Concomitant medication(s) with a known risk to prolong the QT interval and/or known to cause TdP that cannot be discontinued or replaced by safe alternative medication (e.g. within 5 half-lives or 7 days prior to starting trial treatment).
      Inability to determine the QTcF interval.
   Clinically significant cardiac arrhythmias (e.g. ventricular tachycardia), complete left bundle branch block, high-grade Atrioventricular (AV) block (e.g. bifascicular block, Mobitz type II and third degree AV block).
   Uncontrolled arterial hypertension with systolic blood pressure >160 mmHg.
13. Patient is currently receiving any of the following substances within 7 days before randomization:
   Concomitant medications, herbal supplements, and/or fruits (e.g. grapefruit, pummellos, starfruit, Seville oranges) and their juices that are known as strong inhibitors or inducers of CYP3A4/5.
   Medications that have a narrow therapeutic window and are predominantly metabolized through CYP3A4/5.
14. Patient is currently receiving or has received systemic corticosteroids s 2 weeks prior to starting trial treatment, or has not fully recovered from side effects of such treatment. Note: The following uses of corticosteroids are permitted: a short duration (<5 days) of systemic corticosteroids; any duration of topical applications (e.g. for rash), inhaled sprays (e.g. for obstructive airways diseases), eye drops or local injections (e.g. intra-articular).
15. Patient has impairment of GI function or GI disease that may significantly alter the absorption of the oral trial treatments (e.g. uncontrolled ulcerative diseases, uncontrolled nausea, vomiting or diarrhea, malabsorption syndrome, or small bowel resection).
16. Patient has any other concurrent severe and/or uncontrolled medical condition that would, in the Investigator's judgment, cause unacceptable safety risks, contraindicate patient participation in the clinical trial or compromise compliance with the protocol (e.g. chronic pancreatitis, chronic active hepatitis, liver cirrhosis or any other significant liver disease, active untreated or uncontrolled fungal, bacterial or viral infections, active infection requiring systemic anti-bacterial therapy, etc.) or limit life expectancy to ≤5 years.
17. Participation in other studies involving investigational drug(s) within 30 days prior to randomization or within 5 half-lives of the investigational drug(s) (whichever is longer), or participation in any other type of medical research judged not to be scientifically or medically compatible with this trial. If the patient is enrolled or planned to be enrolled in another study that does not involve an investigational drug, the agreement of the Medical Monitor is required to establish eligibility.
18. Pregnant or breast-feeding (lactating) women or women who plan to become pregnant or breast-feed during the trial.

### 5. TREATMENTS

### 5.1. Trial Treatment

For this trial, the term “investigational drug” refers to Novartis drug ribociclib (LEE011); ribociclib is considered an Investigational Medicinal Product (IMP) and will be supplied by Novartis.

The other drugs to be used in this trial are NSAI and goserelin. Whether these ET are considered IMP or Non-IMP is based on regional or country level authorities. In these circumstances, local legislation is followed for the conduct of this trial. ET will be procured locally according to local practice and regulation, or supplied by Novartis (or its designee). ET will be administered according to the current local prescribing information and clinical guidelines.

“Trial treatment” refers to either the combination of ET with ribociclib in the Investigational arm, or to ET alone in the Control arm (given for 60 months since randomization), or to ET (given for 60 months since randomization) in the Investigational arm after discontinuation of ribociclib.

Subsequent ET (or any other anti-cancer treatment) given after the protocol-required 60 months duration (or after premature discontinuation of ET in the trial) will be administered according to the Investigator's clinical judgment and is not considered a trial treatment.

The Reference Safety Information for ribociclib is included in the “Reference Safety Information” section of the ribociclib IB.

### 5.1.1. Schedule and Administration

Randomized patients will receive, starting from C1 D1, either:
Investigational arm:
   Ribociclib 400 mg (2×200 mg tablets by mouth) once daily on days 1 to 21 of a 28-day cycle, followed by 7 days off ribociclib (Days 22 to 28).
And
   ET consisting of:
      For postmenopausal women:
         Letrozole 2.5 mg by mouth daily continuously or anastrozole 1 mg by mouth daily continuously.
      For premenopausal women and men:
         Letrozole 2.5 mg by mouth daily continuously or anastrozole 1 mg by mouth daily continuously, combined with:
         Goserelin 3.6 mg subcutaneously once every 4 weeks.
Control arm:
   ET (same as in the Investigational arm) consisting of:
      For postmenopausal women:
         Letrozole 2.5 mg by mouth daily continuously or anastrozole 1 mg by mouth daily continuously.
      For premenopausal women and men:
         Letrozole 2.5 mg by mouth daily continuously or anastrozole 1 mg by mouth daily continuously, combined with:
         Goserelin 3.6 mg subcutaneously once every 4 weeks.

The trial treatment should be administered as described in this Section and in Section 5.1.2. Randomization and C1D1 should preferably occur on the same day. A window of up to 7 days between randomization and C1D1 is permitted.

C1D1 is defined as the day when the patient takes the first dose of trial treatment after randomization (note: for patients who were receiving ET prior to randomization, C1D1 is considered as the day they receive the first dose of ribociclib [Investigational arm] or the first dose of ET after randomization [Control arm]).

A complete cycle of treatment is defined as 28 days. Cycles must occur as per a schedule calculated considering C1D1 as the starting point. A flexibility window of ±3 day is allowed for the trial assessments (except for ECG when it is required on C1 D1), to accommodate any scheduling issue, but treatment schedule remains fixed based on the calculation considering C1D1 as the starting point. Since cycles are fixed, if for any reason ribociclib begins later than D1 of a cycle, intake should nevertheless stop at D21 and then patient should proceed to the 7 days off period.

If ribociclib dosing is interrupted for >28 days due to ribociclib-related toxicity, it must be permanently discontinued. Details and examples are available in the CRF Completion Guidelines.

TABLE A5
Dose and Treatment Schedule

| | Pharmaceutical form and | | | |
|---|---|---|---|---|
| Trial Treatment | route of administration | Dose | | Frequency and/or Regimen |
| Ribociclib | Tablets for oral use | 400 | mg | Once daily on days 1 to 21 in each |
| (LEE011 200 mg) | | | | 28-day cycle |
| Letrozole 2.5 mg | Tablets for oral use | 2.5 | mg | Once daily given continuously |
| Anastrozole 1 mg | Tablets for oral use | 1 | mg | Once daily given continuously |
| Goserelin 3.6 mg | Implant, in pre-filled | 3.6 | mg | Day 1 ± 3 of each 28-day cycle |
| | syringe and Subcutaneous | | | |
| | use | | | |

Ribociclib will be administered as a flat-fixed dose, and not by body weight or body surface area.

### 5.1.1.1. Guidelines for Discontinuation of Treatment

In the Investigational arm, treatment with ribociclib will continue until (whichever is earlier):
Completion of 36 months of treatment from the randomization date (approximately 39 cycles), regardless of any treatment interruption.
First recurrence (any of the following or combination of local, regional or distant recurrences, or contralateral invasive BC, or second primary non-breast invasive cancer).
Adjustments to trial treatment due to toxicity that result in treatment discontinuation (see Section 5.2.1.2).
Ribociclib dosing was interrupted for >28 days due to ribociclib-related toxicity.
Withdrawal of consent by the patient.
Patient is lost to follow-up.
Death.
Discontinuation from the trial treatment due to any other reason.
Novartis terminates the trial.

In both arms, ET will be given as a trial treatment until (whichever is earlier):
Completion of 60 months since the randomization date.
Local, regional and/or distant recurrence (continuation of ET after contralateral invasive BC, or second primary non-breast invasive cancer is left to the Investigator's best clinical judgment).
Intolerable toxicity.
Withdrawal of consent by the patient.
Patient is lost to follow-up.
Death.
Discontinuation from the trial treatment due to any other reason.
Novartis terminates the trial.

Patients may voluntarily discontinue from the trial treatment for any reason at any time. If a patient decides to discontinue from the trial treatment, the Investigator must make a reasonable effort (e.g. telephone, e-mail, letter) to understand the primary reason for this decision and record this information in the patient's chart and on the appropriate page of the Case Report Form (CRF). They will be considered withdrawn if they state an intention to withdraw consent from trial participation.

The Investigator should discontinue trial treatment for a given patient if he/she believes that continuation would be detrimental to the patient's well-being. Trial treatment must also be discontinued under the following circumstances:
Pregnancy.
Lactation.
Any protocol deviation that results in a significant risk to the patient's safety per Sponsor's judgment.

In case of permanent discontinuation of any of the trial treatments, guidance below should be followed (see also Sections 6.3.2 and 6.3.3):
In the Investigational arm:
   Patients who permanently discontinue ribociclib for any reason will proceed to the 30-Day Post Ribociclib Safety Follow-up visit and will continue on ET within the Treatment Phase of the trial. Once all trial treatments are discontinued, patients will proceed to the End of Treatment (EOT) and the 30-Day Safety Follow-up visits and will remain on the trial for the Follow-up Phase.
   Patients who permanently discontinue ET while receiving ribociclib, must discontinue ribociclib at the same time and will proceed to the EOT and the 30-Day Safety Follow-up visits (see Section 6.3.2.2). They will remain on the trial for the Follow-up Phase.
In the Control arm:
   Patients who permanently discontinue ET for any reason will proceed to the EOT and the 30-Day Safety Follow-up visits, and will remain on the trial for the Follow-up Phase.

### 5.1.2. General Dosing Guidelines

### 5.1.2.1. Ribociclib

Ribociclib should be taken as follows:
Ribociclib is dosed for the first 21 days out of the 28-day cycle. Patients should not resume a new cycle of ribociclib until completion of the 7-day off period.
When visits take place on the scheduled D1 of a cycle (or in the allowed+3 days window) patients must take ribociclib (and ET) in the clinic under the supervision of the Investigator or designee. On all other days patients may take ribociclib (and ET) at home.
Patients should be instructed to take ribociclib and ET together, with a large glass of water (~250 mL or ~8 oz.) at the same time each day. Patients can determine if they prefer morning or early afternoon dosing, but they should maintain a consistent time. Evening doses are strongly not recommended.
Note: for the first cycle, patients in the PK subset (see Section 6.4.3) must take ribociclib and ET in the morning due to PK assessments on C1D15. Afterwards patients can determine if they prefer morning or early afternoon dosing, but should maintain a consistent time.
With or without food.
Patients should be instructed to swallow the tablets whole and not to chew or crush them.
If vomiting occurs during the course of treatment, no re-dosing of the patient is allowed before the next scheduled dose. The occurrence and frequency of any vomiting during a treatment cycle must be noted in the AE section of the CRF. Refer to Section 5.4.3.5.3 for use of antiemetic medications.
Any doses that are missed (not taken within 6 hours of the intended time) should be skipped and should not be replaced or made up on a subsequent day.
Patients must avoid consumption of grapefruit, grapefruit hybrids, pummelos, star fruit, Seville oranges, or products containing the juice of each, during treatment with ribociclib and before its first dose, according to Exclusion Criterion #13. These foods are known as CYP3A4 inhibitors and have a potential to increase exposure to ribociclib (note: oranges and orange juice are allowed).
Herbal or dietary supplements known as strong inhibitors or inducers of CYP3A4/5 or those with a known risk of QT prolongation are not permitted (see Section 5.4.3.1). Multivitamins are permitted.
Patients should be instructed not to take any medication reported as prohibited in Section 5.4 (see also Appendix 4: Concomitant Medications).

### 5.1.2.1.1. Additional Dosing Guidelines for Pharmacokinetic Sampling and ECG Collection

On C1D15 (for patients in the PK subset only; see Section 6.4.3) and/or days of ECG collection (for all patients), the following additional guidelines should be followed:
Pre-dose PK samples should be drawn prior to trial treatment dosing.
Post-dose PK samples should be collected after trial treatment dosing.
If a pre-dose ECG measurement should be collected, then the ECG measurement should occur before dosing of the trial treatment.
If a pre-dose PK sample should also be obtained on the same day as ECG, then the sample should be collected after the ECG and before dosing of the trial treatment (see Section 6.4.2.6 for additional details).
The following will be recorded in the CRF: exact time of ribociclib dosing on the PK collection day, actual time of PK blood sampling, date and time ribociclib was taken on the day before the PK assessment. Any sampling problems (e.g. patient took trial drug before blood sample, scheduled sampling time is missed, sample is not drawn according to the schedule) should be noted as a comment on the CRF.

### 5.1.2.2. Endocrine Therapy

Protocol allowed ET (in both arms) will be administered according to the local clinical guidelines and current local prescribing information.

On scheduled visit days, patients must take ET (and ribociclib in the Investigational arm) in the clinic under the supervision of the Investigator or designee. If a pre-dose ECG measurement is to be collected, then the ECG measurement should occur before dosing the trial treatment (including ET).

Goserelin will be administered subcutaneously on D1±3 of each 28-day cycle, in accordance with the current local prescribing information (Note: 3-month depot formulation of goserelin is not allowed in the trial). In patients receiving monthly goserelin (or other monthly GnRH agonist) prior to randomization, it is acceptable to continue with their treatment schedule unchanged (i.e. without synchronizing with the cycles in the trial) or to synchronize the administration of goserelin with the cycles. Patients receiving 3-monthly goserelin prior to randomization must switch to the monthly formulation. It is recommended that women receiving goserelin are regularly monitored per local institutional clinical guidelines to confirm a postmenopausal value of gonadotropin and sex hormones according to local laboratory.

In the investigational arm, during treatment with ribociclib, Investigators will not be allowed to change NSAI unless intolerable toxicity, patient's request, or any other medically-important event that requires change of NSAI (Investigator should clearly document in the CRF the reason for changing of ET during the treatment with ribociclib). After end of treatment with ribociclib, Investigators will be allowed to change NSAI according to the clinical guidelines and local prescription information (Investigator must document in the CRF the reason for changing ET).

ETs other than NSAI and goserelin are not allowed during the trial treatment phase.

### 5.2. Dose Adjustments and Adverse Event Management

### 5.2.1. Ribociclib

### 5.2.1.1. Dose Levels

For patients who do not tolerate the protocol-specified dosing schedule, one dose adjustment is permitted in order to allow patients to continue ribociclib (see Table A6). These changes must be recorded on the CRF. If a second dose reduction is required to manage a ribociclib-related AE, then ribociclib must be discontinued. No dose re-escalation is permitted.

TABLE A6
Dose Modification Guidelines

| | Ribociclib | |
|---|---|---|
| | Dose | Number of tablets & strength |
| Starting dose | 400 mg | 2 × 200 mg tablets |
| Dose level -1 | 200 mg | 1 × 200 mg tablet |

### 5.2.1.2. Ribociclib Dose Adjustments

Recommendations for dose interruption, reduction of ribociclib in the management of ribociclib related AEs are summarized in Table A7, Table A8, Table A9, Table A10 and Table A11.

Clinical judgment of the treating Investigator should guide the management plan of each patient based on individual benefit/risk assessment.

Unscheduled laboratory assessments may be performed if medically indicated to document a (potential) AE for decision making (e.g. dose adjustments).

Ribociclib must be discontinued in case of:
Events requiring a discontinuation according to Table A7, Table A8, Table A9, Table A10 and Table A11.
Dosing was interrupted for >28 days due to ribociclib-related toxicity.

### 5.2.1.2.1. Dose Adjustments for Hematological and Hepatic Toxicities

TABLE A7
Ribociclib Dose Adjustment, Management Recommendations and Mandatory
Discontinuation for Hematological Adverse Reactions (CTCAE v4.03)

| | |
|---|---|
| Toxicity/Grade | Dose Adjustment and Management Recommendations |

Thrombocytopenia

| | |
|---|---|
| Grade 1 (≥75 × 10⁹/L) | No dose adjustment required. |
| Grade 2 (≥50 × 10⁹/L − <75 × 10⁹/L) | Dose interruption until recovery to grade ≤1. |
| | Re-initiate ribociclib at the same dose level. |
| Grade 3 (≥25 × 10⁹/L − <50 × 10⁹/L) | Dose interruption until recovery to grade ≤1. |
| | Re-initiate ribociclib at the same dose level. |
| | If toxicity recurs at grade 3: temporary dose interruption |
| | until recovery to grade ≤1 and reduce ribociclib to the next |
| | lower dose level. |
| Grade 4 (<25 × 10⁹/L) | Dose interruption until recovery to grade ≤1. |
| | Re-initiate ribociclib at the next lower dose level. |
| | If toxicity recurs at grade 4: discontinue ribociclib |
| | (mandatory). |

Decreased Absolute Neutrophil Count (ANC)

| | |
|---|---|
| Grade 1 (≥1.5 × 10⁹/L) | No dose adjustment required. |
| Grade 2 (≥1.0 − <1.5 × 10⁹/L) | No dose adjustment required. |
| Grade 3 (≥0.5 − <1.0 × 10⁹/L) | Dose interruption until recovery to ≥1.0 × 10⁹/L. |
| | Re-initiate ribociclib at the same dose level. |
| | If toxicity recurs at grade 3: temporary dose interruption |
| | until recovery to ≥1.0 × 10⁹/L. |
| | If resolved in ≤7 days, then re-initiate at the same |
| | dose level. |
| | If resolved in >7 days, then re-initiate ribociclib at |
| | the next lower dose level. |
| Grade 4 (<0.5 × 10⁹/L) | Dose interruption until recovery to ≥1.0 × 10⁹/L. |
| | Re-initiate ribociclib at the next lower dose level. |

Febrile neutropenia

| | |
|---|---|
| Grade 3 | Dose interruption until ANC ≥ 1.0 × 10⁹/L and no fever. Re- |
| ANC < 1.0 × 10⁹/L with a single temperature | initiate at the next lower dose level. |
| of >38.3° C. (101° F.) or a sustained temperature | If febrile neutropenia recurs, discontinue ribociclib |
| of ≥38° C. (100.4° F.) for more than one hour | (mandatory). |
| Grade 4 | Discontinue ribociclib (mandatory). |
| Life-threatening consequences; | |
| urgent intervention indicated | |

Anemia (Hemoglobin)

| | |
|---|---|
| Grade 1 (≥10.0 g/dL) | No dose adjustment required. |
| Grade 2 (≥8.0 − <10.0 g/dL) | No dose adjustment required. |
| Grade 3 (<8.0 g/dL, transfusion indicated) | Dose interruption until recovery to grade ≤2. |
| | Re-initiate ribociclib at the same dose level. |
| Grade 4 | Discontinue ribociclib (mandatory). |
| Life-threatening consequences; | |
| urgent intervention indicated | |

TABLE A8
Ribociclib Dose Adjustment, Management Recommendation and Mandatory
Discontinuation for Hepatic Toxicities (CTCAE v4.03)
HEPATOTOXICITY (BILIRUBIN, SGPT/ALT, SGOT/AST)
TOTAL BILIRUBIN without ALT/AST increase above baseline value

| | |
|---|---|
| Grade 1 (>ULN − 1.5 × ULN) | Maintain dose level with LFTs monitored every two |
| (confirmed 48-72 h later) | weeks. |
| Grade 2 (>1.5 − 3.0 × ULN) | Dose interruption of ribociclib. |
| | If resolved to ≤ grade 1 in ≤21 days, then re-initiate at |
| | the same dose level. |
| | If resolved to ≤ grade 1 in >21-28 days or toxicity recurs, |
| | then re-initiate at the next lower dose level. |
| | Repeat liver enzyme and bilirubin tests twice weekly for 2 |
| | weeks after dose resumption. |
| | If toxicity recurs after a dose reduction, or recovery to ≤ |
| | grade 1 is >28 days, discontinue ribociclib (mandatory). |
| Grade 3 (>3.0 −− 10.0 × | Dose interruption of ribociclib, until resolved to ≤ grade 1, |
| ULN) | then re-initiate at the next lower dose level. |
| | Repeat liver enzyme and bilirubin tests twice weekly for 2 |
| | weeks after dose resumption. |
| | If resolved to ≤ grade 1 in >28 days or toxicity recurs, |
| | discontinue ribociclib (mandatory). |
| Grade 4 (>10.0 × ULN) | Discontinue ribociclib (mandatory). |

Elevated AST or ALT
AST or ALT without bilirubin elevation >2 × ULN

| | |
|---|---|
| Same grade as baseline or increase from baseline | No dose adjustment required, with Liver Function Tests |
| grade 0 to grade 1 (confirmed 48-72 h later) | (LFT) monitored per protocol if same grade as baseline |
| | or every two weeks in case of increase from baseline |
| | grade 0 to 1. |
| Grade 2 (>3.0 − 5.0 × ULN) | Dose interruption of ribociclib. |
| | If resolved to ≤ baseline grade in ≤21 days, then re- |
| | initiate at the same dose level. |
| | If resolved to ≤ baseline grade in >21 days or toxicity |
| | recurs, then re-initiate at the next lower dose level. |
| | Repeat liver enzyme and bilirubin tests twice weekly for 2 |
| | weeks after dose resumption. |
| | If toxicity recurs after a dose reduction or recovery to ≤ |
| | baseline grade is >28 days, discontinue ribociclib |
| | (mandatory). |
| Grade 3 (>5.0 − 20.0 × ULN) | Dose interruption of ribociclib until resolved to ≤ baseline |
| | grade, re-initiate at the next lower dose level. |
| | Repeat liver enzyme and bilirubin tests twice weekly for 2 |
| | weeks after dose resumption. |
| | If recovery to ≤ baseline grade is >28 days, discontinue |
| | ribociclib (mandatory). |
| | If toxicity recurs, discontinue ribociclib (mandatory). |
| Grade 4 (>20.0 × ULN) | Discontinue ribociclib (mandatory) |

AST or ALT and concurrent Bilirubin

| | |
|---|---|
| For patients with normal ALT and AST and total | Discontinue ribociclib (mandatory) |
| bilirubin at baseline: AST or ALT > 3.0 × ULN | |
| combined with total bilirubin > 2 × ULN without | |
| evidence of cholestasis | |
| Or | |
| For patient with elevated AST or ALT or total | |
| bilirubin at baseline: [AST or ALT > 2 × baseline | |
| AND > 3.0 × ULN] OR [AST or ALT > 8.0 × ULN]- | |
| whichever is lower- combined with [total bilirubin > | |
| 2 × baseline AND > 2.0 × ULN] | |

Confounding factors and/or alternative causes for increase of total bilirubin should be excluded before dose interruption/reduction. They include but are not limited to: evidence of liver metastases, evidence of obstruction, such as elevated ALP and GGT typical of gall bladder or bile ducts disease, hyperbilirubinemia due to the indirect component only (i.e. direct bilirubin component ≤1 × ULN) due to hemolysis or Gilbert's Syndrome, other pharmacologic treatment, viral hepatitis, alcoholic or autoimmune hepatitis, other hepatotoxic drugs. For patients with Gilbert's Syndrome, these dose modifications apply to changes in direct bilirubin only. Bilirubin will be fractionated if elevated.
Confounding factors and/or alternative causes for increased transaminases should be excluded before dose interruption/reduction. They include but are not limited to: concomitant medications, herbal preparations or dietary supplements, infection, hepato-biliary disorder or obstruction, liver metastases, and alcohol intake.

Refer to Section 5.2.1.4 for additional follow-up on potential drug-induced liver injury (DILI) cases.

### 5.2.1.2.2. Dose Adjustments for QTcF Prolongation

Refer to Section 6.4.2.6 on guidance on how to collect and interpret ECG.

TABLE A9
Ribociclib Dose Adjustment, Management Recommendation and Mandatory
Discontinuation for QTcF Prolongation (CTCAE v4.03)

| Grade | Dose Adjustment and Management Recommendations |
|---|---|
| For All Grades | 1. Check the quality of the ECG and the QT value and repeat if needed. |
| | 2. Perform analysis of serum electrolytes (potassium, calcium and |
| | magnesium). If outside of the normal range, interrupt ribociclib |
| | administration, correct with supplements or appropriate therapy as soon |
| | as possible, and repeat electrolytes until documented as normal. |
| | 3. Review concomitant medication usage for the potential to inhibit CYP3A4 |
| | and/or to prolong the QT interval. |
| | 4. Check compliance with correct dose and administration of ribociclib. |
| 1* | Perform steps 1-4 as directed in “For All Grades”. |
| QTcF 450-480 msec | No dose adjustment required. |
| 2* | Interrupt ribociclib. Perform steps 1-4 as directed in “For All Grades”. |
| QTcF 481-500 msec | Perform a repeat ECG within one hour of the first QTcF of ≥481 msec. |
| | Repeat ECG as clinically indicated until the QTcF returns to <481 msec. Re- |
| | initiate at the same dose level. No dose adjustment required for first |
| | occurrence. If QTcF ≥481 msec recurs, ribociclib should be reduced by 1 |
| | dose level for the second occurrence. |
| | If QTcF ≥481 msec recurs (third occurrence), ribociclib must be permanently |
| | discontinued. |
| | Repeat ECGs 7 days and 14 days after dose resumption (then as clinically |
| | indicated) for any patients who had therapy interrupted due to QTcF ≥481 |
| | msec. |
| 3* | Interrupt ribociclib. Perform steps 1-4 as directed in “For All Grades.” |
| QTcF ≥501 msec | Perform a repeat ECG within one hour of the first QTcF of ≥501 msec. |
| on at least | If QTcF remains ≥501 msec, consult with a cardiologist (or qualified specialist) |
| two separate ECGs | and repeat cardiac monitoring as indicated until the QTcF returns to <481 |
| | msec. |
| | If QTcF returns to <481 msec, re-initiate at the next lower dose level. |
| | If QTcF remains ≥481 msec after performing steps 1-4 as directed in |
| | “For All Grades,” discontinue ribociclib (mandatory). |
| | If QTcF of ≥501 msec recurs, discontinue ribociclib (mandatory). |
| | Repeat ECGs 7 days and 14 days after dose resumption (then as clinically |
| | indicated) for any patients who had therapy interrupted due to QTcF ≥501 |
| | msec. |
| 4 | Discontinue ribociclib (mandatory). Perform steps 1-4 as directed in “For All |
| [QT/QTcF ≥501 or >60 msec | Grades.” |
| change from baseline] | Obtain local cardiologist (or qualified specialist) consultation and repeat |
| and [TdP or polymorphic | cardiac monitoring as indicated until the QTcF returns to <481 msec. |
| ventricular tachycardia, or | |
| signs/symptoms of serious | |
| arrhythmia] | |

*All values refer to single measure.

### 5.2.1.2.3. Dose Adjustments for Interstitial Lung Disease/Pneumonitis

TABLE A10
Ribociclib Dose Adjustment, Management Recommendation
and Mandatory Discontinuation for Interstitial
Lung Disease (ILD)/Pneumonitis (CTCAE v4.03)

| Grade | Dose Adjustment and Management Recommendations |
|---|---|
| 1 | No dose adjustment required. Initiate appropriate medical |
| Asymptomatic | therapy and monitor as clinically indicated. |
| 2 | Interrupt ribociclib dose until recovery to Grade ≤1, then |
| Symptomatic | resume ribociclib at the next lower dose level*. |
| 3 and 4 | Discontinue ribociclib (mandatory). |
| Severe | |

*An individualized benefit-risk assessment should be performed before resuming ribociclib.

### 5.2.1.2.4. Guidance for All Other Adverse Reactions

The Investigator must use his/her clinical judgment in deciding whether serum electrolytes (particularly potassium, calcium and magnesium) need to be assessed for all other adverse reactions.

In any case that potassium, calcium or magnesium are outside of the normal range, ribociclib must be interrupted and the electrolyte/s rectified using appropriate therapy. Documented normalization of the abnormal electrolyte/s must be available before re-starting ribociclib. Patients who develop renal impairment (not due to other contributing factors) of grade 2 or higher during the Treatment Phase must discontinue treatment with ribociclib.

For all other ribociclib-related AEs, including Toxic Epidermal Necrolysis (TEN), which is a grade 4 event by CTCAE, please follow recommendations in Table A11 (for guidance on dose adjustments for hematological, hepatic, QTcF and ILD/pneumonitis, refer to Table A7, Table A8, Table A9 and Table A10 respectively).

TABLE A11
Ribociclib Dose Adjustment, Management Recommendation and
Mandatory Discontinuation for All Other Adverse Reactions

| Grade | Dose Adjustment and Management Recommendations |
|---|---|
| 1 | No dose adjustment recommended. Initiate appropriate medical |
| | therapy and monitor. |
| 2 | Dose interruption until recovery to grade ≤1. Initiate appropriate |
| | medical therapy and monitor. Re-initiate ribociclib at the same |
| | dose level. |
| | If the same toxicity recurs at grade 2, interrupt ribociclib until |
| | recovery to grade ≤1. Re-initiate ribociclib at the next lower |
| | dose level. |
| 3 | Dose interruption until recovery to grade ≤1. Initiate appropriate |
| | medical therapy and monitor. Re-initiate ribociclib at the next |
| | lower dose level. |
| | If toxicity recurs at grade 2 or 3, discontinue ribociclib |
| | (mandatory). |
| 4 | Discontinue ribociclib (mandatory) and treat with appropriate |
| | medical therapy. |

### 5.2.1.3. Follow-up for Toxicities

Ongoing AEs or abnormal laboratory values at time of ribociclib discontinuation must be followed closely until resolution to baseline values or stabilization of the event. The recommended frequency of this follow-up is weekly for 4 weeks, and subsequently at 4-week intervals (until resolution or stabilization). See section 7.1 for information on the period of observation of AEs and Serious Adverse Events (SAEs).

### 5.2.1.4. Management of Potential Drug-Induced Liver Iniury (DILI) Cases

Increase in transaminases combined with total bilirubin (TBIL) increase may be indicative of DILI, and should be considered as a clinically-important event.

The threshold for potential DILI may depend on the patient's baseline AST/ALT and TBIL value; patients meeting any of the following criteria will require further follow-up as outlined below:
For patients with normal ALT and AST and TBIL value at baseline: AST or ALT >3.0×ULN combined with TBIL >2.0×ULN
For patients with elevated AST or ALT or TBIL value at baseline: (AST or ALT >2× baseline AND >3.0×ULN) OR (AST or ALT >8.0×ULN), whichever is lower, combined with (TBIL >2× baseline AND >2.0×ULN)

Medical assessment needs to ensure that liver test elevations are not caused by cholestasis, defined as: Alkaline Phosphatase (ALP) elevation >2.0×ULN with R value <2 in patients without bone metastasis, or elevation of ALP liver fraction in patients with bone metastasis. (Note: The R value is calculated by dividing the ALT by the ALP, using multiples of the ULN for both values. It denotes the relative pattern of ALT and/or ALP elevation is due to cholestatic or hepatocellular liver injury or mixed type injury).

In the absence of cholestasis, these patients must be immediately discontinued from ribociclib, and repeat LFT as soon as possible, preferably within 48 hours from the awareness of the abnormal results. The evaluation should include laboratory tests, detailed history, physical assessment and the possibility of liver metastasis or new liver lesions, obstructions/compressions, etc.

Hepatic toxicity monitoring includes the following LFTs: albumin, ALT, AST, total bilirubin, direct and indirect bilirubin, ALP (fractionated if ALP is grade 2 or higher), creatine kinase, prothrombin time (PT)/INR and GGT. For patients with Gilbert's syndrome: total and direct bilirubin must be monitored, intensified monitoring applies to changes in direct bilirubin only.

Close observation is recommended in case of AST, ALT, and/or bilirubin increase requiring dose interruption, which involves:
Repeating liver enzyme and serum bilirubin tests two or three times weekly. Frequency of re-testing can decrease to once a week or less if abnormalities stabilize or return to normal values.
Obtaining a more detailed history of current symptoms.
Obtaining a more detailed history of prior and/or concurrent diseases, including history of any pre-existing liver conditions or risk factors.
Obtaining a history of concomitant drug use (including non-prescription medications, herbal and dietary supplements), alcohol use, recreational drug use, and special diets.
Ruling out liver metastases.
Ruling out acute viral hepatitis types A, B, C, D, and E; hepatotropic virus infections (cytomegalovirus, Epstein-Barr, herpes simples); autoimmune or alcoholic hepatitis; non-alcoholic steatohepatitis; hypoxic/ischemic hepatopathy; and biliary tract disease.
Obtaining a history of exposure to environmental chemical agents.
Obtaining additional tests to evaluate liver function, as appropriate (e.g. INR, direct bilirubin).
Considering gastroenterology or hepatology consultations.
Assessing cardiovascular dysfunction or impaired liver oxygenation, including hypotension or right heart failure as possible etiologies for liver dysfunction.
Considering liver biopsy as clinically indicated to assess pathological change and degree of potential liver injury.

All cases of DILI confirmed on repeat testing meeting the laboratory criteria defined above, with no other alternative cause for LFT abnormalities identified, should be considered as “medically significant”, thus met the definition of SAE (Section 7.3), and must be reported as SAE using the term “potential drug-induced liver injury”. All events must be followed up with the outcome clearly documented. Results of tests as well as other clinically important information will be recorded in the CRF.

### 5.2.2. Endocrine Therapy

ET-related AEs will be managed according to local clinical guidelines and Investigator's judgment. In cases when ET needs to be interrupted for more than 4 weeks due to ET-related AEs, the Medical Monitor should be contacted to discuss trial treatment continuation.

### 5.3. Trial Treatment Management

Refer to Section 5.1 for details on the schedule and administration of trial treatment.

### 5.3.1. Packaging and Labeling

Ribociclib (and ET if provided by Novartis) will be provided as open-label patient-specific supply. Medication labels will comply with the legal requirements of each country and be printed in the local language. They will include storage conditions for the drug but will not supply information about the patient.

### 5.3.2. Supply and Storage

Ribociclib (and ET if provided by Novartis) must be received by designated personnel at the trial site, handled and stored safely and properly, and kept in a secured location to which only the Investigator and designated site personnel have access. Upon receipt, the trial treatments should be stored according to the instructions specified on the drug labels, the ribociclib IB or the local product information.

### 5.3.3. Compliance and Accountability

The Investigator or responsible site personnel should instruct the patient to take the trial treatments as per protocol (to promote compliance). Site staff must ensure that the patient clearly understands the directions and follows the schedule adequately. The site personnel will ensure that the appropriate dose of each trial treatment is provided at each cycle.

Compliance will be assessed by the Investigator and/or designated site personnel at each patient visit during the Treatment Phase and information provided by the patient and/or caregiver will be captured in the source documents.

The administration of the trial treatment will be recorded in the appropriate sections of the CRF. Additionally, patients will complete a diary to record their daily intakes. They will be instructed to return all unused ribociclib (and ET if provided by Novartis) (partially used and empty containers) and their diary at each visit during the Treatment Phase. Site staff will perform accountability of the returned drug and will assess compliance with trial treatment.

For ribociclib (and ET if provided by Novartis), the Investigator or designee must maintain an accurate record of the receipt and dispensing of the trial treatment in a drug accountability log. Drug accountability will be reviewed by the trial monitor during site visits and at the completion of the trial.

### 5.3.4. Disposal and Destruction

Ribociclib (and ET if provided by Novartis) can be destroyed at the local Novartis facility, Drug Supply group or third party, as appropriate. Destruction at the investigational site of trial treatment provided by Novartis will only be permitted if authorized by Novartis or designee and if permitted by local regulations.

### 5.4. Concomitant Medications and Treatments

### 5.4.1. General Considerations

The patient must be instructed to notify the investigational site about any new medications she/he takes after signature of the PICF.

All concomitant medications and significant non-drug therapies (including physical therapy, vitamins, herbal/natural medications and blood transfusions) administered from 30 days before randomization until 36 months after the randomization date (up until the 30-Day Post Ribociclib Safety Follow-up visit in the Investigational arm), must be listed on the CRF (unless EOT occurs prior to completing 36 months of treatment; in such case collection will stop at time of the 30-Day Safety Follow-up visit).

For patients experiencing distant disease recurrence, if collection of concomitant medications and non-drug therapies was previously discontinued according to the paragraph above, every attempt should be made to resume collection of medications used to treat pain, depression and anxiety at time of recurrence and for 12 months following distant recurrence, following the same schedule as for post-recurrence PRO collection (see Table A20).

Patients taking concomitant medication chronically should be maintained on the same dose and dose schedule throughout the trial period, as medically feasible and indicated.

Bone-modifying agents (e.g. bisphosphonates, denosumab) are allowed for the treatment of osteoporosis, and when used as adjuvant therapy to reduce bone recurrence and improve BC outcomes, when administered according to the Cancer Care Ontario/American Society of Clinical Oncology Clinical Practice Guideline or the corresponding local guideline.⁴⁶ When these are indicated, it is strongly recommended to initiate bone-modifying agents prior to randomization.

Patients are not permitted to participate in any additional parallel investigational drug or device study.

Participation in other studies involving investigational drug(s) within 30 days prior to randomization or within 5 half-lives of the investigational drug(s) (whichever is longer), or participation in any other type of medical research judged not to be scientifically or medically compatible with this trial, is prohibited. If the patient is enrolled or planned to be enrolled in another study that does not involve an investigational drug, the agreement of the Medical Monitor is required to establish eligibility.

While patients are receiving trial treatment, they are not permitted to:
Receive any other investigational or anti-neoplastic therapy.
Receive hormonal contraception (including use of an intrauterine system) or hormonal medications used as a hormonal replacement therapy for symptoms of menopause, phytoestrogens (as these have potential to reduce efficacy of ET).
   5.4.2. Medications and Treatments Used Concomitantly with ET

For permitted or prohibited medications and treatments used concomitantly with ET, refer to the local clinical guidelines and current local prescribing information.

Appropriate preventive or therapeutic measures for osteopenia, osteoporosis, increases in blood cholesterol or any other ET-related AE should be implemented according to Investigator's judgment and local clinical guidelines.

5.4.3. Medications and Treatments Used Concomitantly with Ribociclib

The sections below provide guidance about ribociclib concomitant medications and treatments.

### 5.4.3.1. Prohibited Concomitant Therapy

The following medications are prohibited during treatment with ribociclib:
Strong inhibitors or inducers of CYP3A4/5 (may significantly increase or decrease ribociclib exposure, respectively).
Substrates of CYP3A4/5 with a narrow therapeutic index (ribociclib may increase exposure to these medications resulting in toxicity to these medications).
Medications with a known risk for QT prolongation and/or TdP (may precipitate QT prolongation and TdP in combination with ribociclib).
Concomitant tamoxifen or toremifene use.

Herbal medications/preparations or dietary supplements that are strong inhibitors or inducers of CYP3A4/5 or those with a known risk of QT prolongation. These include, but are not limited to: St. John's wort, Kava, ephedra (ma huang), gingko biloba, dehydroepiandrosterone (DHEA), yohimbe, saw palmetto, black cohosh and ginseng. Patients should stop using these preparations/medications at least 7 days prior to randomization. It is important to consider potential DDIs when using concomitant medications associated with hot flushes and other anticipated symptoms associated with this indication/use of ET.

Refer to Appendix 4: Concomitant Medications, for a detailed list of prohibited drugs. This list is not comprehensive and is only meant to be used as a guide. Please contact the TRIO Medical Monitor with any questions.

### 5.4.3.2. Concomitant Therapy Requiring Caution

Medications to be used with caution during treatment with ribociclib are listed below:
Medications that carry a possible risk for QT prolongation and/or TdP (may precipitate QT prolongation and TdP).
Moderate inhibitors or inducers of CYP3A4/5 (may increase or decrease ribociclib exposure, respectively).
Sensitive substrates of CYP3A4/5 that do not have narrow therapeutic index (ribociclib may increase exposure to these medications).
Based on in vitro data (refer to the most recent ribociclib IB for details), co-administration of ribociclib with strong inhibitors of Bile Salt Export Pump (BSEP) may lead to intrahepatic cholestasis, and co-administration of ribociclib with sensitive substrates of the renal transporters, MATE1 and OCT2 (has a potential to increase exposure to substrates of these transporters, although no animal or clinical data are available to support these statements).
Sensitive substrates of transporter of Breast Cancer Resistance Protein (BCRP) (has a potential to increase exposure to substrates of these transporters, although no animal or clinical data are available to support these statements).

Please refer to Appendix 4: Concomitant Medications, for a detailed list of drugs that should be used with caution. This list is not comprehensive and is only meant to be used as a guide.

Please contact the TRIO Medical Monitor with any questions. These medications should be excluded if possible. If they must be given based on the Investigator's judgment, then use with caution and consider a ribociclib interruption if the concomitant medication is only needed for a short time.

5.4.3.3. Drugs with QT Prolongation

As far as possible, avoid co-administering medications with a “Known”, “Possible” or “Conditional” risk of TdP or any other medication with the potential to increase the risk of drug-related QT prolongation (e.g. via a potential DDI increasing the exposure of ribociclib or the exposure of the QT prolonging drug).

Medications with a known risk for QT prolongation are prohibited during treatment with ribociclib. If concomitant administration of drugs with a known risk of TdP is required and cannot be avoided, ribociclib must be interrupted.

If during the course of the trial, concomitant administration of a drug with “Possible” or “Conditional” risk of TdP is required, based on the Investigator's assessment and clinical need, ribociclib may be resumed under close clinical and ECG monitoring to ensure patient safety.

A list of drugs associated with QT prolongation and/or TdP is available online (www.qtdrugs.org). Refer to the current ribociclib IB and other drug package insert and Appendix 4: Concomitant Medications, for information on possible interactions with other drugs.

### 5.4.3.4. Permitted Concomitant Therapy

Medications required to treat AEs, manage cancer symptoms, concurrent diseases and supportive care agents, such as pain medications, antiemetics and anti-diarrheals are allowed, with some exceptions as stated in Section 5.4.3.5 and Appendix 4: Concomitant Medications. Potential drug interaction between ribociclib and concomitant medications should always be taken into consideration.

### 5.4.3.5. Considerations About Other Concomitant Treatments

### 5.4.3.5.1. Corticosteroids

Chronic dosing of corticosteroids such as dexamethasone and prednisone is known to lead to induction of CYP3A enzymes, thereby potentially reducing ribociclib drug exposure to sub-therapeutic levels. Systemic corticosteroid treatment should not be given during treatment with ribociclib, except for:
Topical applications (e.g. for rash), inhaled sprays (e.g. for obstructive airways diseases), eye drops or local injections (e.g. intra-articular);
A short duration (<5 days) of systemic corticosteroids s to the anti-inflammatory potency of 4 mg dexamethasone (e.g. for chronic obstructive pulmonary disease or as an antiemetic).

### 5.4.3.5.2. Hematopoietic Growth Factors

Primary prophylactic use of White Blood Cells (WBC) growth factors with ribociclib is not recommended. These are allowed when administered according to the American Society of Clinical Oncology Clinical Practice Guideline or the corresponding local guideline.⁴⁷

### 5.4.3.5.3. Use of Antiemetic Medications

Ribociclib has minimal to low emetogenic potential according to the definition of anti-neoplastic agent emetogenicity.⁴⁶

Antiemetic therapy can be used according to clinical guidelines for anti-neoplastic medications with low to minimal emetogenic potential for treatment and/or prevention of nausea and vomiting as a result of treatment.^{49,50}

Potential drug interaction between ribociclib and antiemetic medications should always be taken into consideration. Example of a prohibited antiemetic medication is ondansetron that in combination with ribociclib may precipitate TdP.

Refer to Sections 5.4.3.1, 5.4.3.2 and Appendix 4: Concomitant Medications, for a list of medications that are allowed (with caution) or prohibited to be used with ribociclib. 5.4.3.5.4. Concomitant Surgery

For patients in the Investigational arm requiring surgery while on trial treatment, the following is recommended:
Relevant laboratory results (e.g. hematology) should be reviewed immediately before surgery.
Schedule the surgery during the week off treatment with ribociclib to allow for hematologic recovery.
Review the protocol guidance on concomitant medications to ensure that patient does not receive a prohibited therapy pre/post-operatively.
Post-operatively, the decision to reinitiate ribociclib should be based on a clinical assessment of satisfactory recovery, according to Investigator's judgment.

### 6. TRIAL VISITS AND ASSESSMENTS

### 6.1. Screening and Randomization

### 6.1.1. Screening

After the patient signs the main PICF, she/he must be recorded in the IRT (on the same date) and this is considered as the start of the Screening Phase (see Section 6.3.1).

At this time, a unique Patient Number (Patient No.) will be assigned in the system. This number is retained as the primary identifier for the patient throughout her/his entire participation in the trial, even if the patient is re-screened. The Patient No. consists of the Site Number (Site No.) with a sequential patient number suffixed to it, so that each patient is numbered uniquely across the entire database.

### 6.1.2. Randomization

Patients will be assigned to one of the two treatment arms in a ratio of 1:1. Randomization will be stratified by the factors reported in Section 8.4.

Randomization may occur as soon as all screening assessments have been conducted (in the appropriate time window) and eligibility status confirmed by the Investigator. Randomization will be done in the IRT as per instructions provided in the applicable User Manual.

### 6.1.3. Screen Failures

Patients that have signed the PICF but are not randomized for any reason will be considered screen failures and will be recorded as such in the IRT. Limited data will be entered in the CRF for screen failed patients, according to the CRF Completion Guidelines.

### 6.1.4. Re-Screening

Re-screening of patients is only allowed once per patient, if the patient was not randomized before (i.e. IRT randomization). In this case, the Patient No. initially assigned will be used and the patient will be identified with this number throughout her/his entire participation to the trial. A new PICF must be signed if the Investigator chooses to re-screen a patient following screen failure.

In case re-screening occurs, all evaluations re-assessed should meet the eligibility criteria. For eligibility purposes, the laboratory tests performed closest to the randomization date will be considered and must meet the eligibility criteria.

For re-screened patients, there is no need to re-draw a sample for ctDNA/ctRNA, unless the patient was on any ongoing anti-cancer treatment when the sample was taken.

### 6.2. Schedule of Visits and Assessments

Table A12 lists all of the protocol required assessments and indicates with an “X”, the visits when they must be performed. All data obtained from these assessments must be supported in the patient's source documentation.

Allowed visit windows are as follows (unless otherwise specified):
Randomization must occur within 42 days of the date of PICF signature (Note: for patients receiving tamoxifen at time of PICF signature, consider the need of a washout period prior to randomization, according to Inclusion Criterion #12).
All screening assessments must occur within 28 days prior to randomization.
Randomization and C1D1 should preferably occur on the same day. A window of up to 7 days between randomization and C1D1 visit is permitted.
For all visits, a ±3 days window is permitted for the applicable assessments (except for ECGs required on C1D1), to take into account scheduling issues.
Assessments that can potentially lead to a trial treatment dose modification (e.g. dose interruption, reduction, discontinuation), should be reviewed by the Investigator prior to dosing.

TABLE A12
Schedule of Visits and Assessments

| | | Screening | | |
|---|---|---|---|---|
| | | Phase | Treatment Phase | |
| | | Visit → | | |
| | | Screening | Cycle 1 | |
| | | Day → | | |
| | | All assessments | | |
| | | done within 28 | | |
| | | days of | | |
| | Protocol | randomization | | |
| Assessment | Section | (unless otherwise | | |
| ↓ | ↓ | stated) | 1 | 15 |

PICF and IRT

| | | | |
|---|---|---|---|
| Main PICF | 6.3.1; | X (within | |
| signature | 9.2.3 | 42 days of | |
| | | randomization) | |
| IRT | 6.1.1; | X (at time | |
| recording | 6.3.2.2; | of PICF and | |
| | 6.3.2.3 | randomization) | |
| IRT drug | NA | | X |
| dispensing (for | | | |
| ribociclib, and | | | |
| ET if supplied | | | |
| by sponsor) | | | |

Patient and BC History

| Demography, | 6.3.1 | X | |
|---|---|---|---|
| medical history, | | | |
| BC history | | | |
| ER/PgR/HER2 | 6.3.1 | X | |
| status known | | | |
| Menopausal | 6.3.1; | X | As clinically indicated |
| status | 6.4.2.5.4 | | |
| Inclusion/ | 4 | X | |
| Exclusion | | | |
| Criteria | | | |

Physical examination

| | | | | |
|---|---|---|---|---|
| Physical | 6.4.2.1 | X | X* | |
| examination | | | | |
| ECOG | 6.4.2.4 | X | X* | |
| Performance | | | | |
| Status | | | | |
| Vital signs, | 6.4.2.2; | X | X* | X |
| Weight | 6.4.2.3 | | | |
| Height | 6.4.2.3 | X | | |

Laboratory assessments

| Hematology | 6.4.2.5.1 | X | X* | X |
|---|---|---|---|---|
| Biochemistry | 6.4.2.5.2 | X | X* | X |
| Coagulation | 6.4.2.5.3 | X | As clinically indicated | |
| Serum | 6.4.2.5.4 | X (within | | |
| pregnancy test | | 14 days of | | |
| (women CBP) | | randomization) | | |
| Serum or urine | 6.4.2.5.4 | | X* | |
| pregnancy test | | | | |
| (women CBP) | | | | |

Recurrence assessment

| | | | |
|---|---|---|---|
| Clinical | 6.4.1.1 | X | X (Every 12 weeks (±2 weeks) during the first 24 months and every 24 weeks |
| evaluation | | | (±3 weeks) thereafter, until confirmation of distant recurrence) |
| (STEEP) | | | |
| Mammography | 6.4.1.1 | X (unless | X (Every 12 months (±4 weeks) and as clinically indicated, |
| (unless | | within 12 months | until confirmation of distant recurrence) |
| bilateral | | | |
| mastectomy) | | of screening) | |
| Additional | 6.4.1.1 | X (if clinically | X (Within 4 weeks after clinical suspicion of local, regional or |
| imaging | | indicated) | distant recurrence or contralateral invasive BC or |
| (CT, MRI, US, | | | second primary non-breast invasive cancer according to |
| PET, bone | | | Appendix 3: Recurrence Detection Guidelines) |
| scan, X-ray, | | | |
| mammography) | | | |
| Histological (or | 6.4.1.1 | X (if clinically | X (Within 4 weeks after clinical suspicion of local, regional or |
| cytological) | | indicated) | distant recurrence or contralateral invasive BC or |
| confirmation of | | | second primary non-breast invasive cancer according to |
| recurrence | | | Appendix 3: Recurrence Detection Guidelines) |

Survival status assessment

| | |
|---|---|
| Survival | 6.4.1.2 |
| status | |

Cardiac assessments

| | | | | |
|---|---|---|---|---|
| Single 12- | 6.4.2.6 | X | X | X |
| lead ECG | | | | |

Adverse Events, concomitant and subsequent medications

| AEs (non- | 7.1; 7.2 | X (only if | X (Continuous up to 36 months since randomization [in the |
|---|---|---|---|
| serious) | | related to trial | Investigational arm until the 30-Day Post Ribociclib |
| | | participation) | Safety Follow-up visit]. See Table A21 for details) |
| SAEs | 7.1; 7.3 | | X (Continuous) |
| Concomitant | 5.4 | X (Continuous, from 30 days before randomization until 36 months since | |
| medications | | randomization [in the Investigational arm until the 30-Day Post | |
| and procedures | | Ribociclib Safety Follow-up visit]) | |
| Subsequent | 6.3.2.3; | | |
| anti-neoplastic | 6.3.2.4; | | |
| therapies | 6.3.3 | | |

Pharmacokinetics

| | | |
|---|---|---|
| PK sample (PK | 6.4.3 | X |
| subset only) | | |

Biomarkers**

| Tumor tissue | 6.4.4.1.1 | X | |
|---|---|---|---|
| (archival): | | | |
| availability | | | |
| for use in | | | |
| trial | | | |
| confirmed | | | |
| Tumor tissue | 6.4.4.1.1 | | X (From local, regional and distant recurrence -unless unsafe |
| at recurrence | | | for the patient per Investigator's discretion-) |
| Blood for | 6.4.4.1.2 | X (up to C1D1, | |
| ctDNA/ctRNA | | prior to dosing) | |
| Blood for | 6.4.4.3 | | X (C1D1, or within first 12 |
| pharmacogenetic | | | weeks of randomization) |
| analysis | | | |
| (optional) | | | |

Patient Reported Outcomes

| | | | |
|---|---|---|---|
| EORTC QLQ- | 6.4.6 | X | X (Every 12 weeks (±2 weeks) during the first 24 months, every 24 weeks |
| C30 | | | (±3 weeks) thereafter until distant recurrence. At EOT [symbol] At |
| EORTC QLQ- | 6.4.6 | X | confirmation of first recurrence and at confirmation of distant |
| BR23 (for | | | recurrence (if the first recurrence was not a distant recurrence), |
| women only) | | | and during first 12 months after confirmation of distant |
| EQ-5D-5L | 6.4.6 | X | recurrence. After confirmation of distant recurrence, for 12 months: |
| HADS | 6.4.6 | X | every [symbol] 12 weeks (±2 weeks) if confirmation of distant |
| | | | recurrence happened during first 24 months after date of |
| | | | randomization, or every [symbol] 24 weeks (±3 weeks) if confirmation of |
| | | | distant recurrence happened after first 24 months after date of randomization) |

Resource Utilization

| Hospitalizations, | 6.4.5 | X (Continuous during Treatment and Follow-up Phases. In case of distant |
|---|---|---|
| Emergency Room | | recurrence, up to 12 months following confirmation of recurrence -same |
| visits, additional | | schedule as for post-recurrence PRO collection-) |
| relevant | | |
| clinic visits | | |

Trial Treatment Administration

| Ribociclib | 5.1.1 | Once daily on days 1 to 21 in each 28-day |
|---|---|---|
| (Investigational | | cycle, for 36 months from randomization |
| arm only) | | |

| | | |
|---|---|---|
| NSAI | 5.1.1 | Once daily given continuously, for 60 months from randomization |
| Goserelin (if | 5.1.1 | Day 1 ± 3 of each 28-day cycle, for 60 months from randomization |
| applicable) | | |

| | | | | | | | | | Follow- |
|---|---|---|---|---|---|---|---|---|---|
| | | | | | | | | | up |
| | Treatment Phase | | | | | | | | Phase |
| | Visit → | | | | | | | | |
| | | | | Cycle 7 until | 30-Day Post | | | | |
| | | | | 36 months from | Ribociclib | From 36 | | | |
| | | | | randomization | Safety | months | | | |
| | | | Cycles 3 | (every 3^{rd} | Follow-up | from | | 30-Day | |
| | | | to 6 (each | cycle from | (Investigational | rando. | | Safety | Follow- |
| | Cycle 2 | | cycle) | C7D1) | arm only) | to EOT | EOT | Follow-up | up |
| | Day → | | | | | | | | |
| | | | | | | | | 30 days | |
| | | | | | | | Within 15 | (±3) | |
| | | | | | 30 days | | days of last | of last | See |
| | | | | | (±3) of | See | dose of | dose of | Sections |
| Assessment | | | | | last dose of | Section | all trial | all trial | 6.3.3, |
| ↓ | 1 | 15 | 1 | 1 | ribociclib | 6.3.2.1 | treatments | treatments | 6.4 |
| | PICF and IRT | | | | | | | | |
| Main PICF | | | | | | | | | |
| signature | | | | | | | | | |
| IRT | | | | | X | | X | | |
| recording | | | | | | | | | |
| IRT drug | X | | X | X | | X (q. 12 | | | |
| dispensing (for | | | | | | weeks) | | | |
| ribociclib, and | | | | | | | | | |
| ET if supplied | | | | | | | | | |
| by sponsor) | | | | | | | | | |
| | Patient and BC History | | | | | | | | |
| Demography, | | | | | | | | | |
| medical history, | | | | | | | | | |
| BC history | | | | | | | | | |
| ER/PgR/HER2 | | | | | | | | | |
| status known | | | | | | | | | |
| Menopausal | As clinically indicated | | | | | | | | |
| status | | | | | | | | | |
| Inclusion/ | | | | | | | | | |
| Exclusion | | | | | | | | | |
| Criteria | | | | | | | | | |
| | Physical examination | | | | | | | | |
| Physical | X | | X | X | | | X | | |
| examination | | | | | | | | | |
| ECOG | X | | X | X | | | X | | |
| Performance | | | | | | | | | |
| Status | | | | | | | | | |
| Vital signs, | X | X | X | X | | | X | | |
| Weight | | | | | | | | | |
| Height | | | | | | | | | |
| | Laboratory assessments | | | | | | | | |
| Hematology | X | X | X | X | | | X | | |
| Biochemistry | X | X | X | X | | | X | | |
| Coagulation | As clinically indicated | | | | | | | | |
| Serum | | | | | | | | | |
| pregnancy test | | | | | | | | | |
| (women CBP) | | | | | | | | | |
| Serum or urine | X | | X | X (See | | | X | | |
| pregnancy test | | | | section | | | | | |
| (women CBP) | | | | 6.4.2.5.4) | | | | | |
| | Recurrence assessment | | | | | | | | |
| Clinical | X (Every 12 weeks (±2 weeks) during the first 24 months and every 24 weeks | | | | | | | | |
| evaluation | (±3 weeks) thereafter, until confirmation of distant recurrence) | | | | | | | | |
| (STEEP) | | | | | | | | | |
| Mammography | X (Every 12 months (±4 weeks) and as clinically indicated, | | | | | | | | |
| (unless | until confirmation of distant recurrence) | | | | | | | | |
| bilateral | | | | | | | | | |
| mastectomy) | | | | | | | | | |
| Additional | X (Within 4 weeks after clinical suspicion of local, regional or | | | | | | | | |
| imaging | distant recurrence or contralateral invasive BC or | | | | | | | | |
| (CT, MRI, US, | second primary non-breast invasive cancer according to | | | | | | | | |
| PET, bone | Appendix 3: Recurrence Detection Guidelines) | | | | | | | | |
| scan, X-ray, | | | | | | | | | |
| mammography) | | | | | | | | | |
| Histological (or | X (Within 4 weeks after clinical suspicion of local, regional or | | | | | | | | |
| cytological) | distant recurrence or contralateral invasive BC or | | | | | | | | |
| confirmation of | second primary non-breast invasive cancer according to | | | | | | | | |
| recurrence | Appendix 3: Recurrence Detection Guidelines) | | | | | | | | |
| | Survival status assessment | | | | | | | | |
| Survival | | | | | | | | | X |
| status | | | | | | | | | |
| | Cardiac assessments | | | | | | | | |
| Single 12-lead | X | X | X | X | | | X | | |
| ECG | | | (C3 and | (if QTcF ≥481 | | | | | |
| | | | C6 only) | msec any time | | | | | |
| | | | | prior to cycle 7) | | | | | |
| | Adverse Events, concomitant and subsequent medications | | | | | | | | |
| AEs (non- | X (Continuous up to 36 months since randomization [in the | | | | | | | | |
| serious) | Investigational arm until the 30-Day Post Ribociclib | | | | | | | | |
| | Safety Follow-up visit]. See Table A21 for details) | | | | | | | | |
| SAEs | X (Continuous) | | | | | | | | X (if trial |
| | | | | | | | | | treatment- |
| | | | | | | | | | related) |
| Concomitant | X (Continuous, from 30 days before randomization | | | | | In case of distant recurrence, every | | | |
| medications | until 36 months since randomization [in the | | | | | attempt should be made to collect | | | |
| and procedures | Investigational arm until the 30-Day Post | | | | | medication used to treat pain, | | | |
| | Ribociclib Safety Follow-up visit]) | | | | | depression and anxiety, at time of | | | |
| | | | | | | recurrence and up to 12 months | | | |
| | | | | | | following confirmation of | | | |
| | | | | | | recurrence (same schedule as for | | | |
| | | | | | | post-recurrence PRO collection) | | | |
| Subsequent | | | | | | | X | X | X |
| anti-neoplastic | | | | | | | | | |
| therapies | | | | | | | | | |
| | Pharmacokinetics | | | | | | | | |
| PK sample (PK | | | | | | | | | |
| subset only) | | | | | | | | | |
| | Biomarkers** | | | | | | | | |
| Tumor tissue | | | | | | | | | |
| (archival): | | | | | | | | | |
| availability | | | | | | | | | |
| for use in | | | | | | | | | |
| trial | | | | | | | | | |
| confirmed | | | | | | | | | |
| Tumor tissue | X (From local, regional and distant recurrence -unless unsafe for the patient per | | | | | | | | |
| at recurrence | Investigator's discretion-) | | | | | | | | |
| Blood for | | | X (C4D1, C13D1, C25D1, then every 48 weeks (±3 weeks) after | | | | | | |
| ctDNA/ctRNA | | | C25D1 and at time of local, regional and distant recurrence) | | | | | | |
| Blood for | X (C1D1, or within first 12 | | | | | | | | |
| pharmacogenetic | weeks of randomization) | | | | | | | | |
| analysis | | | | | | | | | |
| (optional) | | | | | | | | | |
| | Patient Reported Outcomes | | | | | | | | |
| EORTC QLQ- | X (Every 12 weeks (±2 weeks) during the first 24 months, every 24 weeks (±3 weeks) thereafter until | | | | | | | | |
| C30 | distant recurrence. At EOT [symbol] At confirmation of first recurrence and at confirmation of distant recurrence | | | | | | | | |
| EORTC QLQ- | (if the first recurrence was not a distant recurrence), and during first 12 months after confirmation of | | | | | | | | |
| BR23 (for | distant recurrence. After confirmation of distant recurrence, for 12 months: every [symbol] 12 weeks | | | | | | | | |
| women only) | (±2 weeks) if confirmation of distant recurrence happened during first 24 months after date of randomization, | | | | | | | | |
| EQ-5D-5L | or every [symbol] 24 weeks (±3 weeks) if confirmation of distant recurrence happened after first 24 months | | | | | | | | |
| HADS | after date of randomization) | | | | | | | | |
| | Resource Utilization | | | | | | | | |
| Hospitalizations, | X (Continuous during Treatment and Follow-up Phases. In case of distant recurrence, up to 12 months | | | | | | | | |
| Emergency Room | following confirmation of recurrence -same schedule as for post-recurrence PRO collection-) | | | | | | | | |
| visits, additional | | | | | | | | | |
| relevant | | | | | | | | | |
| clinic visits | | | | | | | | | |
| | Trial Treatment Administration | | | | | | | | |
| Ribociclib | Once daily on days 1 to 21 in each 28-day | | | | | | | | |
| (Investigational | cycle, for 36 months from randomization | | | | | | | | |
| arm only) | | | | | | | | | |

| | |
|---|---|
| NSAI | Once daily given continuously, for 60 months from randomization |
| Goserelin (if | Day 1 ± 3 of each 28-day cycle, for 60 months from randomization |
| applicable) | |

*Procedures with an “X*” (i.e. X with an asterisk in superscript) need to be performed only if not done within 14 days prior to C1D1.
**Biomarker sample collection from all patients, except those enrolled in China. [symbol]
indicates data missing or illegible when filed

### 6.3. Trial Phases and Visits

### 6.3.1. Screening Phase

The Screening Phase starts when the patient signs the main PICF and ends when she/he is randomized or screen failed. No trial-specific procedure may be performed until the main PICF is signed (note: randomization must occur within 42 days of the date of PICF signature).

During the Screening Phase, the Investigator must:
Perform all screening procedures within 28 days prior to randomization (see Table A12 for list of assessments to be performed).
Assess the inclusion and exclusion criteria as detailed in Section 4.

The following will be collected at screening:
Patient demographics and other baseline characteristics:
   Menopausal status
   Medical history and ongoing medical conditions
   BC history: including (but may not be limited to) initial diagnosis, stage, ER, PgR, HER2 statuses, histopathological grade per the Nottingham combined histologic grade⁵¹, Ki67 (if available), results of any gene expression test (if available), prior anti-neoplastic therapies.
Collection of SAEs and non-serious AEs (only if related to trial participation).
Collection of medications and relevant non-drug therapies taken within 30 days before randomization.
Confirmation of the availability of the required archival paraffin tumor tissue (see Section 6.4.4.1.1).
Blood for ctDNA/ctRNA (can be taken up to C1D1, prior to dosing).
PRO questionnaires (see Section 6.4.6).

Additionally, the following evaluations will be performed at screening:
Physical examination, vital signs, height, weight.
ECOG Performance Status.
Laboratory evaluations (hematology, biochemistry, coagulation, serum pregnancy test for women of CBP).
Single ECG.
Clinical evaluation for recurrence, mammography (unless done within 12 months prior to screening start) and if clinically indicated, radiological and/or histological/cytological assessments. Refer to Section 6.4.1.1, Appendix 2: Guidelines for Standardized Definitions for Efficacy Endpoints in Adjuvant Breast Cancer Trials (based on STEEP) and Appendix 3: Recurrence Detection Guidelines.

### 6.3.2. Treatment Phase

The Treatment Phase starts when the patient begins with trial treatment (C1D1) and ends at time of the 30-Day Safety Follow-up visit.

### 6.3.2.1. Trial Treatment

After randomization, trial treatment should be started as soon as possible and no later than 7 days after the randomization. Trial treatment will continue until criteria in Section 5.1.1.1 is met. During trial treatment, visits need to be performed according to the following schedule:
Up to completing 36 months of trial treatment, from randomization date: in the schedule specified in Table A12 (i.e. every 28 days ±3 from C1 to C6; thereafter every third cycle starting on C7D1).
After completing 36 months of trial treatment, from randomization date: according to Section 6.4.1.1.1, patients should have clinic visits in the frequency established in that section, to assess for recurrences. Other visits and assessments may be performed according to local clinical guidelines.

For details of assessments to be performed at each visit during the Treatment Phase, see Table A12 and Section 6.4.

### 6.3.2.2. 30-Day Post Ribociclib Safety Follow-up Visit

Patients in the Investigational arm must have safety evaluations performed 30 days after the last dose of ribociclib. The 30-Day Post Ribociclib Safety Follow-up visit will take place 30 days (±3 days) from the last ribociclib dose. AEs, concomitant medications/relevant non-drug therapies and healthcare resource utilization data (if any) will be collected at this visit. No other test or assessment is required.

For patients who permanently discontinue ribociclib at the same time as ET, the 30-Day Safety Follow-up visit will be performed 30 days (±3 days) from the last of all trial treatment and that visit will serve as the 30-Day Post Ribociclib Safety Follow-up.

Ribociclib discontinuation will be recorded in the IRT.

### 6.3.2.3. End of Treatment Visit

Patients who discontinue all trial treatments should undergo an EOT visit within 15 days from the last dose of all trial treatments (i.e. last dose of ET within the trial in the Control arm; last dose of ribociclib and ET or last dose of ET only [if ribociclib previously discontinued] in the Investigational arm).

The following evaluations will be performed at this visit:
Physical examination, vital signs, weight.
ECOG Performance Status.
Laboratory evaluations (hematology, biochemistry, serum or urine pregnancy test for women of CBP)
Single ECG collection.
Collection of:
   Non-serious AEs and/or SAEs (as applicable according to Section 7.1)
   Concomitant medications/relevant non-drug therapies (if applicable according to Section 5.4)
   Healthcare resource utilization data.
Clinical evaluation for recurrence (if applicable according to Section 6.4.1.1).
PRO questionnaires (refer to Section 6.4.6).
Collection of subsequent anti-neoplastic therapies (i.e. surgery, radiotherapy, local and systemic anti-neoplastic medications, if any).

EOT reason will be recorded in the IRT.

If the decision to discontinue the patient occurs at a regularly scheduled visit, that visit may serve as the EOT visit rather than having the patient return for an additional visit.

If a patient fails to return for the EOT visit assessments for unknown reasons, every effort (e.g. telephone, email, letter) should be made to contact them as specified in Section 6.3.5.

After the EOT, patients will proceed with the 30-Day Safety Follow-up visit and subsequently with the Follow-up Phase per sections below.

### 6.3.2.4. 30-Day Safety Follow-up Visit

All patients must have safety evaluations for 30 days after the last dose of all trial treatments.

The 30-Day Safety Follow-up visit will take place 30 days (±3 days) from the last dose of all trial treatments.

SAEs (and non-serious AEs if applicable per Section 7.1), subsequent anti-neoplastic therapies and healthcare resource utilization data (if any) will be collected at this visit. No other test or assessment is required.

If patients refuse to return for the 30-Day Safety Follow-up visit or are unable to do so, every effort should be made to contact them by telephone to determine if any AE was experienced.

Attempts to contact the patient should be documented in the source documents (e.g. dates of telephone calls, registered letters, etc.).

### 6.3.3. Follow-up Phase

The Follow-up Phase starts after the 30-Day Safety Follow-up visit and will continue until death, withdrawal of consent, loss to follow-up or end of trial, whichever is earliest.

Patients will be assessed for recurrence and survival according to Sections 6.4.1.1 and 6.4.1.2. For patients who discontinue trial treatment for reasons other than distant recurrence (or death, lost to follow-up, or withdrawal of consent), evaluations for recurrence will be performed during the Follow-up Phase (according to Sections 6.4.1.1) until documentation of distant disease recurrence, death, withdrawal of consent, loss to follow-up, or end of trial.

Until distant recurrence, follow-up visits will occur at time of the clinical evaluation for recurrence according to the schedule in Section 6.4.1.1 (i.e. every 12 weeks [±2 weeks] following randomization during the first 24 months and every 24 weeks [±3 weeks] thereafter). After documentation of distant recurrence, survival information will be obtained according to the schedule in Section 6.4.1.2 (i.e. every 12 weeks [±2 weeks] following randomization during the first 24 months and every 24 weeks [±3 weeks] thereafter).

Selected PROs must continue to be performed during this phase according to Section 6.4.6 and collection of healthcare utilization data will be done according to Section 6.4.5.

During the Follow-up Phase, use of all subsequent anti-neoplastic therapies initiated after trial treatment discontinuation will be recorded.

SAEs that are deemed by the Investigator to be related to the trial treatment will continue to be reported.

### 6.3.4. Withdrawal of Consent

Patients may voluntarily withdraw consent to participate in the trial for any reason at any time.

Withdrawal of consent occurs only when a patient:
Does not want to participate in the trial anymore, and
Does not allow further collection of personal data

In this situation, the Investigator should make a reasonable effort (e.g. telephone, e-mail, letter) to understand the primary reason for the patient's decision to withdraw his/her consent and record this information.

Trial treatment must be discontinued and no further assessments conducted, and the data that would have been collected at subsequent visits will be considered missing.

Further attempts to contact the patient are not allowed unless safety findings require communicating or follow-up.

All efforts should be made to complete the assessments prior to withdrawal. A final evaluation at the time of the patient's withdrawal should be made and will be considered the EOT visit (for patients that withdraw prior to completing trial treatment).

Novartis will continue to keep and use collected trial information (including any data resulting from the analysis of a patient's samples until their time of withdrawal) according to applicable law.

All biological samples not yet analyzed at the time of withdrawal will no longer be used, unless permitted by applicable law. They will be stored according to applicable legal requirements.

### 6.3.5. Lost to Follow-up

For patients whose status is unclear because they failed to appear for trial visits without stating an intention to withdraw consent, the Investigator should show “due diligence” by contacting the patient, family or family physician and will document in the source documents the steps taken to contact the patient, e.g. dates of telephone calls, registered letters, etc. A patient should not be considered lost to follow-up until due diligence has been completed.

### 6.4. Description of Trial Assessments

### 6.4.1. Efficacy Assessments

Efficacy assessment for the primary endpoint of iDFS (and for the secondary and exploratory efficacy endpoints) will include detection of locoregional relapse, distant relapse, ipsilateral and contralateral invasive BCs and second primary non-breast invasive cancer (see Appendix 2: Guidelines for Standardized Definitions for Efficacy Endpoints in Adjuvant Breast Cancer Trials (based on STEEP) and Appendix 3: Recurrence Detection Guidelines).

Efficacy assessments also include evaluation of the survival status done according to Section 6.4.1.2.

Efficacy assessments are fixed according to the calendar, taking as a reference the randomization date (not the date of a previous assessment), regardless of treatment interruptions.

If a patient discontinues trial treatment for reasons other than distant disease recurrence (or death, lost to follow-up, or withdrawal of consent), recurrence assessments should continue as per the schedule in Table A12 and Table A13 until distant disease recurrence, withdrawal of consent by the patient, patient is lost to follow-up, death or end of trial. Follow-up for recurrence will not be discontinued at the event of local or regional recurrence, contralateral invasive BC event or second primary non-breast invasive cancer.

### 6.4.1.1. Recurrence Assessments

Recurrences (according to STEEP System) will be reported as local, regional, distant, contralateral invasive BC and second primary non-breast invasive cancer (excluding basal or squamous cell carcinomas of the skin) (see Appendix 2: Guidelines for Standardized Definitions for Efficacy Endpoints in Adjuvant Breast Cancer Trials (based on STEEP)).

The imaging assessments described in sections 6.4.1.1.1 and 6.4.1.1.2 are aligned with the standard of care imaging assessments done in EBC patients. No additional radiological examinations are required as part of this protocol.

### 6.4.1.1.1. Clinical Evaluation and Mammography

Detection of recurrence will be done by clinical evaluation that includes medical history and physical examination. Clinical evaluation for recurrence will be done as follows:
At screening within 28 days period prior to randomization.
Subsequently every 12 weeks (±2 weeks) following randomization (using the randomization date as the reference date and not the date of the previous assessment) during the first 24 months and every 24 weeks (±3 weeks) thereafter.
Note: The 12-week (or 24 week) interval should be respected regardless of whether trial treatment is temporarily withheld or unscheduled assessments performed. A window of ±2 or 3 weeks (as detailed above) is permitted to take into account scheduling issues.

Scheduled mammography should be done (unless bilateral mastectomy):
At screening (unless done within 12 months prior to screening start).
Subsequently every 12 months (±4 weeks) after randomization (if mammography was done at screening) or every 12 months (±4 weeks) from the date of the latest mammography done prior to screening, if applicable. Mammography should also be done as clinically indicated.
An interval of no more than 12 months (+4 weeks) between mammography procedures should be maintained.

If recurrence is suspected during clinical evaluation or scheduled mammography, it has to be evaluated according to Section 6.4.1.1.2.

Clinical evaluation for recurrence and mammography should continue until documentation of distant disease recurrence, death, withdrawal of consent, loss to follow-up, or end of trial.

Patients with local and/or regional recurrence in the absence of distant disease should continue with the follow-up for recurrence according to this section.

### 6.4.1.1.2. Additional Imaging Assessments and Confirmation of Recurrence

Recurrence suspected during clinical evaluation or scheduled mammography has to be evaluated by additional medical imaging (as clinically indicated). In addition, every attempt should be made to confirm recurrence histologically (or cytologically, when applicable) as defined in this section and in Appendix 3: Recurrence Detection Guidelines, unless the biopsy poses an unacceptable risk to the patient.

Recurrence that is suspected clinically only but is not confirmed as described in Appendix 3: Recurrence Detection Guidelines, will not be reported as confirmed recurrence in the CRF and will not be considered as an iDFS event per STEEP System.

Imaging assessments (excluding scheduled mammography, although mammography can be used to evaluate recurrence suspected during clinical evaluation) will be performed after clinical evaluation, within 4 weeks after clinical suspicion of recurrence. Findings from radiological evaluations (evaluation of suspicion of recurrence or unconfirmed findings) will be recorded in the CRF.

Any of the following radiological assessments may be used for evaluation after clinical suspicion of recurrence:
Chest, abdomen, pelvis computed tomography (CT) or magnetic resonance imaging (MRI)
Brain CT or MRI
Whole body bone scan (bone scintigraphy)
Localized bone CT, MRI or X-ray, for any lesions identified on the whole body bone scan that are not visible on the chest, abdomen, pelvis CT or MRI
CT or MRI of other sites (e.g. neck, extremities)
Ultrasound (US)
Fludeoxyglucose (¹⁸F) positron emission tomography (FDG-PET), PET-CT
Mammography (used to evaluate recurrence suspected during clinical evaluation)

CT with intravenous (i.v.) contrast is a preferred imaging methodology performed after clinical suspicion of recurrence. However, if a patient is known to have a contraindication to i.v. contrast media or develops a contraindication during the trial, a non-contrast spiral CT of chest (MRI is not recommended due to respiratory artifacts) and contrast-enhanced MRI (if possible) of abdomen and pelvis should be performed.

A whole body bone scan (e.g. Tc-99m bone scan, sodium fluoride PET [NaF-PET] bone scan) can be done to identify location of recurrence in bone tissue. If bone metastases were identified by a bone scan, it has to be confirmed histologically (preferred) or radiographically (by X-ray, CT, MRI or FDG-PET-CT) if biopsy confirmation is unsafe (per Investigator's discretion).

Imaging only can be used to confirm distant recurrence in bone tissue (CT, MRI or FDG-PET-CT) and central nervous system (CT or MRI, both with i.v. contrast) if histological confirmation (or cytological, when applicable) poses unacceptable risk for a patient.

In case of skin, subcutaneous or other superficial lesions for which imaging may not be informative according to Investigator's judgment, it is acceptable to not perform imaging procedures to evaluate such lesions. Histological or cytological confirmation is required and sufficient in these cases.

TABLE A13
Imaging Collection Plan

| | Screening | |
|---|---|---|
| Procedure | Phase | Treatment Phase and Follow-up Phase |
| Any of the following: | Not | Within 4 weeks of clinical suspicion of recurrence* (including |
| CT or MRI (with | mandated | local, regional, distant), or contralateral invasive BC or second |
| contrast unless | | primary non-BC. |
| contraindicated), | | Note: In case of skin, subcutaneous or other superficial lesions |
| bone scan, US, | | for which imaging may not be informative, it is acceptable to |
| PET, plain X-ray | | not perform imaging procedures to evaluate such lesions. |
| films, mammography | | |
| Mammography | Mandatory | Every 12 months (±4 weeks) after randomization (if |
| (unless bilateral | unless done | mammography was done at screening) or every 12 months |
| mastectomy) | within 12 | (±4 weeks) from the date of the latest mammography done prior |
| | months prior | to screening, if applicable. Mammography should also be done |
| | to screening | as clinically indicated. |
| | start | An interval of no more than 12 months (+4 weeks) between |
| | | mammography procedures should be maintained. |
| | | Mammography should continue until documentation of distant |
| | | disease recurrence, death, withdrawal of consent, loss to |
| | | follow-up, or end of trial. |

*Clinical evaluation of recurrence: assessed by scheduled clinical evaluations for recurrence every 12 weeks (±2 weeks) for the first 24 months, then every 24 weeks (±3 weeks) thereafter, or at any time when recurrence is suspected.
Note:
Imaging data will be collected and stored in accordance with local regulations. The local Investigator's assessment will be used for the primary/secondary endpoint analysis and for treatment decision making. In the future, central review of the imaging data may be performed retrospectively from locally stored images, if deemed necessary by the Sponsor.

In case of recurrence that is suspected clinically and/or through imaging, every attempt should be made to confirm it histologically (or cytologically, when applicable), as defined in Table A14 and Appendix 3: Recurrence Detection Guidelines, unless the procedure poses an unacceptable risk to the patient. Histological/cytological confirmation of recurrence should be performed within 4 weeks after clinical suspicion of recurrence. All findings and results should be captured in the appropriate CRFs. If the biopsy was not possible, the reason for this should be documented in the CRF.

TABLE A14
Histology/Cytology Collection Plan

| | Screening | Treatment Phase and Post Treatment |
|---|---|---|
| Procedure | Phase | Follow-up Phase |
| Histological | Not | Within 4 weeks of clinical suspicion of |
| or cytological | mandated | recurrence* (including local, regional, |
| evaluation of | | distant), contralateral invasive BC or |
| recurrence | | second primary non-BC. |
| | | Clinical evaluations for recurrence with or |
| | | without radiologic and/or histologic/ |
| | | cytologic confirmation should continue until |
| | | documentation of distant disease recurrence, |
| | | death, withdrawal of consent, loss to follow- |
| | | up, or end of trial. |

*Clinical evaluation of recurrence: assessed by scheduled clinical evaluations for recurrence every 12 weeks (±2 weeks) for the first 24 months, then every 24 weeks (±3 weeks) thereafter, or at any time when recurrence is suspected.

6.4.1.2. Survival Status Assessment Survival information during the Follow-up Phase can be obtained by clinical visits, telephone calls or other means, according to the schedule in Table A12.

For patients still being followed-up for recurrence according to Section 6.4.1.1, no specific additional visit/call to assess survival is required.

Survival status will be assessed according to the following schedule:
If 30-Day Safety Follow-up takes place during the first 24 months after randomization: every 12 weeks (±2 weeks) until 24 months after date of randomization and every 24 weeks (±3 weeks) thereafter.
If 30-Day Safety Follow-up takes place after the first 24 months after date of randomization: every 24 weeks (±3 weeks).

Survival assessments will continue until death, withdrawal of consent, lost to follow-up or end of trial, whichever is earliest.

### 6.4.2. Safety Assessments

Safety of patients will be monitored by assessing physical examinations, ECOG Performance Status, height, weight, vital signs, ECG, laboratory assessments (including hematology, biochemistry, and coagulation), as well as collection of AEs at every applicable visit. For details on AE collection and reporting see Section 7.

### 6.4.2.1. Physical Examination

Physical examination is to be performed according to the visit schedule as outlined in Table A12.

At screening, the physical examination comprises a total body examination that should include: general appearance, skin, neck (including thyroid), eyes, ears, nose, throat, lungs, heart, abdomen, back, lymph nodes, extremities, vascular and neurological review. If indicated, rectal, external genitalia, breast and pelvis exams will be performed. Information about the physical examination must be present in the source documentation at the study site.

Significant findings that were present prior to the signing of the main PICF consent must be included in the Medical History page on the patient's CRF. Significant new findings that begin or worsen after informed consent must be recorded on the AE page of the patient's CRF.

After screening, a symptom/sign-oriented physical examination is acceptable, in addition to the examination directed to the detection of recurrences (see Section 6.4.1.1.1).

### 6.4.2.2. Vital Signs

Vital signs (body temperature, pulse rate, blood pressure) will be monitored as per the visit schedule (see Table A12). Blood pressure (systolic and diastolic) and pulse should be measured after the patient has been sitting for approximately five minutes.

### 6.4.2.3. Height and Weight

Height will be measured at screening only. Weight will be measured at screening and at subsequent time points as specified in Table A12.

### 6.4.2.4. Performance Status

The performance status will be assessed according to the ECOG Performance Status Scale⁵² as specified in Table A15, following the schedule given in Table A12.

TABLE A15
ECOG Performance Status

| Score | Description |
|---|---|
| 0 | Fully active, able to carry on all pre-disease performance without |
| | restriction |
| 1 | Restricted in physically strenuous activity but ambulatory and able |
| | to carry out work of a light or sedentary nature e.g. light house |
| | work, office work |
| 2 | Ambulatory and capable of all self-care but unable to carry out any |
| | work activities. Up and about more than 50% of waking hours |
| 3 | Capable of only limited self-care, confined to bed or chair more |
| | than 50% of waking hours |
| 4 | Completely disabled. Cannot carry on any self-care. Totally |
| | confined to bed or chair |
| 5 | Dead |

### 6.4.2.5. Laboratory Evaluations

Clinical laboratory analyses (hematology, biochemistry, coagulation, and other required tests) are performed by the local laboratory (see Table A16). The only exception is home pregnancy tests, which may be done according to Section 6.4.2.5.4 from C7.

The Investigator is responsible for reviewing all laboratory reports for patients in the trial and evaluating any abnormalities for clinical significance.

TRIO must be provided with a copy of the laboratory's certification and a tabulation of the normal ranges and units of each parameter collected in the CRF. Additionally, if at any time a patient has laboratory parameters obtained from a different (outside) laboratory, TRIO must be provided with a copy of the certification and a tabulation of the normal ranges and units for this laboratory as well.

Unscheduled local laboratory assessments may be performed if medically indicated to document a (potential) AE for decision making (e.g. dose modifications).

Abnormal laboratory parameters that are clinically significant, induce signs or symptoms, require therapeutic intervention (e.g. hematologic abnormality that requires transfusion) or require changes in trial treatment, will be recorded on the AE CRF page, as applicable according to Section 7.2.3. The severity of laboratory data will be graded using the CTCAE version 4.03.

Additional analyses are left to the discretion of the Investigator.

TABLE A16
Clinical Laboratory Parameters Collection Plan

| Test | |
|---|---|
| Category | Test Name |
| Hematology | WBC count with differential (neutrophils, lymphocytes, |
| | monocytes, eosinophils, and basophils), differential other |
| | (if available), hemoglobin and platelet count. |
| Biochemistry | Sodium, potassium, uric acid, urea or Blood Urea Nitrogen, |
| | creatinine, eGFR*, fasting glucose, corrected calcium**, |
| | magnesium, phosphorous and albumin. |
| | AST (SGOT), ALT (SGPT), TBIL, direct bilirubin, GGT |
| | and ALP, amylase, lipase and Lactate Dehydrogenase |
| | (LDH). |
| Coagulation | aPTT, INR |
| Other Tests | Serum and urine pregnancy test. |
| (as | FSH, estradiol for confirmation of menopausal status |
| applicable) | (pre/post). |
| | Note: FSH and estradiol are not recorded in CRF, but used |
| | to determine menopausal status |

*eGFR is only required at screening and will be calculated using the 4-variable MDRD formula: eGFR in mL/min per 1.73 m² = 175 × SerumCr^{-1.154} × age^{-0.203} × 1.212 (if patient is black) × 0.742 (if female).
**If corrected calcium is not provided by the local laboratory, a recommended formula is: Corrected calcium = (0.8 × [normal albumin − patient's albumin]) + serum calcium.

### 6.4.2.5.1. Hematology

Hematology tests are to be performed according to the schedule outlined in Table A12. For details of the hematology panel, see Table A16.

### 6.4.2.5.2. Biochemistry

Biochemistry tests are to be performed according to the schedule in Table A12. For details of the biochemistry panel, see Table A16.

### 6.4.2.5.3. Coagulation

INR and aPTT are to be performed according to the schedule outlined in Table A12.

### 6.4.2.5.4. Pregnancy Tests and Assessments of Menopausal Status/Fertility

When applicable, FSH and estradiol are to be performed according to Table A12 and Table A16. If applicable, FSH and estradiol will be done at screening for eligibility and for confirmation of the menopausal status (see Inclusion Criterion #3).

A serum pregnancy test must be performed for all women of CBP at screening. During the Treatment Phase, pregnancy test will be done as follows:
Every cycle from C1 thru C6: urine or serum pregnancy test, according to Table A12.
From C7 up until 36 months from randomization:
   In the Investigational arm: urine or serum pregnancy test will be done monthly. It is acceptable that urine tests are done by the patient at home and that the results are promptly communicated to the Investigator or designee (e.g. by phone). On the months when trial visits are scheduled according to Table A12, pregnancy tests can be done at the site.
   In the Control arm: urine or serum pregnancy tests every 3^{rd} cycle.
EOT visit: urine or serum pregnancy test.

### 6.4.2.6. Electrocardiogram

Standard 12-lead ECG assessments will be performed (in the supine position) after the patient has been resting for approximately 10 minutes prior, at each time point indicated in Table A17.

All ECGs collected during the trial (including unscheduled ECGs with clinically relevant findings) will be transmitted to a central laboratory and will be centrally reviewed by an independent reviewer. For each ECG transmitted, an ECG report will be sent from the central laboratory to the site.

At screening, the central laboratory ECG assessment will be used to define patient's eligibility according to Inclusion Criterion #15. Note: In order to ensure the ECG evaluation is received by the site from the central laboratory for eligibility assessment, it is advisable to perform the ECG at least 3 business days prior to the scheduled randomization date.

After randomization, the Investigator (or other site's qualified physician) will assess the ECGs and will determine the QTcF duration and any potential ribociclib dose adjustment (see Section 5.2.1.2.2). If an ECG report from the central laboratory shows a QTcF ≥481 msec, ribociclib dosing should be interrupted (if not previously done) and patient should be managed according to Section 5.2.1.2.2. Interpretation of the tracing must be made by a site's qualified physician and documented on the CRF. Local cardiologist ECG assessment may be performed at any time during the trial at the discretion of the Investigator.

When a pre-dose ECG is to be collected, then the ECG measurement must occur before dosing of the trial treatment.

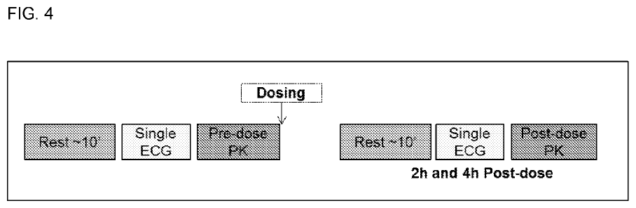

If a pre-dose PK sample is to be obtained on the same day as ECG, then the sample should be collected after the ECG and before dosing of the trial treatment. See FIG. 4 and Section 5.1.2.1.1 for additional guidance on the timing of ECG in relation to ribociclib dosing and PK sampling, at C1 D15.

TABLE A17
ECG Collection Plan

| Cycle | Patients | Day | Time | ECG Type |
|---|---|---|---|---|
| Screening | All | −28 to −1 | Anytime | Single 12 Lead |
| 1 | All | Day 1 | Pre-dose¹ | Single 12 Lead |
| | All | Day 15² | Pre-dose¹ | Single 12 Lead |
| | All | | 2 h post-dose | Single 12 Lead |
| | | | (±15 min) | |
| | All | | 4 h post-dose | Single 12 Lead |
| | | | (±15 min) | |
| 2 | All | Day 1 | Pre-dose¹ | Single 12 Lead |
| | All | Day 15 | Pre-dose¹ | Single 12 Lead |
| | | | 2 h post-dose | Single 12 Lead |
| | | | (±15 min) | |
| 3 | All | Day 1 | Pre-dose¹ | Single 12 Lead |
| 6 | All | Day 1 | Pre-dose¹ | Single 12 Lead |
| 7 and | Patients | Day 1 | Pre-dose | Single 12 Lead |
| thereafter | with | | | |
| every 3^{rd} | QTcF | | | |
| cycle | ≥481 | | | |
| (i.e. 7, 10, 13 | msec | | | |
| etc.) until 36 | at any | | | |
| months from | time | | | |
| rando- | prior to | | | |
| mization³ | cycle 7 | | | |

| | | |
|---|---|---|
| EOT | Anytime | Single 12 Lead |
| Unscheduled ECG | Anytime | Single 12 Lead |

¹The exact date and time of dosing must be recorded on the appropriate CRF.
²ECG assessment is to be done prior to PK sampling.
³Or until ribociclib discontinuation (in Investigational arm) or ET discontinuation (in Control arm), in cases where treatment is discontinued prior to completing 36 months.

The QTcF values using Fridericia's correction (formula is provided below) will be reviewed centrally.

$$QTcF = \frac{QT}{\sqrt[3]{RR}}$$

If any of the ECG reading after randomization includes a new clinically-relevant abnormal ECG finding or a QTcF value of 481 msec, ribociclib must be interrupted, ECG must be repeated and management guidelines detailed in Table A9 must be followed.

An unscheduled ECG may be repeated at the discretion of the Investigator at any time during the trial and as clinically indicated.

Each ECG tracing should be labeled with the trial number, patient number, date, and kept in the source documents at the trial site. Clinically significant ECG abnormalities present at screening when the patient signed PICF should be reported on the Medical History CRF page. New or worsened clinically significant findings occurring after informed consent must be recorded on the AE CRF page.

### 6.4.3. Pharmacokinetics Assessments

Approximately 130 patients from the Investigational arm will be considered as the PK subset. Plasma samples for ribociclib determination will be obtained from these patients at the time points indicated in Table A18.

TABLE A18
PK Blood Collection Schedule in Patients in the PK Subset

| | | Scheduled Time | Blood |
|---|---|---|---|
| | | Point Relative to | Volume |
| Cycle | Day | Dosing (Sampling Window) | (mL) |
| 1 | 15 | Pre-dose (0 h)^{a} | 2 |
| 1 | 15 | 2 h post-dose (±15 min) | 2 |
| 1 | 15 | 4 h post-dose (±30 min) | 2 |

^{a}Collect PK sample immediately before drug administration.

Refer to Sections 5.1.2.1.1 and 6.4.2.6 for additional guidance on the timing of collection of PK samples in relation to ribociclib dosing and ECG at C1D15.

All efforts will be made to obtain the PK samples at the scheduled nominal time relative to dosing. However, samples obtained within the sampling window of the planned time will be considered protocol compliant.

Refer to Section 5.1.2.1.1 for guidance on data to be collected on the CRF on the PK sampling day. If vomiting occurs during the course of treatment, no re-dosing of the patient is allowed, and medication should resume on the next day. The occurrence and frequency of any vomiting must be noted in the AE section of the CRF. In addition, the date and exact time of vomiting should only be recorded if it occurs within 4 hours of dosing on the day of PK sampling.

In the event the pre or post-dose samples are not collected on C1D15, every effort should be made to collect makeup samples between Day 16 and Day 21 of C1, following the same rules as described above.

PK samples will be analyzed using a liquid chromatography-tandem mass spectrometry (LC-MS/MS) method with a lower limit of quantification (LLOQ) of approximate 1.00 ng/mL. Any results below the LLOQ and any missing samples will be recorded accordingly. Leftover plasma from analysis may be used for exploratory metabolite assessments or other bioanalytical purposes (e.g. cross check between different sites, stability assessment).

Instructions for collection, preparation and shipment can be found in the applicable Laboratory Manual.

### 6.4.4. Biomarkers

Genetic aberrations that lead to an upregulation in the CDK signaling are observed frequently in BC. Investigating aberrations of the genes implicated in CDK pathway in patients enrolled in this trial will, therefore, allow for the assessment of the potential predictive and/or prognostic biomarkers in patients with EBC. Biomarkers indicative of resistance to treatment and mechanisms underlying disease recurrence will also be investigated. While the goal of the biomarkers analysis is to provide supportive data for the clinical trial, there may be circumstances when a decision is made to stop a collection, or not perform or discontinue an analysis due to either practical or strategic reasons. For example, there may be inadequate sample number, issues related to the quality of the sample or issues related to the assay that preclude analysis. Alternatively, there may be insufficient efficacy to allow for correlative analyses due to a limited number of samples. Therefore, depending on the results obtained during the trial, collection/analysis of some samples may be omitted at the discretion of Novartis.

Tumor and blood samples will be collected according to Table A19 from all patients, except those enrolled in China. Biomarker samples collection is fixed according to the calendar, regardless of any treatment interruption.

All assessments will be performed by a Novartis designated laboratory. Instructions for collection, preparation and shipment can be found in the applicable Laboratory Manual.

TABLE A19
Biomarker Sample Collection Plan

| | | | | |
|---|---|---|---|---|
| Sample Type | Requirement | Visit | Time point | Volume |

Tumor samples (from all patients, except those enrolled in China)

| | | | | |
|---|---|---|---|---|
| Archival paraffin tumor | Mandatory | At screening: | NA | N/A |
| tissue from surgical | | confirmation of | | |
| specimen (or, in patients | | sample's | | |
| who underwent | | availability for | | |
| neoadjuvant therapy and | | submission to | | |
| had a pathological | | central | | |
| complete response, at | | laboratory | | |
| the time of the initial | | | | |
| diagnosis or before the | | | | |
| administration of | | | | |
| neoadjuvant therapy) | | | | |
| (Tumor block [preferred] | | | | |
| OR 20 unstained slides [15 | | | | |
| for patients who received | | | | |
| neoadjuvant therapy]). | | | | |
| Archival paraffin tissue | If available | | | |
| from the breast tumor at | | | | |
| time of the initial | | | | |
| diagnosis | | | | |
| (Tumor block [preferred] | | | | |
| OR 20 unstained slides) | | | | |
| Paraffin tumor tissue | Mandatory | At local, regional | Within 4 weeks of | N/A |
| from recurrence | (unless not safe | and distant | local, regional | |
| (Tumor block [preferred] | for the patient) | recurrence until | and distant | |
| OR 20 unstained slides) | | confirmed distant | recurrence and | |
| | | recurrence | must be before | |
| | | | new treatment is | |
| | | | initiated | |

Blood samples (from all patients, except those enrolled in China)

| | | | | |
|---|---|---|---|---|
| Blood for ctDNA/ctRNA | Mandatory | Screening (up to | Pre-dose | 4 × 10 mL |
| | | C1D1, prior to | | |
| | | dosing), C4D1, | | |
| | | C13D1 and | | |
| | | C25D1 | | |
| | | Every 48 weeks | Pre-dose | 4 × 10 mL |
| | | (±3 weeks) after | | |
| | | C25D1 until | | |
| | | distant | | |
| | | recurrence | | |
| | | At local, regional | Within 4 weeks of | 4 × 10 mL |
| | | and distant | confirmed local, | |
| | | recurrence | regional and | |
| | | | distant | |
| | | | recurrence, and | |
| | | | must be before | |
| | | | new anti- | |
| | | | neoplastic | |
| | | | treatment is | |
| | | | initiated | |
| Blood for | Optional. | C1D1 or next | Any time | 6 mL |
| pharmacogenetic | Requires | available visit | | |
| analysis | patient's consent | within first 12 | | |
| | | weeks of | | |
| | | randomization | | |

### 6.4.4.1. Biomarker Assessments for Pathway Inhibition and Efficacy

### 6.4.4.1.1. Assessments in Tumor Tissue (Mandatory)

Tumor tissue samples will be collected for identifying biomarkers that may be predictive of benefit from ribociclib and exploring mechanisms underlying disease recurrence. Tumor tissue will be used to assess molecular alterations of several genes shown to be associated with HR-positive BC and cell cycle such as, but not limited, to CDK4, CDK6, CCND1, CCNE1, p53, PIK3CA, RB1.¹′ Protein (e.g. Ki67, pRb by IHC) and/or gene expression will also be assessed in tumor material. Broad panels or whole exome sequencing may be used in order to detect any additional mutation and assess their potential impact on clinical outcome.

Required tumor samples are (see also Table A19):
Mandatory archival paraffin tumor tissue from curative surgical specimen (see Inclusion Criterion #7): this may be a tumor block (preferred) or 20 unstained slides (15 slides for patients who received neoadjuvant therapy). Since archival samples may not be located at the institution where diagnosis was rendered, the investigational sites will be responsible for locating them and preparing the blocks or unstained slides (or having them prepared), if necessary.
If available, tumor tissue sample from the tumor biopsy specimen at the time of the initial diagnosis, or before the administration of neoadjuvant therapy. This sample is mandatory for patients who underwent neoadjuvant therapy and had a pathological complete response (see Inclusion Criterion #7).
Mandatory (unless unsafe for the patient per Investigator's discretion) tumor biopsy at the time of recurrence and before start of new anti-neoplastic therapy. For patients having synchronous local/regional and distant recurrence, if feasible and safe for the patient, a sample from both the local/regional recurrence and a distant site is required.

This tumor biopsy will be sent to a designated laboratory who will review the biopsy to assure quality and sufficient tumor tissue quantity before the initiation of new anti-neoplastic therapy. If the quality or quantity of the biopsy will not be satisfactory, the Investigator will receive notification, and another biopsy attempt will be made (if safe for the patient) before the start of new anti-neoplastic therapy.

### 6.4.4.1.2. Assessments in Blood (Mandatory)

Mandatory blood samples (4×10 mL) for plasma ctDNA/ctRNA will be collected as per Table A19.

ctDNA/ctRNA plasma samples will be used to assess mutations, copy number variations and expression of genes that are relevant to HR-positive BC (e.g. PIK3CA, ESR1) and the CDK4/6 pathway and/or cancer using the most sensitive and advanced technology at the time of testing. Use of ctDNA/ctRNA in plasma samples offers a unique opportunity to monitor mutations and gene expressions in a non-invasive manner during the course of treatment, allowing for the detection of changes in tumor burden and monitoring response to treatment and disease recurrence.⁵³⁻⁵⁶. Evaluation of mutations at time of recurrence may provide information regarding potential mechanisms of resistance to hormone and/or CDK4/6 targeted therapy.

### 6.4.4.2. Additional Biomarker Assessments (Optional)

This trial includes optional biomarker components that are hypothesis generating (i.e. discovery based research) and/or supported by an exploratory objective.

When remaining samples are available (tumor, blood, DNA, RNA) in sufficient quantity and quality and if allowed per local regulation, samples may be stored for up to 15 years from end of the trial and further analyzed to address scientific questions related to ribociclib or cancer, including research to help develop ways to detect, monitor or treat cancer. A decision to perform such exploratory biomarker research trials would be based on outcome data from this trial or from new scientific findings related to the drug class or disease, as well as reagent and assay availability.

These studies will only be performed in patients giving consent to this optional part of the trial.

### 6.4.4.3. Pharmacogenetic Assessments (Optional)

Whole blood will be collected for genomic DNA extraction for genome-wide association studies (GWAS), such as genotyping of human leukocyte antigen (HLA) and CYP3A4 to explore the inherited genetic factors (e.g. CYP3A4 polymorphism) that may affect the response (both adverse [e.g. neutropenia] and therapeutic effects) to treatment. Patient data from this trial may be combined with other trials to increase statistical power.

This sample will only be taken from patients giving consent to this optional part of the trial.

### 6.4.5. Resource Utilization Evaluations

Healthcare resource utilization will be evaluated in this trial as an exploratory endpoint to characterize the impact of trial treatment on this aspect of healthcare. In addition, these data may be used to support assessments to characterize the economic impact of trial treatment regimens.

Healthcare resource utilization data should be captured throughout the Treatment and Follow-up Phases, as described in Table A12.

Every effort should be made to collect data around health care utilization post-recurrence.

These data will be collected for 12 months following distant recurrence and should follow the same schedule as for post-recurrence PRO collection (see Table A20).

Hospitalization data of interest will focus on those hospitalizations reported as SAEs, as required in Section 7.3.1. Hospitalizations as a result of the following reasons are not to be reported:
Routine treatment or monitoring of the studied indication not associated with any deterioration in condition.
Elective or pre-planned treatment for a pre-existing condition that is unrelated to the indication under study and has not worsened since signing the informed consent.
Social reasons and respite care in the absence of any deterioration in the patient's general condition.

Information related to the length of stay (e.g. dates of admission, discharge), hospital ward facilities used (e.g. emergency department, intensive care unit, general ward, etc.), reasons for hospitalization as associated with the trial treatment regimen, disease and/or disease progression, or any other reason will be of interest; and hospital discharge information will be evaluated.

Additionally, data on the following will be collected:
Visits to the Emergency Room which do not result in subsequent hospitalization, along with the reason/cause for the visit.
Additional visits (non protocol required) to the treating Investigator due to BC-related reasons or treatment-related AEs.

### 6.4.6. Patient Reported Outcomes

The following questionnaires will be administered to the patient at the visits indicated in Table A12 and Table A20:
European Organization for Research and Treatment of Cancer's (EORTC) core quality of life questionnaire (EORTC QLQ-C30, version 3.0)
EORTC BC specific quality of life questionnaire (EORTC QLQ-BR23, version 1.0)
Generic health utility measure EuroQoL 5-level instrument (EQ-5D-5L)
Hospital Anxiety and Depression Scale (HADS) questionnaire

Collection of all PRO measures has a ±3-day window unless otherwise indicated. Their collection is fixed according to the calendar, taking as a reference the randomization date (not the date of a previous assessment), regardless of treatment interruptions.

The EORTC QLQ-C30, version 3.0, EORTC QLQ-BR23, version 1.0, and the EQ-5D-5L will be used to evaluate PRO measures of health-related QOL, functioning, disease symptoms and treatment-related side effects. The EORTC QLQ-C30, EORTC QLQ-BR23 and the EQ-5D-5L are recognized reliable and valid measures frequently used in clinical trials of patients with BC.⁵⁷⁻⁵⁹ In addition, the HADS questionnaire will be used to determine the levels of anxiety and depression experienced by the patient.⁶⁰

All paper questionnaires should be administered according to the guidance below:
Questionnaires will be in the patient's local language.
Will be administered to the patient at the beginning of the applicable visit, prior to any interaction with the Investigator including any tests, treatments or receipt of results from any tests to avoid biasing the patient's perspective.
Patients should be given sufficient space and time to complete all questionnaires and all administered questionnaires should be reviewed for completeness. If missing responses are noted, patients should be encouraged to complete any missing responses.
If the patient is not capable to complete the questionnaires personally, the designated study coordinator may obtain patient responses by reading out loud each question, followed by the corresponding response categories, and entering the patient's response. No help should be provided to the patient by any person other than the designated study coordinator. The coordinator should not influence patient's responses. The study coordinator cannot rephrase the question or translate the question into simpler language; the question has to be read verbatim.
Attempts should be made to collect responses to all questionnaires for all patients, including from those who discontinue prior to the trial completion. If a patient refuses to complete questionnaires or in the case a local language should not be available for the patient to complete questionnaires, this should be documented in source records. Patient's refusal or if a local language is not available to complete questionnaires are not protocol deviations.
Investigators should not encourage the patient to change responses reported in questionnaires.

Completed questionnaires, including both responses to the questions and any unsolicited comments written by the patient, should be reviewed and assessed by the Investigator before or during the visit, for responses which may indicate potential AEs or SAEs. If an AE or SAE is confirmed then the physician should record the event as instructed in Section 7 of this protocol.

After completion at each applicable time point, questionnaires will be entered in the CRF by the site staff.

Refer to Appendix 5. Patient Reported Outcomes for a sample of each questionnaire and more information about them.

TABLE A20
Patient Reported Outcomes (PRO) Collection Plan

| Questionnaire | Visit | Day | Time |
|---|---|---|---|
| EORTC | Screening | Day −28 to −1 | For assessments |
| QLQ-C30 | Treatment | After randomization | before |
| EORTC QLQ- | and | every 12 weeks (±2 | recurrence, |
| BR23 | Follow-up | weeks) during the | questionnaires |
| EQ-5D-5L | Phases | first 24 months, | should be |
| HADS | (including | every 24 weeks | administered |
| | EOT) | (±3 weeks) | prior to |
| | | thereafter until | any clinical |
| | | distant recurrence. | assessments, |
| | | At EOT. | drug dosing or |
| | | At confirmation | diagnostic |
| | | of first | testing; for the |
| | | recurrence. | assessment at |
| | | At confirmation | recurrence, |
| | | of distant | questionnaires |
| | | recurrence | should |
| | | (if the first | be administered |
| | | recurrence was | within |
| | | not a distant | 30 days of |
| | | recurrence). | clinical |
| | | During first 12 | suspicion of |
| | | months after | recurrence. |
| | | confirmation | |
| | | of distant | |
| | | recurrence: | |
| | | Every 12 weeks | |
| | | (±2 weeks) if | |
| | | confirmation of distant | |
| | | recurrence happened | |
| | | during first 24 | |
| | | months after date of | |
| | | randomization, or | |
| | | Every 24 weeks | |
| | | (±3 weeks) if | |
| | | confirmation of distant | |
| | | recurrence happened after | |
| | | first 24 months after | |
| | | date of randomization. | |

### 7. SAFETY MONITORING AND REPORTING

### 7.1. Period of Observation

Refer to Table A21 for the period of observation for AEs (non-serious) and SAEs.

TABLE A21
Reporting Period for AEs and SAEs

| Period | AEs (non-serious) | SAEs |
|---|---|---|
| From: Main PICF | Only if related | All (regardless |
| signature | to trial | of relationship) |
| To: C1D1 (prior to dosing) | participation | |
| From: C1D1 (during/ | All (regardless | All (regardless |
| after dosing) | of relationship) | of relationship) |
| To: 36 months from | | |
| randomization^{a} | | |
| From: 36 months from | Not reportable | All (regardless |
| randomization | | of relationship) |
| To: 30-Day Safety | | |
| Follow-up visit | | |
| From: 30-Day Safety | Not reportable | Only if related |
| Follow-up visit | | to trial |
| To: End of trial | | treatment |

^{a}In the Investigational arm, collection according to 2^{nd} row in the Table should continue up until the 30-Day Post Ribociclib Safety Follow-up visit. In cases that this visit takes place prior to completing 36 months of ribociclib treatment due to premature ribociclib discontinuation, collection will extend until 36 months from randomization.
If ET (in Control arm) or ribociclib + ET (in Investigational arm) are discontinued prior to completing 36 months of treatment, collection of all AEs (regardless of relationship) will stop at time of the 30-Day Safety Follow-up visit.

### 7.2. Adverse Events

### 7.2.1. Definition and Reportinq

An AE is defined as the appearance of (or worsening of any pre-existing) undesirable sign(s), symptom(s), or medical condition(s).

AEs (including laboratory abnormalities that constitute AEs) should be described using a diagnosis whenever possible, rather than individual underlying signs and symptoms. When a clear diagnosis cannot be identified, each sign or symptom should be reported as a separate AE.

The occurrence of AEs should be sought by non-directive questioning of the patient. AE also may be detected when they are volunteered by the patient during the screening process or between visits, or through physical examination, laboratory test, or other assessments.

As far as possible, each AE should be evaluated to determine:
Severity grade (per NCI CTCAE v4.03. Refer to Table A22 for severity grade scale for events not listed in NCI CTCAE v4.03)
Duration (start and end dates)
Relationship to the trial treatments
Action taken with respect to trial treatment
Other action taken (e.g. concomitant medications or non-drug therapy given)
Whether it is serious, where an SAE is defined as in Section 7.3.1, and which seriousness criteria have been met
Outcome.

Adverse reactions are defined as all untoward and unintended responses to an IMP related to any dose administered. “Responses to an IMP” means that a causal relationship between a medicinal product and an adverse event is at least a reasonable possibility, i.e. the relationship cannot be ruled out. For the assessment of AE causality, Investigators should use their knowledge of the patient, details about the AE, the circumstances surrounding the event and an evaluation of any potential alternative causes to determine whether or not an AE is considered to be related to the trial treatment or not.

TABLE A22
Adverse Event Severity Grading Scale for Events
Not Listed in NCI CTCAE v4.03

| Grade | Description |
|---|---|
| 1 | Mild; asymptomatic or mild symptoms; |
| | clinical or diagnostic observations only; |
| | intervention not indicated. |
| 2 | Moderate; minimal, local or noninvasive |
| | intervention indicated; limiting age-appropriate |
| | instrumental Activities of Daily Living (ADL)*. |
| 3 | Severe or medically significant but not |
| | immediately life-threatening; hospitalization or |
| | prolongation of hospitalization indicated; |
| | disabling; limiting self care ADL**. |
| 4 | Life-threatening consequences; urgent |
| | intervention indicated. |
| 5 | Death related to AE. |

*Instrumental ADL refer to preparing meals, shopping for groceries or clothes, using the telephone, managing money, etc.
**Self care ADL refer to bathing, dressing and undressing, feeding self, using the toilet, taking medications, and not bedridden.

An AE should be followed until its resolution, until it is judged to be permanent, the patient is lost follow-up, or the patient withdraws consent. Assessment should be made at each visit (or more frequently, if necessary) of any changes in:
Severity
Suspected relationship to the trial treatment
Action taken with trial treatment
Interventions required to treat it
Outcome

### 7.2.2. Worsening of a Pre-Existing Condition

Worsening of a pre-existing condition after PICF signature should be recorded in the AE CRF.

Conditions that were already present at the time of informed consent should be recorded in the Medical History page of the patient's CRF.

### 7.2.3. Laboratory Test Abnormalities

Laboratory test abnormalities that constitute an AE in their own right (i.e. are considered clinically significant, induce signs or symptoms, require therapeutic intervention [e.g. hematologic abnormality that requires transfusion] or require changes in trial treatment), should be recorded on the AE CRF. Laboratory abnormalities, that do not meet the definition of an AE, should not be reported as AEs. A Grade 3 or 4 event as per CTCAE does not automatically indicate a SAE unless it meets the definition of serious as defined below and/or as per

Investigator's discretion. A dose hold of medication for the laboratory test abnormality may be required by the protocol in which case the abnormality would still, by definition, be an AE and must be reported as such.

The same period of observation as for other non-serious AEs or SAEs applies to laboratory test abnormalities that qualify as AE/SAE (see Table A21: Reporting Period for AEs and SAEs). Whenever possible, a diagnosis, rather than a symptom should be provided (e.g. anemia instead of low hemoglobin). Laboratory abnormalities that meet the criteria for AEs should be followed until they have returned to normal or an adequate explanation of the abnormality is found. When an abnormal laboratory or test result corresponds to a sign/symptom of an already reported AE, it is not necessary to separately record the lab/test result as an additional event.

### 7.2.4. Recurrent Adverse Events

A recurrent AE is one that resolves between patient evaluation time points and subsequently recurs. Each recurrence of an AE should be recorded separately on the AE CRF.

### 7.2.5. Death

Death should be considered an outcome and not a distinct event. The event or condition that caused or contributed to the fatal outcome should be recorded as the single medical concept on the AE CRF.

Information about any deaths (related to an AE or not) will also be collected through a Death CRF.

### 7.2.6. Recurrence of Breast Cancer

BC recurrence (including those resulting in fatal outcomes), if documented by use of appropriate method (for example, confirmed recurrence per STEEP Criteria), should not be reported as an AE, including SAE. Progression of malignancy should not be reported as an AE, including SAE. AEs separate from the progression of malignancy (e.g. deep vein thrombosis at the time of progression or hemoptysis concurrent with finding of disease progression, etc.) will be reported as per usual guidelines used for such events with proper attribution regarding relatedness to the drug.

### 7.2.7. Second Primary Malignancy

Any second primary malignancy must be reported as an AE; if the event meets at least one seriousness criteria it must be reported as SAE.

### 7.3. Serious Adverse Events

### 7.3.1. Definition

A SAE is defined as any adverse event meeting at least one of the following conditions:
Is fatal
Is life-threatening (i.e. the adverse event, in the view of the Investigator, places the patient at immediate risk of death at the time of occurrence)
Requires inpatient hospitalization or prolongation of existing hospitalization
Results in persistent or significant disability/incapacity
Constitutes a congenital anomaly/birth defect
Is medically significant, i.e. defined as an event that jeopardizes the patient or may require medical or surgical intervention to prevent one of the outcomes listed above

Note that hospitalizations for the following reasons should not be reported as SAEs:
Routine treatment or monitoring of the studied indication, not associated with any deterioration in condition.
Elective or pre-planned treatment for a pre-existing condition that is unrelated to the indication under study and has not worsened since signing the informed consent.
Social reasons and respite care in the absence of any deterioration in the patient's general condition.
Treatments occurring on an emergency outpatient basis that does not result in hospital admission and involves an event not fulfilling any of the definitions of a SAE given above.

All cases of confirmed DILI (see section 5.2.1.4) must be reported as an SAE regardless of the severity.

### 7.3.2. Reporting

To ensure patient safety, every SAE, regardless of suspected causality, occurring after the patient has provided informed consent and until at least the 30-Day Safety Follow-up visit (see Table A20), must be immediately reported to Novartis, without undue delay and under no circumstance later than 24 hours of learning of its occurrence.

Any additional information for the SAE including complications, progression of the initial SAE, and recurrent episodes must be immediately reported as follow-up to the original episode no later than 24 hours after the Investigator receives the follow-up information. An SAE occurring at a different time interval or otherwise considered completely unrelated to a previously reported one should be reported separately as a new event.

Any SAEs experienced after the 30-Day Safety Follow-up visit should be immediately reported to Novartis no later than 24 hours of learning of its occurrence if the Investigator suspects a causal relationship to the trial treatment.

Information about all SAEs is collected and recorded on the Serious Adverse Event Report Form; all applicable sections of the form must be completed in order to provide a clinically thorough report. The Investigator must assess and record the relationship of each SAE to each specific trial treatment (if there is more than one), complete the SAE Report Form in English, and submit the completed form to Novartis no later than 24 hours of learning about the occurrence. Detailed instructions regarding the SAE submission process and requirements for signatures are to be found in the Investigator folder provided to each site.

Follow-up information is submitted in the same way as the original SAE Report. Each re-occurrence, complication, or progression of the original event should be reported as a follow-up to that event regardless of when it occurs. The follow-up information should describe whether the event has resolved or continues, if and how it was treated, and whether the patient continued or was discontinued from trial treatment or participation.

If the SAE is thought to be related to ribociclib and is not previously documented in the IB or package Insert (new occurrence), a Novartis Chief Medical Office and Patient Safety (CMO&PS) department associate may urgently require further information from the Investigator for Health Authority reporting. Novartis may need to issue an Investigator Notification (IN), to inform all Investigators involved in any trial with the same drug that this SAE has been reported.

Suspected Unexpected Serious Adverse Reactions (SUSARs) will be collected and reported to the CAs and IEC/IRBs in accordance with Directive 2001/20/EC or as per national regulatory requirements in participating countries.

### 7.4. Pregnancies

To ensure patient safety, each pregnancy occurring while the patient is on trial treatment must be immediately reported to Novartis, without undue delay and under no circumstance later than 24 hours of learning of its occurrence.

The pregnancy should be followed up to determine outcome, including spontaneous or voluntary termination, details of the birth, and the presence or absence of any birth defects, congenital abnormalities, or maternal and/or newborn complications. Pregnancy follow-up should be recorded on the same form and should include an assessment of the possible relationship to the investigational any pregnancy outcome.

Any SAE experienced during pregnancy (e.g. an event in the fetus, an event in the mother during or after the pregnancy, or a congenital anomaly/birth defect in the child) must be reported separately as an SAE.

### 7.5. Warnings and Precautions

No evidence available at the time of the approval of this clinical trial protocol indicate that special warnings or precautions were appropriate, other than those noted in the provided ribociclib IB. Additional safety information collected between IB updates will be communicated in the form of IN. This information will be included in the PICF and should be discussed with the patient during the trial as needed.

### 7.6. Independent Data Monitoring Committee

This trial will institute an IDMC which will function independently of all other individuals associated with the conduct of this clinical trial, including the site Investigators participating in the trial. The IDMC will be constituted prior to the randomization of the first patient.

The IDMC will be responsible for reviewing the safety results and overseeing the safety data accruing in the trial at regular intervals of approximately every six months, provided that sufficient patients have been randomized. Additionally, the IDMC will perform a safety review when the first patient has completed 12 months of trial treatment. In addition, if requested by the IDMC Chair, additional safety reviews may be performed. The IDMC will also review efficacy data at the planned interim analyses.

It is expected that the IDMC will consist at a minimum of two physicians with appropriate disease area qualifications, one patient advocate and one statistician. There will be a meeting with the IDMC describing their roles and responsibilities and discussing potential data format and process issues prior to the finalization of IDMC charter and the interim analysis plan.

### 8. STATISTICAL METHODS AND DATA ANALYSIS

It is planned that the data from all sites participating in the trial will be combined, so that an adequate number of patients are available for analysis. A designated vendor will perform the interim and final analyses.

Any data analyses performed independently by any Investigator should be submitted to Novartis before publication or presentation.

### 8.1. Analysis Sets

### 8.1.1. Full Analysis Set

The Full Analysis Set (FAS) comprises all patients to whom trial treatment has been assigned by randomization. According to the intent-to-treat principle, patients will be analyzed according to the treatment arm they were randomized to and strata they have been assigned to during the randomization procedure.

### 8.1.2. Safety Set

The Safety Set includes all randomized patients who received any trial treatment (i.e. at least one dose of ribociclib or ET). Patients will be analyzed according to the trial treatment actually received.

The actual treatment received corresponds to:
Ribociclib+ET if patients took at least one dose of ribociclib
ET if ribociclib was never received

### 8.1.3. Per-Protocol Set

The Per-Protocol Set (PPS) consists of a subset of the patients in the FAS who are compliant with requirements of the protocol. All protocol deviations or conditions leading to exclusion from the PPS will be detailed in specific trial document(s). Sensitivity analyses of the primary endpoint of iDFS may be performed using data from the PPS if the FAS and PPS differ and if the primary analysis is significant.

### 8.1.4. Pharmacokinetics Analysis Set

The Pharmacokinetics Analysis Set (PAS) consists of all patients who received at least one dose of ribociclib and have at least one evaluable post-dose concentration measurement.

### 8.2. Patient Demographics/Other Baseline Characteristics

Demographic and other baseline data including disease characteristics/prognostic data will be listed and summarized descriptively by treatment arm for the FAS.

Categorical data will be presented as frequencies and percentages. For continuous data, mean, standard deviation, median, minimum, and maximum will be presented.

Relevant medical histories and current medical conditions at baseline will be summarized by system organ class, preferred term and treatment arm.

### 8.3. Treatments (Trial Treatment, Concomitant Therapies, Compliance)

The Safety Set will be used for the analyses in this section. Categorical data will be summarized as frequencies and percentages. For continuous data, mean, standard deviation, median, minimum, and maximum will be presented.

The duration of exposure in months to ribociclib and ET as well as the dose intensity (computed as the ratio of actual cumulative dose received and actual duration of exposure) and the relative dose intensity (computed as the ratio of dose intensity and planned dose intensity) will be summarized by means of descriptive statistics. The cumulative doses and average daily doses of ribociclib and ET for each patient will be summarized using descriptive statistics (e.g. mean, standard deviation and median).

The number of patients with dose adjustments (reduction, interruption, or permanent discontinuation) and the reasons will be summarized by treatment arm and all dosing data will be listed.

Concomitant medications and significant non-drug therapies prior to and after the start of the trial treatment will be listed and summarized according to the Anatomical Therapeutic Chemical (ATC) classification system, by treatment arm.

### 8.4. Primary Objective

The primary objective of this trial is to compare iDFS (per STEEP System)³⁵ for ribociclib and ET versus ET in pre and postmenopausal women and men with HR-positive, HER2-negative EBC.

### 8.4.1. Variable

The primary efficacy variable of the trial is iDFS, defined as the time from the date of randomization to the date of the first event of local invasive breast recurrence, regional invasive recurrence, distant recurrence, death (any cause), contralateral invasive BC, or second primary non-breast invasive cancer (excluding basal and squamous cell carcinomas of the skin). iDFS events will be assessed locally. Censoring conventions are provided below in Section 8.4.3.

### 8.4.2. Statistical Hypothesis, Model, and Method of Analysis

Assuming proportional hazards for iDFS, the following statistical hypotheses will be tested to address the primary efficacy objective:

$${H}_{0} : {θ}_{1} \geq 1 vs . {H}_{a} : {θ}_{1} < 1$$

where θ₁ is the iDFS hazard ratio (ribociclib+ET versus ET).

The primary efficacy analysis to test this hypothesis and compare the two treatment arms will consist of a stratified log-rank test at an overall one-sided 2.5% level of significance, based on the randomization stratification factors:
Menopausal status: premenopausal women and men vs. postmenopausal women
The AJCC 8^{th} edition Anatomic Stage Group: Anatomic Stage Group II vs. Anatomic Stage Group III
Prior neoadjuvant/adjuvant chemotherapy: yes vs. no
Geographical region: North America/Western Europe/Oceania vs. RoW

The primary efficacy variable, iDFS, will be analyzed at three interim analyses (first interim that may allow the trial to stop due to futility and second and third interim that may allow the trial to declare superior efficacy) and final analysis of a group sequential design, using a Lan-DeMets (O'Brien-Fleming) alpha spending function and a non-binding Lan-DeMets (O'Brien-Fleming) beta spending function. Analyses will be based on the FAS population according to the treatment arm and strata assigned at randomization. The iDFS distribution will be estimated using the Kaplan-Meier method, and Kaplan-Meier curves will be presented for each treatment arm. The iDFS at the end of each year along with 95% confidence intervals (CI) will be presented for each of the two treatment arms. The hazard ratio for iDFS will be calculated, along with its 95% Cl, from a stratified Cox model using the same stratification factors as for the log-rank test.

If the primary efficacy analysis is statistically significant, additional descriptive analyses of iDFS will also be performed approximately two years after the primary iDFS analysis and at end of trial.

### 8.4.3. Handling of Missing Values/Censoring/Discontinuations

iDFS will be censored if no iDFS event is observed prior to the analysis cut-off date. The censoring date will be the date of last recurrence assessment on or prior to data cut-off.

### 8.4.4. Supportive Analyses

As sensitivity analyses performed in the FAS, the hazard ratio and 95% Cl for iDFS will be obtained from:
An unstratified and covariate unadjusted Cox model.
A stratified and covariate adjusted Cox model. The covariates to be included will be detailed in the SAP.

iDFS will also be analyzed based on the PPS, using the same analysis conventions as in the primary efficacy analysis, if the FAS and PPS differ and if the primary analysis is significant.

If the primary analysis is statistically significant, subgroup analyses to assess the homogeneity of the treatment effect across demographic and baseline disease characteristics (e.g. stage, menopausal status, prior anti-cancer treatment, etc.) will be performed. The subgroups to be considered will be defined in the SAP.

Patterns of censored data will be examined by the treatment arms using descriptive statistics (the numbers of censored patients and reasons for censoring).

### 8.5. Secondary Objectives

### 8.5.1. Secondary Efficacy Objectives

All secondary efficacy objectives will be analyzed at the primary analysis for iDFS (2^{nd} or 3^{rd} interim analyses if the efficacy boundary is crossed or final iDFS analysis) and at the time of end of trial (see Section 3.3). Each secondary efficacy endpoint will be analyzed in the FAS population according to the randomized treatment arm and strata assigned at randomization. The distribution of each efficacy objective will be estimated using the Kaplan-Meier method and compared between the two treatments arms using a stratified log-rank test at one-sided 2.5% level of significance, using strata assigned at randomization. The hazard ratio will be calculated, along with its 95% Cl, using a stratified Cox model based on strata assigned at randomization. Secondary endpoints will not be adjusted for multiple comparisons.

### 8.5.1.1. Recurrence-Free Survival

RFS is defined as the time from date of randomization to date of first event of local invasive breast recurrence, regional invasive recurrence, distant recurrence, or death (any cause).

Patients who do not have a RFS event will be censored at the last recurrence assessment on or prior to the data cut-off.

### 8.5.1.2. Distant Disease-Free Survival

DDFS is defined as the time from date of randomization to date of first event of distant recurrence, death (any cause), or second primary non-breast invasive cancer (excluding basal and squamous cell carcinomas of the skin). Patients who do not have a DDFS event will be censored at the last recurrence assessment on or prior to the data cut-off.

### 8.5.1.3. Overall Survival

OS is defined as the time from date of randomization to date of death due to any cause. If a patient is not known to have died, then OS will be censored at the latest date the patient was known to be alive (on or prior to the data cut-off).

OS will be analyzed at the primary analysis for iDFS (2^{nd} or 3^{rd} interim analyses if the efficacy boundary is crossed or the final iDFS analysis), approximately two years after the primary iDFS analysis, and at the time of end of trial (See Section 3.3). The expected numbers of OS events at the time of the 2^{nd} and 3^{rd} interim analyses, the final iDFS analysis, two years thereafter and end of trial are approximately 187, 225, 271, 474 and 641 respectively, assuming a 5-year OS rate of 87% in the control arm, a hazard ratio of 0.85 between the two arms and assuming approximately 10% of patients will be lost to follow-up for OS at the time of the final iDFS analysis (i.e. hazard rate of lost to follow-up=0.004 per month).

### 8.5.2. Patient Reported Outcomes

The physical functioning sub-scale score of the EORTC QLQ-C30 will be the primary PRO variable of interest. The global health status/QoL scale score of the EORTC QLQ-C30, emotional functioning and social functioning sub-scale scores of the EORTC QLQ-C30, the BC symptoms scale of the EORTC QLQ-BR23, the VAS of the EQ-5D-5L, the anxiety domain and depression domain scores of HADS will be the secondary PRO variables of interest. The FAS will be used for analyzing PRO data.

Descriptive statistics will be used to summarize the PRO scores and absolute change from baseline scored scales at each scheduled assessment.

As the primary analysis of PROs and to best utilize the repeated PRO assessments, PRO scores in all sub-scales obtained from EORTC QLQ-C30, the breast symptoms score of QLQ-BR23, the VAS of EQ-5D-5L and the anxiety domain and depression domain scores of HADS will be analyzed using a repeated measures model for longitudinal data to assess the treatment effect over time. The differences in least square means between treatment and control arm, and the corresponding 2-sided 95% Cl at selected time points will be presented.

### 8.5.2.1. Handling of Missing Values/Censoring/Discontinuations

Missing items data in a scale will be handled based on each instrument manual. No imputation will be applied if the total or subscale scores are missing at a visit.

For the repeated measures analysis, patients with baseline and at least one non-missing post-baseline assessments will be included. All available data will be used in the repeated measures models for longitudinal data which assume that the missing scores at any time point are missing-at-random. Additional sensitivity analysis may be performed to assess the possible violation of missing-at-random assumption for the missing data mechanism if deemed appropriate. Details will be specified in the SAP.

### 8.5.3. Safety Objectives

### 8.5.3.1. Analysis Set and Grouping for the Analyses

For all safety analyses, the Safety Set will be used. All listings and tables will be presented by treatment arm.

The overall observation period will be divided into three mutually exclusive segments:
1. Pre-treatment period: from day of patient's informed consent signature to the day before first dose of trial treatment (any dose of ribociclib or ET on or after randomization)
2. On-treatment period: from day of first dose of trial treatment to 30 days after last dose of trial treatment
3. Post-treatment period: from 31 days after last dose of trial treatment

### 8.5.3.2. Adverse Events

Summary tables for AEs will include only AEs that started or worsened during the on-treatment period, the treatment-emergent AEs.

The incidence of treatment-emergent AEs (new or worsening from baseline) will be summarized by system organ class and/or preferred term, severity (based on CTCAE grades v4.03), type of AE, relation to trial treatment.

SAEs and non-serious AEs during the on-treatment period will be tabulated. All deaths (on-treatment and post-treatment) will be summarized.

All AEs, deaths and SAEs (including those from the pre and post-treatment periods) will be listed and those collected during the pre-treatment and post-treatment periods will be flagged.

### 8.5.3.3. Laboratory Abnormalities

Grading of laboratory values will be assigned programmatically as per NCI CTCAE version 4.03, The calculation of CTCAE grades will be based on the observed laboratory values only, clinical assessments will not be taken into account.

CTCAE Grade 0 will be assigned for all non-missing values not graded as 1 or higher. Grade 5 will not be used. Missing values will not be graded.

For laboratory tests where grades are not defined by CTCAE, results will be categorized as low/normal/high based on laboratory normal ranges.

The following summaries will be generated separately for hematology and biochemistry tests:
Listing of all laboratory data with values flagged to show the corresponding CTCAE v4.03 grades if applicable and the classifications relative to the laboratory normal ranges

For laboratory tests where grades are defined by CTCAE
Worst post-baseline CTCAE grade (regardless of the baseline status). Each patient will be counted only once for the worst grade observed post-baseline.
Shift tables using CTCAE grades to compare baseline to the worst on-treatment value

For laboratory tests where grades are not defined by CTCAE,
Shift tables using the “low”/“normal”/“high”/“low and high” classification to compare baseline to the worst on-treatment value.

In addition to the above-mentioned tables and listings, other exploratory analyses, for example figures plotting time course of raw or change in laboratory tests over time or box plots might be specified in the SAP.

### 8.5.3.4. Other Safety Data

### 8.5.3.4.1. ECG

Categorical Analysis of QT/QTc interval data based on the number of patients meeting or exceeding predefined limits in terms of absolute QT/QTc intervals or changes from baseline will be presented. In addition, a listing of these patients will be produced by treatment arm.

### 8.5.3.4.2. Vital Signs

Data on vital signs will be tabulated and listed, notable values will be flagged.

### 8.5.3.5. Supportive Analyses for Secondary Objectives

Main safety analyses will be repeated in subgroups of patients by treatment arm. The subgroups to be considered will be defined in the SAP.

### 8.5.3.6. Tolerability

Tolerability will be studied in terms of dose reductions and drug interruptions due to AE.

Reasons for dose reductions and interruptions will be listed and summarized by treatment arm. Tolerability of ET during both on-treatment period and post-treatment period (Section 8.5.3.1) will be summarized.

### 8.5.4. Pharmacokinetics Objectives

PK analysis will be done using the PAS. PK concentrations of ribociclib will be summarized using descriptive statistics by visit and scheduled time point. All PK concentration data will be listed as appropriate.

All concentrations below the LLOQ will be displayed in listings as zero with a flag and handled as zero in any calculations of summary statistics, but handled as missing for the calculation of the geometric means and their CV.

A model-based approach may be used to explore the potential relationships between efficacy endpoints or safety endpoints and ribociclib concentrations. Exploratory PK analysis may be conducted.

### 8.6. Exploratory Objectives

Exploratory endpoints will not be adjusted for multiple comparisons, and details may be documented in separate SAP.

### 8.6.1. Loco-Regional Recurrence Free Survival

LRRFS is defined as the time from date of randomization to date of first event of local (ipsilateral) invasive breast recurrence, regional invasive recurrence or death due to any cause. Patients who do not have a LRRFS event will be censored at the last recurrence on or prior to the data cut-off.

LRRFS will be analyzed at the primary analysis for iDFS (2^{nd} or 3^{rd} interim analyses if the efficacy boundary is crossed or the final iDFS analysis) and at the time of end of trial (See Section 3.3), if there is a sufficient number of events. LRRFS data will be listed and summarized by treatment arm.

### 8.6.2. Resource Utilization

Data relating to resource utilization (described in Section 6.4.5) will be used to support health economic evaluations.

Trial-specific analyses will focus on descriptive statistics of hospitalizations and hospital facility visits, occurring during the Treatment and Follow-up Phases and will be summarized by treatment arm. Summary statistics for hospitalizations will include the number of patients hospitalized, the total and average number and duration of hospitalizations, reason for hospitalization, type of hospital facility (e.g. emergency department, intensive care unit, general ward unit, etc.) and discharge summary.

Details of data analysis will be specified in the SAP as appropriate.

### 8.6.3. Use of Subsequent Anti-Neoplastic Therapy

Use of subsequent anti-neoplastic therapy will be characterized by incidence of subsequent anti-neoplastic therapy and by time to first anti-neoplastic therapy and time to first systemic anti-neoplastic therapy. These data will be listed and summarized by treatment arm. Duration of first systemic anti-neoplastic therapy may be explored in the two treatment arms, if there are a sufficient number of events.

### 8.6.4. Biomarkers

As a project standard, Novartis will analyze only biomarkers collected in the trial database. For exploratory markers, since the studies are not adequately powered to assess specific biomarker-related hypotheses, the goal of these exploratory statistical analyses should be considered as the generation of new scientific hypotheses. These hypotheses may be compared with results found in literature as well as verified with data derived from subsequent clinical trials. No adjustment for multiple comparisons is planned for exploratory analyses. Furthermore, additional post hoc exploratory assessments are expected and may be performed. There may be circumstances when a decision is made to stop sample collection, or not perform or discontinue the analysis of blood/archival tumor samples/fresh tumor biopsies/fine needle aspirates due to either practical or strategic reasons (e.g. issues related to the quality and/or quantity of the samples or issues related to the assay). Under such circumstances, the number of samples may be inadequate to perform a rigorous data analysis and the available data will only be listed and potentially summarized.

### 8.6.4.1. Outline of the Data Analysis

Additional analyses that may be performed after the completion of the end-of-trial Clinical Study Report (CSR) will be documented in separate reports. These analyses may include but are not limited to the meta-analysis of data from this trial combined with data from other trials or the analysis of biomarkers generated from samples collected during the trial but analyzed after the database lock and completion of the CSR. The data analysis will be described in an addendum of the SAP or in a stand-alone analysis plan document, as appropriate.

### 8.6.4.2. Data Analysis Principles

### 8.6.4.2.1. Analysis Sets

The FAS will be used for all biomarker analysis. Unless otherwise specified, all statistical analyses of biomarker data will be performed on patients with biomarker data.

### 8.6.4.2.2. Basic Tables, Figures and Listings

IHC markers data (e.g. Ki67) will be listed by marker and treatment arm and summarized using summary statistics (mean, standard deviation, median, minimum, maximum).

Gene expression data and Next Generation Sequencing data (also known as deep sequencing data) will be generated on an oncology specific set of genes. As these data are extensive and complex, only those genes associated with BC and the key pathways of interest will be shown. To understand the relationship with response Kaplan Meier curves, median iDFS and 95% Cl may be generated for the most interesting genes using altered vs. non-altered status. All these analyses will be detailed in the SAP or separate biomarker analysis plan.

Blood samples will be collected to assess gene expression and alteration in ctDNA/ctRNA. Data will be generated from samples at baseline, on treatment, post treatment and at time of recurrence. The gene expression and alteration of genes relevant to the CDK pathway and BC (e.g. ESR1, PIK3CA) will be assessed as feasible with the available technology for ctDNA/ctRNA at time of assessment.

Known mutation and amplifications in the displayed genes, as defined by the COSMIC (Catalogue of Somatic Mutations in Cancer) database will be listed and summarized by treatment arm and overall using counts and percentages.

Those genes whose alteration status is assessed in both tumor tissue and ctDNA will be compared using 2×2 concordance table to assess their agreement.

Finally, alteration data collected at recurrence will be listed simultaneously with the baseline sample data of the same type. Only positions where there is discordance between the two sources will be displayed.

Any additional exploratory analyses on biomarkers will be detailed in the SAP or separate biomarker analysis plan.

### 8.6.4.2.3. Advanced Analysis Methods

Advanced analysis methods may be performed to explore relationship between biomarkers and trial endpoints and to identify patterns in the data. Further details will be provided in the SAP or separate biomarker analysis plan.

8.7. Interim and Final iDFS Analyses

Three interim analyses are planned after approximately 200, 350 and 425 of the approximately 500 targeted iDFS events (i.e. at approximately 40%, 70% and 85% information fractions, respectively) have been documented. These interim analyses are expected to occur around 27, 35 and 39 months from the date of first patient randomized in the trial. The primary intent of the first interim analysis is to stop early for lack of efficacy (futility). There is no intent to carry out an analysis to declare superior efficacy at the time of the first interim analysis. The second and third interim analyses will allow the trial to declare outstanding efficacy. The second interim analysis will only be carried out after all patients have been randomized and (if not withdrawn early) have at least one post-baseline recurrence assessment as per Section 6.4.1.1.

An alpha-spending function according to a three-look (Lan-DeMets) group sequential design with (O'Brien-Fleming) type stopping boundary (as implemented in East 6.4) will be used to construct the efficacy stopping boundaries.⁶¹

A Lan-DeMets (O'Brien-Fleming) type stopping boundary as implemented in East 6.4 will be used as a beta-spending function to determine the non-binding futility boundary. The choice of non-binding nature of the futility stopping boundary ensures that the efficacy stopping boundaries are not affected.

Based on the choice of beta-spending function described above and if the first interim analysis is performed exactly at 200 iDFS events, the futility boundary expressed on the p-value scale (or the Z-statistic scale) at the first interim is calculated as p=0.6752 (or Z=0.4544). The observed (i.e. nominal) p-value has to be greater than p-value scale efficacy boundary=0.6752 (or the observed Z-statistic has to be >Z-statistic scale boundary=0.4544) to conclude futility. If the second interim analysis is performed exactly at 350 iDFS events, the efficacy boundary in terms of p-value scale (or Z-statistic scale) at the efficacy interim is calculated as p=0.0074 (or Z=−2.438). The observed (i.e. nominal) p-value has to be smaller than 0.0074 to conclude superior efficacy at the second interim analysis. Similarly, if the third interim analysis is performed exactly at 425 iDFS events, the efficacy boundary in terms of p-value scale (or Z-statistic scale) at the efficacy interim is calculated as p=0.0129 (or Z-statistic scale=−2.2296). The observed p-value has to be smaller than 0.0129 to conclude superior efficacy at the third interim analysis.

Since the observed number of events at the interim analyses may not be exactly equal to the planned 200, 350 and 425 iDFS events, the efficacy and futility boundaries will need to be re-calculated using the pre-specified α- and β-spending functions and based on the actual number of observed events at interim and the total number of targeted events to calculate the exact information fraction. The observed p-value (or Z-test statistic) at the interim analyses will then be compared against the re-calculated efficacy (or futility) boundary.

If the trial continues to the final iDFS analysis, the final iDFS analysis will be performed when approximately 500 iDFS events have been documented. If exactly 200, 350 and 425 events are observed at the interim analyses, the trial continued and exactly 500 events are obtained at the final analysis, the observed p-value will have to be less than 0.0202 (or the observed Z-statistics has to be <−2.0503) to declare statistical significance. In practice, the final analysis will be based on the actual number of iDFS events documented at the cut-off date for the final iDFS analysis and the alpha already spent at the interim analyses. The boundary for the final analysis will be derived accordingly from the pre-specified α-spending function such that the overall significance level across all analyses is maintained at 0.025.

The statistical properties of the group sequential design are summarized in Table A23 below.

TABLE A23
Simulated Probabilities to Stop for Efficacy or
Futility at the Interim or Final iDFS Analysis

| | | | Simulated | | Simulated | |
|---|---|---|---|---|---|---|
| | | | cumulative | | incremental | |
| | | | probabilities (%) | | probabilities (%) | |
| | | # iDFS | Stop for | Stop for | Stop for | Stop for |
| Scenario | Look | events | efficacy | futility | efficacy | futility |
| Under H₀ | Interim 1 | 200 | | 32.7 | | 32.7 |
| (HR = 1) | Interim 2 | 350 | 0.8 | | 0.8 | |
| | Interim 3 | 425 | 1.5 | — | 0.7 | |
| | Final | 500 | 2.3 | | 0.8 | |
| Under Hₐ | Interim 1 | 200 | | 0.4 | | 0.4 |
| (HR = 0.73) | Interim 2 | 350 | 68.4 | | 68.4 | |
| | Interim 3 | 425 | 84.8 | | 16.3 | |
| | Final | 500 | 93.1 | | 8.3 | |
| Under | Interim 1 | 200 | | 0.8 | | 0.8 |
| other Hₐ | Interim 2 | 350 | 54.0 | | 54.0 | |
| (HR = 0.76) | Interim 3 | 425 | 73.3 | | 19.3 | |
| | Final | 500 | 85.2 | | 12.0 | |
| Under | Interim 1 | 200 | | 2.1 | | 2.1 |
| other Hₐ | Interim 2 | 350 | 35.3 | | 35.3 | |
| (HR = 0.80) | Interim 3 | 425 | 53.5 | | 18.2 | |
| | Final | 500 | 67.9 | | 14.4 | |

Note:
Simulation is performed in East 6.4 with number of simulations = 10,000 and randomization seed = 123

The results of the interim analyses will be provided to the IDMC by the independent statistician who is not part of trial management.

The projected timing of interim and final analyses of iDFS is summarized in Table A24.

At the time of interim analyses for iDFS, the IDMC will determine that either (i) iDFS analysis has crossed the pre-specified boundary for efficacy, or (ii) the trial needs to be terminated due to any cause including futility or safety reasons. Further details will be described in the IDMC Charter.

TABLE A24
Estimated Timelines for Interim and Final Analyses

| Months after randomization of | # iDFS | Cumulative Power against |
|---|---|---|
| the first patient (approximation) | Events | a hazard ratio of 0.73 |
| 27 | 200 (40%) | 0 |
| 35 | 350 (70%) | 68.4% |
| 39 | 425 (85%) | 84.8% |
| 44 | 500 | 93.1% |

Calculated using East 6.4

### 8.8. Sample Size Calculation

The sample size calculation is based on the primary variable iDFS. The hypothesis to be tested and details of the testing strategy are described in Sections 8.4.2 and 8.7. In the trial, the enrollment of patients is expected to be approximately 40% for the Anatomic Stage II and 60% for the Anatomic Stage III. The 5-year iDFS rate for the patients with Anatomic Stage II (excluding low risk patients) is assumed to be approximately 79% (based on data from a retrospective study assessing the prognostic effect of Ki67 and other disease characteristics in HR-positive, HER2-negative EBC⁶²) and 72% (based on data for patients with 4 lymph nodes treated with Als in the EBCTCG meta-analysis³⁰) for the Anatomic Stage III patients, respectively. Based on these assumptions and the expected distribution of patients across the anatomical stages, the overall 5-year iDFS of the control arm is expected to be approximately 74.8%. It is expected that treatment with ribociclib in addition to standard ET will result in a 27% reduction in the hazard rate for iDFS, i.e. an expected hazard ratio of 0.73. 500 iDFS events will provide a power of approximately 93% and 85% when the overall hazard ratio is 0.73 and 0.76, respectively. This calculation assumes analysis by a one-sided log-rank test at the overall 2.5% level of significance, patients randomized to the two treatment arms in a 1:1 ratio, and a 4-look group sequential design with a Lan-DeMets (O'Brien-Fleming) alpha spending function and a Lan-DeMets (O'Brien-Fleming) beta spending function to define a non-binding futility rule at the interim analyses, using an information fraction of 40% for the first interim analysis (futility only) and an information fraction of 70% and 85% for the second and third interim analyses (efficacy only), respectively. The final analysis will be done after approximately 500 iDFS events have been documented.

Assuming that enrollment will continue at a rate of approximately 170 patients per month starting in Q3-2020 and a 15% dropout rate by the time of the final iDFS analysis (i.e. dropout hazard rate=0.00629 per month), a total of 5,000 patients will need to be randomized to observe the targeted 500 iDFS events at about 44 months after the randomization date of the first patient. These calculations were made using the software package East 6.4.

### Sample Size Justification for PK Collection

It is planned to have approximately 100 evaluable patients for PK characterization in this trial. Assuming an approximately 20% of the patients are non-evaluable for PK due to issues such as sample collection and handling, it is planned to collect PK samples for ribociclib from approximately 130 patients in the Investigational arm. The choice of sample size and sampling time points were selected based on historical data and PK profile of ribociclib. No formal statistical inference is planned.

### 9. ADMINISTRATIVE, ETHICAL AND REGULATORY STANDARDS

### 9.1. Quality Control and Assurance

Before trial initiation, at a site initiation visit or at an Investigator's meeting, authorized representatives of the Sponsor will review the protocol and CRFs with the Investigators and their staff. During the trial, Sponsor representatives will visit the sites to perform ongoing data verification in order to confirm that data entered into the CRF by authorized site personnel are accurate, complete, and verifiable from source documents; that the safety and rights of participants are being protected; and that the trial is being conducted in accordance with the currently approved protocol, other trial agreements, International Council for Harmonization for Good Clinical Practices (ICH-GCP), and applicable regulatory requirements.

The Investigator must give the monitor access to all relevant source documents to allow for confirmation of their consistency with the CRF entries. Novartis monitoring standards require full verification for the presence of informed consent, adherence to the inclusion/exclusion criteria and documentation of SAEs. Additional actions taken by the monitor to ensure that the trial is conducted and documented properly are performed according to the trial-specific monitoring plan.

The Investigator/institution may also be subject to an audit by Novartis or designee or to inspections by CA during the conduct of the trial or after the end of the trial. The auditors/inspectors will review documentation at the site to ensure that the trial is conducted, and data is generated, documented (recorded), and reported, in compliance with the protocol, GCP, and the applicable regulatory requirement(s). During these activities, the Investigator/institution will permit authorized representatives of the Sponsor and CA to examine (and when required by applicable law, to copy) clinical records.

Investigators and their relevant personnel should be available as necessary during the monitoring visits, audits and regulatory inspections.

### 9.2 Ethical Considerations

### 9.2.1. Ethical Conduct of the Trial

This clinical trial was designed, shall be implemented and reported in accordance with the ICH-GCP, with applicable local regulations (including European Directive 2001/20/EC and 2005/28/EC and US Code of Federal Regulations Title 21), and with the ethical principles laid down in the Declaration of Helsinki. This trial will be conducted under ethical, scientific, and medical standards that protect the rights and welfare of participants.

### 9.2.2. Responsibilities of the Investigator and IRB/IEC

The protocol, the proposed PICF, other relevant documents, and any information to be given to the patient must be reviewed and approved by a properly constituted IRB/IEC before being used at site. An affirmative decision of the IRB/IEC that the clinical trial has been reviewed and may be conducted at the institution/site within the constraints set forth by the IRB, the institution, GCP, and the applicable regulatory requirements must be obtained. This decision will be made in writing to the Investigator and a copy of this decision will be provided to the Sponsor.

Prior to the trial's start at a site, the Investigator is required to sign a protocol signature page confirming his/her agreement to conduct the trial in accordance with these documents and all of the instructions and procedures found in this protocol.

The Investigator will agree to make required progress reports to the IRB/IEC, as well as report any SAEs, life-threatening problems or deaths if applicable per local regulations. The IRB/IEC will be informed of SUSARs in other clinical trials conducted with the trial drug by the Investigator or the Sponsor according to the local regulations. The IRB/IEC must be informed by the Investigator of the termination of the trial.

### 9.2.3. Informed Consent Procedures

Eligible patients may only be included in the trial after being given pertinent information about the intended purpose of the trial, the trial treatment, the procedures and possible benefits and hazards to which the patient will be exposed, and after providing written (witnessed by an impartial witness, where required by law or regulation), IRB/IEC-approved informed consent, or, if incapable of doing so, after such consent has been provided by a legally acceptable representative of the patient, if permitted by local regulation. A legally acceptable representative is an individual or other body authorized under applicable law to consent, on behalf of a prospective patient, to the patient's participation in the trial. In cases where the patient's representative gives consent, the patient should be informed about the trial to the extent possible given his/her understanding. If the patient is capable of doing so, he/she should indicate assent by personally signing and dating the written informed consent document or a separate assent form.

The approved PICF will be read and dated and signed by the patient or her/his legal representative and an impartial witness when legally required, and the Investigator. The Investigator will keep the original PICF on site and the patient will be provided with an original copy (or a photocopy) of the signed PICF. Informed consent must be obtained before conducting any trial-specific procedures, for example before any clinical procedures are performed solely for the purpose of determining eligibility. Procedures that are to be performed as part of the practice of medicine and which would be done whether or not trial entry was contemplated, such as for diagnosis or treatment of a disease or medical condition, may be performed and the results subsequently used for determining trial eligibility without first obtaining consent. The process of obtaining informed consent should be documented in the patient source documents. The date when a patient's informed consent was actually obtained will be captured in their CRFs.

Novartis (or authorized representatives) will provide to Investigators, in a separate document, a proposed PICF that is considered appropriate for this trial and complies with the ICH-GCP guideline and regulatory requirements.

The PICF will include a statement by which the patient allows the Sponsor's duly authorized personnel and representatives, the IRB/IEC, and the CA to have direct access to their data that will be processed in accordance with applicable regulations, e.g. Health Insurance Portability and Accountability Act (HIPAA) and General Data Protection Regulation (GDPR).

Any changes to this PICF suggested by the Investigator must be agreed by Novartis (or authorized representatives) before submission to the IRB/IEC, and a copy of the approved version must be provided to the monitor after IRB/IEC approval.

Women of CBP should be informed that taking the trial drug may involve unknown risks to the fetus if pregnancy were to occur during the trial and agree that in order to participate in the trial they will adhere to the contraception requirement for the duration of the trial. If there is any question that the patient will not reliably comply with this requirement, they should not be entered in the trial.

If important new information becomes available that may be relevant to the patient's consent and willingness to continue participation in the trial, the PICF will be revised and submitted to IRB/IEC and CA (if applicable) for approval/favorable opinion. The new information will be then discussed with the patient in a timely manner and if she/he agrees to continue participation in the trial, the revised PICF will be signed and dated and the patient will receive a copy.

The patient may withdraw from the trial at anytime without prejudicing future medical treatment. In any case, withdrawal should be documented on patient's clinical records.

### 9.2.3.1. Additional Consent

There are two optional consents from the patient: one for additional biomarker assessments to be performed when residual samples are remaining (see Section 6.4.4.2) and another for the collection of an additional blood sample to be collected for pharmacogenetic assessment (see Section 6.4.4.3).

In order to participate in these sub-studies, the patient must give his/her explicit consent. If a patient opts not to participate in the optional assessments, this in no way affects the patient's ability to participate in the main research trial.

### 9.2.4. Protocol Compliance

Investigators assert they will apply due diligence to avoid protocol deviations. Under no circumstances should the Investigator contact Novartis, TRIO or its agents, to request approval of a protocol deviation, as no authorized deviations are permitted.

If the Investigator feels a protocol deviation would improve the conduct of the trial this must be considered for a protocol amendment, and unless such an amendment is agreed upon by Novartis and approved by the IRB/IEC and CA it cannot be implemented.

Notwithstanding the need for approval of formal protocol amendments, the Investigator is expected to take any immediate action required for the safety of any patient included in this trial, even if this action represents a deviation from the protocol. In such cases, TRIO must be notified of this action and the IRB/IEC at the trial site must be informed according to local regulations (e.g. United Kingdom requires the notification of urgent safety measures within 3 days) but not later than 10 working days.

All significant protocol deviations will be recorded and reported in the CSR.

### 9.2.5. Amendments to the Protocol

Any change or addition to the protocol can only be made in a written protocol amendment that must be approved by Novartis, CA where required, and the IRB/IEC. Only amendments that are required to eliminate an immediate hazard to patients for patient safety may be implemented prior to IRB/IEC approval.

### 9.3. Discontinuation of the Trial

Novartis reserves the right to discontinue this trial under specific conditions.

### 9.4. Data Handling and Record Keeping

### 9.4.1. Trial Documentation, Record Keeping and Retention of Documents

Each participating site will maintain appropriate medical and research records for this trial, in compliance with Section 4.9 of the ICH E6 GCP, and regulatory and institutional requirements for the protection of confidentiality of subjects.

Essential documents are all information, original records of clinical findings, observations, or other activities in a clinical trial necessary for the reconstruction and evaluation of the trial. Examples of these original documents and data records include, but are not limited to, hospital records, clinical and office charts, laboratory notes, memoranda, patients' diaries or evaluation checklists, pharmacy dispensing records, recorded data from automated instruments, copies or transcriptions certified after verification as being accurate and complete, microfiches, photographic negatives, microfilm or magnetic media, x-rays, and patients files and records kept at the pharmacy, at the laboratories, and medico-technical departments involved in the clinical trial. Changes to Essential Documents must be traceable, should not obscure the original entry, and should be explained if necessary (e.g. via an audit trail).

The Investigator/institution must maintain the trial documents as specified in section 8 of the ICH E6 GCP and as required by applicable regulations and/or guidelines. The Investigator/institution must take measures to prevent accidental or premature destruction of these documents.

Essential documents (written and electronic) must be retained for a period of not less than fifteen (15) years from the completion of the trial unless Novartis provides written permission to dispose of them or requires their retention for an additional period of time because of applicable laws, regulations and/or guidelines.

### 9.4.2. Data Collection

The trial electronic CRF is the primary data collection instrument for the trial. Data reported on the CRF, that are derived from source documents, should be consistent with the source documents or the discrepancies should be explained. All data requested on the CRF must be recorded. Any missing data must be explained. An audit trail will be maintained by the system. The designated site staff will enter the data required by the protocol into the CRF within 7 calendar days of availability. The CRFs have been built using fully validated secure web-enabled software that conforms to 21 CFR Part 11 requirements. Investigator site staff will not be given access to the Electronic Data Capture (EDC) system until they have been trained. Automatic validation programs check for data discrepancies in the CRFs and, allow modification or verification of the entered data by the Investigator staff.

The Principal Investigator is responsible for assuring that the data entered into CRF and all other required reports is complete, accurate, and that entry and updates are performed in a timely manner.

PRO data will be recorded by patients on paper forms and entered in the CRF by site staff. Blood samples for laboratory data will be collected and processed locally. ECG data will be sent to a central laboratory for processing, and the results will be sent electronically to sites and Novartis (or authorized representatives).

Biomarkers blood samples and tumor samples will be collected by sites and sent to the Novartis designated central laboratory for processing and the results will be sent electronically to Novartis (or authorized representatives).

### 9.4.3. Database Management

Novartis (or authorized representatives) will review the data entered by investigational staff for completeness and accuracy. This is an open-label trial. Investigators, patients, TRIO and the Sponsor will have full knowledge of the treatment allocation. In order to minimize the potential impact of trial treatment knowledge, until the final iDFS analysis is conducted, no aggregate statistical analyses (efficacy or safety across the trial) shall be performed by treatment arm (other than the analyses as specified in the protocol).

Electronic data queries stating the nature of the problem and requesting clarification will be created for discrepancies and missing values and sent to the investigational site via the EDC system. Designated site staff is required to respond to queries within specified timelines and to make any necessary changes to the data.

Concomitant treatments, prior medications and systemic anti-cancer therapies entered into the database will be coded using the World Health Organization (WHO) Drug Reference List, which employs the Anatomical Therapeutic Chemical classification system. Medical history/current medical conditions and AEs will be coded using the Medical Dictionary for Regulatory Activities (MedDRA) terminology.

Randomization codes and data about all trial treatments dispensed to the patient and all IRT assigned dosage changes will be tracked in a system supplied by a vendor(s), who will also manage the IRT database. The data will be sent electronically to Novartis personnel (or authorized representatives).

After data have been verified to be complete and accurate, the database will be declared locked. Authorization is required prior to making any database changes to locked data, by joint written agreement between TRIO and Novartis' Global Head of Biostatistics and Data Management and Global Head of Clinical Development.

After database lock, the Investigator will receive electronic or paper copies of the patient data for archiving at the investigational site.

### 9.4.4. Data Protection

The patient's personal data and Investigator's personal data will be utilized in the conduct of this trial and shall be treated in compliance with all applicable laws and regulations, e.g. HIPAA and GDPR.

When processing or archiving personal data pertaining to the Investigator and/or to the patients, the TRIO, Novartis, and their representatives shall take all appropriate measures to safeguard and prevent access to this data by any unauthorized third party.

### 9.4.5. Data Confidentiality

The Investigator must ensure confidentiality of the patients; patients must not be identified in any documents submitted to TRIO or Novartis (or representatives), for example, signed PICFs and patient enrollment logs with patients' names must be kept strictly confidential. Such documents must be kept at site to enable patient identification.

The data collection system for this trial uses built-in security features to encrypt all data for transmission in both directions, preventing unauthorized access to confidential participant information. Access to the system will be controlled by a sequence of individually assigned user identification codes and passwords, made available only to authorized personnel who have completed prerequisite training.

Prior to entering key sensitive personally identifiable information (patient initials and exact Date of Birth), the system will prompt site to verify that this data is allowed to be collected. If the site indicates that country rules or ethics committee standards do not permit collection of these items, the system will not solicit patient initials. Year of birth will be solicited (in the place of exact date of birth) to establish that the patient satisfies protocol age requirements and to enable appropriate age-related normal ranges to be used in assessing laboratory test results.

All information concerning the trial, and not previously published, is considered confidential and proprietary information. This confidential information shall remain the sole property of Novartis and shall not be disclosed to others without prior written consent from Novartis. Information shall not be used except in the performance of this trial.

### 9.5. Steering Committee

The SC will be established comprising Investigators with broad experience in the treatment of BC who are participating in the trial (but not being members of the IDMC), a patient advocate and Novartis and TRIO representatives from the Clinical Trial Team.

The SC will ensure transparent management of the trial according to the protocol through recommending and approving modifications as circumstances require. The SC will review the clinical trial protocol and any amendment as appropriate. Together with the Clinical Trial Team, the SC will also develop recommendations for publications of trial results including authorship rules. The details of the role of the SC will be defined in a SC charter.

### 9.6. Financial Disclosures

Financial disclosures are to be provided by trial personnel who are directly involved in the treatment or evaluation of patients at the site, prior to trial start.

### 9.7. Insurance of Liabilities

If required, the Investigator may forward to the IRB/IEC a copy of the insurance document provided by the Sponsor, in order to cover his/her liabilities, and those of any other participating parties.

### 9.8. Use of Information and Publication

Novartis is committed to following high ethical standards for reporting trial results for its innovative medicine, including the timely communication and publication of clinical trial results, whatever their outcome. Novartis assures that the key design elements of this protocol will be posted on the publicly accessible database, e.g. www.clinicaltrials.gov before trial start. In addition, results of interventional clinical trials in adult patients are posted on www.novartisclinicaltrials.com, a publicly accessible database of clinical trial results within 1 year of trial completion (i.e. last patient last visit [LPLV]).

No publication, abstract or presentation of the trial will be made without the approval of Novartis and the SC (if applicable).

Novartis follows the International Committee of Medical Journal Editors authorship guidelines (www.icmje.org) and other specific guidelines of the journal or congress to which the publication will be submitted.

Authors will not receive remuneration for their writing of a publication, either directly from Novartis or through the professional medical writing agency. Author(s) may be requested to present poster or oral presentation at scientific congress; however, there will be no honorarium provided for such presentations.

As part of its commitment to full transparency in publications, Novartis supports the full disclosure of all funding sources for the trial and publications, as well as any actual and potential conflicts of interest of financial and non-financial nature by all authors, including medical writing/editorial support, if applicable.

For the Novartis Guidelines for the Publication of Results from Novartis-sponsored Research, please refer to www.novartis.com.

### 10. REFERENCES

1. Torre L A, Bray F, Siegel R L, et al: Global cancer statistics, 2012. C A Cancer J Clin 65:87-108, 2015
2. GLOBOCAN: Estimated Cancer Incidence, Mortality and Prevalence Worldwide 2012 [Internet][cited 2018 Jun. 26] Available from:
http://globocan.iarc.fr/old/bar_sex_site.asp?selection=3152&title=Breast&statistic=2&populations=6&wind ow=1&grid=1&color1=5&color1e=&color2=4&color2e=&submit=% C2% AOExecute % C2%A0
3. Siegel R L, Miller K D, Jemal A: Cancer statistics, 2018. C A Cancer J Clin 68:7-30, 2018
4. Ferlay J, Steliarova-Foucher E, Lortet-Tieulent J, et al: Cancer incidence and mortality patterns in Europe: estimates for 40 countries in 2012. Eur J Cancer Oxf Engl 1990 49:1374-1403, 2013
5. Eggemann H, Ignatov A, Smith B J, et al: Adjuvant therapy with tamoxifen compared to aromatase inhibitors for 257 male breast cancer patients. Breast Cancer Res Treat 137:465-470, 2013
6. SEER Cancer Statistics Review, 1975-2015 [Internet]. Bethesda, M D, National Cancer Institute, 2018Available from: https://seer.cancer.gov/csr/1975_2015/
7. Anampa J, Makower D, Sparano J A: Progress in adjuvant chemotherapy for breast cancer: an overview. BMC Med 13:195, 2015
8. Early Breast Cancer Trialists' Collaborative Group (EBCTCG): Effects of chemotherapy and hormonal therapy for early breast cancer on recurrence and 15-year survival: an overview of the randomised trials. Lancet Lond Engl 365:1687-1717, 2005
9. Hammond MEH, Hayes D F, Dowsett M, et al: American Society of Clinical Oncology/College Of American Pathologists guideline recommendations for immunohistochemical testing of estrogen and progesterone receptors in breast cancer. J Clin Oncol Off J Am Soc Clin Oncol 28:2784-2795, 2010
10. Senkus E, Kyriakides S, Ohno S, et al: Primary breast cancer: ESMO Clinical Practice Guidelines for diagnosis, treatment and follow-up. Ann Oncol Off J Eur Soc Med Oncol 26 Suppl 5:v8-30, 2015
11. National Comprehensive Cancer Network. Breast Cancer (Version 1.2019) [Internet]. NCCN, 2019[cited 2019 May 6] Available from: https://www.nccn.org/professionals/physician_gls/pdf/breast.pdf
12. Francis P A, Pagani 0, Fleming G F, et al: Tailoring Adjuvant Endocrine Therapy for Premenopausal Breast Cancer. N Engl J Med 379:122-137, 2018
13. Goss P E, Ingle J N, Pritchard K I, et al: Extending Aromatase-Inhibitor Adjuvant Therapy to 10 Years. N Engl J Med 375:209-219, 2016
14. Zagouri F, Sergentanis T N, Azim H A, et al: Aromatase inhibitors in male breast cancer: a pooled analysis. Breast Cancer Res Treat 151:141-147, 2015
15. Pan H, Gray R, Braybrooke J, et al: 20-Year Risks of Breast-Cancer Recurrence after Stopping Endocrine Therapy at 5 Years. N Engl J Med 377:1836-1846, 2017
16. Holm K, Staaf J, Jbnsson G, et al: Characterisation of amplification patterns and target genes at chromosome 11q13 in CCND1-amplified sporadic and familial breast tumours. Breast Cancer Res Treat 133:583-594, 2012
17. Cancer Genome Atlas Network: Comprehensive molecular portraits of human breast tumours. Nature 490:61-70, 2012
18. Beroukhim R, Mermel C H, Porter D, et al: The landscape of somatic copy-number alteration across human cancers. Nature 463:899-905, 2010
19. Thangavel C, Dean J L, Ertel A, et al: Therapeutically activating R B: reestablishing cell cycle control in endocrine therapy-resistant breast cancer. Endocr Relat Cancer 18:333-345, 2011
20. Bosco E E, Wang Y, Xu H, et al: The retinoblastoma tumor suppressor modifies the therapeutic response of breast cancer. J Clin Invest 117:218-228, 2007
21. Turner N C, Ro J, Andre F, et al: Palbociclib in Hormone-Receptor-Positive Advanced Breast Cancer. N Engl J Med 373:209-219, 2015
22. Loibl S, Turner N C, Ro J, et al: Palbociclib (PAL) in combination with fulvestrant (F) in pre-/peri-menopausal (PreM) women with metastatic breast cancer (MBC) and prior progression on endocrine therapy—results from Paloma-3. J Clin Oncol 34:524-524, 2016
23. Goetz M P, Toi M, Campone M, et al: MONARCH 3: Abemaciclib As Initial Therapy for Advanced Breast Cancer. J Clin Oncol Off J Am Soc Clin Oncol 35:3638-3646, 2017
24. Hortobagyi G N, Stemmer S M, Burris H A, et al: Ribociclib as First-Line Therapy for H R-Positive, Advanced Breast Cancer. N Engl J Med 375:1738-1748, 2016
25. Slamon D J, Neven P, Chia S, et al: Phase 11 Randomized Study of Ribociclib and Fulvestrant in Hormone Receptor-Positive, Human Epidermal Growth Factor Receptor 2-Negative Advanced Breast Cancer: MONALEESA-3. J Clin Oncol Off J Am Soc Clin Oncol JCO2018789909, 2018
26. Finn R S, Martin M, Rugo H S, et al: Palbociclib and Letrozole in Advanced Breast Cancer. N Engl J Med 375:1925-1936, 2016
27. Verzenio® (abemaciclib) Significantly Reduced the Risk of Cancer Returning in People with High Risk HR+, HER2—Early Breast Cancer [Internet]. Eli Lilly Co [cited 2020 Aug. 4] Available from: https://investor.IIIly.com/news-releases/news-release-details/verzenior-abemaciclib-significantly-reduced-risk-cancer
28. Pfizer Provides Update on Phase 3 PALLAS Trial of IBRANCE® (palbociclib) Plus Endocrine Therapy in HR+, HER2—Early Breast Cancer I Pfizer [Internet][cited 2020 Aug. 4] Available from: https://www.pfizer.com/news/press-release/press-release-detail/pfizer-provides-update-phase-3-pallas-trial-ibrancer
29. Tripathy D, Im S-A, Colleoni M, et al: Ribociclib plus endocrine therapy for premenopausal women with hormone-receptor-positive, advanced breast cancer (MONALEESA-7): a randomised phase 3 trial [Internet]. Lancet Oncol 0, 2018[cited 2018 Jun. 29] Available from: https://www.thelancet.com/journals/lanonc/article/PIIS1470-2045(18)30292-4/abstract
30. Early Breast Cancer Trialists' Collaborative Group (EBCTCG): Aromatase inhibitors versus tamoxifen in early breast cancer: patient-level meta-analysis of the randomised trials. Lancet Lond Engl 386:1341-1352, 2015
31. Lipton A, Demers L M, Harvey H A, et al: Letrozole (CGS 20267). A phase I study of a new potent oral aromatase inhibitor of breast cancer. Cancer 75:2132-2138, 1995
32. Trunet P F, Bhatnagar A S, Chaudri H A, et al: Letrozole (CGS 20267), a new oral aromatase inhibitor for the treatment of advanced breast cancer in postmenopausal patients. Acta Oncol Stockh Swed 35 Suppl 5:15-18, 1996
33. Edavana V K, Dhakal I B, Williams S, et al: Potential role of UGT1A4 promoter SNPs in anastrozole pharmacogenomics. Drug Metab Dispos Biol Fate Chem 41:870-877, 2013
34. Plourde P V, Dyroff M, Dowsett M, et al: ARIMIDEX: a new oral, once-a-day aromatase inhibitor. J Steroid Biochem Mol Biol 53:175-179, 1995
35. Hudis C A, Barlow W E, Costantino J P, et al: Proposal for standardized definitions for efficacy end points in adjuvant breast cancer trials: the STEEP system. J Clin Oncol Off J Am Soc Clin Oncol 25:2127-2132, 2007
36. Ribociclib Investigator's Brochure
37. Dowsett M, Nielsen T O, A'Hern R, et al: Assessment of Ki67 in breast cancer: recommendations from the International Ki67 in Breast Cancer working group. J Natl Cancer Inst 103:1656-1664, 2011
38. Bustreo S, Osella-Abate S, Cassoni P, et al: Optimal Ki67 cut-off for luminal breast cancer prognostic evaluation: a large case series study with a long-term follow-up. Breast Cancer Res Treat 157:363-371, 2016
39. Tashima R, Nishimura R, Osako T, et al: Evaluation of an Optimal Cut-Off Point for the Ki-67 Index as a Prognostic Factor in Primary Breast Cancer: A Retrospective Study [Internet]. PLoS ONE 10, 2015[cited 2018 Aug. 13] Available from: https://www.ncbi.nlm.nih.gov/pmc/articles/PMC4503758/
40. Krop I, Ismaila N, Andre F, et al: Use of Biomarkers to Guide Decisions on Adjuvant Systemic Therapy for Women With Early-Stage Invasive Breast Cancer: American Society of Clinical Oncology Clinical Practice Guideline Focused Update. J Clin Oncol 35:2838-2847, 2017
41. Overview I Tumour profiling tests to guide adjuvant chemotherapy decisions in early breast cancer Guidance I NICE [Internet][cited 2019 May 6] Available from: https://www.nice.org.uk/guidance/dg34
42. Giuliano A E, Ballman K V, McCall L, et al: Effect of Axillary Dissection vs No Axillary Dissection on 10-Year Overall Survival Among Women With Invasive Breast Cancer and Sentinel Node Metastasis: The ACOSOG Z0011 (Alliance) Randomized Clinical Trial. JAMA 318:918-926, 2017
43. Lyman G H, Somerfield M R, Bosserman L D, et al: Sentinel Lymph Node Biopsy for Patients With Early-Stage Breast Cancer: American Society of Clinical Oncology Clinical Practice Guideline Update. J Clin Oncol 35:561-564, 2016
44. Curigliano G, Burstein H J, P Winer E, et al: De-escalating and escalating treatments for early-stage breast cancer: the St. Gallen International Expert Consensus Conference on the Primary Therapy of Early Breast Cancer 2017. Ann Oncol Off J Eur Soc Med Oncol 28:1700-1712, 2017
45. Early Breast Cancer Trialists' Collaborative Group (EBCTCG): Long-term outcomes for neoadjuvant versus adjuvant chemotherapy in early breast cancer: meta-analysis of individual patient data from ten randomised trials. Lancet Oncol 19:27-39, 2018
46. Dhesy-Thind S, Fletcher G G, Blanchette P S, et al: Use of Adjuvant Bisphosphonates and Other Bone-Modifying Agents in Breast Cancer: A Cancer Care Ontario and American Society of Clinical Oncology Clinical Practice Guideline [Internet]. J Clin Oncol, 2017[cited 2018 Dec. 19] Available from: http://ascopubs.org/doi/abs/1 0.1200/JCO.2016.70.7257
47. Smith T J, Bohlke K, Lyman G H, et al: Recommendations for the Use of WBC Growth Factors: American Society of Clinical Oncology Clinical Practice Guideline Update. J Clin Oncol Off J Am Soc Clin Oncol 33:3199-3212, 2015
48. Grunberg S M, Warr D, Gralla R J, et al: Evaluation of new antiemetic agents and definition of antineoplastic agent emetogenicity—state of the art. Support Care Cancer 19 Suppl 1:S43-47, 2011
49. Roila F, Molassiotis A, Herrstedt J, et al: 2016 MASCC and ESMO guideline update for the prevention of chemotherapy- and radiotherapy-induced nausea and vomiting and of nausea and vomiting in advanced cancer patients. Ann Oncol Off J Eur Soc Med Oncol 27:v119-v133, 2016
50. National Comprehensive Cancer Network. Antiemesis (Version 3.2018) [Internet]. NCCN, 2018[cited 2018 Jul. 12] Available from: https://www.nccn.org/professionals/physician_gls/pdf/antiemesis.pdf
51. Elston C W, Ellis 10: Pathological prognostic factors in breast cancer. 1. The value of histological grade in breast cancer: experience from a large study with long-term follow-up. Histopathology 41:154-161, 2002
52. Oken M M, Creech R H, Tormey D C, et al: Toxicity and response criteria of the Eastern Cooperative Oncology Group. Am J Clin Oncol 5:649-655, 1982
53. Dawson S-J, Rosenfeld N, Caldas C: Circulating tumor DNA to monitor metastatic breast cancer. N Engl J Med 369:93-94, 2013
54. Esposito A, Bardelli A, Criscitiello C, et al: Monitoring tumor-derived cell-free DNA in patients with solid tumors: clinical perspectives and research opportunities. Cancer Treat Rev 40:648-655, 2014
55. Hurvitz S, Abad M F, Rostorfer R, et al: Interim results from neoMONARCH: A neoadjuvant phase 11 study of abemaciclib in postmenopausal women with HR+/HER2−breast cancer (BC) [Internet]. Ann Oncol 27, 2016[cited 2018 Jun. 26] Available from: https://academic.oup.com/annonc/article/27/suppl_6/LBA13/2800510
56. Murtaza M, Dawson S-J, Pogrebniak K, et al: Multifocal clonal evolution characterized using circulating tumour DNA in a case of metastatic breast cancer. Nat Commun 6:8760, 2015
57. Aaronson N K, Ahmedzai S, Bergman B, et al: The European Organization for Research and Treatment of Cancer QLQ-C30: a quality-of-life instrument for use in international clinical trials in oncology. J Natl Cancer Inst 85:365-376, 1993
58. Sprangers M A, Groenvold M, Arraras J I, et al: The European Organization for Research and Treatment of Cancer breast cancer-specific quality-of-life questionnaire module: first results from a three-country field study. J Clin Oncol Off J Am Soc Clin Oncol 14:2756-2768, 1996
59. Rabin R, de Charro F: EQ-5D: a measure of health status from the EuroQol Group. Ann Med 33:337-343, 2001
60. Zigmond A S, Snaith R P: The hospital anxiety and depression scale. Acta Psychiatr Scand 67:361-370, 1983
61. Lan KKG, DeMets D L: Discrete Sequential Boundaries for Clinical Trials. Biometrika 70:659-663, 1983
62. Fasching P A, Gass P, Huberle L, et al: Prognostic effect of Ki-67 in common clinical subgroups of patients with HER2-negative, hormone receptor-positive early breast cancer [Internet]. Breast Cancer Res Treat, 2019[cited 2019 May 20] Available from: https://doi.org/10.1007/s10549-019-05198-9
63. Amin M B, Edge S, Greene F, et al (eds): AJCC Cancer Staging Manual [Internet](ed 8). Springer International Publishing, 2017[cited 2018 Jun. 29] Available from: //www.springer.com/la/book/9783319406176

### 11. APPENDICES

### Appendix 1: Guidelines for Anatomic Stage Groups for Breast Cancer Staging

Guidelines for Anatomic Stage Groups according to AJCC Ed. 8^{th} applicable to this trial are presented in Table A25 below (modified from 63).

TABLE A25
AJCC 8th Edition Anatomic Stage Groups (not including Stage Group IV)

| Anatomic Stage Group | T-category | N-category |
|---|---|---|
| 0 | Tis | N0 |
| IA | T1 | N0 |
| IB | T0-1 | N1mi |
| IIA | T0-1 | N1 |
| | T2 | N0 |
| IIB | T2 | N1 |
| | T3 | N0 |
| IIIA | T0-3 | N2 |
| | T3 | N1 |
| IIIB | T4 | N0-2 |
| IIIC | Any T | N3 |

Note:
T2, T3, T4 tumors with nodal micrometastases (N1mi) are staged using the N1 category.

### Appendix 2: Guidelines for Standardized Definitions for Efficacy Endpoints in Adjuvant Breast Cancer Trials (based on STEEP)

Definitions for iDFS endpoints applicable to this study are presented in Table A26 below.

TABLE A26
Proposed Standardized Definitions for Breast Cancer Clinical Trial End Points in the Adjuvant Setting

| | | | | | | | | | | Second |
|---|---|---|---|---|---|---|---|---|---|---|
| | Invasive | | | | | | | | | Primary |
| | Ipsilateral | Local/ | | | | | | | | Invasive |
| | Breast | Regional | | | Death | Death | Invasive | | | Cancer |
| | Tumor | Invasive | Distant | Death | From | From | Contra- | Ipsi- | Contra- | (non- |
| End | Recur- | Recur- | Recur- | From | Non-BC | Unknown | lateral | lateral | lateral | breast) |
| Point | rence | rence | rence* | BC | Cause | Cause | BC** | DCIS | DCIS | *** |
| OS | | | | X | X | X | | | | |
| iDFS | X | X | X | X | X | X | X | | | X |
| DDFS | | | X | X | X | X | | | | X |
| RFS | X | X | X | X | X | X | | | | |
| LRRFS | X | X | | X | X | X | | | | |

Modified from Table 2 in ³⁵
NOTE:
Lobular carcinoma in situ is not included as an event in these definitions as is it not generally considered to be a direct precursor of breast cancer.
Abbreviations: BC, breast cancer; DCIS, ductal carcinoma in situ; OS, overall survival; iDFS, invasive disease-free survival-invasive; DDFS, distant disease-free survival; DRFS, distant relapse-free survival; LRRFS: loco-regional recurrence-free survival: RFS, recurrence-free survival.
*Site of first metastasis also should be reported, using the appropriate common data element term.
**The term “contralateral invasive breast cancer” is preferred to “second primary breast cancer,” as it is less ambiguous. Ipsilateral invasive breast cancers are presumed to be a recurrence.
*** Second primary non-breast cancers should not include squamous or basal cell skin cancers, or new in situ carcinomas of any site.

### Appendix 3: Recurrence Detection Guidelines

Recurrences will be detected by scheduled or unscheduled clinical evaluations. Suspected recurrence has to be evaluated by medical imaging and confirmed histologically (or cytologically, when applicable) unless there is an unacceptable risk to the patient or otherwise specified according to Section 6.4.1.1.2.

Recurrences will be classified into local, regional or distant recurrence, or contralateral invasive breast cancer or second primary non-breast invasive cancer according to the following guidelines:
Local invasive breast cancer recurrence or ipsilateral invasive breast tumor recurrence: invasive breast cancer involving the same breast as the original primary tumor. Ductal and lobular carcinoma in situ and tumors in the contralateral breast and/or contralateral lymph nodes are not considered a local invasive recurrence.
Local recurrence has to be confirmed histologically. Histological type of local invasive breast cancer recurrence should be recorded in the CRF.
Regional breast cancer recurrence: invasive breast cancer in the ipsilateral axilla, regional lymph nodes (all levels), chest wall, or skin of the ipsilateral breast. A tumor in the contralateral breast is not considered a regional recurrence.
Regional recurrence has to be confirmed histologically (preferred) or cytologically. The specific site of regional recurrence should be recorded in the CRF.
Distant recurrence: distant metastasis of breast cancer (bones, distant lymph nodes, internal organs, CNS, bone marrow, etc.) or any areas of invasive breast cancer recurrence that are not local or regional.
Distant recurrence has to be confirmed histologically (preferred) or cytologically unless there is an unacceptable risk to the patient due to the procedure.
If bone metastases were identified by a bone scan, it has to be confirmed histologically (preferred) or radiographically (CT, MRI or FDG-PET-CT) if biopsy confirmation is not possible.
Metastases in the central nervous system has to be confirmed histologically (preferred), cytologically or radiographically (CT or MRI, both with IV contrast) if biopsy confirmation is not possible.
All other sites of metastases have to be confirmed histologically (preferred) or cytologically unless there is an unacceptable risk to the patient due to the procedure.
Location of first three most dominant distant metastases should be reported in the CRF with preference given to visceral (including brain) metastases, than to bone, than distant lymph node or skin metastases.
Contralateral invasive breast cancer: any invasive breast cancer in the contralateral breast with or without contralateral lymph nodes involvement. Contralateral invasive breast cancer has to be confirmed histologically. In situ/non-invasive contralateral breast cancers are not included in contralateral invasive breast cancers. Histological type and specific site of contralateral invasive breast cancer should be recorded in the CRF.
Second primary non-breast invasive cancer: any second primary non-breast invasive cancers. Second primary non-breast invasive cancer has to be confirmed histologically. In situ/non-invasive cancers and basal or squamous cell carcinomas of the skin are not considered as second primary non-breast invasive cancers. Histological type and location of second primary non-breast invasive cancer should be recorded in the CRF.

### Appendix 4: Concomitant Medications

In general, the use of any concomitant medication deemed necessary for the care of the patient is permitted in this study, except as specifically prohibited below. Combination administration of study drugs could result in drug-drug interactions (DDI) that could potentially lead to reduced activity or enhanced toxicity of the concomitant medication and/or ribociclib.

The following lists are based on Novartis PK Sciences Memorandum, Drug-Drug Interaction (DDI) and Co-Medication Considerations for Novartis Clinical Trials (release date: January 2018), which was compiled from the Indiana University School of Medicine's P450 Drug Interaction Table (https://drug-interactions.medicine.iu.edu/Main-Table.aspx) and supplemented with the FDA Draft Guidance for Industry, Drug Interaction Studies—Study Design, Data Analysis, and Implications for Dosing and Labeling (February 2012) (fda.gov/downloads/drugs/guidancecomplianceregulatoryinformation/guidances/ucm292362.pdf) and the University of Washington's Drug Interaction Database (www.druginteractioninfo.org). For current lists of medications that may cause QT prolongation and/or TdP, refer to the CredibleMeds® website (https://crediblemeds.org/).

These lists are not comprehensive and are only meant to be used as a guide. Please contact the TRIO Medical Monitor with any questions. Note that these lists may include agents that are considered prohibited per section 5.4.3; their inclusion in the tables below must not be interpreted as an allowance for their use.

TABLE A27
List of Prohibited Medications During Ribociclib Treatment

| Category | Drug Name |
|---|---|
| Strong CYP3A4/5 | Atazanavir/ritonavir, boceprevir, |
| inhibitors | clarithromycin, cobicistat, conivaptan, |
| | danoprevir/ritonavir, darunavir/ritonavir, |
| | elvitegravir/ritonavir, grapefruit juice, |
| | idelalisib, indinavir, indinavir/ritonavir, |
| | itraconazole, ketoconazole, |
| | lopinavir/ritonavir, mibefradil, |
| | nefazodone, nelfinavir, |
| | ombitasvir/paritaprevir/dasabuvir/ |
| | ritonavir (VIEKIRA PAK), posaconazole, |
| | ritonavir, saquinavir/ritonavir, telaprevir, |
| | telithromycin, tipranavir/ritonavir, |
| | troleandomycin, voriconazole |
| Strong CYP3A4/5 | Apalutamide, carbamazepine³, |
| inducers | enzalutamide, lumacaftor, mitotane, |
| | phenobarbital, phenytoin³, rifabutin, |
| | rifampin (rifampicin)³, St. John's wort |
| | (hypericum perforatum)^{2,3} |
| CYP3A4/5 | Alfentanil, astemizole, cisapride, |
| substrates | cyclosporine, diergotamine |
| with NTI¹ | (dihydroergotamine), ergotamine, fentanyl, |
| | lomitapide⁵, lovastatin, nicardipine, |
| | nisoldipine, pimozide, quinidine, |
| | simvastatin, sirolimus, tacrolimus |
| Medications | Amiodarone, anagrelide, arsenic trioxide, |
| with a known risk | astemizole, azithromycin, bepridil |
| for QT | chloroquine, cocaine chlorpromazine, |
| prolongation⁴ | cilostazol, ciprofloxacin, cisapride, |
| | citalopram, clarithromycin, disopyramide, |
| | dofetilide, domperidone, donepezil, |
| | dronedarone, droperidol, erythromycin, |
| | escitalopram, flecainide, fluconazole, |
| | gatifloxacin, grepafloxacin, halofantrine, |
| | haloperidol, ibutilide, levofloxacin, |
| | levomepromazine, levosulpiride, |
| | levomethadyl, mesoridazine methadone, |
| | moxifloxacin, ondansetron, oxaliplatin, |
| | papaverine HCI (intra-coronary), |
| | pentamidine, pimozide, probucol, |
| | procainamide, propofol, quinidine, |
| | roxithromycin, sevoflurane, sotalol, |
| | sparfloxacin, sulpiride, sultopride, |
| | terlipressin, terodiline, terfenadine, |
| | thioridazine, vandetanib |
| Herbal | Herbal preparations/medications or |
| preparations/ | dietary supplements known as strong |
| medications | inducers or inhibitors of CYP3A4/5 or |
| or dietary | those with a known risk of QT prolongation |
| supplements | are prohibited throughout the study. |
| | These include, but are not limited to: St. |
| | John's wort, Kava, ephedra (ma huang), |
| | gingko biloba, dehydroepiandrosterone |
| | (DHEA), yohimbe, saw palmetto black |
| | cohosh and ginseng. Patients should stop |
| | using these herbal preparations/medications |
| | or dietary supplements 7 days prior |
| | to first dose of study drug. |
| Other | Other investigational therapies must |
| investigational | not be used while the patient is on the |
| and | study. Anti-cancer therapy (chemotherapy, |
| antineoplastic | hormonal therapy, including but not |
| therapies | limited to all SERMS (including raloxifene), |
| | biologic or radiation therapy, and |
| | surgery) other than the trial treatments |
| | must not be given to patients while the |
| | patient is on the study medication. If such |
| | agents are required for a patient, then |
| | the patient must be discontinued study drug. |

¹NTI = narrow therapeutic index drugs whose exposure-response indicates that increases in their exposure levels by the concomitant use of potent inhibitors may lead to serious safety concerns (e.g. TdP) or drugs which have <2-fold difference in the minimum toxic concentrations and minimum effective concentrations in the blood.
²Herbal product
³P-gp inducer
⁴The list provided is as of December 2019. Check https www.crediblemeds.org/healthcare-providers/drug-list for the most updated list.
⁵Drug has warning for risk of hepatotoxicity. As far as possible, avoid co-administration of QT prolonging drugs or any other drugs with the potential to increase the risk of drug-related QT prolongation (e.g. via a potential DDI that increases the exposure of ribociclib or the exposure of the QT prolonging drug). A definitive list of drugs with a known risk, possible risk, or conditional risk of QT prolongation and/or TdP is available online at qtdrugs.org.
Source: Novartis PK Sciences Memorandum: Drug-Drug Interactions (DDI) and Co-medication Considerations for Novartis Clinical Trials (January 2018), which is compiled from Indiana University “Clinically Relevant” Flockhart Table ™, University of Washington Drug Interaction Database, and FDA Drug Development and Drug Interactions: Table of Substrates, Inhibitors and Inducers.

TABLE A28
List of Medications to be Used with Caution
During Ribociclib Treatment

| Category | Drug Name |
|---|---|
| Moderate | Aprepitant, amprenavir, asafoetida resin |
| CYP3A4/5 | (Ferula asafoetida) cimetidine, crizotinib, |
| inhibitors | diltiazem, faldaprevir, imatinib, isavuconazole, |
| | netupitant, nilotinib, tofisopam, Schisandra |
| | sphenanthera (nan wu wei zi), verapamil |
| Moderate | Bosentan, dabrafenib, efavirenz, etravirine, |
| CYP3A4/5 | genistein, lopinavir⁵, modafinil, |
| inducers | nafcillin, telotristat |
| Sensitive | Alpha-dihydroergocryptine, apixaban, |
| CYP3A4/5 | aprepitant, atorvastatin, avanafil, bosutinib, |
| substrates¹ | brotizolam, budesonide, buspirone, |
| | cannabinoids⁶, cannabidiol⁶, cobimetinib, |
| | darifenacin, dasatininb, ebastine, eletriptan, |
| | eplerenone, everolimus, felodipine, |
| | fluticasone, grazoprevir, ibrutnib, |
| | isavuconazole, ivabradine, ivacaftor, |
| | lumefantrine, lurasidone, maraviroc, |
| | midazolam, midostaurin, naloxegol, neratinib, |
| | perospirone, quetiapine, ridaforolimus, |
| | rivaroxaban, sildenafil, simeprevir, |
| | ticagrelor, tilidine, tolvaptan, triazolam, |
| | ulipristal, vardenafil, venetoclax, vicriviroc, |
| | voclosporin |
| BSEP inhibitors | Alectinib, atorvastatin, bromocriptine, |
| | candesartan, clobetasol, clofaziminie, |
| | dabigatran, dipyridamole, glyburide, |
| | grazoprevir, ledipasvir, mifepristone, |
| | pioglitazone, reserpine, rifamycin, |
| | simeprevir, telmisartan, timcodar, |
| | troglitazone, velpatasvir |
| Medications | Alfuzosin, apomorphine, aripiprazole, |
| that carry a | artenimol + piperaquine, asenapine, |
| possible | atazanavir, atomoxetine, bedaquiline, |
| risk for QT | bendamustine, bortezomib, bosutinib, |
| prolongation² | buprenorphine, cabozantinib, capecitabine, |
| | ceritinib, clomipramine, crizotinib, |
| | clozapine, cyamemazine (cyamepromazine), |
| | dabrafenib, dasatinib, degarilix, |
| | delamanid, desipramine, dexmedetomidine, |
| | dolasetron, efavirenz, eliglustat, |
| | epirubicin, eribulin mesylate, |
| | ezogabine(retigabine), famotidine, |
| | felbamate, fingolimod, flupentixol, |
| | gemifloxacin, granisetron, |
| | hydrocodone-ER, iloperidone, |
| | imipramine (melipramine), isradipine, |
| | ketanserin, lapatinib, lenvatinib, leuprolide, |
| | loperamide, lithium, melperone, |
| | midostaurin, mifepristone, mirabegron, |
| | mirtazapine, moexipril/HCTZ, necitumumab, |
| | nilotinib, norfloxacin, nortriptyline, |
| | nusinersen, ofloxacin, olanzapine, |
| | osimertinib, oxytocin, paliperidone, |
| | palonosetron, panabinostat, pasireotide, |
| | pazopanib, perflutren lipid microspheres, |
| | perphenazine, pilsicainide, pimavanserin, |
| | pipamperone, promethazine, |
| | prothipendyl, quetiapine, ranolazine, |
| | rilpivirine, risperidone, romidepsin, |
| | sertindole, sorafenib, sunitinib, |
| | tamoxifen, telavancin, tetrabenazine, |
| | tipiracil/trifluridine, tizanidine, tolterodine, |
| | toremifene, trimipramine, tropisetron, |
| | vardenafil, vemurafenib, venlafaxine, |
| | vorinostat, ziprasidone |
| MATE1/2 | Acyclovir, cephalexin, cimetidine, |
| substrates³ | fexofenadine, ganciclovir, glycopyrronium, |
| | metformin, pindolol, plisicainide, |
| | ranitidine, topotecan, varenicline |
| OCT1/2 | Amantadine, , carboplatin, cisplatin, |
| substrates⁴ | cephalexin, cephradine, ipratropium, |
| | lamivudine, linagliptin, metformin, |
| | oxaliplatin, oxybutynin, phenformin, |
| | picoplatin, pilsicainide, pindolol, |
| | ranitidine, sorafenib, tropisetron, |
| | trospium, umeclidinium, and zidovudine |
| BCRP substrates | Daunorubicin, dolutegravir, doxorubicin, |
| | hematoporphyrin, imatinib, methotrexate, |
| | mitoxantrone, pitavastatin, rosuvastatin, |
| | irinotecan, ethinyl estradiol, sulfasalazine, |
| | sofosbuvir, tenofovir, topotecan, venetoclax |

¹Sensitive substrates include drugs whose plasma AUC values have been shown to increase 5-fold or higher when co-administered with a potent inhibitor.
²The list provided is as of January 2018. Check https www crediblemeds.org/healthcare-providers/drug-list for the most updated list.
³MATE1 and MATE2 share considerable substrate specificity.
⁴OCT1 and OCT2 share considerable substrate specificity.
⁵Lopinavir and atazanavir is prohibited when combined with ritonavir.
⁶Based data that, exposure of cannabidiol (CBD), tetrahydrocannabinol (THC), 11-hydroxy THC, increased by ~2-3 folds when co-administered with ketoconazole (CYP3A4 inhibitor); Stott et al, Springerplus. 2013; 2: 236
Source: Novartis PK Sciences Memorandum: Drug-Drug Interactions (DDI) and Co-medication Considerations for Novartis Clinical Trials (January 2018), which is compiled from Indiana University “Clinically Relevant” Flockhart Table ™, University of Washington Drug Interaction Database, and FDA Drug Development and Drug Interactions: Table of Substrates, Inhibitors and Inducers.

### Appendix 5. Patient Reported Outcomes

### EORTC QLQ-C30 and EORTC QLQ-BR23

The EORTC QLQ-C30 contains 30 items and is composed of both multi-item scales and single-item measures. These include five functional scales (physical, role, emotional, cognitive and social functioning), three symptom scales (fatigue, nausea/vomiting, and pain), six single items (dyspnea, insomnia, appetite loss, constipation, diarrhea and financial impact) and a global health status/QoL scale.¹⁷

The EORTC QLQ-BR23 is used for women only in conjunction with the EORTC QLQ-C30 and provides information on an additional 23 items specifically related to breast cancer. It incorporates five multi-item scales to assess systemic therapy side effects, arm symptoms, breast symptoms, body image and sexual functioning. In addition, single items assess sexual enjoyment, hair loss and future perspective.

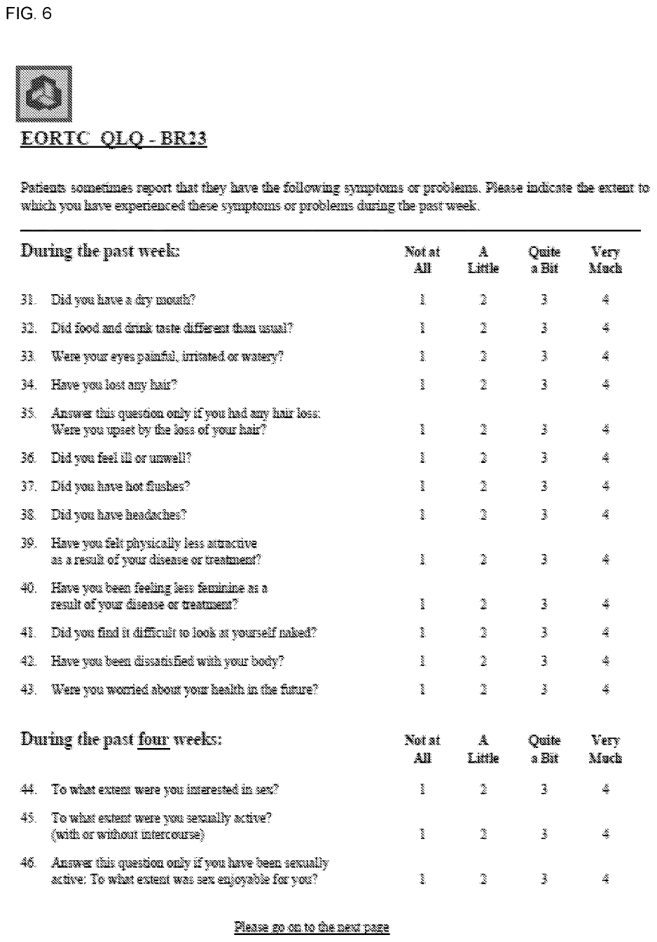

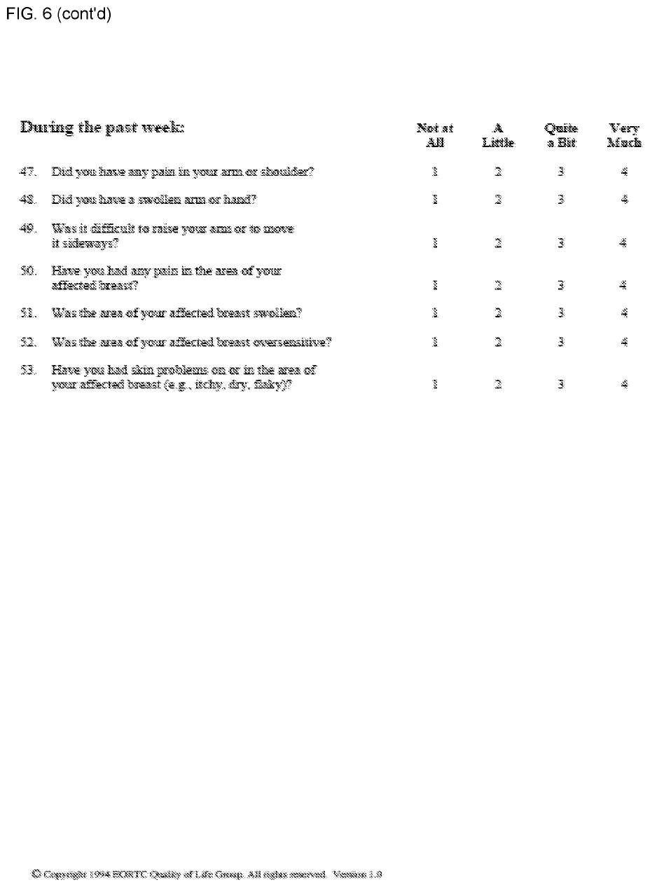

All of the scales and single-item measures range in score from 0 to 100. A high scale score represents a higher response level. Thus, a high score for a functional scale represents a high/healthy level of functioning; a high score for the global health status/QoL represents a high QoL, but a high score for a symptom scale/item represents a high level of symptomatology/problems. All scoring will follow the scoring procedures defined by the EORTC Scoring Manual. Examples of these two questionnaires are found in FIGS. 5 and 6. The questionnaires provided here are a sample for information purposes only. Questionnaires for patient completion in the trial will be provided by TRIO to be used as source documents.

### EQ-5D-5L

The EQ-5D-5L is a standardized measure of health utility that provides a single index value for one's health status. The EQ-5D-5L is frequently used for economic evaluations of health care and has been shown to be a valid and reliable instrument.⁵⁹ The EQ-5D-5L contains one item for each of five dimensions of health-related QOL (HRQOL, i.e. mobility, self-care, usual activities, pain or discomfort, and anxiety or depression). Response options for each item vary from having no problems (e.g. “ . . . no problems walking about”), moderate problems (e.g. “ . . . some problems walking about”), or extreme problems (e.g. “ . . . unable to walk about”). Patient responses to the five dimensions of HRQOL reflect a specific health state that corresponds to a population preference weight for that state on a continuous scale of 0 (death) to 1 (perfect health). A visual analog scale (VAS), ranging from 0 to 100 is also included to capture patient's rating of their overall health status. Higher scores of the EQ-5D-5L represent better health states. All scoring and handling of data will follow the User's Guide defined by the EuroQoL Group.⁵⁹

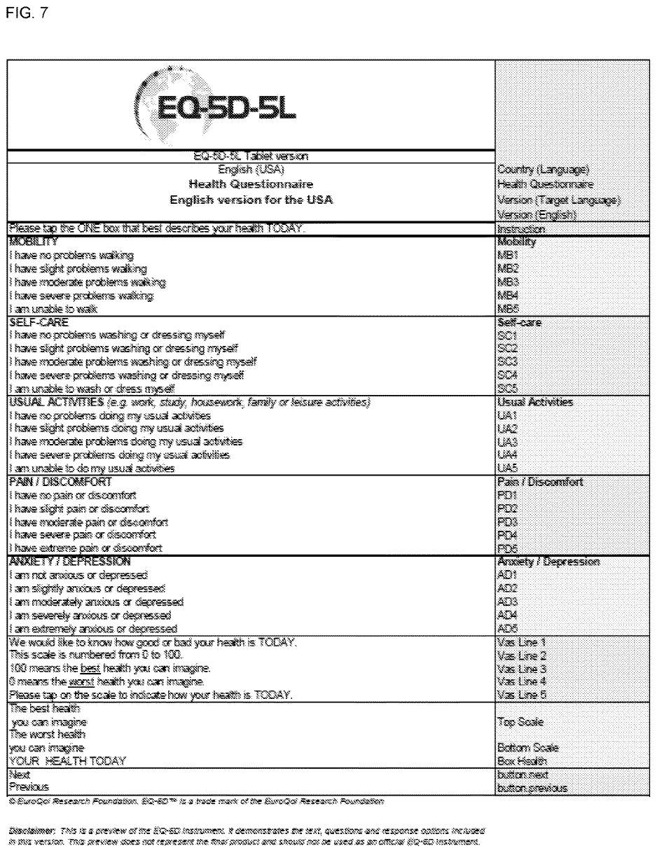

An example of this questionnaire is found in FIG. 7. The questionnaire provided here is a sample for information purposes only. Questionnaires for patient completion in the trial will be provided by TRIO to be used as source documents.

### HADS

The HADS is a 14-item, self-completed questionnaire designed to screen for depression and anxiety in and outside of the hospital and in community settings. The HADS takes approximately 10 minutes to complete and has been widely used to measure depression and anxiety in adults. Although the term “hospital” refers to the setting in which the instrument was first developed, many studies conducted throughout the world have confirmed that it is valid when used in community settings and primary care medical practice.

Even-numbered questions relate to depression, and odd-numbered questions relate to anxiety.

All items are scored on a scale from 3 to 0, with a maximum score of 21 for each subscale. A score of 11 or higher indicates the probable presence of a mood disorder, and a score of 8 to 10 is suggestive of a mood disorder. To facilitate interpretation, depression and anxiety are independent measures and can be mapped into four ranges: normal (0-7), mild (8-10), moderate (11-15), and severe (16-21). For missing data, a score for a single item may be inferred by using the mean of the remaining six items. However, if more than one item is missing, the questionnaire should not be scored.

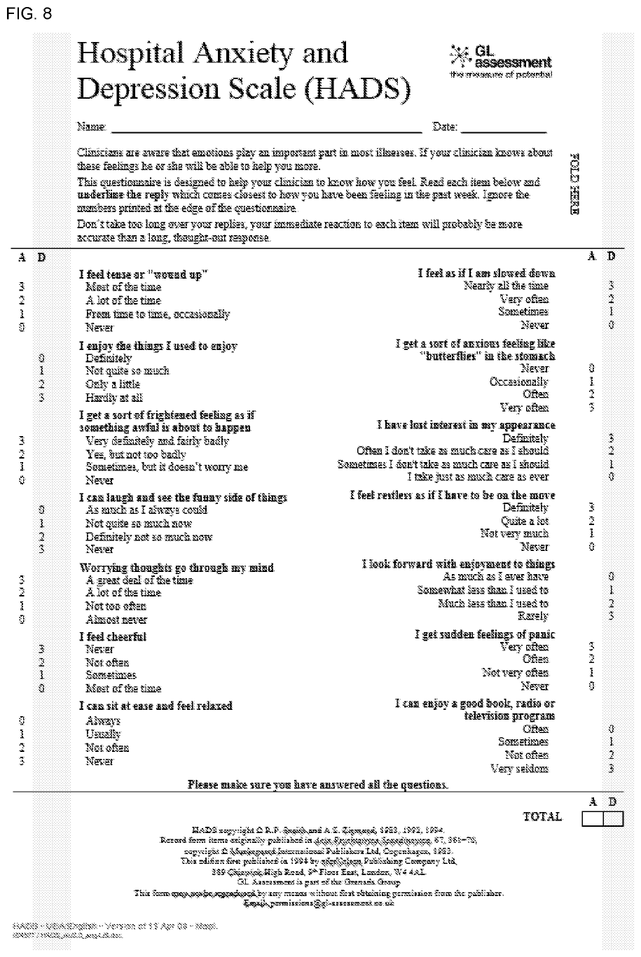

An example of this questionnaire is found in FIG. 8. The questionnaire provided here is a sample for information purposes only. Questionnaires for patient completion in the trial will be provided by TRIO to be used as source documents.

### VIII—APPENDIX B—FIRST INTERPRETABLE RESULTS

### 1. Executive Summary

TABLE B1-1
(a) Summary of the Results

| Key parameter | Definition | Outcome |
|---|---|---|
| Primary | To compare invasive | Efficacy boundary |
| Endpoint | disease-free | was crossed at |
| | survival (iDFS) for | IA-3 |
| | ribociclib + ET | Source: Table B4.1-1, |
| | versus ET Only | Table B4.1-2 |
| Secondary | OS, RFS, DDFS | Source: Table B4.2-1, |
| Endpoint | | Table B4.2-2, |
| | | Table B4.2-3 |
| Safety | TEAE | Frequency of TEAE |
| | | Source: Table B5.1-1 |
| Safety | Serious TEAE, | Frequency of Serious TEAE |
| | Death | Source: Table B5.1-2, |
| | | Table B5.1-7, |
| | | Table B5.1-8 |

Study CLEE011012301C is a phase III, multicenter, randomized, open-label trial to evaluate efficacy and safety of a 3-year regimen of ribociclib (RIB) dosed at 400 mg 3 weeks on/one week off in combination with a 5-year regimen of endocrine therapy (letrozole/anastrazole) (ET) and goserelin (for premenopausal women)) as an adjuvant treatment in patients with hormone receptor-positive, HER2-negative, early breast cancer
A total of 5101 patients were randomized in a 1:1 ratio to RIB+ET arm (n=2549), or ET Only arm (n=2552) between 10 Jan. 2019 and 20 Apr. 2021. Randomization was stratified by:
   AJCC Anatomic Stage: II [2154 (42.2%)] vs III [2947 (57.8%)]
   Menopausal Status: Premenopausal/Men [2253 (44.2%)] vs Postmenopausal [2848 (55.8%)]
   Prior Chemotherapy: Yes [4432 (86.9%)] vs No [669 (13.1%)]
   Region: North America/Western Europe/Oceania [3128 (61.3%)] vs ROW[1973 (39.7%)]
Based on the protocol pre-specified third interim analysis (IA3) review of the data on Mar. 21, 2023 by the IDMC, the efficacy boundary was met. Hence the committee recommended to stop the study for early efficacy, and as a result this interim efficacy analysis is now considered as the final analysis of the study to demonstrate the superiority of RIB+ET vs ET, in terms of iDFS. However, efficacy data will continue to be collected till approximately 5 years after last patient randomized. This document describes the first interpretable results (FIR) of the IA3, performed on the database that was locked on 22 Feb. 2023.
This FIR presents key efficacy and safety results of IA3 of the study without including any clinical interpretation.
The IA3 cutoff date (11 Jan. 2023) was planned based on the projection of approximately 425 iDFS events (~85% of the total 500 events). At the time of the database lock, 426 iDFS events (85.2% of the total 500 events) were documented up to the cutoff date. The median duration of follow-up was 34 months (from randomization to data cutoff). The minimum follow-up was 21 months. The median follow-up for iDFS is 27.7 months for both arms.
Treatment groups were balanced in terms of demography and baseline characteristics
There were 2549 patients randomized to the RIB+ET arm and 2526 (99.1%) were treated. There were 2552 patients randomized to the ET only arm and 2442 (95.7%) were treated.
   23 (0.9%) patients on RIB+ET arm and 110 (4.3%) patients on ET only arm discontinued prior to any treatment.
   There are 1984 (77.8%) patients still on treatment on RIB+ET arm and 1826 (71.6%) on ET only arm.
In the RIB+ET arm 515 (20.2%) patients completed the 3 year regimen of RIB
   The most frequent discontinuation of RIB was due to adverse events, 477 patients (18.7%).
Discontinuation due to patient decision, withdrawal of consent or lost to follow up was reported in 239 (10.2%) patients in the RIB+ET arm and 282 (11.1%) patients in ET only arm

### Primary Efficacy Endpoint

The study met its Primary objective. With 426 iDFS events in the study (189 [7.4%] in the RIB arm and 237 [9.3%] in the ET arm), there was an approximate 25.2% relative reduction in the risk of an iDFS event (HR 0.748 with 95% CI: (0.618,0.906); one-sided stratified log-rank test p-value=0.0014). The results crossed the pre-specified Lan-DeMets (O'Brien-Fleming) stopping boundary (p-value less than 0.0128 in order to claim superior efficacy).
   3-year iDFS rates are 90.4%, 95% CI (88.6%, 91.9%) for RIB+ET and 87.1%, 95% CI (85.3%, 88.8%) for ET only.
In the subgroup of patients with Anatomical Stage II EBC, there were 49/1011 (4.8%) iDFS events in the RIB+ET arm and 65/1034 (6.3%) in the ET only arm, with an iDFS hazard ratio 0.761 with 95% CI: (0.525, 1.103).
   3-year iDFS rates are 94.2%, 95% CI (92.1%, 95.7%) for RIB+ET and 91.2%, 95% CI (88.5%, 93.3%) for ET only.
In the subgroup of patients with Anatomical Stage III EBC, there were 140/1528 (9.2%) iDFS events in the RIB+ET arm and 172/1512 (11.4%) in the ET only arm, with an iDFS hazard ratio of ratio 0.74 with 95% CT: (0.592, 0.925).
   3-year iDFS rates are 87.3%, 95% CI (84.3%, 89.8%) for RIB+ET and 84.1%, 95% CI (81.4%, 86.4%) for ET only.
Overall the iDFS improvement was generally consistent across all pre-specified subgroups including menopausal status and nodal status
   N0: HR is 0.630 and 95% CI is (0.341, 1.165) for RIB+ET vs ET only arm
   N1-N3: HR is 0.771 and 95% CI is (0.630, 0.944) for RIB+ET vs ET only arm
   Pre-menopausal and men: HR is 0.722 and 95% CI is (0.530, 0.983) for RIB+ET vs ET only arm
   Post-menopausal: HR is 0.781 and 95% CI is (0.613, 0.997) for RIB+ET vs ET only arm
Per the study protocol, iDFS was only formally tested in the overall study population

### Secondary Efficacy Endpoints

Recurrence Free Survival (RFS): There were 159/2549 (6.2%) RFS events reported in the RIB+ET arm vs 207/2552 (8.1%) events in the ET only arm. The RFS HR is 0.719, 95% CI (0.584, 0.884) for RIB+ET vs ET Only. One sided nominal p-value is 0.0008.
Distant Disease Free Survival (DDFS): There were 167/2549 (6.6%) DDFS events reported in the RIB+ET arm vs 212/2552 (8.3%) events in the ET only arm. The DDFS HR is 0.739, 95% CI (0.603, 0.905) for RIB+ET vs ET Only. One sided nominal p-value is 0.0017.
Overall survival (OS): There were 61/2549 (2.4%) OS events reported in the RIB+ET arm vs 73/2552 (2.9%) events in the ET only arm. The OS HR is 0.759, 95% CI (0.539, 1.068) for RIB+ET vs ET Only. One sided nominal p-value is 0.0563.
The p-values for the secondary efficacy endpoints are nominal and not adjusted for multiplicity

### Safety and Tolerability

The median duration of exposure to study treatment was 30.1 months in the RIB+ET arm, and 30.0 months in the ET Only arm (from start of treatment to last treatment as per data cut-off date)
   515 (20.2%) patients completed the 3-year RIB regimen and 1450 (56.9%) patients had achieved 2 years or more.
RIB dose reduction occurred in 1071 (42.4%) patients. 566 (22.4%) patients required RIB dose reduction due to AE
The following treatment-emergent adverse events (TEAE) were reported in the RIB+ET arm and ET Only arm, respectively:

| | |
|---|---|
| Any TEAE | 97.9% vs 87.1% |
| Grade ≥3 TEAE | 62.6% vs 17.8% |
| Serious TEAE | 13.3% vs 9.9% |
| TEAEs leading to treatment discontinuation | 20.7% vs 5.3% |

A total of 19 (0.8%) and 9 (0.4%) patients died on treatment (up to 30 days after end of treatment) in the RIB+ET and ET Only arms respectively. The main causes of on-treatment deaths in RIB+ET arm and ET Only arm, respectively, were

| | |
|---|---|
| Adverse event | 0.5% vs. 0.2% |
| Disease recurrence | 0.3% vs. 0.2% |
| Other | 0% vs. <0.1% |

Among the on-treatment deaths due to adverse events, 6 (0.2%) patients in the RIB+ET arm and 1 (0.0%) patients in the ET only arm died due to Covid-19.
The main causes of overall deaths during the study (on-treatment & post-treatment) on RIB+ET arm and ET Only arm, respectively, were

| | |
|---|---|
| Disease recurrence/progression | 1.6% vs. 2.5% |
| Adverse event | 0.5% vs. 0.2% |
| Other | 0.2% vs. 0.4% |

Grade 3/4 hematologic lab abnormalities in RIB+ET arm and ET Only arm, respectively, were

| | |
|---|---|
| Neutrophils (10E9/L) Hypo | 44.6% vs. 1.7% |
| Leukocytes (10E9/L) Hypo | 27.3% vs. 0.6% |
| Lymphocytes (10E9/L) Hypo | 19.4% vs. 7.0% |

Grade 3/4 biochemical lab abnormalities in RIB+ET arm and ET Only arm, respectively, were

Alanine Aminotransferase (IU/L) Hyper 7.9% vs. 1.0%
Aspartate Aminotransferase (IU/L) Hyper Grade 5.1% vs 1.1%
Gamma glutamyl transferase (IU/K) Hyper 3.7% vs. 3.6%

Notable ECG values based on Central assessments
   >60 ms increase from baseline in QTcF interval in RIB+ET arm and ET Only arm, respectively, were 0.8% ovs. 0.1%
   QTcF >480 ms in RIB+ET arm and ET Only arm, respectively, were 0.4% vs. 0.2%
   QTcF >500 ms in RIB+ET arm and ET Only arm, respectively, were 0.1% vs. 0.0%
(ALT or AST) >3×ULN & TBL >2×ULN & ALP <2×ULN: A total of 8 (0.3%) and 1 (0.0%) patients were reported in RIB+ET arm and ET Only arm, respectively.

### 2 Additional Information

None.

### 3 Patients Studied

### 3.1 Subject Disposition

TABLE B3.1-1
Subject disposition (Full analysis set)

| | Ribociclib + ET | | ET only |
|---|---|---|---|
| | N = 2549 | | N = 2552 |
| | n (%) | | n (%) |
| Untreated | 23 (0.9) | | 110 (4.3) |
| Treated | 2526 (99.1) | | 2442 (95.7) |
| Treatment ongoing | 1984 (77.8) | | 1826 (71.6) |
| | Ribociclib | NSAI | NSAI |
| Number of patients who | 1377 (54.0) | 542 (21.3) | 617 (24.2) |
| discontinued the component | | | |
| Primary reason for | | | |
| discontinuation | | | |
| Adverse event | 477 (18.7) | 118 (4.6) | 105 (4.1) |
| Completed | 515 (20.2) | 0 | 0 |
| Death | 3 (0.1) | 5 (0.2) | 3 ( 0.1) |
| Disease relapse | 109 (4.3) | 142 (5.6) | 186 (7.3) |
| Lost to follow-up | 6 (0.2) | 8 (0.3) | 12 (0.5) |
| Other | 14 ( 0.5) | 8 (0.3) | 8 (0.3) |
| Patient decision to discontinue | 139 (5.5) | 123 (4.8) | 116 (4.5) |
| treatment | | | |
| Physician decision | 24 ( 0.9) | 25 (1.0) | 26 (1.0) |
| Protocol deviation | 7 (0.3) | 5 (0.2) | 7 (0.3) |
| Withdrawal by subject | 80 (3.1) | 108 (4.2) | 154 (6.0) |

### 3.2 Analysis Sets

TABLE B3.2-1
Analysis Set Sample Sizes (Full analysis set)

| | ET + ribociclib | ET only | Total |
|---|---|---|---|
| | N = 2549 | N = 2552 | N = 5101 |
| Analysis set | n (%) | n (%) | n (%) |
| Full analysis set | 2549 (100) | 2552 (100) | 5101 (100) |
| Safety analysis set | 2524 (99.0) | 2442 (95.7) | 4968 (97.4) |
| Per-protocol set | 2496 (97.9) | 2424 (95.0) | 4920 (96.5) |

Note:
Denominator for percentages is based on the number of patients in the full analysis set.

### 3.3 Demographics and Other Baseline Characteristics

TABLE B3.3-1
Demographics and baseline characteristics (Full analysis set)

| | ET + ribociclib | ET only | Total |
|---|---|---|---|
| | N = 2549 | N = 2552 | N = 5101 |
| Characteristic | n (%) | n (%) | n (%) |
| Age group | | | |
| <45 | 611 (24.0) | 591 (23.2) | 1202 (23.6) |
| 45 to 54 | 849 (33.3) | 895 (35.1) | 1744 (34.2) |
| 55 to 64 | 682 (26.8) | 700 (27.4) | 1382 (27.1) |
| >=65 | 407 (16.0) | 366 (14.3) | 773 (15.2) |
| Missing | 0 | 0 | 0 |
| Age (years) | | | |
| n | 2549 | 2552 | 5101 |
| Mean | 52.9 | 52.7 | 52.8 |
| SD | 10.75 | 10.77 | 10.76 |
| Min | 24 | 24 | 24 |
| Median | 52.0 | 52.0 | 52.0 |
| Max | 90 | 89 | 90 |
| Gender | | | |
| Male | 11 (0.4) | 9 (0.4) | 20 (0.4) |
| Female | 2538 (99.6) | 2543 (99.6) | 5081 (99.6) |
| Unknown | 0 | 0 | 0 |
| Undifferentiated | 0 | 0 | 0 |
| Missing | 0 | 0 | 0 |
| Race | | | |
| White | 1876 (73.6) | 1868 (73.2) | 3744 (73.4) |
| Black or African American | 42 (1.6) | 47 (1.8) | 89 (1.7) |
| Asian | 341 (13.4) | 334 (13.1) | 675 (13.2) |
| Native Hawaiian or Other | 3 (0.1) | 1 (0.0) | 4 (0.1) |
| Pacific Islander | | | |
| American Indian or Alaska | 4 (0.2) | 3 (0.1) | 7 (0.1) |
| Native | | | |
| Other | 145 (5.7) | 172 (6.7) | 317 (6.2) |
| Missing | 138 (5.4) | 127 (5.0) | 265 (5.2) |
| Ethnicity | | | |
| Hispanic or Latino | 212 (8.3) | 223 (8.7) | 435 (8.5) |
| Not Hispanic or Latino | 2076 (81.4) | 2054 (80.5) | 4130 (81.0) |
| Unknown | 172 (6.7) | 201 (7.9) | 373 (7.3) |
| Missing | 89 (3.5) | 74 (2.9) | 163 (3.2) |
| Region | | | |
| Asia | 281 (11.0) | 290 (11.4) | 571 (11.2) |
| Europe | 1505 (59.0) | 1506 (59.0) | 3011 (59.0) |
| North America/Australia | 624 (24.5) | 612 (24.0) | 1236 (24.2) |
| Latin America | 139 (5.5) | 144 (5.6) | 283 (5.5) |
| Missing | 0 | 0 | 0 |
| ECOG performance status | | | |
| 0 | 2106 (82.6) | 2132 (83.5) | 4238 (83.1) |
| 1 | 440 (17.3) | 418 (16.4) | 858 (16.8) |
| Missing | 3 (0.1) | 2 (0.1) | 5 (0.1) |
| Weight (kg) | | | |
| n | 2534 | 2542 | 5076 |
| Mean | 72.4 | 72.2 | 72.3 |
| SD | 16.20 | 15.53 | 15.86 |
| Min | 38 | 41 | 38 |
| Median | 70.0 | 70.0 | 70.0 |
| Max | 166 | 169 | 169 |
| Height (cm) | | | |
| n | 2523 | 2522 | 5045 |
| Mean | 162.9 | 162.7 | 162.8 |
| SD | 6.78 | 6.85 | 6.81 |
| Min | 140 | 140 | 140 |
| Median | 163.0 | 163.0 | 163.0 |
| Max | 198 | 191 | 198 |
| BMI (kg/mÂ²) | | | |
| n | 2518 | 2521 | 5039 |
| Mean | 27.3 | 27.3 | 27.3 |
| SD | 5.81 | 5.70 | 5.76 |
| Min | 16 | 15 | 15 |
| Median | 26.3 | 26.5 | 26.4 |
| Max | 56 | 59 | 59 |

Weight and height are the last non-missing assessments on or before the date of randomization.
BMI: body mass index is calculated based on raw data measurements.

TABLE B3.3-2
Diagnosis and Extent of Cancer (Full analysis set)

| | ET + ribociclib | ET only | Total |
|---|---|---|---|
| | N = 2549 | N = 2552 | N = 5101 |
| Characteristic | n (%) | n (%) | n (%) |
| Tumor Location | | | |
| Right | 1277 (50.1) | 1258 (49.3) | 2535 (49.7) |
| Left | 1271 (49.9) | 1287 (50.4) | 2558 (50.1) |
| Bilateral | 1 (0.0) | 7 (0.3) | 8 (0.2) |
| Missing | 0 | 0 | 0 |
| Histopathological grade at | | | |
| diagnosis - n (%) | | | |
| GX | 30 (1.2) | 32 (1.3) | 62 (1.2) |
| G1 | 218 (8.6) | 240 (9.4) | 458 (9.0) |
| G2 | 1458 (57.2) | 1451 (56.9) | 2909 (57.0) |
| G3 | 521 (20.4) | 549 (21.5) | 1070 (21.0) |
| Not Done | 292 (11.5) | 258 (10.1) | 550 (10.8) |
| Missing | 30 (1.2) | 22 (0.9) | 52 (1.0) |
| T stage at diagnosis - n (%) | | | |
| TX | 175 (6.9) | 173 (6.8) | 348 (6.8) |
| T0 | 4 (0.2) | 7 (0.3) | 11 (0.2) |
| Tis | 2 (0.1) | 3 (0.1) | 5 (0.1) |
| T1 | 471 (18.5) | 442 (17.3) | 913 (17.9) |
| T2 | 1181 (46.3) | 1235 (48.4) | 2416 (47.4) |
| T3 | 471 (18.5) | 472 (18.5) | 943 (18.5) |
| T4 | 200 (7.8) | 184 (7.2) | 384 (7.5) |
| Missing | 45 (1.8) | 36 (1.4) | 81 (1.6) |
| N stage at diagnosis - n (%) | | | |
| NX | 272 (10.7) | 264 (10.3) | 536 (10.5) |
| N0 | 694 (27.2) | 737 (28.9) | 1431 (28.1) |
| N1 | 1050 (41.2) | 1049 (41.1) | 2099 (41.1) |
| N2 | 332 (13.0) | 292 (11.4) | 624 (12.2) |
| N3 | 151 (5.9) | 175 (6.9) | 326 (6.4) |
| Missing | 50 (2.0) | 35 (1.4) | 85 (1.7) |
| M stage at diagnosis - n (%) | | | |
| M0 | 2549 (100) | 2552 (100) | 5101 (100) |
| M1 | 0 | 0 | 0 |
| Missing | 0 | 0 | 0 |
| Ki67 score at initial diagnosis | | | |
| n | 1861 | 1908 | 3769 |
| Mean | 27.1 | 27.1 | 27.1 |
| SD | 19.88 | 19.50 | 19.69 |
| Min | 0 | 0 | 0 |
| Median | 20.0 | 20.5 | 20.0 |
| Max | 99 | 100 | 100 |
| Ki67 category at initial | | | |
| diagnosis | | | |
| <=14% | 508 (19.9) | 508 (19.9) | 1016 (19.9) |
| >14% | 1353 (53.1) | 1400 (54.9) | 2753 (54.0) |
| <=20% | 938 (36.8) | 954 (37.4) | 1892 (37.1) |
| >20% | 923 (36.2) | 954 (37.4) | 1877 (36.8) |
| Missing | 688 (27.0) | 644 (25.2) | 1332 (26.1) |
| Histopathological grade on | | | |
| surgical specimen - n (%) | | | |
| GX | 32 (1.3) | 30 (1.2) | 62 (1.2) |
| G1 | 213 (8.4) | 217 (8.5) | 430 (8.4) |
| G2 | 1460 (57.3) | 1432 (56.1) | 2892 (56.7) |
| G3 | 684 (26.8) | 702 (27.5) | 1386 (27.2) |
| Not Done | 159 (6.2) | 168 (6.6) | 327 (6.4) |
| Missing | 1 (0.0) | 3 (0.1) | 4 (0.1) |
| T stage on surgical specimen - | | | |
| n (%) | | | |
| TX | 20 (0.8) | 9 (0.4) | 29 (0.6) |
| T0 | 56 (2.2) | 52 (2.0) | 108 (2.1) |
| Tis | 16 (0.6) | 19 (0.7) | 35 (0.7) |
| T1 | 774 (30.4) | 761 (29.8) | 1535 (30.1) |
| T2 | 1162 (45.6) | 1198 (46.9) | 2360 (46.3) |
| T3 | 427 (16.8) | 422 (16.5) | 849 (16.6) |
| T4 | 92 (3.6) | 91 (3.6) | 183 (3.6) |
| Missing | 2 (0.1) | 0 | 2 (0.0) |
| N stage on surgical specimen - | | | |
| n (%) | | | |
| NX | 2 (0.1) | 5 (0.2) | 7 (0.1) |
| N0 | 378 (14.8) | 418 (16.4) | 796 (15.6) |
| N1 | 1062 (41.7) | 1039 (40.7) | 2101 (41.2) |
| N2 | 733 (28.8) | 690 (27.0) | 1423 (27.9) |
| N3 | 372 (14.6) | 399 (15.6) | 771 (15.1) |
| Missing | 2 (0.1) | 1 (0.0) | 3 (0.1) |
| Ki67 score on surgical | | | |
| specimen | | | |
| n | 1269 | 1332 | 2601 |
| Mean | 20.6 | 20.9 | 20.7 |
| SD | 17.82 | 18.15 | 17.99 |
| Min | 0 | 0 | 0 |
| Median | 15.0 | 15.0 | 15.0 |
| Max | 99 | 98 | 99 |
| Ki67 category on surgical | | | |
| specimen | | | |
| <=14% | 541 (21.2) | 577 (22.6) | 1118 (21.9) |
| >14% | 728 (28.6) | 755 (29.6) | 1483 (29.1) |
| <=20% | 817 (32.1) | 864 (33.9) | 1681 (33.0) |
| >20% | 452 (17.7) | 468 (18.3) | 920 (18.0) |
| Missing | 1280 (50.2) | 1220 (47.8) | 2500 (49.0) |
| Time since initial diagnosis | | | |
| (months) | | | |
| n | 2517 | 2528 | 5045 |
| Mean | 11.8 | 11.8 | 11.8 |
| SD | 3.53 | 3.58 | 3.55 |
| Min | 1 | 1 | 1 |
| Median | 11.7 | 11.7 | 11.7 |
| Max | 23 | 27 | 27 |
| Predominant histology - n (%) | | | |
| Invasive ductal carcinoma | 1857 (72.9) | 1881 (73.7) | 3738 (73.3) |
| NOS | | | |
| Invasive lobular | 455 (17.9) | 450 (17.6) | 905 (17.7) |
| Carcinoma medullary | 1 (0.0) | 1 (0.0) | 2 (0.0) |
| Mucinous | 17 (0.7) | 16 (0.6) | 33 (0.6) |
| Papillary | 18 (0.7) | 12 (0.5) | 30 (0.6) |
| Tubular | 5 (0.2) | 3 (0.1) | 8 (0.2) |
| Ductal Carcinoma In Situ | 1 (0.0) | 0 | 1 (0.0) |
| Lobular Carcinoma In Situ | 0 | 0 | 0 |
| Other | 194 (7.6) | 189 (7.4) | 383 (7.5) |
| Missing | 1 (0.0) | 0 | 1 (0.0) |
| Prior surgery - n (%) | | | |
| Mastectomy | 1664 (65.3) | 1691 (66.3) | 3355 (65.8) |
| Breast conserving surgery | 978 (38.4) | 963 (37.7) | 1941 (38.1) |
| Axillary lymph node dissection | 2165 (84.9) | 2149 (84.2) | 4314 (84.6) |
| Sentinel lymph node biopsy | 926 (36.3) | 920 (36.1) | 1846 (36.2) |
| Other | 143 (5.6) | 162 (6.3) | 305 (6.0) |
| Missing | 0 | 0 | 0 |
| HER2 ISH result prior to | | | |
| surgery (reported only if | | | |
| performed) - n (%) | | | |
| Amplification | 4 (0.2) | 7 (0.3) | 11 (0.2) |
| Non-Amplification | 612 (24.0) | 653 (25.6) | 1265 (24.8) |
| Equivocal | 19 (0.7) | 13 (0.5) | 32 (0.6) |
| Unknown | 6 (0.2) | 11 (0.4) | 17 (0.3) |
| HER2 ISH result from the | | | |
| surgical specimen (reported | | | |
| only if performed) - n (%) | | | |
| Amplification | 2 (0.1) | 1 (0.0) | 3 (0.1) |
| Non-Amplification | 417 (16.4) | 423 (16.6) | 840 (16.5) |
| Equivocal | 1 (0.0) | 1 (0.0) | 2 (0.0) |
| Unknown | 2 (0.1) | 2 (0.1) | 4 (0.1) |
| HER2 IHC score prior to | | | |
| surgery (reported only if | | | |
| performed) - n (%) | | | |
| 0 | 856 (33.6) | 881 (34.5) | 1737 (34.1) |
| 1+ | 862 (33.8) | 813 (31.9) | 1675 (32.8) |
| 2+ | 464 (18.2) | 480 (18.8) | 944 (18.5) |
| 3+ | 5 (0.2) | 5 (0.2) | 10 (0.2) |
| Unknown | 21 (0.8) | 21 (0.8) | 42 (0.8) |
| HER2 IHC score from the | | | |
| surgical specimen (reported | | | |
| only if performed) - n (%) | | | |
| 0 | 625 (24.5) | 610 (23.9) | 1235 (24.2) |
| 1+ | 513 (20.1) | 516 (20.2) | 1029 (20.2) |
| 2+ | 235 (9.2) | 262 (10.3) | 497 (9.7) |
| 3+ | 1 (0.0) | 3 (0.1) | 4 (0.1) |
| Unknown | 6 (0.2) | 10 (0.4) | 16 (0.3) |
| Estrogen receptor (ER) IHC | | | |
| status prior to surgery | | | |
| (reported only if performed) - | | | |
| n (%) | | | |
| Positive | 2342 (91.9) | 2328 (91.2) | 4670 (91.6) |
| Negative | 8 (0.3) | 16 (0.6) | 24 (0.5) |
| Unknown | 0 | 1 (0.0) | 1 (0.0) |
| Estrogen receptor (ER) IHC | | | |
| status from the surgical | | | |
| specimen (reported only if | | | |
| performed) - n (%) | | | |
| Positive | 1369 (53.7) | 1403 (55.0) | 2772 (54.3) |
| Negative | 2 (0.1) | 5 (0.2) | 7 (0.1) |
| Unknown | 3 (0.1) | 3 (0.1) | 6 (0.1) |
| Progesterone receptor (PR) | | | |
| IHC status prior to surgery | | | |
| (reported only if performed) - | | | |
| n (%) | | | |
| Positive | 2051 (80.5) | 2034 (79.7) | 4085 (80.1) |
| Negative | 287 (11.3) | 295 (11.6) | 582 (11.4) |
| Unknown | 12 (0.5) | 16 (0.6) | 28 (0.5) |
| Progesterone receptor (PR) | | | |
| IHC status from the surgical | | | |
| specimen (reported only if | | | |
| performed) - n (%) | | | |
| Positive | 1133 (44.4) | 1149 (45.0) | 2282 (44.7) |
| Negative | 236 (9.3) | 255 (10.0) | 491 (9.6) |
| Unknown | 6 (0.2) | 8 (0.3) | 14 (0.3) |
| ER/PR combination statuses - | | | |
| n (%) | | | |
| ER+/PR+ | 2172 (85.2) | 2132 (83.5) | 4304 (84.4) |
| ER+/PR− | 359 (14.1) | 392 (15.4) | 751 (14.7) |
| ER−/PR+ | 3 (0.1) | 12 (0.5) | 15 (0.3) |
| ER+/UNK | 10 (0.4) | 13 (0.5) | 23 (0.5) |
| UNK/PR+ | 2 (0.1) | 2 (0.1) | 4 (0.1) |
| UNK/PR− | 1 (0.0) | 1 (0.0) | 2 (0.0) |
| UNK/UNK | 2 (0.1) | 0 | 2 (0.0) |
| AJCC 8th ed. anatomic stage - | | | |
| n (%) | | | |
| Stage 0 | 0 | 0 | 0 |
| Stage I | 9 (0.4) | 5 (0.2) | 14 (0.3) |
| Stage II | 1011 (39.7) | 1034 (40.5) | 2045 (40.1) |
| Stage III | 1528 (59.9) | 1512 (59.2) | 3040 (59.6) |
| Stage IV | 0 | 0 | 0 |
| Missing | 1 (0.0) | 1 (0.0) | 2 (0.0) |
| Genomic test | | | |
| Endopredict | 23 (0.9) | 28 (1.1) | 51 (1.0) |
| Mammaprint | 46 (1.8) | 51 (2.0) | 97 (1.9) |
| Oncotype DX | 120 (4.7) | 129 (5.1) | 249 (4.9) |
| Pam50 | 38 (1.5) | 29 (1.1) | 67 (1.3) |
| Other | 109 (4.3) | 103 (4.0) | 212 (4.2) |
| EndoPredict EPclin Risk Score | | | |
| High risk | 19 (0.7) | 21 (0.8) | 40 (0.8) |
| Low risk | 4 (0.2) | 7 (0.3) | 11 (0.2) |
| Missing | 2526 (99.1) | 2524 (98.9) | 5050 (99.0) |
| Mammaprint | | | |
| High risk | 22 (0.9) | 33 (1.3) | 55 (1.1) |
| Low risk | 24 (0.9) | 18 (0.7) | 42 (0.8) |
| Missing | 2503 (98.2) | 2501 (98.0) | 5004 (98.1) |
| Oncotype DX Breast | | | |
| Recurrence Score | | | |
| Recurrence score >= 26 | 33 (1.3) | 43 (1.7) | 76 (1.5) |
| Recurrence score < 26 | 87 (3.4) | 86 (3.4) | 173 (3.4) |
| Missing | 2429 (95.3) | 2423 (94.9) | 4852 (95.1) |
| Prosigna/PAM50 | | | |
| High risk | 24 (0.9) | 19 (0.7) | 43 (0.8) |
| Intermediate risk | 10 (0.4) | 7 (0.3) | 17 (0.3) |
| Low risk | 4 (0.2) | 3 (0.1) | 7 (0.1) |
| Missing | 2511 (98.5) | 2523 (98.9) | 5034 (98.7) |

Subjects may have had more than one prior surgery, but are only counted once per category.
T stage category T1 collects T1mi, T1a, T1b, and T1c. Category T4 collects T4a, T4b, T4c, and T4d.
N stage category N0 collects N0 and N0(i+). Category N1 collects N1, N1a, N1c, and N1mi. Category N2 collects N2a, N2b, and N2c. Category N3 collects N3a, N3b, and N3c.
AJCC 8th ed. category Stage 1 collects Stage IA and Stage IB. Category Stage II collects Stage IIA and Stage IIB. Category Stage III collects Stage IIIA, Stage IIIB, and Stage IIIC. Stage is derived using TNM from surgery for patients having not received neo-/adjuvant treatment, or as worst stage derived using TNM at diagnosis and TNM from surgery for patients having received neo-/adjuvant treatment.
Patients may have had more than one Genomic test type but are only counted once per type.

TABLE B3.3-3
(f) Anatomic Stage Group at randomization (Full analysis set)

| | ET + ribociclib | ET only | Total |
|---|---|---|---|
| | N = 2549 | N = 2552 | N = 5101 |
| Characteristic | n (%) | n (%) | n (%) |
| Anatomic Stage I | 9 (0.4) | 5 (0.2) | 14 (0.3) |
| Anatomic Stage IIA | 479 (18.8) | 521 (20.4) | 1000 (19.6) |
| N1 | 267 (10.5) | 280 (11.0) | 547 (10.7) |
| N0 with | 212 (8.3) | 241 (9.4) | 453 (8.9) |
| Grade 3 | 104 (4.1) | 109 (4.3) | 213 (4.2) |
| Grade 2 with any of the following | 76 (3.0) | 102 (4.0) | 178 (3.5) |
| Ki67 >= 20% | 70 (2.7) | 91 (3.6) | 161 (3.2) |
| Oncotype DX Breast Recurrence | 4 (0.2) | 10 (0.4) | 14 (0.3) |
| score >= 26 | | | |
| Prosigna/PAM50 as high risk | 6 (0.2) | 3 (0.1) | 9 (0.2) |
| MammaPrint as high risk | 2 (0.1) | 4 (0.2) | 6 (0.1) |
| EndoPredict EPclin Risk | 3 (0.1) | 5 (0.2) | 8 (0.2) |
| Score as high risk | | | |
| Grade 2 with none of the | 28 (1.1) | 26 (1.0) | 54 (1.1) |
| above risk factors | | | |
| Anatomic Stage IIB | 532 (20.9) | 513 (20.1) | 1045 (20.5) |
| Anatomic Stage III | 1528 (59.9) | 1512 (59.2) | 3040 (59.6) |
| Missing stage | 1 (0.0) | 1 (0.0) | 2 (0.0) |

AJCC 8th ed. category Stage I collects Stage IA and Stage IB. Category Stage III collects Stage IIIA, Stage IIIB, and Stage IIIC. Stage is derived using TNM from surgery for patients having not received neo-/adjuvant treatment, or as worst stage derived using TNM at diagnosis and TNM from surgery for patients having received neo-/adjuvant treatment.
Ki67 based on surgical specimen record. If surgical specimen for Ki67 unavailable, then score at diagnosis is used for the summary.

TABLE B3.3-4
(g) Prior antineoplastic therapy (Full analysis set)

| | ET + ribociclib | ET only | Total |
|---|---|---|---|
| | N = 2549 | N = 2552 | N = 5101 |
| Therapy | n (%) | n (%) | n (%) |
| Number of patients who received any | 2423 (95.1) | 2439 (95.6) | 4862 (95.3) |
| prior anti-neoplastic medications | | | |
| Chemotherapy | 2249 (88.2) | 2245 (88.0) | 4494 (88.1) |
| Anthracyclines | 2014 (79.0) | 2037 (79.8) | 4051 (79.4) |
| Taxanes | 2147 (84.2) | 2132 (83.5) | 4279 (83.9) |
| Other | 2190 (85.9) | 2189 (85.8) | 4379 (85.8) |
| Endocrine therapy | 1824 (71.6) | 1801 (70.6) | 3625 (71.1) |
| Aromatase inhibitors | 1601 (62.8) | 1592 (62.4) | 3193 (62.6) |
| Anti-estrogens | 344 (13.5) | 341 (13.4) | 685 (13.4) |
| Gonadotropin-releasing hormone | 670 (26.3) | 620 (24.3) | 1290 (25.3) |
| analogues | | | |
| Other | 2 (0.1) | 4 (0.2) | 6 (0.1) |
| Biologic/targeted therapy | 2 (0.1) | 9 (0.4) | 11 (0.2) |
| Other | 9 (0.4) | 6 (0.2) | 15 (0.3) |
| Therapy setting | | | |
| Adjuvant | 2160 (84.7) | 2150 (84.2) | 4310 (84.5) |
| Adjuvant chemotherapy | 1223 (48.0) | 1220 (47.8) | 2443 (47.9) |
| Neo-adjuvant | 1129 (44.3) | 1148 (45.0) | 2277 (44.6) |
| Neo-adjuvant chemotherapy | 1085 (42.6) | 1095 (42.9) | 2180 (42.7) |
| Lowest ATC class | | | |
| Antineoplastic and | 2423 (95.1) | 2439 (95.6) | 4862 (95.3) |
| immunomodulating agents | | | |
| Dermatologicals | 0 | 1 (0.0) | 1 (0.0) |
| Musculo-skeletal system | 4 (0.2) | 2 (0.1) | 6 (0.1) |
| Systemic hormonal preparations, | 2 (0.1) | 4 (0.2) | 6 (0.1) |
| excl. sex hormones and insulins | | | |
| Various | 2 (0.1) | 5 (0.2) | 7 (0.1) |
| Number of patients who received any | 2292 (89.9) | 2302 (90.2) | 4594 (90.1) |
| prior anti-neoplastic radiotherapy | | | |
| Time since end of last radiotherapy | | | |
| (months) | | | |
| n | 2292 | 2302 | 4594 |
| Mean | 3.1 | 3.1 | 3.1 |
| SD | 2.48 | 2.50 | 2.49 |
| Min | 0 | 0 | 0 |
| Median | 2.3 | 2.3 | 2.3 |
| Max | 14 | 14 | 14 |
| Location of last radiotherapy | | | |
| Breast | 1035 (40.6) | 1004 (39.3) | 2039 (40.0) |
| Chest wall | 1209 (47.4) | 1210 (47.4) | 2419 (47.4) |
| Axillary lymph node | 1010 (39.6) | 973 (38.1) | 1983 (38.9) |
| Supraclavicular lymph node | 1066 (41.8) | 1079 (42.3) | 2145 (42.1) |
| Internal mammary lymph node | 374 (14.7) | 410 (16.1) | 784 (15.4) |
| Other | 287 (11.3) | 310 (12.1) | 597 (11.7) |
| Duration of prior endocrine therapy | | | |
| (months) | | | |
| n | 1818 | 1794 | 3612 |
| Mean | 3.8 | 3.9 | 3.8 |
| SD | 2.78 | 2.77 | 2.77 |
| Min | 0 | 0 | 0 |
| Median | 3.1 | 3.2 | 3.2 |
| Max | 17 | 18 | 18 |
| Number of patients who received any | 2548 (100) | 2552 (100) | 5100 (100) |
| prior surgery | | | |
| Time since end of last surgery | | | |
| (months) | | | |
| n | 2548 | 2552 | 5100 |
| Mean | 7.9 | 7.9 | 7.9 |
| SD | 3.80 | 3.76 | 3.78 |
| Min | 0 | 0 | 0 |
| Median | 7.9 | 7.8 | 7.8 |
| Max | 18 | 21 | 21 |
| Type of surgery | | | |
| Biopsy | 679 (26.6) | 673 (26.4) | 1352 (26.5) |
| Therapy | n (%) | n (%) | n (%) |
| Not Biopsy | 2542 (99.7) | 2549 (99.9) | 5091 (99.8) |

Anti-neoplastic medications are coded using the WHO-DD Version: Sep, 2022.
Subjects may have multiple prior anti-neoplastic therapy types but are only counted once per therapy or sub-therapy type.
Subjects may have multiple radiotherapy locations based on the last reported date but are only counted once per radiotherapy location.
Subjects may have both Adjuvant and Neo-adjuvant prior therapy settings but are only counted once per therapy setting.
Time since end of last radiotherapy = (randomization date - end date of radiotherapy) in months.
Time since end of last surgery = (randomization date - date of last surgery) in months.

### 3.4 Subject Exposure

TABLE B3.4-1
(h) Extent of Exposure (Safety set)

| | Study Treatment | | |
|---|---|---|---|
| | Ribociclib | | |
| | N = 2524 | ET + ribociclib | ET only |
| | n (%) | N = 2524 | N = 2444 |
| Duration of exposure | | | |
| 0 - 3 months | 304 (12.0) | 122 (4.8) | 168 (6.9) |
| 3 - 6 months | 164 (6.5) | 84 (3.3) | 79 (3.2) |
| 6 - 9 months | 91 (3.6) | 58 (2.3) | 50 (2.0) |
| 9 - 12 months | 51 (2.0) | 48 (1.9) | 55 (2.3) |
| 12 - 15 months | 51 (2.0) | 40 (1.6) | 45 (1.8) |
| 15 - 18 months | 52 (2.1) | 34 (1.3) | 43 (1.8) |
| 18 - 21 months | 90 (3.6) | 57 (2.3) | 65 (2.7) |
| 21 - 24 months | 272 (10.8) | 318 (12.6) | 281 (11.5) |
| 24 - 27 months | 110 (4.4) | 103 (4.1) | 92 (3.8) |
| 27 - 30 months | 324 (12.8) | 381 (15.1) | 349 (14.3) |
| 30 - 33 months | 337 (13.4) | 188 (7.4) | 189 (7.7) |
| 33 - 36 months | 665 (26.3) | 491 (19.5) | 460 (18.8) |
| 36 - 39 months | 13 (0.5) | 364 (14.4) | 338 (13.8) |
| 39 - 42 months | 0 | 193 (7.6) | 179 (7.3) |
| 42 - 45 months | 0 | 34 (1.3) | 42 (1.7) |
| 45 - 48 months | 0 | 9 (0.4) | 9 (0.4) |
| Duration of exposure | | | |
| (months) | | | |
| n | 2524 | 2524 | 2444 |
| Mean | 23.1 | 28.0 | 27.3 |
| SD | 12.23 | 10.82 | 11.48 |
| Min | 0 | 0 | 0 |
| Median | 27.4 | 30.1 | 30.0 |
| Max | 37 | 47 | 48 |

### 4 Efficacy Results

### 4.1 Primary Efficacy Results

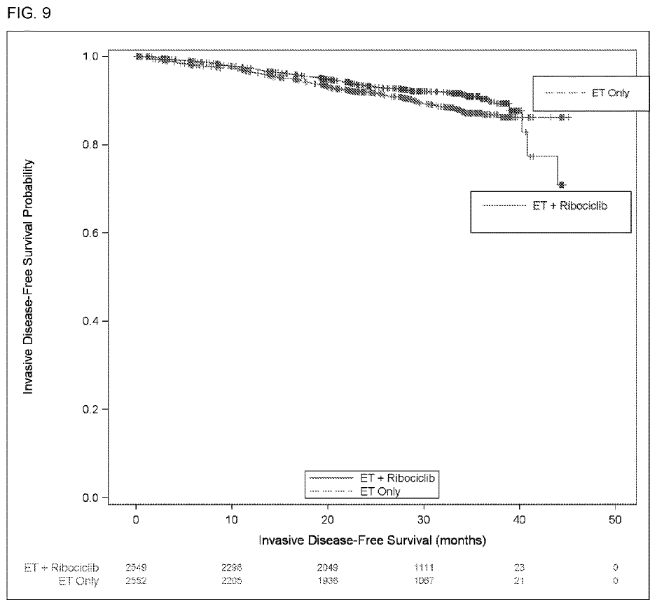

### (i) See FIG. 9 for Primary Invasive Disease-Free Survival Analysis—Kaplan-Meier Plot (Full Analysis Set)

TABLE B4.1-1
(j) Log-rank test result for iDFS (Full analysis set)

| Treatment | n/N (%) | Comparison | Z-statistic | p-value* |
|---|---|---|---|---|
| ET + Ribociclib | 189/2549 (7.4) | vs. ET Only | −2.9847 | 0.0014 |
| ET Only | 237/2552 (9.3) | | | |

n is the number of iDFS events.
N = total number of patients included in the analysis.
*1-sided p-value for log-rank test stratified by premenopausal women and men vs. postmenopausal women, anatomic stage group II vs. anatomic stage group III, prior neo-/adjuvant chemotherapy (yes vs. no) and North America/Western Europe/Oceania vs. rest of world.

TABLE B4.1-2
(k) 1-2 Cox regression results for iDFS (Full analysis set)

| | | | Hazard | |
|---|---|---|---|---|
| Treatment | n/N (%) | Comparison | Ratio | 95% CI |
| ET + Ribociclib | 189/2549 (7.4) | vs. ET Only | 0.748 | (0.618, 0.906) |
| ET Only | 237/2552 (9.3) | | | |

TABLE B4.1-3
(l) Kaplan-Meier Estimates for Invasive Disease Free Survival (Full analysis set)

| | | | Patients with event at | | | | Cumulative | |
|---|---|---|---|---|---|---|---|---|
| | | | time point (censored) | | | | information | |
| | | # patients | | | | | KM % | |
| Treatment | Time | at risk | n (m) | | % | | estimate | 95% CI |
| ET + Ribociclib | Month 6 | 2350 | 27 | (172) | 1.1 | (6.7) | 98.9 | (98.4, 99.2) |
| (N = 2549) | Month 12 | 2274 | 38 | (38) | 1.5 | (1.5) | 97.3 | (96.5, 97.9) |
| | Month 18 | 2193 | 43 | (38) | 1.7 | (1.5) | 95.4 | (94.5, 96.2) |
| | Month 24 | 1718 | 43 | (432) | 1.7 | (16.9) | 93.4 | (92.2, 94.3) |
| | Month 30 | 1111 | 21 | (586) | 0.8 | (23.0) | 92.0 | (90.8, 93.1) |
| | Month 36 | 311 | 10 | (790) | 0.4 | (31.0) | 90.4 | (88.6, 91.9) |
| | Month 42 | 12 | 6 | (293) | 0.2 | (11.5) | 77.3 | (59.5, 88.1) |
| | Month 48 | 0 | 1 | (11) | 0.0 | (0.4) | NE | (NE, NE) |
| ET Only | Month 6 | 2240 | 47 | (265) | 1.8 | (10.4) | 98.0 | (97.3, 98.5) |
| (N = 2552) | Month 12 | 2166 | 37 | (37) | 1.4 | (1.4) | 96.3 | (95.5, 97.0) |
| | Month 18 | 2071 | 49 | (46) | 1.9 | (1.8) | 94.1 | (93.1, 95.0) |
| | Month 24 | 1631 | 46 | (394) | 1.8 | (15.4) | 91.9 | (90.7, 93.0) |
| | Month 30 | 1067 | 38 | (526) | 1.5 | (20.6) | 89.3 | (87.8, 90.6) |
| | Month 36 | 286 | 17 | (764) | 0.7 | (29.9) | 87.1 | (85.3, 88.8) |
| | Month 42 | 13 | 3 | (270) | 0.1 | (10.6) | 86.2 | (83.9, 88.1) |
| | Month 48 | 0 | 0 | (13) | 0.0 | (0.5) | NE | (NE, NE) |

NE = Not estimable.
n: the number of events
m: the number of patients censored

TABLE B4.1-4
(m) Invasive Disease Free Survival - Type and Site of First iDFS
Event (Full analysis set)

| | ET + ribociclib | ET only |
|---|---|---|
| | N = 2549 | N = 2552 |
| | n (%) | n (%) |
| Type of first iDFS event | | |
| Invasive ipsilateral breast tumor recurrence | 8 (0.3) | 7 (0.3) |
| Local/regional invasive recurrence | 19 (0.7) | 35 (1.4) |
| Distant recurrence | 120 (4.7) | 170 (6.7) |
| Invasive contralateral breast cancer | 7 (0.3) | 9 (0.4) |
| Death | 13 (0.5) | 7 (0.3) |
| Primary cause of death adverse event | 12 (0.5) | 3 (0.1) |
| Primary cause of death disease recurrence/progression | 1 (0.0) | 0 |
| Primary cause of death other | 0 | 4 (0.2) |
| Second primary non-breast invasive cancer | 30 (1.2) | 28 (1.1) |
| Site(s) of iDFS event recurrence (excluding death and | | |
| second primary non-breast invasive cancer) | | |
| Ipsilateral breast | 8 (0.3) | 6 (0.2) |
| Ipsilateral chest wall/skin | 10 (0.4) | 15 (0.6) |
| Ipsilateral axilla | 4 (0.2) | 7 (0.3) |
| Regional lymph nodes | 9 (0.4) | 17 (0.7) |
| Contralateral breast (with or without contralateral | 7 (0.3) | 9 (0.4) |
| lymph nodes) | | |
| Bone | 71 (2.8) | 96 (3.8) |
| Liver | 34 (1.3) | 53 (2.1) |
| Lung/pleura | 22 (0.9) | 37 (1.4) |
| Central nervous system | 11 (0.4) | 16 (0.6) |
| Distant lymph nodes | 17 (0.7) | 23 (0.9) |
| Other | 11 (0.4) | 8 (0.3) |

Patients may have multiple iDFS recurrence sites counted in the table, but are only counted once per patient.

TABLE B4.1-5
(n) Primary Invasive Disease Free Survival Analysis - Censoring
by Treatment Arm (Full analysis set)

| | Number of patients |
|---|---|
| | N = 5101 |
| | n (%) |
| ET + ribociclib (N = 2549) | |
| Number of patients with iDFS event | 189 (7.4) |
| Number of patients censored | 2360 (92.6) |
| Reason for censoring | |
| Ongoing without event | 2134 (83.7) |
| Withdrew consent | 213 (8.4) |
| Lost to follow-up | 13 (0.5) |
| ET only (N = 2552) | |
| Number of patients with iDFS event | 237 (9.3) |
| Number of patients censored | 2315 (90.7) |
| Reason for censoring | |
| Ongoing without event | 1972 (77.3) |
| Withdrew consent | 324 (12.7) |
| Lost to follow-up | 19 (0.7) |

Censoring date is the last assessment before the earliest of the following: analysis cut-off date, date of consent withdrawal, or date of last contact.
% is based on N in each arm.

TABLE B4.1-6
(o) Sensitivity analyses of iDFS (Full analysis set)

| | | | | Hazard ratio |
|---|---|---|---|---|
| Sensitivity analysis | Treatment | n/N | p-value | (95% CI) |
| Primary analysis | ET + Ribociclib | 189/2549 | 0.0014 | 0.748 (0.618, 0.906) |
| | ET only | 237/2552 | | |
| Primary analysis (PPS) | ET + Ribociclib | 187/2496 | 0.0015 | 0.749 (0.618, 0.907) |
| | ET only | 235/2424 | | |
| Primary analysis per CRF | ET + Ribociclib | 189/2549 | 0.0012 | 0.744 (0.614, 0.901) |
| | ET only | 237/2552 | | |
| Unstratified logrank test | ET + Ribociclib | 189/2549 | 0.0023 | 0.759 (0.627, 0.919) |
| and Cox model | ET only | 237/2552 | | |
| Stratified Cox model | | | | |
| adjusting for baseline | | | | |
| covariates [a] | ET + Ribociclib | 189/2549 | 0.0014 | 0.762 (0.628, 0.923) |
| | ET only | 237/2552 | | |
| Actual event [b] | ET + Ribociclib | 186/2549 | 0.0019 | 0.752 (0.620, 0.912) |
| | ET only | 232/2552 | | |
| Backdating [c] | ET + Ribociclib | 189/2549 | 0.0014 | 0.748 (0.618, 0.906) |
| | ET only | 237/2552 | | |
| Censoring for | ET + Ribociclib | 186/2549 | 0.0031 | 0.763 (0.629, 0.927) |
| antineoplastic therapy [d] | ET only | 226/2552 | | |
| Clinical recurrence [e] | ET + Ribociclib | 189/2549 | 0.0014 | 0.748 (0.618, 0.906) |
| | ET only | 237/2552 | | |
| Censoring covid death [f] | ET + Ribociclib | 183/2549 | 0.0006 | 0.727 (0.600, 0.882) |
| | ET only | 236/2552 | | |

CI = Confidence interval.
[a] Baseline covariates included in the Cox proportional hazard model are Age category (<45, 45 - 54, and 55 - 64 vs. >64 years of age), ER/PR status (ER+/PR+ vs. other), and ET type (Letrozole vs. Anastrozole).
[b] Analysis excludes the event whenever it occurred after missing >=2 tumor assessments.
[c] Analysis uses the date of the next scheduled assessment for events occurring after missing >=1 assessment.
[d] Analysis performed by censoring patients at start of new antineoplastic therapy.
[e] Analysis considering treatment discontinuation due to disease recurrence as iDFS event without confirmation of recurrence.
[f] Analysis performed by censoring patients with COVID death.
p-values were calculated based on log-rank test for [b], [c] and [d]. For [a], it is calculated based on wald test statistic of the HR.

TABLE B4.1-7
(p) Cox regression for iDFS - subgroup analysis (Full analysis set)

| | | | Number of | Number of | Hazard | |
|---|---|---|---|---|---|---|
| Factor | Subgroup | Treatment | patients | events | Ratio | 95% CI |
| Menopausal | Pre-menopausal | ET + | 1126 | 71 | 0.722 | (0.530, |
| status* | women and men | Ribociclib | | | | 0.983) |
| | | ET Only | 1132 | 93 | | |
| | Post-menopausal | ET + | 1423 | 118 | 0.781 | (0.613, |
| | women | Ribociclib | | | | 0.997) |
| | | ET Only | 1420 | 144 | | |
| AJCC stage* | AJCC stage II | ET + | 1011 | 49 | 0.761 | (0.525, |
| | | Ribociclib | | | | 1.103) |
| | | ET Only | 1034 | 65 | | |
| | AJCC stage III | ET + | 1528 | 140 | 0.740 | (0.592, |
| | | Ribociclib | | | | 0.925) |
| | | ET Only | 1512 | 172 | | |
| Prior neo-/ | Yes | ET + | 2249 | 167 | 0.729 | (0.596, |
| adjuvant | | Ribociclib | | | | 0.893) |
| chemotherapy* | | ET Only | 2245 | 215 | | |
| | No | ET + | 300 | 22 | 1.038 | (0.575, |
| | | Ribociclib | | | | 1.875) |
| | | ET Only | 307 | 22 | | |
| Region* | NA/WE/O | ET + | 1563 | 111 | 0.759 | (0.591, |
| | | Ribociclib | | | | 0.974) |
| | | ET Only | 1565 | 139 | | |
| | ROW | ET + | 986 | 78 | 0.757 | (0.562, |
| | | Ribociclib | | | | 1.019) |
| | | ET Only | 987 | 98 | | |
| Anatomic | IIA | ET + | 479 | 13 | 0.500 | (0.258, |
| Stage** | | Ribociclib | | | | 0.968) |
| | | ET Only | 521 | 27 | | |
| | IIB | ET + | 532 | 36 | 0.930 | (0.589, |
| | | Ribociclib | | | | 1.467) |
| | | ET Only | 513 | 38 | | |
| | IIIA | ET + | 939 | 75 | 0.792 | (0.580, |
| | | Ribociclib | | | | 1.080) |
| | | ET Only | 894 | 85 | | |
| | IIIB | ET + | 168 | 18 | 0.678 | (0.355, |
| | | Ribociclib | | | | 1.295) |
| | | ET Only | 149 | 20 | | |
| | IIIC | ET + | 421 | 47 | 0.685 | (0.472, |
| | | Ribociclib | | | | 0.994) |
| | | ET Only | 469 | 67 | | |
| Gender | Women | ET + | 2538 | 188 | 0.758 | (0.625, |
| | | Ribociclib | | | | 0.918) |
| | | ET Only | 2543 | 236 | | |
| Prior adjuvant | Yes | ET + | 1223 | 63 | 0.671 | (0.486, |
| chemotherapy | | Ribociclib | | | | 0.927) |
| | | ET Only | 1220 | 89 | | |
| | No | ET + | 1326 | 126 | 0.814 | (0.642, |
| | | Ribociclib | | | | 1.032) |
| | | ET Only | 1332 | 148 | | |
| Prior | Yes | ET + | 1085 | 111 | 0.785 | (0.610, |
| neoadjuvant | | Ribociclib | | | | 1.011) |
| chemotherapy | | ET Only | 1095 | 132 | | |
| | No | ET + | 1464 | 78 | 0.717 | (0.535, |
| | | Ribociclib | | | | 0.961) |
| | | ET Only | 1457 | 105 | | |
| Prior radiation | Yes | ET + | 2292 | 170 | 0.739 | (0.605, |
| therapy | | Ribociclib | | | | 0.903) |
| | | ET Only | 2302 | 218 | | |
| | No | ET + | 257 | 19 | 0.980 | (0.519, |
| | | Ribociclib | | | | 1.851) |
| | | ET Only | 250 | 19 | | |
| Prior endocrine | Yes | ET + | 1824 | 127 | 0.756 | (0.598, |
| therapy | | Ribociclib | | | | 0.955) |
| | | ET Only | 1801 | 157 | | |
| | No | ET + | 725 | 62 | 0.774 | (0.556, |
| | | Ribociclib | | | | 1.079) |
| | | ET Only | 751 | 80 | | |
| Prior | Yes | ET + | 1664 | 147 | 0.853 | (0.683, |
| mastectomy | | Ribociclib | | | | 1.066) |
| | | ET Only | 1691 | 165 | | |
| | No | ET + | 885 | 42 | 0.548 | (0.375, |
| | | Ribociclib | | | | 0.802) |
| | | ET Only | 861 | 72 | | |
| Race | Asian | ET + | 341 | 18 | 0.523 | (0.290, |
| | | Ribociclib | | | | 0.942) |
| | | ET Only | 334 | 29 | | |
| | Non-Asian | ET + | 2070 | 160 | 0.807 | (0.654, |
| | | Ribociclib | | | | 0.995) |
| | | ET Only | 2091 | 192 | | |
| Region | Europe | ET + | 1505 | 122 | 0.875 | (0.686, |
| | | Ribociclib | | | | 1.116) |
| | | ET Only | 1506 | 138 | | |
| | North | ET + | 624 | 47 | 0.697 | (0.475, |
| | America/Australia | Ribociclib | | | | 1.025) |
| | | ET Only | 612 | 58 | | |
| | Asia | ET + | 281 | 9 | 0.338 | (0.157, |
| | | Ribociclib | | | | 0.728) |
| | | ET Only | 290 | 24 | | |
| | Latin America | ET + | 139 | 11 | 0.691 | (0.323, |
| | | Ribociclib | | | | 1.477) |
| | | ET Only | 144 | 17 | | |
| Age category 1 | <45 | ET + | 611 | 39 | 0.683 | (0.449, |
| | | Ribociclib | | | | 1.038) |
| | | ET Only | 591 | 50 | | |
| | 45 to 54 | ET + | 849 | 52 | 0.750 | (0.524, |
| | | Ribociclib | | | | 1.075) |
| | | ET Only | 895 | 69 | | |
| | 55 to 64 | ET + | 682 | 64 | 0.839 | (0.601, |
| | | Ribociclib | | | | 1.170) |
| | | ET Only | 700 | 76 | | |
| | >=65 | ET + | 407 | 34 | 0.723 | (0.460, |
| | | Ribociclib | | | | 1.137) |
| | | ET Only | 366 | 42 | | |
| Age category 2 | <Median | ET + | 1216 | 71 | 0.727 | (0.535, |
| | | Ribociclib | | | | 0.989) |
| | | ET Only | 1264 | 95 | | |
| | >=Median | ET + | 1333 | 118 | 0.775 | (0.607, |
| | | Ribociclib | | | | 0.990) |
| | | ET Only | 1288 | 142 | | |
| Type of NSAI | Letrozole | ET + | 1729 | 127 | 0.790 | (0.624, |
| | | Ribociclib | | | | 0.999) |
| | | ET Only | 1674 | 154 | | |
| | Anastrozole | ET + | 797 | 62 | 0.702 | (0.505, |
| | | Ribociclib | | | | 0.976) |
| | | ET Only | 768 | 83 | | |
| Hormone | ER+/PR+ | ET + | 2172 | 140 | 0.726 | (0.582, |
| receptor status | | Ribociclib | | | | 0.906) |
| | | ET Only | 2132 | 180 | | |
| | ER−/PR+ | ET + | 3 | 1 | 506E6 | (0.000, |
| | | Ribociclib | | | | NE) |
| | | ET Only | 12 | 0 | | |
| | ER+/PR− | ET + | 359 | 48 | 0.908 | (0.615, |
| | | Ribociclib | | | | 1.339) |
| | | ET Only | 392 | 54 | | |
| Nodal status | N0 | ET + | 285 | 16 | 0.630 | (0.341, |
| | | Ribociclib | | | | 1.165) |
| | | ET Only | 328 | 28 | | |
| | N1-N3 | ET + | 2261 | 173 | 0.771 | (0.630, |
| | | Ribociclib | | | | 0.944) |
| | | ET Only | 2219 | 208 | | |
| Tumor category | T0 | ET + | 8 | 1 | 324E5 | (0.000, |
| | | Ribociclib | | | | NE) |
| | | ET Only | 6 | 0 | | |
| | T1-T3 | ET + | 2345 | 164 | 0.742 | (0.605, |
| | | Ribociclib | | | | 0.910) |
| | | ET Only | 2360 | 212 | | |
| | >T3 | ET + | 189 | 22 | 0.827 | (0.460, |
| | | Ribociclib | | | | 1.486) |
| | | ET Only | 181 | 23 | | |
| Histological | Grade 1 | ET + | 213 | 9 | 0.778 | (0.328, |
| grade at time of | | Ribociclib | | | | 1.846) |
| surgery | | ET Only | 217 | 12 | | |
| | Grade 2 | ET + | 1460 | 102 | 0.749 | (0.577, |
| | | Ribociclib | | | | 0.973) |
| | | ET Only | 1432 | 125 | | |
| | Grade 3 | ET + | 684 | 61 | 0.776 | (0.555, |
| | | Ribociclib | | | | 1.085) |
| | | ET Only | 702 | 78 | | |
| Ki67 status from | Ki67 <= 20 | ET + | 1199 | 76 | 0.801 | (0.593, |
| archival tumor | | Ribociclib | | | | 1.083) |
| | | ET Only | 1236 | 95 | | |
| | Ki67 > 20 | ET + | 920 | 82 | 0.746 | (0.559, |
| | | Ribociclib | | | | 0.996) |
| | | ET Only | 938 | 105 | | |
| Histological | Ductal | ET + | 1858 | 123 | 0.675 | (0.536, |
| subtype | | Ribociclib | | | | 0.850) |
| | | ET Only | 1881 | 174 | | |
| | Lobular | ET + | 455 | 47 | 1.053 | (0.696, |
| | | Ribociclib | | | | 1.592) |
| | | ET Only | 450 | 43 | | |
| | Other | ET + | 235 | 19 | 0.887 | (0.473, |
| | | Ribociclib | | | | 1.661) |
| | | ET Only | 221 | 20 | | |
| BMI at | >=25 | ET + | 1505 | 124 | 0.848 | (0.667, |
| screening | | Ribociclib | | | | 1.079) |
| | | ET Only | 1522 | 142 | | |
| | <25 | ET + | 1013 | 64 | 0.635 | (0.462, |
| | | Ribociclib | | | | 0.873) |
| | | ET Only | 999 | 93 | | |

*Randomization stratification factor from eCRF.
**Subgroup AJCC Stage (IIA vs IIB vs IIIA vs IIIB vs IIIC) derived from eCRF data.
NE = Not estimable.

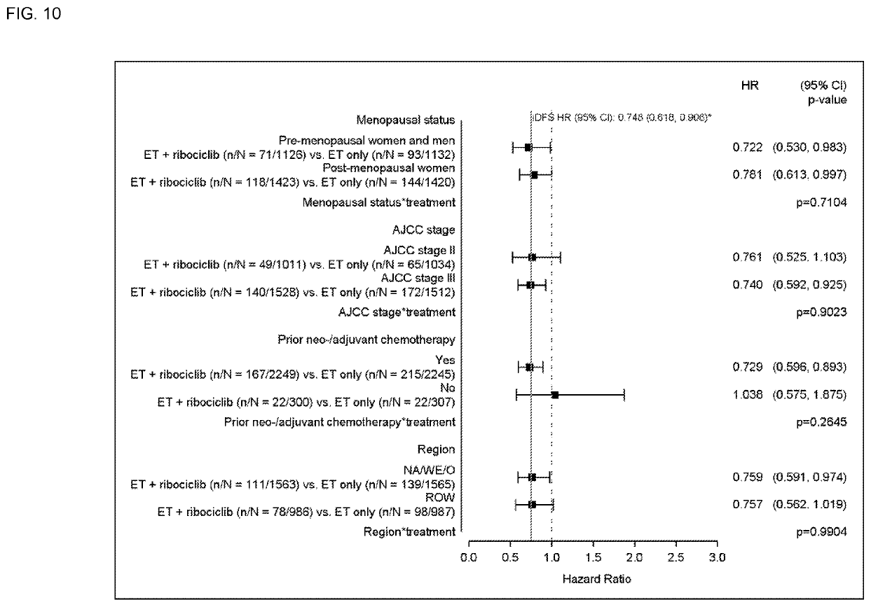

### (q) See FIG. 10 for Forest Plot of iDFS by Stratum(Per eCRF) (Full Analysis Set)

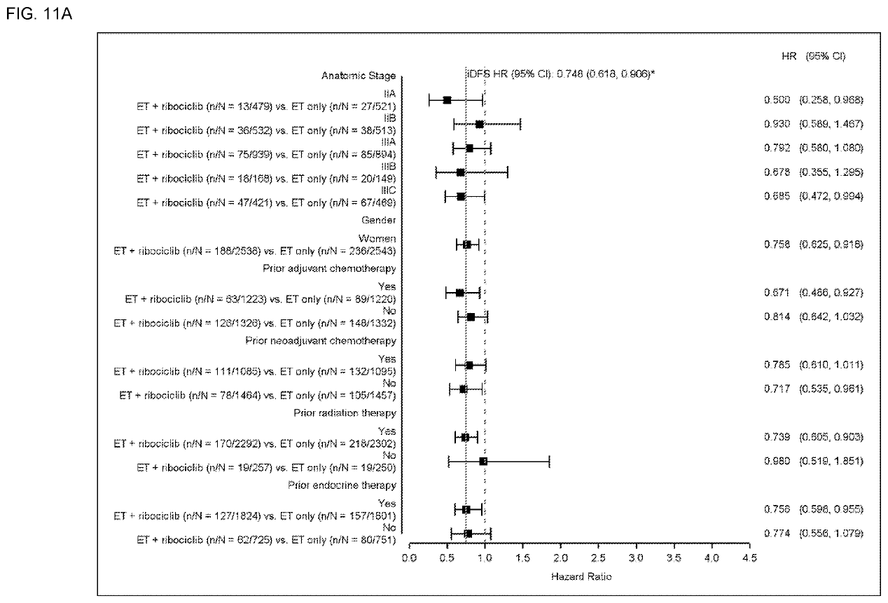

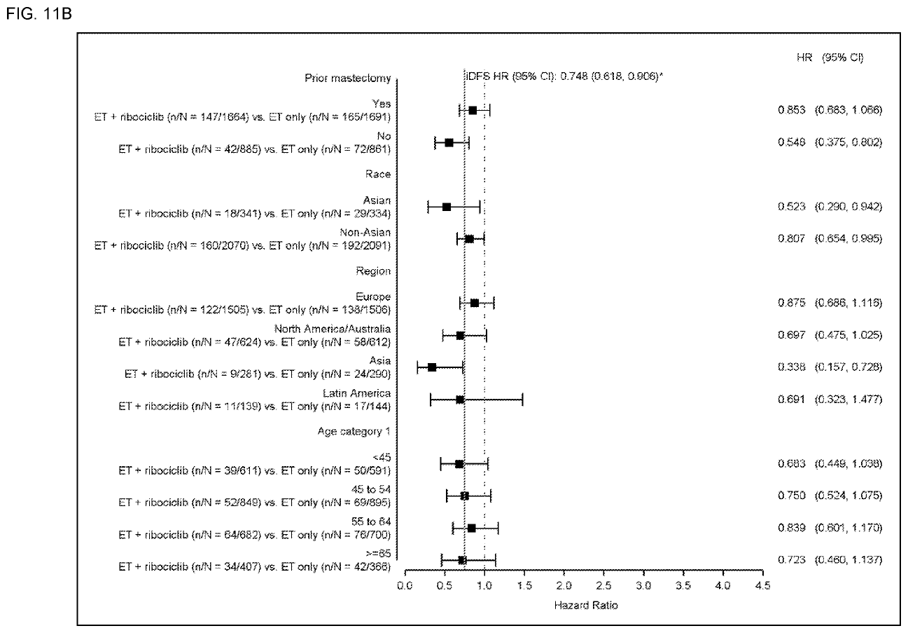

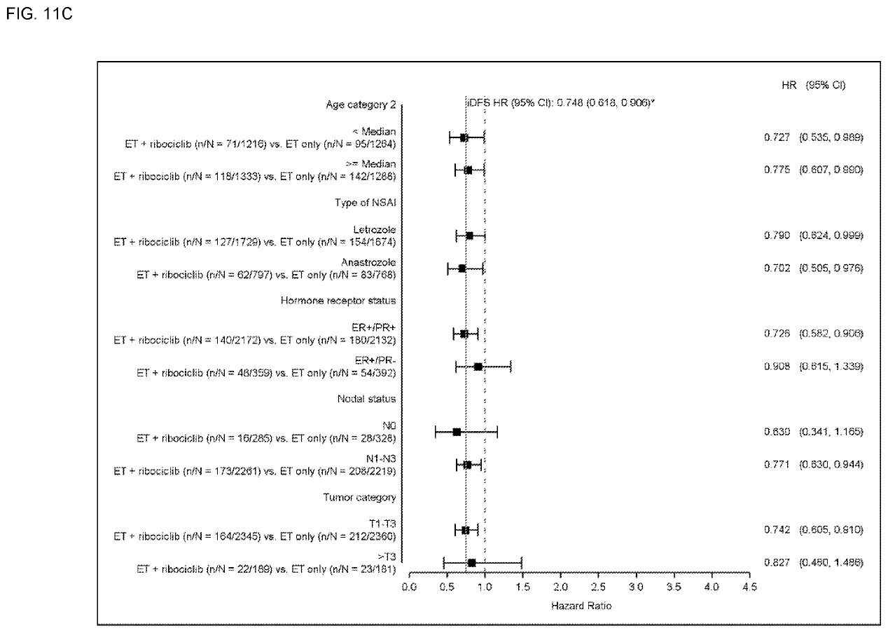

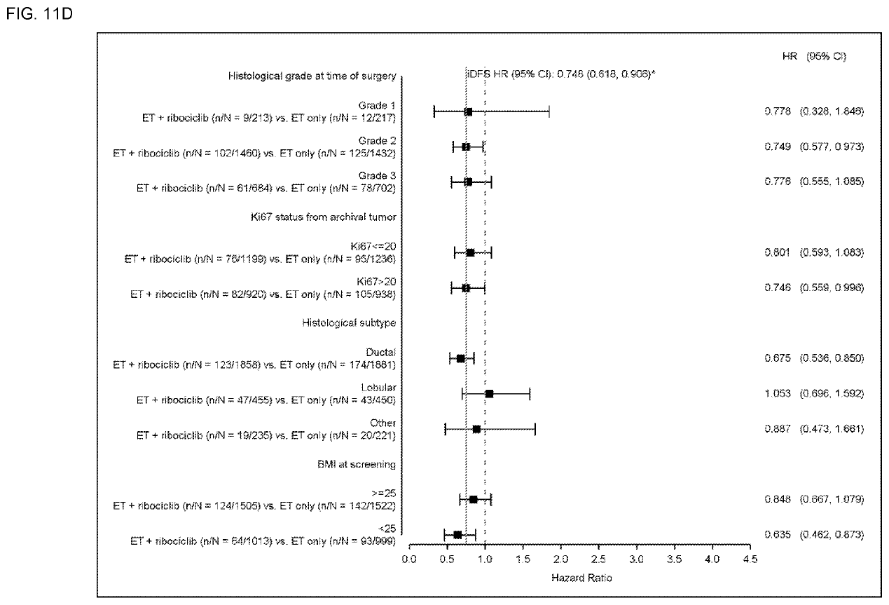

See FIGS. 11A-D for Forest Plots of iDFS—subgroup analysis (Full analysis set). Subgroups with not estimable HRs were removed from forest plot. *-Hazard rate in group ET+ribociclib versus hazard rate in group ET only is computed using the Cox proportional hazards model with treatment as a single covariate and premenopausal women and men vs. postmenopausal women, anatomic stage group II vs. anatomic stage group, prior neo-/adjuvant chemotherapy (yes vs. no) and North America/Western Europe/Oceania vs. rest of world as stratification factors. The group ET only is the reference in the hazard ratio calculation.

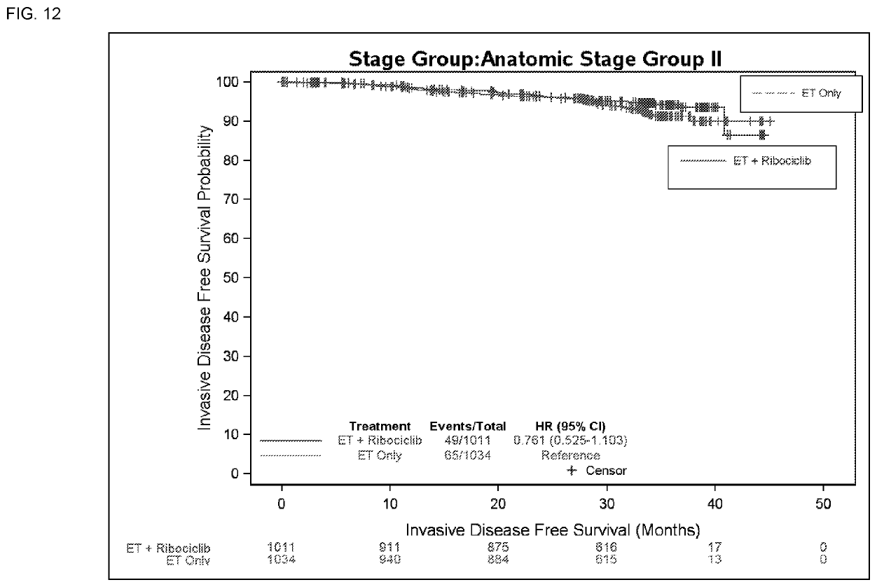

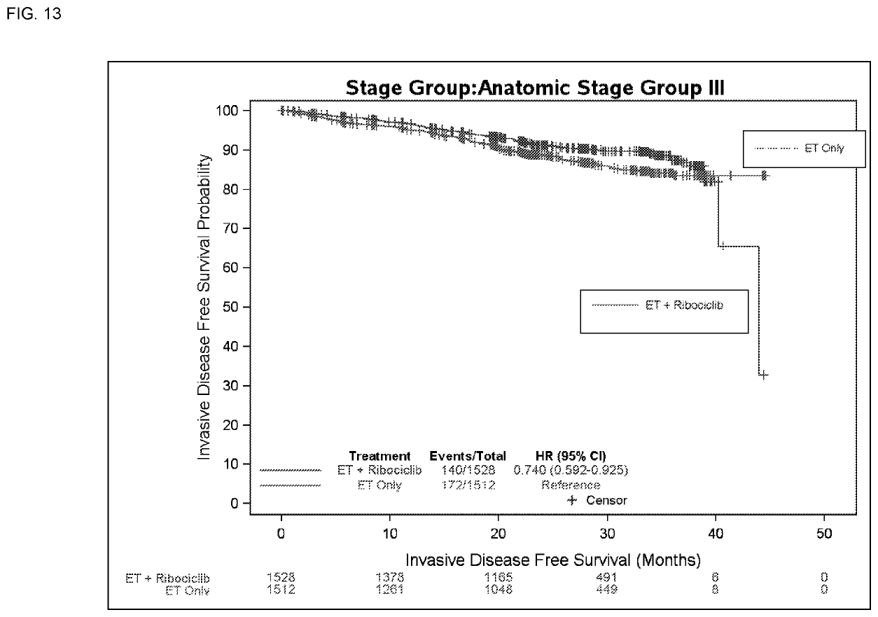

FIGS. 12 and 13 for iDFS—Kaplan-Meier survival curves of iDFS by Anatomic Stage II and III, respectively.

### 4.2 Secondary Efficacy Results

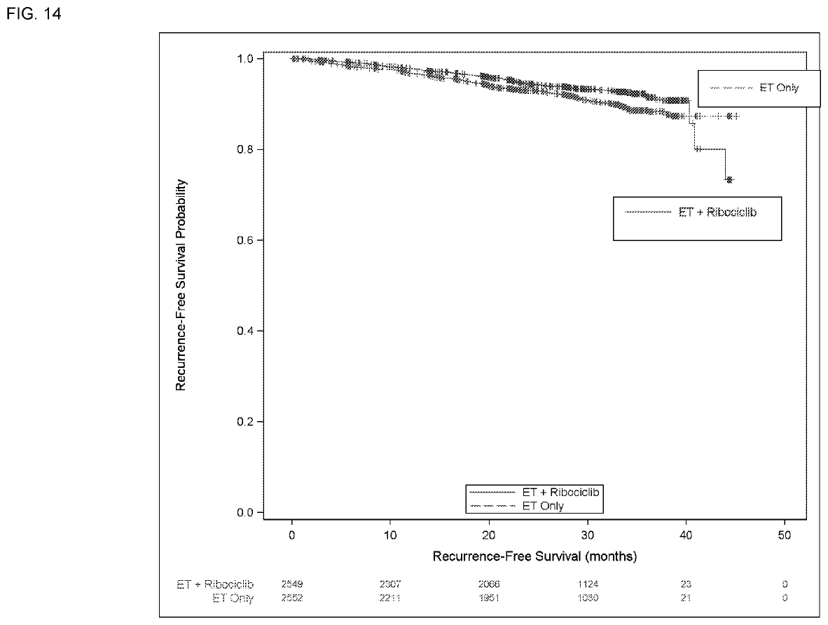

See FIG. 14 for Kaplan-Meier for RFS (Full Analysis set)

TABLE B4.2-1
Cox regression results for RFS (Full analysis set)

| | | | Hazard | |
|---|---|---|---|---|
| Treatment | n/N (%) | Comparison | Ratio | 95% CI |
| ET + Ribociclib | 159/2549 (6.2) | vs. ET Only | 0.719 | (0.584, 0.884) |
| ET Only | 207/2552 (8.1) | | | |

n is the number of RFS events.
N = total number of patients included in the analysis.
Hazard rate in group ET + ribociclib versus hazard rate in group ET only is computed using the Cox proportional hazards model with treatment as a single covariate and premenopausal women and men vs. postmenopausal women, anatomic stage group II vs. anatomic stage group III, prior neo-/adjuvant chemotherapy (yes vs. no) and North America/Western Europe/Oceania vs. rest of world as stratification factors. The group ET only is the reference in the hazard ratio calculation.

TABLE B4.2-2
Log-rank result for DDFS (Full analysis set)

| Treatment | n/N (%) | Comparison | Z-statistic | p-value* |
|---|---|---|---|---|
| ET + Ribociclib | 159/2549 (6.2) | vs. ET Only | −3.144 | 0.0008 |
| ET Only | 207/2552 (8.1) | | | |

n is the number of OS events.
N = total number of patients included in the analysis.
*1-sided p-value for log-rank test stratified by premenopausal women and men vs. postmenopausal women, anatomic stage group II vs. anatomic stage group III, prior neo-/adjuvant chemotherapy (yes vs. no) and North America/Western Europe/Oceania vs. rest of world.

TABLE B4.2-3
Kaplan-Meier for Recurrence Free Survival (Full analysis set)

| | | | Patients with event at | | | | Cumulative | |
|---|---|---|---|---|---|---|---|---|
| | | | time point (censored) | | | | information | |
| | | # patients | | | | | KM % | |
| Treatment | Time | at risk | n (m) | | % | | estimate | 95% CI |
| ET + Ribociclib | Month 6 | 2358 | 19 | (172) | 0.7 | (6.7) | 99.2 | (98.8, 99.5) |
| (N = 2549) | Month 12 | 2283 | 33 | (42) | 1.3 | (1.6) | 97.8 | (97.1, 98.3) |
| | Month 18 | 2210 | 31 | (42) | 1.2 | (1.6) | 96.5 | (95.6, 97.1) |
| | Month 24 | 1733 | 42 | (435) | 1.6 | (17.1) | 94.4 | (93.4, 95.3) |
| | Month 30 | 1124 | 18 | (591) | 0.7 | (23.2) | 93.2 | (92.1, 94.3) |
| | Month 36 | 314 | 10 | (800) | 0.4 | (31.4) | 91.7 | (90.1, 93.1) |
| | Month 42 | 12 | 5 | (297) | 0.2 | (11.7) | 80.0 | (61.1, 90.4) |
| | Month 48 | 0 | 1 | (11) | 0.0 | (0.4) | NE | (NE, NE) |
| ET Only | Month 6 | 2247 | 40 | (265) | 1.6 | (10.4) | 98.3 | (97.7, 98.7) |
| (N = 2552) | Month 12 | 2174 | 35 | (38) | 1.4 | (1.5) | 96.7 | (95.9, 97.4) |
| | Month 18 | 2086 | 39 | (49) | 1.5 | (1.9) | 95.0 | (94.0, 95.8) |
| | Month 24 | 1646 | 40 | (400) | 1.6 | (15.7) | 93.0 | (91.9, 94.0) |
| | Month 30 | 1080 | 32 | (534) | 1.3 | (20.9) | 90.8 | (89.4, 92.0) |
| | Month 36 | 290 | 17 | (773) | 0.7 | (30.3) | 88.6 | (86.8, 90.2) |
| | Month 42 | 13 | 4 | (273) | 0.2 | (10.7) | 87.3 | (85.0, 89.3) |
| | Month 48 | 0 | 0 | (13) | 0.0 | (0.5) | NE | (NE, NE) |

NE = Not estimable.
n: the number of events
m: the number of patients censored

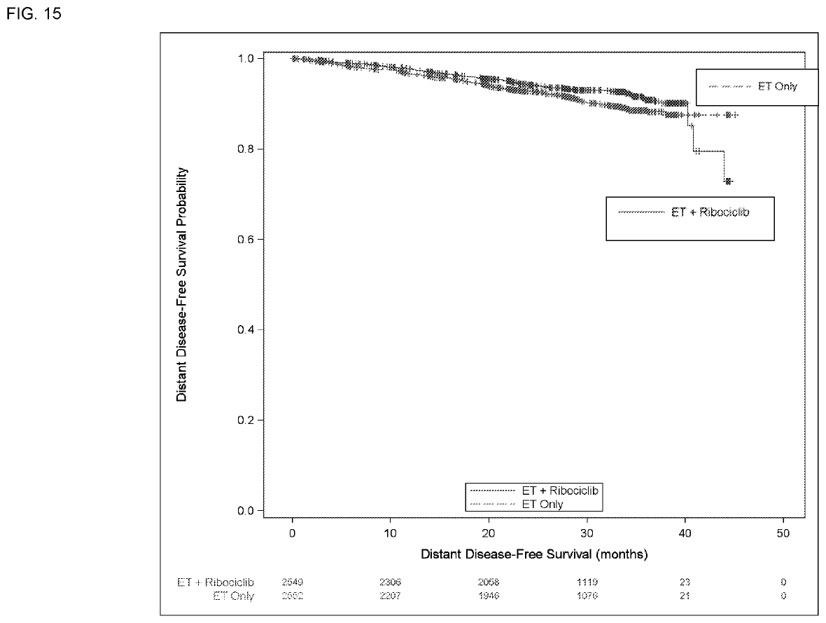

See FIG. 15 for Kaplan-Meier for DDFS (Full analysis set).

FIGS. 4.2-2 Kaplan-Meier for DDFS (Full analysis set)

TABLE B4.2-4
Cox regression model results for DDFS (Full analysis set)

| | | | Hazard | |
|---|---|---|---|---|
| Treatment | n/N (%) | Comparison | Ratio | 95% CI |
| ET + Ribociclib | 167/2549 (6.6) | vs. ET Only | 0.739 | (0.603, |
| ET Only | 212/2552 (8.3) | | | 0.905) |

n is the number of DDFS events.
N = total number of patients included in the analysis.
Hazard rate in group ET + ribociclib versus hazard rate in group ET only is computed using the Cox proportional hazards model with treatment as a single covariate and premenopausal women and men vs. postmenopausal women, anatomic stage group II vs. anatomic stage group III, prior neo-/adjuvant chemotherapy (yes vs. no) and North America/Western Europe/Oceania vs. rest of world as stratification factors. The group ET only is the reference in the hazard ratio calculation.

TABLE B4.2-5
Log-rank result for DDFS (Full analysis set)

| Treatment | n/N (%) | Comparison | Z-statistic | p-value* |
|---|---|---|---|---|
| ET + Ribociclib | 167/2549 (6.6) | vs. ET Only | −2.930 | 0.0017 |
| Treatment | n/N (%) | Comparison | Z-statistic | p-value* |
| ET Only | 212/2552 (8.3) | | | |

n is the number of DDFS events.
N = total number of patients included in the analysis.
*1-sided p-value for log-rank test stratified by premenopausal women and men vs. postmenopausal women, anatomic stage group II vs. anatomic stage group III, prior neo-/adjuvant chemotherapy (yes vs. no) and North America/Western Europe/Oceania vs. rest of world.

TABLE B4.2-6
Kaplan-Meier for Distant Disease Free Survival (Full analysis set)

| | | | Patients with event at | | | | Cumulative | |
|---|---|---|---|---|---|---|---|---|
| | | | time point (censored) | | | | information | |
| | | # patients | | | | | KM % | |
| Treatment | Time | at risk | n (m) | | % | | estimate | 95% CI |
| ET + Ribociclib | Month 6 | 2352 | 25 | (172) | 1.0 | (6.7) | 99.0 | (98.5, 99.3) |
| (N = 2549) | Month 12 | 2280 | 30 | (42) | 1.2 | (1.6) | 97.7 | (97.0, 98.2) |
| | Month 18 | 2199 | 39 | (42) | 1.5 | (1.6) | 96.0 | (95.1, 96.7) |
| | Month 24 | 1729 | 36 | (434) | 1.4 | (17.0) | 94.3 | (93.2, 95.1) |
| | Month 30 | 1119 | 21 | (589) | 0.8 | (23.1) | 93.0 | (91.8, 94.0) |
| | Month 36 | 311 | 11 | (797) | 0.4 | (31.3) | 90.8 | (89.0, 92.4) |
| | Month 42 | 12 | 4 | (295) | 0.2 | (11.6) | 79.5 | (60.9, 89.9) |
| | Month 48 | 0 | 1 | (11) | 0.0 | (0.4) | NE | (NE, NE) |
| ET Only | Month 6 | 2244 | 42 | (266) | 1.6 | (10.4) | 98.2 | (97.6, 98.7) |
| (N = 2552) | Month 12 | 2368 | 37 | (39) | 1.4 | (1.5) | 96.5 | (95.7, 97.2) |
| | Month 18 | 2080 | 37 | (51) | 1.4 | (2.0) | 94.9 | (93.9, 95.7) |
| | Month 24 | 1643 | 42 | (395) | 1.6 | (15.5) | 92.8 | (91.7, 93.8) |
| | Month 30 | 1076 | 37 | (530) | 1.4 | (20.8) | 90.3 | (88.8, 91.5) |
| | Month 36 | 288 | 14 | (774) | 0.5 | (30.3) | 88.6 | (86.8, 90.1) |
| | Month 42 | 13 | 3 | (272) | 0.1 | (10.7) | 87.6 | (85.4, 89.4) |
| | Month 48 | 0 | 0 | (13) | 0.0 | (0.5) | NE | (NE, NE) |

NE = Not estimable.
n: the number of events
m: the number of patients censored

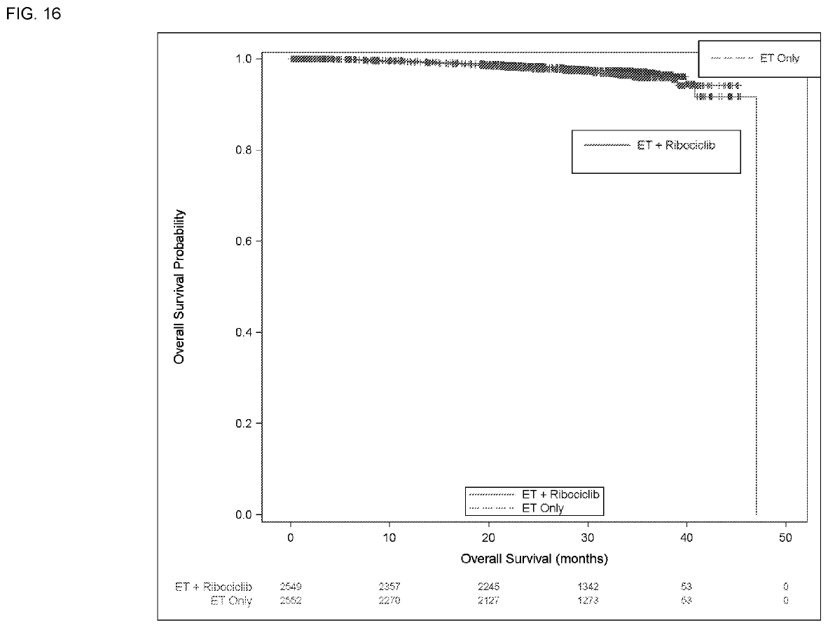

See FIG. 16 for Kaplan-Meier for OS (Full analysis set).

TABLE B4.2-7
Cox regression model results for OS (Full analysis set)

| Treatment | n/N (%) | Comparison | Hazard Ratio | 95% CI |
|---|---|---|---|---|
| ET + Ribociclib | 61/2549 (2.4) | vs. ET Only | 0.759 | (0.539, 1.068) |
| Treatment | n/N (%) | Comparison | Hazard Ratio | 95% CI |
| ET Only | 73/2552 (2.9) | | | |

n is the number of OS events.
N = total number of patients included in the analysis.
Hazard rate in group ET + ribociclib versus hazard rate in group ET only is computed using the Cox proportional hazards model with treatment as a single covariate and premenopausal women and men vs. postmenopausal women, anatomic stage group Il vs. anatomic stage group III, prior neo-/adjuvant chemotherapy (yes vs. no) and North America/Western Europe/Oceania vs. rest of world as stratification factors. The group ET only is the reference in the hazard ratio calculation.

TABLE B4.2-8
Log-rank result for OS (Full analysis set)

| Treatment | n/N (%) | Comparison | Z-statistic | p-value* |
|---|---|---|---|---|
| ET + Ribociclib | 61/2549 (2.4) | vs. ET Only | −1.5871 | 0.0563 |
| ET Only | 73/2552 (2.9) | | | |

n is the number of OS events.
N = total number of patients included in the analysis.
*1-sided p-value for log-rank test stratified by premenopausal women and men vs. postmenopausal women, anatomic stage group II vs. anatomic stage group III, prior neo-/adjuvant chemotherapy (yes vs. no) and North America/Western Europe/Oceania vs. rest of world.

TABLE B4.2-9
Kaplan-Meier for Overall Survival (Full analysis set)

| | | | Patients with event at | | | | Cumulative | |
|---|---|---|---|---|---|---|---|---|
| | | | time point (censored) | | | | information | |
| | | # patients | | | | | KM % | |
| Treatment | Time | at risk | n (m) | | % | | estimate | 95% CI |
| ET + Ribociclib | Month 6 | 2405 | 2 | (142) | 0.1 | (5.6) | 99.9 | (99.7, 100.0) |
| (N = 2549) | Month 12 | 2337 | 9 | (59) | 0.4 | (2.3) | 99.5 | (99.2, 99.7) |
| | Month 18 | 2303 | 12 | (22) | 0.5 | (0.9) | 99.0 | (98.5, 99.4) |
| | Month 24 | 1905 | 14 | (384) | 0.5 | (15.1) | 98.4 | (97.8, 98.8) |
| | Month 30 | 1338 | 13 | (554) | 0.5 | (21.7) | 97.6 | (96.8, 98.2) |
| | Month 36 | 451 | 4 | (883) | 0.2 | (34.6) | 97.2 | (96.3, 97.9) |
| | Month 42 | 21 | 6 | (424) | 0.2 | (16.6) | 91.7 | (82.5, 96.1) |
| | Month 48 | 0 | 1 | (20) | 0.0 | (0.8) | 0.0 | (NE, NE) |
| ET Only | Month 6 | 2303 | 5 | (244) | 0.2 | (9.6) | 99.8 | (99.5, 99.9) |
| (N = 2552) | Month 12 | 2256 | 11 | (36) | 0.4 | (1.4) | 99.3 | (98.9, 99.6) |
| | Month 18 | 2209 | 15 | (32) | 0.6 | (1.3) | 98.6 | (98.1, 99.0) |
| | Month 24 | 1823 | 15 | (371) | 0.6 | (14.5) | 97.9 | (97.2, 98.4) |
| | Month 30 | 1273 | 11 | (539) | 0.4 | (21.1) | 97.3 | (96.5, 97.9) |
| | Month 36 | 385 | 14 | (874) | 0.5 | (34.2) | 95.8 | (94.5, 96.7) |
| | Month 42 | 22 | 2 | (361) | 0.1 | (14.1) | 94.2 | (90.8, 96.4) |
| | Month 48 | 0 | 0 | (22) | 0.0 | (0.9) | NE | (NE, NE) |

NE = Not estimable.
n: the number of events
m: the number of patients censored

TABLE B4.2-10
OS Sensitivity Analysis: Cox regression model results for OS Censoring
COVID 19 Death (Full analysis set)

| Treatment | n/N (%) | Comparison | Hazard Ratio | 95% CI |
|---|---|---|---|---|
| ET + Ribociclib | 55/2549 (2.4) | vs. ET Only | 0.691 | (0.486, 0.984) |
| ET Only | 72/2552 (2.9) | | | |

n is the number of OS events.
N = total number of patients included in the analysis.
Hazard rate in group ET + ribociclib versus hazard rate in group ET only is computed using the Cox proportional hazards model with treatment as a single covariate and premenopausal women and men vs. postmenopausal women, anatomic stage group II vs. anatomic stage group III, prior neo-/adjuvant chemotherapy (yes vs. no) and North America/Western Europe/Oceania vs. rest of world as stratification factors. The group ET only is the reference in the hazard ratio calculation.

TABLE B4.2-11
OS Sensitivity Analysis: Log-rank result for OS Censoring
COVID 19 Death (Full analysis set)

| Treatment | n/N (%) | Comparison | Z-statistic | p-value* |
|---|---|---|---|---|
| ET + Ribociclib | 55/2549 (2.4) | vs. ET Only | −2.059 | 0.0197 |
| ET Only | 72/2552 (2.9) | | | |

n is the number of OS events.
N = total number of patients included in the analysis.
*1-sided p-value for log-rank test stratified by premenopausal women and men vs. postmenopausal women, anatomic stage group II vs. anatomic stage group III, prior neo-/adjuvant chemotherapy (yes vs. no) and North America/Western Europe/Oceania vs. rest of world.

### 5 Safety Results

### 5.1 Adverse Events

TABLE B5.1-1
Treatment-Emergent Adverse Events Summary (Safety set)

| | ET + ribociclib | ET only | Total |
|---|---|---|---|
| | N = 2524 | N = 2444 | N = 4968 |
| Number of patients with at least one | n (%) | n (%) | n (%) |
| TEAE | 2470 (97.9) | 2128 (87.1) | 4598 (92.6) |
| Serious TEAE | 336 (13.3) | 242 (9.9) | 578 (11.6) |
| TEAEs related to study treatment | 2362 (93.6) | 1534 (62.8) | 3896 (78.4) |
| (Ribociclib and/or ET) | | | |
| TEAE related to Ribociclib | 2282 (90.4) | 0 | 2282 (45.9) |
| TEAE related to ET | 1609 (63.7) | 1534 (62.8) | 3143 (63.3) |
| Serious TEAE related to study treatment | 66 (2.6) | 12 (0.5) | 78 (1.6) |
| (Ribociclib and/or ET) | | | |
| Serious TEAE related to Ribociclib | 57 (2.3) | 0 | 57 (1.1) |
| Serious TEAE related to ET | 29 (1.1) | 12 (0.5) | 41 (0.8) |
| TEAE leading to treatment discontinuation | 523 (20.7) | 129 (5.3) | 652 (13.1) |
| (Ribociclib and/or ET) | | | |
| TEAE leading to treatment discontinuation of | 495 (19.6) | 0 | 495 (10.0) |
| Ribociclib | | | |
| TEAE leading to treatment discontinuation of ET | 147 (5.8) | 129 (5.3) | 276 (5.6) |
| TEAE leading to dose reduction | 515 (20.4) | 0 | 515 (10.4) |
| TEAE leading to dose interruption | 1823 (72.2) | 182 (7.4) | 2005 (40.4) |
| TEAE requiring additional therapy | 1926 (76.3) | 1576 (64.5) | 3502 (70.5) |
| Grade 3 or worse TEAE | 1579 (62.6) | 436 (17.8) | 2015 (40.6) |
| TEAE of special interest | 2160 (85.6) | 1140 (46.6) | 3300 (66.4) |

Note:
Only one occurrence of AE is counted per patient.

TABLE B5.1-2
Serious Treatment-Emergent Adverse Events, Irrespective of Causality,
By Preferred Term and Worst Toxicity Grade (≥0.5% in each arm)

| | ET + ribociclib | | | | | | | | ET only | | | | | |
|---|---|---|---|---|---|---|---|---|---|---|---|---|---|---|
| | N = 2524 | | | | | | | | N = 2444 | | | | | |
| | All | | | | | | | | All | | | | | |
| | Grades | | Grade 3 | | Grade 4 | | Grade 5 | | Grades | | Grade 3 | | Grade 4 | Grade 5 |
| Preferred Term | n (%) | | n (%) | | n (%) | | n (%) | | n (%) | | n (%) | | n (%) | n (%) |
| Number of patients with | 336 | (13.3) | 232 | (9.2) | 44 | (1.7) | 12 | (0.5) | 242 | (9.9) | 180 | (7.4) | 25 (1.0) | 4 (0.2) |
| at least one TEAE | | | | | | | | | | | | | | |
| COVID-19 | 20 | (0.8) | 13 | (0.5) | 0 | | 3 | (0.1) | 12 | (0.5) | 8 | (0.3) | 0 | 1 (0.0) |
| Pneumonia | 15 | (0.6) | 12 | (0.5) | 0 | | 1 | (0.0) | 7 | (0.3) | 6 | (0.2) | 0 | 0 |
| Pulmonary embolism | 13 | (0.5) | 11 | (0.4) | 1 | (0.0) | 1 | (0.0) | 2 | (0.1) | 2 | (0.1) | 0 | 0 |

| | | | | | | | | | | | | |
|---|---|---|---|---|---|---|---|---|---|---|---|---|
| Dyspnoea | 13 | (0.5) | 10 | (0.4) | 0 | 0 | 5 | (0.2) | 3 | (0.1) | 0 | 0 |

Preferred terms are sorted in descending frequency based on frequency in ET + ribociclib arm.
MedDRA Version 25.1 has been used for reporting.

TABLE B5.1-3
Treatment-Emergent Adverse Events Leading to Treatment Discontinuation by System
Organ Class and Preferred Term and Worst Toxicity Grade (>0.5% in any arm)

| | ET + ribociclib | | | | | | | ET only | | | | | |
|---|---|---|---|---|---|---|---|---|---|---|---|---|---|
| | N = 2524 | | | | | | | N = 2444 | | | | | |
| | All | | | | | | | All | | | | | |
| | Grades | | Grade 3 | | Grade 4 | | Grade 5 | Grades | | Grade 3 | | Grade 4 | Grade 5 |
| Preferred Term | n (%) | | n (%) | | n (%) | | n (%) | n (%) | | n (%) | | n (%) | n (%) |
| Number of patients with | 523 | (20.7) | 202 | (8.0) | 35 | (1.4) | 6 (0.2) | 129 | (5.3) | 35 | (1.4) | 5 (0.2) | 3 (0.1) |
| at least one TEAE | | | | | | | | | | | | | |
| Alanine aminotransferase | 176 | (7.0) | 77 | (3.1) | 19 | (0.8) | 0 | 2 | (0.1) | 1 | (0.0) | 0 | 0 |
| increased | | | | | | | | | | | | | |
| Aspartate aminotransferase | 71 | (2.8) | 29 | (1.1) | 7 | (0.3) | 0 | 0 | | 0 | | 0 | 0 |
| increased | | | | | | | | | | | | | |

| | | | | | | | | | | | | |
|---|---|---|---|---|---|---|---|---|---|---|---|---|
| Arthralgia | 34 | (1.3) | 4 | (0.2) | 0 | 0 | 46 | (1.9) | 9 | (0.4) | 0 | 0 |

| | | | | | | | | | | | |
|---|---|---|---|---|---|---|---|---|---|---|---|
| Fatigue | 24 | (1.0) | 5 | (0.2) | 0 | 0 | 2 | (0.1) | 0 | 0 | 0 |

| | | | | | | | | | | | |
|---|---|---|---|---|---|---|---|---|---|---|---|
| Neutropenia | 16 | (0.6) | 13 | (0.5) | 2 | (0.1) | 0 | 0 | 0 | 0 | 0 |

| Neutrophil count decreased | 14 | (0.6) | 12 | (0.5) | 0 | 0 | 0 | | 0 | 0 | 0 |
|---|---|---|---|---|---|---|---|---|---|---|---|
| Nausea | 13 | (0.5) | 1 | (0.0) | 0 | 0 | 2 | (0.1) | 0 | 0 | 0 |

Preferred terms are sorted in descending frequency based on frequency in ET + ribociclib arm.
MedDRA Version 25.1 has been used for reporting.

TABLE B5.1-4
Treatment-Emergent Adverse Events Leading to Ribociclib Discontinuation
Irrespective of Causality, By Preferred Term and Worst Toxicity Grade
(>0.5% in any arm)

| | ET + Ribociclib | | | |
|---|---|---|---|---|
| | N = 2524 | | | |
| Preferred Term | All Grades | Grade 3 | Grade 4 | Grade 5 |
| Number of patients with at least one TEAE | 495 (19.6%) | 189 (7.49%) | 33 (1.31%) | 5 (0.20%) |
| Alanine aminotransferase increased | 175 (6.93%) | 77 (3.05%) | 17 (0.67%) | 0 (0.00%) |
| Aspartate aminotransferase increased | 65 (2.61%) | 27 (1.07%) | 5 (0.20%) | 0 (0.00%) |
| Fatigue | 20 (0.79%) | 5 (0.20%) | 0 (0.00%) | 0 (0.00%) |
| Neutropenia | 16 (0.53%) | 13 (0.52%) | 2 (8.08%) | 0 (0.00%) |
| Neutrophil count decreased | 13 (0.52%) | 11 (0.44%) | 0 (0.00%) | 0 (0.00%) |
| Arthralgia | 13 (0.52%) | 0 (0.00%) | 0 (0.00%) | 0 (0.00%) |

TABLE B5.1-5
Treatment-Emergent Adverse Events of Special Interest by grouping and maximum grade (Safety set) (>1% in any arm)

| | ET + ribociclib | | | | | | | ET only | | | | | |
|---|---|---|---|---|---|---|---|---|---|---|---|---|---|
| | N = 2524 | | | | | | | N = 2444 | | | | | |
| | All | | | | | | | All | | | | | |
| AESI Grouping | grades | | Grade 3 | | Grade 4 | | Grade 5 | Grades | | Grade 3 | | Grade 4 | Grade 5 |
| Preferred term | n (%) | | n (%) | | n (%) | | n (%) | n (%) | | n (%) | | n (%) | n (%) |
| Hepatobiliary toxicity | 641 | (25.4) | 166 | (6.6) | 43 | (1.7) | 0 (0.0) | 260 | (10.6) | 36 | (1.5) | 1 (0.0) | 0 (0.0) |
| Alanine aminotransferase | 478 | (18.9) | 154 | (6.1) | 31 | (1.2) | 0 (0.0) | 128 | (5.2) | 15 | (0.6) | 1 (0.0) | 0 (0.0) |
| increased | | | | | | | | | | | | | |
| Aspartate aminotransferase | 408 | (16.2) | 96 | (3.8) | 16 | (0.6) | 0 (0.0) | 131 | (5.4) | 12 | (0.5) | 0 (0.0) | 0 (0.0) |
| increased | | | | | | | | | | | | | |
| Gamma-glutamyltransferase | 114 | (4.5) | 24 | (1.0) | 3 | (0.1) | 0 (0.0) | 63 | (2.6) | 20 | (0.8) | 0 (0.0) | 0 (0.0) |
| increased | | | | | | | | | | | | | |
| Blood alkaline phosphatase | 75 | (3.0) | 3 | (0.1) | 0 | (0.0) | 0 (0.0) | 60 | (2.5) | 2 | (0.1) | 0 (0.0) | 0 (0.0) |
| increased | | | | | | | | | | | | | |
| Blood bilirubin increased | 66 | (2.6) | 4 | (0.2) | 1 | (0.0) | 0 (0.0) | 27 | (1.1) | 1 | (0.0) | 0 (0.0) | 0 (0.0) |
| ILD_Pneumonitis | 39 | (1.5) | 0 | (0.0) | 0 | (0.0) | 0 (0.0) | 19 | (0.8) | 2 | (0.1) | 0 (0.0) | 0 (0.0) |
| Infections | 1199 | (47.5) | 115 | (4.6) | 9 | (0.4) | 7 (0.3) | 836 | (34.2) | 67 | (2.7) | 3 (0.1) | 2 (0.1) |
| COVID-19 | 477 | (18.9) | 18 | (0.7) | 0 | (0.0) | 3 (0.1) | 314 | (12.8) | 11 | (0.5) | 0 (0.0) | 1 (0.0) |
| Urinary tract infection | 155 | (6.1) | 9 | (0.4) | 0 | (0.0) | 0 (0.0) | 121 | (5.0) | 5 | (0.2) | 0 (0.0) | 0 (0.0) |
| Nasopharyngitis | 121 | (4.8) | 0 | (0.0) | 0 | (0.0) | 0 (0.0) | 84 | (3.4) | 0 | (0.0) | 0 (0.0) | 0 (0.0) |
| Upper respiratory tract | 118 | (4.7) | 2 | (0.1) | 0 | (0.0) | 0 (0.0) | 57 | (2.3) | 0 | (0.0) | 0 (0.0) | 0 (0.0) |
| infection | | | | | | | | | | | | | |
| Sinusitis | 62 | (2.5) | 0 | (0.0) | 0 | (0.0) | 0 (0.0) | 38 | (1.6) | 0 | (0.0) | 0 (0.0) | 0 (0.0) |
| Herpes zoster | 58 | (2.3) | 3 | (0.1) | 0 | (0.0) | 0 (0.0) | 51 | (2.1) | 2 | (0.1) | 0 (0.0) | 0 (0.0) |
| Suspected COVID-19 | 40 | (1.6) | 1 | (0.0) | 0 | (0.0) | 0 (0.0) | 21 | (0.9) | 1 | (0.0) | 0 (0.0) | 0 (0.0) |
| Pneumonia | 36 | (1.4) | 12 | (0.5) | 0 | (0.0) | 1 (0.0) | 18 | (0.7) | 7 | (0.3) | 0 (0.0) | 0 (0.0) |
| Cystitis | 34 | (1.3) | 0 | (0.0) | 0 | (0.0) | 0 (0.0) | 24 | (1.0) | 0 | (0.0) | 0 (0.0) | 0 (0.0) |
| Oral herpes | 34 | (1.3) | 0 | (0.0) | 0 | (0.0) | 0 (0.0) | 12 | (0.5) | 0 | (0.0) | 0 (0.0) | 0 (0.0) |
| Cellulitis | 33 | (1.3) | 12 | (0.5) | 0 | (0.0) | 0 (0.0) | 17 | (0.7) | 8 | (0.3) | 0 (0.0) | 0 (0.0) |
| Influenza | 30 | (1.2) | 2 | (0.1) | 0 | (0.0) | 0 (0.0) | 20 | (0.8) | 0 | (0.0) | 0 (0.0) | 0 (0.0) |
| Tooth infection | 30 | (1.2) | 2 | (0.1) | 0 | (0.0) | 0 (0.0) | 20 | (0.8) | 0 | (0.0) | 0 (0.0) | 0 (0.0) |
| Bronchitis | 29 | (1.1) | 1 | (0.0) | 0 | (0.0) | 0 (0.0) | 29 | (1.2) | 0 | (0.0) | 0 (0.0) | 0 (0.0) |
| Gastroenteritis | 29 | (1.1) | 1 | (0.0) | 0 | (0.0) | 0 (0.0) | 17 | (0.7) | 0 | (0.0) | 0 (0.0) | 0 (0.0) |
| Respiratory tract infection viral | 29 | (1.1) | 1 | (0.0) | 0 | (0.0) | 0 (0.0) | 27 | (1.1) | 0 | (0.0) | 0 (0.0) | 0 (0.0) |
| Conjunctivitis | 28 | (1.1) | 0 | (0.0) | 0 | (0.0) | 0 (0.0) | 12 | (0.5) | 0 | (0.0) | 0 (0.0) | 0 (0.0) |
| Mastitis | 27 | (1.1) | 7 | (0.3) | 0 | (0.0) | 0 (0.0) | 17 | (0.7) | 4 | (0.2) | 0 (0.0) | 0 (0.0) |
| Myelosuppression - Anaemia | 209 | (8.3) | 7 | (0.3) | 0 | (0.0) | 0 (0.0) | 74 | (3.0) | 5 | (0.2) | 1 (0.0) | 0 (0.0) |
| Anaemia | 203 | (8.0) | 7 | (0.3) | 0 | (0.0) | 0 (0.0) | 70 | (2.9) | 5 | (0.2) | 1 (0.0) | 0 (0.0) |
| Myelosuppression - | 583 | (23.1) | 193 | (7.6) | 4 | (0.2) | 0 (0.0) | 110 | (4.5) | 8 | (0.3) | 1 (0.0) | 0 (0.0) |
| Leukopenia | | | | | | | | | | | | | |
| Leukopenia | 329 | (13.0) | 90 | (3.6) | 0 | (0.0) | 0 (0.0) | 49 | (2.0) | 2 | (0.1) | 0 (0.0) | 0 (0.0) |
| White blood cell count | 243 | (9.6) | 91 | (3.6) | 1 | (0.0) | 0 (0.0) | 37 | (1.5) | 5 | (0.2) | 1 (0.0) | 0 (0.0) |
| decreased | | | | | | | | | | | | | |
| Lymphopenia | 65 | (2.6) | 11 | (0.4) | 1 | (0.0) | 0 (0.0) | 16 | (0.7) | 0 | (0.0) | 0 (0.0) | 0 (0.0) |
| Lymphocyte count decreased | 59 | (2.3) | 17 | (0.7) | 2 | (0.1) | 0 (0.0) | 25 | (1.0) | 1 | (0.0) | 0 (0.0) | 0 (0.0) |
| Myelosuppression - | 1568 | (62.1) | 1054 | (41.8) | 52 | (2.1) | 0 (0.0) | 110 | (4.5) | 17 | (0.7) | 3 (0.1) | 0 (0.0) |
| Neutropenia | | | | | | | | | | | | | |
| Neutropenia | 1034 | (41.0) | 665 | (26.3) | 33 | (1.3) | 0 (0.0) | 71 | (2.9) | 12 | (0.5) | 1 (0.0) | 0 (0.0) |
| Neutrophil count decreased | 606 | (24.0) | 421 | (16.7) | 19 | (0.8) | 0 (0.0) | 40 | (1.6) | 5 | (0.2) | 2 (0.1) | 0 (0.0) |
| Myelosuppression - | 161 | (6.4) | 6 | (0.2) | 0 | (0.0) | 0 (0.0) | 53 | (2.2) | 2 | (0.1) | 1 (0.0) | 0 (0.0) |
| Thrombocytopenia | | | | | | | | | | | | | |
| Thrombocytopenia | 110 | (4.4) | 5 | (0.2) | 0 | (0.0) | 0 (0.0) | 43 | (1.8) | 0 | (0.0) | 1 (0.0) | 0 (0.0) |
| Platelet count decreased | 52 | (2.1) | 1 | (0.0) | 0 | (0.0) | 0 (0.0) | 10 | (0.4) | 2 | (0.1) | 0 (0.0) | 0 (0.0) |
| QT interval prolongation | 132 | (5.2) | 23 | (0.9) | 1 | (0.0) | 1 (0.0) | 30 | (1.2) | 13 | (0.5) | 0 (0.0) | 0 (0.0) |
| Electrocardiogram QT | 106 | (4.2) | 5 | (0.2) | 0 | (0.0) | 0 (0.0) | 16 | (0.7) | 1 | (0.0) | 0 (0.0) | 0 (0.0) |
| prolonged | | | | | | | | | | | | | |
| Renal toxicity | 144 | (5.7) | 6 | (0.2) | 1 | (0.0) | 0 (0.0) | 49 | (2.0) | 0 | (0.0) | 0 (0.0) | 0 (0.0) |
| Blood creatinine increased | 91 | (3.6) | 2 | (0.1) | 0 | (0.0) | 0 (0.0) | 21 | (0.9) | 0 | (0.0) | 0 (0.0) | 0 (0.0) |
| Glomerular filtration rate | | | | | | | | | | | | | |
| decreased | 40 | (1.6) | 2 | (0.1) | 0 | (0.0) | 0 (0.0) | 9 | (0.4) | 0 | (0.0) | 0 (0.0) | 0 (0.0) |
| Blood urea increased | 26 | (1.0) | 0 | (0.0) | 0 | (0.0) | 0 (0.0) | 24 | (1.0) | 0 | (0.0) | 0 (0.0) | 0 (0.0) |
| Reproductive toxicity | 32 | (1.3) | 8 | (0.3) | 0 | (0.0) | 0 (0.0) | 25 | (1.0) | 5 | (0.2) | 0 (0.0) | 0 (0.0) |
| Mastitis | 27 | (1.1) | 7 | (0.3) | 0 | (0.0) | 0 (0.0) | 17 | (0.7) | 4 | (0.2) | 0 (0.0) | 0 (0.0) |
| Second Primary Malignancies | 46 | (1.8) | 25 | (1.0) | 3 | (0.1) | 0 (0.0) | 45 | (1.8) | 20 | (0.8) | 8 (0.3) | 0 (0.0) |

AESI categories are presented in alphabetical order; preferred terms are sorted within AESI category by descending frequency on the ET + ribociclib arm.
AESIs may fall under more than one category but are only reported once per AESI category.
MedDRA Version 25.1 has been used for reporting.
AESI groupings per eCRS Version dated 16 Dec. 2022.

TABLE B5.1-7

| | Deaths Overall Summary (Safety set) | | |
|---|---|---|---|
| | ET + ribociclib | | |
| Number of patients | N = 2524 | N = 2444 | N = 4968 |
| Number of patients who died overall | 60 (2.4) | 74 (3.0) | 134 (2.7) |
| Primary reason of death | | | |
| Disease Recurrence/Progression | 41 (1.6) | 61 (2.5) | 102 (2.1) |
| Adverse Event | 13 (0.5) | 4 (0.2) | 17 (0.3) |
| Covid-19 | 3 (0.1) | 1 (0.0) | 4 (0.1) |
| Covid-19 Pneumonia | 3 (0.1) | 0 | 3 (0.1) |
| Acute Myocardial Infarction | 1 (0.0) | 0 | 1 (0.0) |
| Brain Oedema | 1 (0.0) | 0 | 1 (0.0) |
| Cardiac Arrest | 1 (0.0) | 0 | 1 (0.0) |
| Cardiogenic Shock | 1 (0.0) | 0 | 1 (0.0) |
| Pneumonia | 1 (0.0) | 0 | 1 (0.0) |
| Pulmonary Embolism | 1 (0.0) | 0 | 1 (0.0) |
| Road Traffic Accident | 1 (0.0) | 0 | 1 (0.0) |
| Cardiac Failure Congestive | 0 | 1 (0.0) | 1 (0.0) |
| Myocardial Infarction | 0 | 1 (0.0) | 1 (0.0) |
| Sepsis | 0 | 1 (0.0) | 1 (0.0) |
| Other | 6 (0.2) | 9 (0.4) | 15 (0.3) |

6 patients died due to COVID 19 were not vaccinated. 1 patient in ET + ribociclib arm was vaccinated.

TABLE B5.1-8
Deaths on Treatment Summary (Safety set)

| | ET + ribociclib | ET only | Total |
|---|---|---|---|
| Number of patients | N = 2524 | N = 2444 | N = 4968 |
| Number of patients who | 19 (0.8) | 9 (0.4) | 28 (0.6) |
| died on treatment | | | |
| Primary reason of death | | | |
| Disease Recurrence/ | 7 (0.3) | 4 (0.2) | 11 (0.2) |
| Progression | | | |
| Adverse Event | 12 (0.5) | 4 (0.2) | 16 (0.3) |
| Covid-19 | 3 (0.1) | 1 (0.0) | 4 (0.1) |
| Covid-19 Pneumonia | 3 (0.1) | 0 | 3 (0.1) |
| Brain Oedema | 1 (0.0) | 0 | 1 (0.0) |
| Cardiac Arrest | 1 (0.0) | 0 | 1 (0.0) |
| Cardiogenic Shock | 1 (0.0) | 0 | 1 (0.0) |
| Pneumonia | 1 (0.0) | 0 | 1 (0.0) |
| Pulmonary Embolism | 1 (0.0) | 0 | 1 (0.0) |
| Road Traffic Accident | 1 (0.0) | 0 | 1 (0.0) |
| Cardiac Failure Congestive | 0 | 1 (0.0) | 1 (0.0) |
| Myocardial Infarction | 0 | 1 (0.0) | 1 (0.0) |
| Sepsis | 0 | 1 (0.0) | 1 (0.0) |
| Other | 0 | 1 (0.0) | 1 (0.0) |

6 patients died due to COVID 19 were not vaccinated. 1 patient in ET + ribociclib arm was vaccinated.

### 5.2 Laboratory Data

TABLE B5.2-1
(r) Worst On-Treatment CTCAE Grade for Hematology Laboratory Data (Safety set)

| | ET + ribociclib | | | | | | ET only | | | | | |
|---|---|---|---|---|---|---|---|---|---|---|---|---|
| | N = 2524 | | | | | | N = 2444 | | | | | |
| | Grade 1/2 | | Grade 3 | | Grade 4 | | Grade 1/2 | | Grade 3 | | Grade 4 | |
| Parameter | n (%) | | n (%) | | n (%) | | n (%) | | n (%) | | n (%) | |
| Activated Partial Thromboplastin | 9 | (0.4) | 0 | | 0 | | 8 | (0.3) | 0 | | 0 | |
| Time (sec) Hyper Grade | | | | | | | | | | | | |
| Hemoglobin (g/L) Hyper Grade | 90 | (3.6) | 14 | (0.6) | 0 | | 152 | (6.2) | 18 | (0.7) | 0 | |
| Hemoglobin (g/dL) Hypo Grade | 1156 | (45.8) | 11 | (0.4) | 0 | | 611 | (25.0) | 6 | (0.3) | 0 | |
| Leukocytes (10E9/L) Hyper Grade | 0 | | 49 | (1.9) | 0 | | 0 | | 32 | (1.3) | 0 | |
| Leukocytes (10E9/L) Hypo Grade | 1718 | (68.1) | 678 | (26.9) | 9 | (0.4) | 1091 | (44.6) | 13 | (0.5) | 3 | (0.1) |
| Lymphocytes (10E9/L) Hyper | 33 | (1.3) | 74 | (2.9) | 0 | | 56 | (2.3) | 62 | (2.5) | 0 | |
| Grade | | | | | | | | | | | | |
| Lymphocytes (10E9/L) Hypo | 1967 | (77.9) | 404 | (16.0) | 84 | (3.3) | 1990 | (81.4) | 95 | (3.9) | 65 | (2.7) |
| Grade | | | | | | | | | | | | |
| Neutrophils (10E9/L) Hypo Grade | 1235 | (48.9) | 1071 | (42.4) | 55 | (2.2) | 807 | (33.0) | 34 | (1.4) | 7 | (0.3) |
| Platelets (10E9/L) Hypo Grade | 696 | (27.6) | 9 | (0.4) | 2 | (0.1) | 307 | (12.6) | 7 | (0.3) | 1 | (0.0) |
| Prothrombin Intl. Normalized Ratio | 5 | (0.2) | 1 | (0.0) | 0 | | 3 | (0.1) | 2 | (0.1) | 0 | |
| (sec) Hyper Grade | | | | | | | | | | | | |

Baseline is defined as the last non-missing value prior to the start date of study treatment.
Percentages are based on N.
Patients are counted only for the worst grade observed post-baseline.
Laboratory assessments performed more than 30 days after last study treatment administration date are not summarized.
CTCAE Version 4.03 is used for reporting.

TABLE B5.2-2
(s) Worst On-Treatment CTCAE Grade for Biochemistry Laboratory Data (Safety set)

| | ET + ribociclib | | | | | | ET only | | | | | |
|---|---|---|---|---|---|---|---|---|---|---|---|---|
| | N = 2524 | | | | | | N = 2444 | | | | | |
| | Grade 1/2 | | Grade 3 | | Grade 4 | | Grade 1/2 | | Grade 3 | | Grade 4 | |
| Parameter | n (%) | | n (%) | | n (%) | | n (%) | | n (%) | | n (%) | |
| Alanine Aminotransferase (IU/L) | 900 | (35.7) | 161 | (6.4) | 37 | (1.5) | 817 | (33.4) | 23 | (0.9) | 1 | (0.0) |
| Hyper Grade | | | | | | | | | | | | |
| Albumin (g/dL) Hypo Grade | 178 | (7.1) | 14 | (0.6) | 0 | | 138 | (5.7) | 8 | (0.3) | 0 | |
| Alkaline Phosphatase (IU/L) | 882 | (34.9) | 4 | (0.2) | 0 | | 875 | (35.8) | 5 | (0.2) | 0 | |
| Hyper Grade | | | | | | | | | | | | |
| Amylase (IU/L) Hyper Grade | 346 | (13.7) | 24 | (1.0) | 8 | (0.3) | 326 | (13.3) | 32 | (1.3) | 2 | (0.1) |
| Aspartate Aminotransferase | 952 | (37.7) | 108 | (4.3) | 19 | (0.8) | 761 | (31.1) | 26 | (1.1) | 1 | (0.0) |
| (IU/L) Hyper Grade | | | | | | | | | | | | |
| Bilirubin (umol/L) Hyper Grade | 226 | (9.0) | 7 | (0.3) | 2 | (0.1) | 231 | (9.5) | 1 | (0.0) | 0 | |
| Calcium Corrected (mmol/L) | 244 | (9.7) | 5 | (0.2) | 11 | (0.4) | 338 | (13.8) | 9 | (0.4) | 21 | (0.9) |
| Hyper Grade | | | | | | | | | | | | |
| Calcium Corrected (mmol/L) | 494 | (19.6) | 6 | (0.2) | 32 | (1.3) | 369 | (15.1) | 8 | (0.3) | 39 | (1.6) |
| Hypo Grade | | | | | | | | | | | | |
| Creatinine (mg/dL) Hyper Grade | 797 | (31.6) | 9 | (0.4) | 2 | (0.1) | 267 | (10.9) | 0 | | 1 | (0.0) |
| Direct Bilirubin (umol/L) Hyper | 442 | (17.5) | 27 | (1.1) | 11 | (0.4) | 420 | (17.2) | 16 | (0.7) | 4 | (0.2) |
| Grade | | | | | | | | | | | | |
| Gamma Glutamyl Transferase | 857 | (34.0) | 85 | (3.4) | 8 | (0.3) | 748 | (30.6) | 82 | (3.4) | 5 | (0.2) |
| (IU/L) Hyper Grade | | | | | | | | | | | | |
| Glomerular Filtration Rate | 1038 | (41.1) | 14 | (0.6) | 8 | (0.3) | 651 | (26.6) | 4 | (0.2) | 5 | (0.2) |
| (mL/min/1.73m2) Hypo Grade | | | | | | | | | | | | |
| Glucose (mmol/L) Hyper Grade | 1452 | (57.5) | 45 | (1.8) | 27 | (1.1) | 1381 | (56.5) | 34 | (1.4) | 23 | (0.9) |
| Glucose (mmol/L) Hypo Grade | 219 | (8.7) | 3 | (0.1) | 21 | (0.8) | 163 | (6.7) | 1 | (0.0) | 22 | (0.9) |
| Lipase (IU/L) Hyper Grade | 385 | (15.3) | 62 | (2.5) | 12 | (0.5) | 384 | (15.7) | 66 | (2.7) | 11 | (0.5) |
| Magnesium (mmol/L) Hyper | 83 | (3.3) | 28 | (1.1) | 1 | (0.0) | 100 | (4.1) | 26 | (1.1) | 2 | (0.1) |
| Grade | | | | | | | | | | | | |
| Magnesium (mmol/L) Hypo | 380 | (15.1) | 4 | (0.2) | 5 | (0.2) | 368 | (15.1) | 0 | | 11 | (0.5) |
| Grade | | | | | | | | | | | | |
| Phosphate (mmol/L) Hypo Grade | 190 | (7.5) | 12 | (0.5) | 6 | (0.2) | 162 | (6.6) | 11 | (0.5) | 2 | (0.1) |
| Potassium (mmol/L) Hyper Grade | 346 | (13.7) | 15 | (0.6) | 2 | (0.1) | 372 | (15.2) | 14 | (0.6) | 6 | (0.3) |
| Potassium (mmol/L) Hypo Grade | 257 | (10.2) | 14 | (0.6) | 8 | (0.3) | 201 | (8.2) | 21 | (0.9) | 14 | (0.6) |
| Sodium (mmol/L) Hyper Grade | 264 | (10.5) | 3 | (0.1) | 1 | (0.0) | 281 | (11.5) | 2 | (0.1) | 0 | |
| Sodium (mmol/L) Hypo Grade | 258 | (10.2) | 24 | (1.0) | 7 | (0.3) | 220 | (9.0) | 16 | (0.7) | 10 | (0.4) |

| | | | | | | | | | | |
|---|---|---|---|---|---|---|---|---|---|---|
| Urate (mg/dL) Hyper Grade | 756 | (30.0) | 0 | 42 | (1.7) | 693 | (28.4) | 0 | 49 | (2.0) |

Baseline is defined as the last non-missing value prior to the start date of study treatment.
Percentages are based on N.
Patients are counted only for the worst grade observed post-baseline.
Laboratory assessments performed more than 30 days after last study treatment administration date are not summarized.
CTCAE Version 4.03 is used for reporting.

TABLE B5.2-3
Liver Function Parameters (Safety set)

| | Ribociclib + ET | ET Only |
|---|---|---|
| | N = 2524 | N = 2444 |
| | n/m (%) | n/m (%) |
| ALT or AST > 3*ULN and TBL > 2*ULN | 14/2504 ( 0.6%) | 1/2386 (0.0%) |
| Biochemistry Hy's law: ALT or AST > | 8/2496 (0.3%) | 1/2378 (0.0%) |
| 3*ULN and TBL > 2*ULN and ALP < | | |
| 2*ULN | | |

Biochemistry Hy's law are based on concurrent occurrence of the individual abnormalities at the same study visit
ALP: Alkaline phosphatase
ALT: Alanine aminotransferase
AST: Aspartate aminotransferase
TBL: Total bilirubin
ULN: Upper limit of normal
Based on worst on-treatment values per Novartis Liver Toxicity guidelines.
n: Number of subjects who meet the designated criterion.
m: Number of subjects at risk for a specific category. This is the number of patients with a non-missing value at baseline and at least one non-missing post-baseline value.
N: Total number of patients in the treatment group in this analysis set.

### 5.3 Notable ECG Values

TABLE B5.3-1
(u) Notable ECG Values-Central Assessments (Safety set)

| | ET + ribociclib | ET only | Total |
|---|---|---|---|
| | N = 2524 | N = 2444 | N = 4968 |
| ECG Value | n/m (%) | n/m (%) | n/m (%) |
| QTcF interval | | | |
| New value > 450 | 238/2489 (9.6) | 66/2367 (2.8) | 304/4856 (6.3) |
| New value > 480 | 10/2505 (0.4) | 4/2380 (0.2) | 14/4885 (0.3) |
| New value > 500 | 3/2505 (0.1) | 1/2380 (0.0) | 4/4885 (0.1) |
| Increase from | 476/2505 (19.0) | 179/2380 (7.5) | 655/4885 (13.4) |
| baseline of > 30 | | | |
| Increase from | 19/2505 (0.8) | 2/2380 (0.1) | 21/4885 (0.4) |
| baseline of > 60 | | | |

Patients are counted based on any notable ECG post-baseline value.
ECG assessments based on central laboratory results only.
Baseline is defined as the last assessment on or before start of study treatment. For any replicate/triplicate ECGs per time point, the average of these measurements would be calculated for baseline.
n: Number of subjects who meet the designated criterion.
m: Number of subjects at risk for a specific category. For new abnormality post-baseline, this is the number of patients with both baseline and post-baseline evaluations, and baseline not meeting the criteria. For abnormal change from baseline, it is the number of patients with both baseline and post-baseline evaluations.
N: Total number of subjects in the treatment group in this analysis set.

### APPENDIX

TABLE B14

| | Total |
|---|---|
| | N = 5101 |
| | n (%) |
| Duration of follow-up | |
| 0-3 months | 308 (6.0) |
| 3-6 months | 86 (1.7) |
| 6-12 months | 114 (2.2) |
| 12-18 months | 78 (1.5) |
| 18-24 months | 705 (13.8) |
| 24-30 months | 1083 (21.2) |
| 30-36 months | 1401 (27.5) |
| 36-42 months | 1215 (23.8) |
| 42-48 months | 110 (2.2) |
| 48-54 months | |
| Duration of follow-up | |
| n | 5101 |
| Mean | 29.0 |
| SD | 10.57 |
| Min | 0 |
| Median | 30.6 |
| Max | 48 |
| Randomization (recruitment) period | 27.33 |
| Duration between randomization and | |
| data cut-off date | |
| n | 5101 |
| Mean | 32.6 |
| SD | 5.97 |
| Min | 21 |
| Median | 34.0 |
| Max | 48 |

Duration of follow-up is calculated as {min(Cut-off date, death date, date of dropout from study) − Date of randomization + 1}/30.4375 (months).
Randomization (recruitment) period = (Date of last patient randomized − Date of first patient randomized + 1)/30.4375 (months).
Duration between randomization and data cut-off date = (Cut-off date − Date of randomization + 1)/30.4375 (months).

TABLE B14.2-1.2
(w) Duration of Follow-up for iDFS (Full analysis set)

| | Total |
|---|---|
| | N = 5101 |
| | n (%) |
| Duration of follow-up | |
| 0-3 months | 416 (8.2) |
| 3-6 months | 95 (1.9) |
| 6-12 months | 150 (2.9) |
| 12-18 months | 176 (3.5) |
| 18-24 months | 915 (17.9) |
| 24-30 months | 1171 (23.0) |
| 30-36 months | 1581 (31.0) |
| 36-42 months | 572 (11.2) |
| 42-48 months | 25 (0.5) |
| Duration of follow-up | |
| n | 5101 |
| Mean | 25.97 |
| SD | 10.715 |
| Min | 0.0 |
| Median | 27.70 |
| Max | 45.1 |

Duration of follow-up is calculated as (Date of event or last adequate recurrence assessment date − randomization date + 1)/30.4375 (months).

TABLE B14.2-1.3
(x) Duration of Follow-up for OS (Full analysis set)

| | Treatment A | Treatment B | Total |
|---|---|---|---|
| | N = 2549 | N = 2552 | N = 5101 |
| Duration of Follow-up | | | |
| 0-3 months | [symbol] | [symbol] | [symbol] |
| 3-5 months | [symbol] | [symbol] | [symbol] |
| 6-12 months | [symbol] | [symbol] | [symbol] |
| 12-18 months | [symbol] | [symbol] | [symbol] |
| 18-24 months | [symbol] | [symbol] | [symbol] |
| 24-30 months | [symbol] | [symbol] | [symbol] |
| 35-36 months | [symbol] | [symbol] | [symbol] |
| 36-52 months | [symbol] | [symbol] | [symbol] |
| 42-48 months | [symbol] | [symbol] | [symbol] |
| Duration of follow-up | | | |
| n | 2549 | 2552 | 2501 |
| Mean | 25.7 | 27.2 | 27.9 |
| SD | 3.45 | [symbol] | 10.14 |
| Min | 0 | 0 | 0 |
| Median | 30.4 | [symbol] | 30.4 |
| Max | 47 | 45 | 47 |

Duration of follow-up is calculated as (Date of event or last contact date − randomization date + 1}/ [symbol] months) . [symbol]
indicates data missing or illegible when filed

1.-6. (canceled)

7. A method of maintaining remission in an adult patient who had previously been diagnosed with HR+/HER2−stage II or stage III early breast cancer, comprising administering to the patient a treatment comprising a dose of ribociclib or a pharmaceutically acceptable salt thereof, in combination with an aromatase inhibitor.

8.-18. (canceled)

19. The method of claim 7, wherein the dose of ribociclib is 200 mg/day or 400 mg/day.

20.-21. (canceled)

22. The method of claim 19, wherein ribociclib is administered as ribociclib succinate, and the total dose of ribociclib is 400 mg/day.

23.-97. (canceled)

98. The method of claim 22, wherein the treatment reduces the risk of invasive disease.

99. (canceled)

100. The method of claim 98, wherein the treatment reduces the risk of invasive disease corresponding to a hazard ratio of less than or equal to 0.78 when the risk is calculated relative to patients that received the treatment except for administration of ribociclib.

101.-117. (canceled)

118. A method of treating HR+/HER2− stage II or stage III early breast cancer in an adult patient, comprising providing adjuvant treatment by administering ribociclib in combination with an aromatase inhibitor, wherein the patient at start of the adjuvant treatment is in complete remission, and the administration of ribociclib in combination with aromatase inhibitor maintains the adult patient in complete remission for at least 3 months.

119.-129. (canceled)

130. The method of claim 118, wherein ribociclib and aromatase inhibitor are administered for a treatment duration, and the administration maintains the adult patient in complete remission for at least the treatment duration.

131. The method of claim 118, wherein a dose of 400 mg ribociclib is orally administered once daily on days 1-21 of a 28-day cycle and aromatase inhibitor is administered once daily on each day of the 28-day cycle.

132. The method of claim 118, wherein a dose of 400 mg ribociclib is orally administered once daily on days 1-21 of a 28-day cycle for a treatment duration of up to three years and aromatase inhibitor is administered once daily on each day of the 28-day cycle for the up to three years.

133.-206. (canceled)

207. The method of claim 118, wherein the treatment reduces the risk of invasive disease in the adult patient corresponding to a hazard ratio of less than or equal to 0.78 when the risk is calculated relative to other patients having received the same treatment as the adult patient except that the other patients did not receive the ribociclib.

208-372. (canceled)
